(19) United States
(12) Patent Application Publication
(10) Pub. No.: US 2025/0099576 A1
(43) Pub. Date: Mar. 27, 2025

(54) Recombinant RNA Molecules Comprising Untranslated Regions or Segments Encoding Spike Protein from the Omicron Strain of Severe Acute Respiratory Coronavirus-2

(71) Applicant: GlaxoSmithKline Biologicals SA, Rixensart (BE)

(72) Inventors: Giulietta MARUGGI, Rockville, MD (US); Kambiz MOUSAVI, Rockville, MD (US); Newton WAHOME, Rockville, MD (US); Jason William WESTERBECK, Rockville, MD (US); Magdalena Aleksandra ZWIERZYNA, Siena (IT); Yoo-Ah KIM, Rockville, MD (US); Amirali YAZDI, Wavre (BE); Yamina BENNASSER, Rockville, MD (US)

(73) Assignee: GlaxoSmithKline Biologicals SA, Rixensart (BE)

(21) Appl. No.: 18/964,896

(22) Filed: Dec. 2, 2024

Related U.S. Application Data
(63) Continuation of application No. PCT/IB2023/056264, filed on Jun. 16, 2023.
(60) Provisional application No. 63/353,568, filed on Jun. 18, 2022.
(60) Provisional application No. 63/413,844, filed on Oct. 6, 2022.
(60) Provisional application No. 63/380,640, filed on Oct. 24, 2022.

(51) Int. Cl. A61K 39/215; A61K 9/51; A61K 39/00; C07K 14/005; C07K 16/10; C12P 19/34

(52) U.S. Cl. CPC A61K 39/215; A61K 9/5123; C07K 14/005; C07K 16/1003; C12P 19/34; A61K 2039/505; A61K 2039/53; A61K 2039/55555; C07K 2317/76; C12N 2770/20022; C12N 2770/20034

(57) ABSTRACT

Provided herein are 3′ and 5′ UTRs that provide an amount, duration, or both of protein expression from a recombinant RNA. Compositions of matter, methods, or uses of said 3′ and 5′ UTRs are provided. Provided herein are RNA segments that encode a SARS-CoV-2 omicron spike protein, which provide for omicron-strain-specific immunogenic compositions. Compositions of matter, methods, or uses of said RNA segments that encode a SARS-CoV-2 omicron spike protein are provided.

### CROSS-REFERENCE TO RELATED APPLICATIONS

This application claims the priority under Article 8(1) to: U.S. Provisional Application No. 63/353,568, filed Jun. 18, 2022; U.S. Provisional Application No. 63/413,844, filed Oct. 5, 2022; and U.S. Provisional Application No. 63/380,640, filed Oct. 24, 2022. The complete contents of the above-listed applications are hereby incorporated herein by reference in their entireties for all purposes.

### SEQUENCE LISTING

The instant application contains a Sequence Listing, which has been submitted electronically in computer readable form in an XML format and is hereby incorporated by reference in its entirety. Said XML file, created on Jun. 15, 2023, is named “70039Wo01_SL_1.xml” and is 1,039,811 bytes in size.

### BACKGROUND OF THE INVENTION

### Field of the Invention

Provided herein are compositions, methods, and uses that deliver an ribonucleic acid encoding a protein, and cause expression the protein, including those that elicit an immune response. These compositions, methods, and uses comprise a recombinant RNA comprising 3′ and 5′ UTRs. Independently provided herein are compositions, methods, and uses that elicit an immune response against the severe acute respiratory coronavirus 2 (SARS-CoV-2) omicron (a.k.a. strain B1.1.529) including the three sub-strains BA.1 (a.k.a. B.1.1.529.1), BA.1.1 (a.k.a. B.1.1.529.1.1), BA.2 (a.k.a. B.1.1.529.2), and BA.3 (a.k.a. B.1.1.529.3).

### Further Background

Ribonucleic acid-based (RNA-based) vaccines have emerged as the modality of choice for immunizing against severe acute respiratory coronavirus-2 (SARS-CoV2), the virus that caused the coronavirus disease 2019 (COVID-19). As of March 2022, COVID-19 has resulted in over six million deaths worldwide, with over 960,000 in the United States and 1,860,000 in Europe. As the waves of infections from the delta and omicron impacted the population, it was determined that the neutralizing antibody titers from the primary immunizations were not sufficient to fully prevent infections the newly emerging strains and to protect against reinfection. Boosting with messenger RNA encoding the earlier strains (i.e. the original strain identified in Wuhan, China (Wuhan-strain)) became a strategy, which, for example, increased neutralizing antibody titers and thereby protection. However, the elevations in neutralizing antibody after boosting last between two to four months and thereafter, the levels drop below those thought necessary to provide best protection. Rates of prevention by the earlier generations of vaccines are diminishing as newer strains emerge. It is possible that as the virus mutates into newer and more predominant strains, it will escape the preventative influence of the first-generation of vaccines that are based on said earlier strains. There remains a need for omicron-strain-specific immunogenic compositions.

Also needed are modifications to the backbone of the mRNA to provide for a durable immune response. Said modifications to the backbone may be provided independently of the omicron-specific modifications or in combination therewith.

The RNA, such as in vaccines, contain a segment that encodes a gene of interest (i.e. a segment that encodes a heterologous polypeptide or a segment that encodes an immunogen, e.g. a segment that encodes coronavirus spike protein), a 5′ untranslated region (5′ UTR), an optional 3′ untranslated region (3′ UTR), an optional 3′ poly(adenosine monophosphate) (3′ poly(A)) tail, and an optional 5′ cap.

The 5′ UTR is upstream (i.e. 5′) of the gene of interest; whereas, the 3′ UTR is downstream (i.e. 3′) of the gene of interest. The 5′ UTR begins at the transcription start site and ends one nucleotide before the translation initiation sequence (i.e. a sequence of 5′-adenosine, uridine, guanosine-3′ (5′-AUG-3′)) of the coding region of the gene of interest. The 5′ UTR can thereby affect the expression of the gene of interest through a range of intracellular mechanisms, including but not limited to pre-initiation complex regulation, closed-loop regulation, upstream open reading frame regulations (i.e. reinitiation), internal ribosome entry sites, microRNA binding sites, and others, which may operate in combination with the 5′ cap. The 3′ UTR follows the translation termination codon of the coding region of the gene of interest. The 3′ UTR may provide regulation regions that post-transcriptionally influence gene expression, including translation efficiency (i.e. AU-rich elements (AREs)), localization, stability of the RNA, polyadenylation, and circularization of the RNA to promote translation through interactions with the 5′ UTR and native cellular proteins such as poly(A) binding proteins (PABP). In summary, both the 3′ and 5′ UTR influence expression of the coding region of the gene of interest, and thereby the protein, through a variety of mechanisms.

As noted above, neutralizing antibody levels against SARS-CoV-2 drop below levels that are thought to provide best protection about 2-4 months after boosting with antigens derived from the Wuhan strain. Applicant suggests this duration might be due to the profile of antigen expression after primary or boosting vaccinations. Applicant suggests that UTRs are needed, which increase the amount of protein expressed, duration of protein expression, or both (i.e. area under the curve) from the gene of interest that is delivered by a recombinant RNA molecule.

### BRIEF SUMMARY OF THE INVENTION

In one aspect, the inventors discovered 3′ and 5′ UTRs that provide such amount, duration, or both of protein expression from a recombinant RNA. In another aspect, the inventors discovered RNA segments that encode SARS-CoV-2 omicron spike proteins, which provide for omicron-strain-specific immunogenic compositions. Accordingly, the inventors researched and identified the following aspects and embodiments, including the following compositions of matter, methods, or uses:

A recombinant ribonucleic acid (RNA) molecule comprising a 5′ untranslated region (5′ UTR), a segment that encodes a heterologous polypeptide, a 3′ untranslated region (3′ UTR), and a 3′ poly(adenosine monophosphate) (poly(A)) tail; wherein:
a) the 5′ UTR is 5′ of the segment that encodes the heterologous polypeptide;
b) the 3′ UTR is 3′ of the segment that encodes the heterologous polypeptide;
c) the 3′ poly(A) is 3′ of the 3′ UTR; and
d) i) the 5′ UTR comprises a sequence that has at least 95% identity to SEQ ID NO: 5 and the 3′ UTR comprises a sequence that has at least 95% identity to SEQ ID NO: 6;
   ii) the 5′ UTR comprises a sequence that has at least 95% identity to SEQ ID NO: 7 and the 3′ UTR comprises a sequence that has at least 95% identity to SEQ ID NO: 8; or
   iii) the 5′ UTR comprises a sequence that has at least 95% identity to SEQ ID NO: 13 and the 3′ UTR a sequence that has at least 95% identity to SEQ ID NO: 14.

A formulation comprising lipid nanoparticles (LNPs) and recombinant RNA molecules of any one of the preceding claims; wherein:
i) the LNPs comprise lipids;
ii) the lipids comprise first lipids and cholesterol;
iii) the first lipid comprises a tertiary amine and has a pKa from 5.0 to 7.6;
iv) at least half of the first lipids are neutrally charged when the first lipids are at a pH that is above the pKa; and
v) at least half of the first lipids are positively charged when the first lipids are at a pH that is below the pKa.

A method of eliciting an immune response against the immunogen in a subject, the method comprising administering to the subject an effective amount to elicit the immune response of the recombinant RNA molecule or the formulation.

A method of manufacturing the recombinant RNA molecule, the method comprising admixing an RNA polymerase, triphosphate nucleotides, and a template nucleic acid comprising a sequence of the recombinant RNA molecule, thereby obtaining an admixture; the admixing being under conditions wherein the RNA polymerase produces the recombinant RNA molecule from the template nucleic acid.

A method of manufacturing the formulation, the method comprising:
a. admixing the recombinant RNA molecules with an aqueous buffer, thereby obtaining an aqueous recombinant RNA solution;
b. admixing the lipids in an organic solvent, thereby obtaining a lipid solution;
c. admixing the lipid solution and the recombinant RNA solution with at least a T-mixer, a microfluidics mixer, or an impinging jet mixer, thereby obtaining a first solution, wherein the first solution comprises the LNPs, wherein at least some of the recombinant RNA molecules are encapsulated in the LNPs; and
d. purifying the LNPs to obtain the at least 80% of the recombinant RNA molecules being encapsulated by the LNPs.

A use of the recombinant RNA molecule for the manufacture of a medicament for delivering the segment that encodes the heterologous polypeptide, the method comprising admixing the recombinant RNA molecule with a pharmaceutically acceptable vehicle.

A use of the recombinant RNA molecule for the manufacture of a medicament for preventing a disease caused by a pathogen, the pathogen comprising the immunogen; the use comprising admixing the recombinant RNA molecule with a pharmaceutically acceptable delivery vehicle.

A use of the recombinant RNA molecule for the manufacture of a medicament for treating a disease caused by a pathogen, the pathogen comprising the immunogen; the use comprising admixing the recombinant RNA molecule with a pharmaceutically acceptable delivery vehicle.

A use of the formulation for the manufacture of a medicament for delivering a segment that encodes the heterologous polypeptide, the method comprising admixing the formulation with a pharmaceutically acceptable vehicle.

A use of formulation for the manufacture of a medicament for preventing a disease caused by a pathogen, the pathogen comprising the immunogen; the use comprising admixing the formulation with a pharmaceutically acceptable delivery vehicle.

A use of the formulation for the manufacture of a medicament for treating a disease caused by a pathogen, the pathogen comprising the immunogen; the use comprising admixing the formulation with a pharmaceutically acceptable delivery vehicle.

A composition for use in preventing infection by a pathogen in a subject, the composition comprising (A) a pharmaceutically acceptable vehicle and (B)(i) the recombinant RNA molecule or (B)(ii) the formulation; the pathogen comprising the immunogen.

A composition for use in eliciting an immune response against a pathogen in a subject, the composition comprising (A) a pharmaceutically acceptable vehicle and (B)(i) the recombinant RNA molecule or (B)(ii) the formulation; the pathogen comprising the immunogen.

A composition for use in treating infection by a pathogen in a subject, the composition comprising (A) a pharmaceutically acceptable vehicle and (B)(i) the recombinant RNA molecule or (B)(ii) the formulation; the pathogen comprising the immunogen.

A recombinant RNA molecule comprising a first sequence or a second sequence, wherein the first sequence has at least 90% identity to SEQ ID NO: 29 and the second sequence has at least 97% identity to SEQ ID NO: 30.

A formulation comprising lipid nanoparticles (LNPs) and recombinant RNA molecules; wherein:
i) the LNPs comprise lipids;
ii) the lipids comprise first lipids and cholesterol;
iii) the first lipid comprises a tertiary amine and has a pKa from 5.0 to 7.6;
iv) at least half of the first lipids are neutrally charged when the first lipids are at a pH that is above the pKa; and
v) at least half of the first lipids are positively charged when the first lipids are at a pH that is below the pKa.

A method of eliciting an immune response against the immunogen in a subject, the method comprising administering to the subject an effective amount to elicit the immune response of the recombinant RNA molecule or the formulation.

A method of manufacturing the recombinant RNA molecule, the method comprising admixing an RNA polymerase, triphosphate nucleotides, and a template nucleic acid comprising a sequence of the recombinant RNA molecule, thereby obtaining an admixture; the admixing being under conditions wherein the RNA polymerase produces the recombinant RNA molecule from the template nucleic acid.

A method of manufacturing the formulation, the method comprising:
a. admixing the recombinant RNA molecules with an aqueous buffer, thereby obtaining an aqueous recombinant RNA solution;
b. admixing the lipids in an organic solvent, thereby obtaining a lipid solution;
c. admixing the lipid solution and the recombinant RNA solution with at least a T-mixer, a microfluidics mixer, or an impinging jet mixer, thereby obtaining a first solution, wherein the first solution comprises the LNPs, wherein at least some of the recombinant RNA molecules are encapsulated in the LNPs; and
d. purifying the LNPs to obtain the at least 80% of the recombinant RNA molecules being encapsulated by the LNPs.

A use of the recombinant RNA molecule for the manufacture of a medicament for preventing coronavirus disease-2019 (COVID-19); the use comprising admixing the recombinant RNA molecule with a pharmaceutically acceptable delivery vehicle.

A use of the recombinant RNA molecule for the manufacture of a medicament for reducing the likelihood of at least one symptom of COVID-19; the use comprising admixing the recombinant RNA molecule with a pharmaceutically acceptable delivery vehicle.

A use of formulation for the manufacture of a medicament for preventing COVID-19; the use comprising admixing the formulation with a pharmaceutically acceptable delivery vehicle.

A use of the formulation for the manufacture of a medicament for reducing the likelihood of at least one symptom of COVID-19; the use comprising admixing the recombinant RNA molecules with a pharmaceutically acceptable delivery vehicle.

### DESCRIPTION OF DRAWINGS

The detailed description of preferred embodiments of the invention will be better understood when read in conjunction with the appended drawings. For the purpose of illustrating the invention, the drawings show certain, but not all, preferred embodiments. It should be understood that embodiments of the invention are not limited to the precise arrangements and instrumentalities of those shown in the drawings.

FIG. 1 shows the percent of baby hamster kidney (BHK) cells expressing firefly luciferase from transfected mRNA under the control of the following human UTRs: beta-actin gene (ACTB, Accession NO: NM_001101.5; SEQ ID NOs: 15 & 16), albumin gene (ALB or UTR1, Accession NOs: NG_056261.1 & NM_000477.7; SEQ ID NOs: 1 & 2), adenosine triphosphate (ATP) synthase beta subunit gene (ATPSB or UTR2, Accession NOs: AH002618.2 & NM_001686.4; SEQ ID NOs: 3 & 4), fibroblast activation protein messenger ribonucleic acid (mRNA) (FAP or UTR 3, SEQ ID NOs: 5 & 6, Accession NOs: U09278.2 & NM_001291807.3), H4 clustered histone 15 mRNA (HIST2H4A or UTR4, SEQ ID NOs: 7 & 8; Accession NO: NR_168448.1), glyceraldehyde-3-phosphate dehydrogenase gene (GAPDH or UTR5, Accession NOs: NG_007073.2 & NR_152150.2; SEQ ID NOs: 9 & 10), heat shock protein family A (Hsp70) member 8 gene (HSPA8 or UTR6, Accession NOs: NG_029473.1 & NM_153201.4; SEQ ID NOs: 11 & 12), interleukin-2 gene (IL-2 or UTR7; Accession NOs: AH002842.2 & NM_000586.4; SEQ ID NOs: 13 & 14), and transferrin gene (TF, Accession NO: NG_013080.2; SEQ ID NOs: 17 & 18), with untransfected mouse thymus cells and BHK cells as controls. BHK cells lack an innate immune response and thereby demonstrate gene expression from the UTR without inhibitory influence from the innate immune response. UTRs 3 and 4 stimulate firefly luciferase expression as well as ACTB UTR does when an innate immune response is not present. Beta actin is expressed ubiquitously in cells. Fluorescence was detected 18 hours after transfection. The mRNA had cap-0 capping and uridines, but no N1-methylpseudouridines.

FIG. 2 depicts the median fluorescent intensity of the BHK cells in FIG. 1.

FIG. 3 shows the total fluorescence intensity of all of the BHK cells as treated in FIG. 1 regardless of whether they expressed firefly luciferase or not.

FIG. 4 depicts the percent of human embryonic kidney 293 (HEK293) cells expressing firefly luciferase from transfected mRNA under the control of the following human UTRs: ACTB, UTRs1-7, TF, and eukaryotic translation elongation factor 2 (EEF2) mRNA (Accession NO: NM_001961.4; SEQ ID NOs: 19 & 20). Fluorescence was detected 18 hours after transfection. The mRNA had cap-0 capping and uridines, but no N1-methylpseudouridines. UTRs 2-6 outperformed ACTB UTRs in triggering more cells to express firefly luciferase.

FIG. 5 depicts the median fluorescent intensity of the HEK293 cells in FIG. 4.

FIG. 6 shows the total fluorescence intensity of all of the HEK293 cells as treated in FIG. 4 regardless of whether they expressed firefly luciferase or not.

FIG. 7 depicts the percent of human embryonic kidney 293 (HEK293) cells expressing firefly luciferase from transfected mRNA under the control of the following human UTRs: ACTB, UTRs1-7, TF, and eukaryotic translation elongation factor 2 (EEF2) mRNA (Accession NO: NM_001961.4). Fluorescence was detected 18 hours after transfection. The mRNA had cap-1 capping and N1-methylpseudouridines, but no uridines. UTRs 1, 2, 3, 4, 6, and 7 outperformed ACTB UTRs in triggering more cells to express firefly luciferase.

FIG. 8 depicts the median fluorescent intensity of the HEK293 cells in FIG. 7. At least cells treated with mRNA comprising UTRs 1, 2, 3, 4, 6, and 7, which drove firefly luciferase expression, expressed more firefly luciferase than those cells treated with mRNA comprising ACTB UTRs, which drove firefly luciferase expression. Cells treated with mRNA comprising UTR 5 and EEF UTRs, which drove firefly luciferase expression, had expression that was equal to or greater than that of cells treated with mRNA comprising ACTB UTRs.

FIG. 9 shows the total fluorescence intensity of all of the HEK293 cells as treated in FIG. 7 regardless of whether they expressed firefly luciferase or not.

FIGS. 10 and 11 depict the dose response curve and area under the curve, respectively, of expression of spike protein from SARS-CoV-2 omicron strain encoded by 12.5 ng, 25 ng, 50 ng, and 100 ng of KM72 (SEQ ID NO: 64), KM64 (SEQ ID NO: 42), KM65 (SEQ ID NO: 44), KM61 (SEQ ID NO: 34), KM62 (SEQ ID NO: 37), or KM63 (SEQ ID NO: 40) RNA per well on the cell membrane surface of HEK293 cells. Three prime and 5′ UTR 4 (in KM62) and 3′ and 5′ UTR 7 (in KM63) drove cell membrane surface expression of SARS CoV-2 omicron spike protein as well as UTRs from CureVac's second SARS-CoV-2 candidate (SEQ ID NOs: 27 and 28, which were in KM72), BioNTech's BNT162b2 SARS-CoV-2 FDA-approved product (Accession NOs: NM_000558, OL521838, NM_001130, SEQ ID NOs: 19 and 20, which were in KM64), and Moderna's mRNA-1273 SARS-CoV-2 FDA-approved product (SEQ ID NOs: 23 and 24, which were in KM65). The SARS-COV-2 omicron spike protein mRNA sequences were the same across constructs and were designed using commercially available software that optimized only for a codon adaptation index of greater than or equal to 0.7.

FIGS. 12 and 13 show the dose response curve and area under the curve, respectively, of expression of whole cell spike protein from the same treatments as in FIGS. 10 and 11.

FIGS. 14 and 15 depict the dose response curve and area under the curve, respectively, of expression of spike protein from SARS-CoV-2 omicron strain encoded by 12.5 ng, 25 ng, 50 ng, and 100 ng of KM69 (SEQ ID NO: 56), KM70 (SEQ ID NO: 59), KM71 (SEQ ID NO: 62), or KM96 (SEQ ID NO: 94) RNA per well on the cell membrane surface of HEK293 cells. Three prime and 5′ UTR 4 (in KM 70) and 3′ and 5′ UTR 7 (in KM71) drove cell membrane surface expression of SARS CoV-2 omicron spike protein as well as a UTR from BioNTech's BNT162b2 SARS-CoV-2 FDA-approved product (in KM96). The SARS-COV-2 omicron spike protein mRNA sequences were the same across constructs and were designed using the CR2 algorithm.

FIGS. 16 and 17 show the dose response curve and area under the curve, respectively, of expression of whole cell spike protein from the same treatments as in FIGS. 14 and 15.

FIGS. 18 and 19 depict the dose response curve and area under the curve, respectively, of expression of spike protein from SARS-CoV-2 omicron strain encoded by 12.5 ng, 25 ng, 50 ng, and 100 ng of KM62 (SEQ ID NO:37), KM67 (SEQ ID NO:50), or KM70 (SEQ ID NO: 59) RNA per well on the cell membrane surface of HEK293 cells. Design with the CR1 (in KM67) and CR2 (in KM70) algorithms resulted in significantly more surface expression of SARS-CoV-2 omicron strain than achieved with CAI 0.7 (in KM62), a commercially available software that optimized only for a codon adaptation index of greater than or equal to 0.7. KM62, KM67, and KM70 comprise 5′ and 3′ UTR4.

FIGS. 20 and 21 show the dose response curve and area under the curve, respectively, of expression of whole cell spike protein from the same treatments as in FIGS. 18 and 19.

FIGS. 22 and 23 depict the dose response curve and area under the curve, respectively, of expression of spike protein from SARS-CoV-2 omicron strain encoded by 12.5 ng, 25 ng, 50 ng, and 100 ng of KM63 (SEQ ID NO: 40), KM68 (SEQ ID NO: 53), or KM71 (SEQ ID NO: 62) RNA per well on the cell membrane surface of HEK293 cells. Design with the CR1 (in KM68) and CR2 (in KM71) algorithms resulted in significantly more surface expression of SARS-CoV-2 omicron strain than achieved with CAI 0.7 (in KM63), a commercially available software that optimized only for a codon adaptation index of greater than or equal to 0.7. KM63, KM68, and KM71 comprise 5′ and 3′ UTR7.

FIGS. 24 and 25 show the dose response curve and area under the curve, respectively, of expression of whole cell spike protein from the same treatments as in FIGS. 22 and 23.

FIGS. 26 and 27 show the dose response curve of spike protein from SARS-CoV-2 Wuhan strain encoded by 12.5 ng, 25 ng, 50 ng, and 100 ng of VAC3 (SEQ ID NO: 96), KM87 (SEQ ID NO: 84), KM88 (SEQ ID NO: 87), KM89 (SEQ ID NO: 90), or JW48 (SEQ ID NO: 92) RNA per well on the cell membrane surface of HEK293 cells and on the whole cells, respectively. Three prime and 5′ UTRs 4 and 7 (in KM88 and KM89 respectively) drove cell membrane surface expression and whole cell expression of SARS CoV-2 Wuhan spike protein as well as UTRs from CureVac's second SARS-CoV-2 candidate (in VAC3) and Moderna's mRNA-1273 SARS-CoV-2 FDA-approved product (in JW48). The mRNA sequences for KM87, KM88, and KM89 that encoded SARS-CoV-2 Wuhan spike protein were the same and were designed using commercially available software that optimized only for a codon adaptation index of greater than or equal to 0.7. The mRNA sequence for JW48 that encoded SARS-CoV-2 Wuhan spike protein were designed using IDT's online codon optimization tool. The mRNA sequences for VAC3 that encoded SARS-CoV-2 Wuhan spike protein were obtained from positions 17-3838 of the sequence in The World Health Organization: International Nonproprietary Names Programme. Messenger RNA Encoding the Full-Length SARS-CoV-2 Spike Glycoprotein; p. 11868, which is further described in Edo Kon, Uri Elia, and Dann Peer, “Principles for designing an optimal mRNA lipid nanoparticle vaccine,” Curr. Opin. Biotechnol. 2022, 73:329-336.

FIG. 28 depicts the dose response curve of angiotensin-converting enzyme 2 (ACE2) protein binding with SARS-CoV-2 Wuhan spike protein, which was expressed and released from HEK293 cells administered 62.5 ng, 125 ng, 250 ng, and 500 ng of VAC3 (SEQ ID NO: 96), KM87 (SEQ ID NO: 84), KM88 (SEQ ID NO: 87), KM89 (SEQ ID NO: 90), or JW48 (SEQ ID NO: 92) RNA per well. Three prime and 5′ UTRs 4 and 7 (in KM88 and KM89 respectively) drove Wuhan spike protein expression as well as UTRs from CureVac's second SARS-CoV-2 candidate (in VAC3) and Moderna's mRNA-1273 SARS-CoV-2 FDA-approved product (in JW48).

FIGS. 29 and 30 show the dose response curve of spike protein from SARS-CoV-2 omicron strain encoded by 12.5 ng, 25 ng, 50 ng, and 100 ng of KM71 (SEQ ID NO: 62), KM68 (SEQ ID NO: 53), KM70 (SEQ ID NO: 59), KM62 (SEQ ID NO: 37), KM65 (SEQ ID NO: 44), KM63 (SEQ ID NO: 40), KM66 (SEQ ID NO: 47), or KM61 (SEQ ID NO: 34) RNA per well on the cell membrane surface and the whole cell, respectively, of HEK293 cells. Encoding with CR1 (in KM68) and CR2 (in KM70 and 71) with 3′ and 5′ UTR4 (in KM70) and 3′ and 5′ UTR7 (in KM71 and KM68) drove significantly more cell membrane surface expression and whole cell expression of SARS CoV-2 omicron spike protein cell when compared to when the expression was driven by omicron spike protein sequences that were designed using commercially available software that optimized only for a codon adaptation index of greater than or equal to 0.7 and where the expression was driven by UTR3, UTR4, UTR7, and Moderna UTRs in mRNA-1273 SARS-CoV-2 FDA-approved product (in KM61, KM62, KM63, and KM65 respectively).

FIG. 31 depicts the dose response curve of angiotensin-converting enzyme 2 (ACE2) protein binding with SARS-CoV-2 Wuhan spike protein, which was expressed and released from HEK293 cells administered 62.5 ng, 125 ng, 250 ng, and 500 ng of KM71 (SEQ ID NO: 62), KM68 (SEQ ID NO: 53), KM70 (SEQ ID NO: 59), KM62 (SEQ ID NO: 37), KM65 (SEQ ID NO: 44), KM63 (SEQ ID NO: 40), KM66 (SEQ ID NO: 47), or KM61 (SEQ ID NO: 34) RNA per well. Encoding with CR1 (in KM68) and CR2 (in KM70 and 71) with 3′ and 5′ UTR4 (in KM70) and UTR7 (in KM71 and KM68) drove significantly more omicron spike protein expression as measured by ACE2 binding when compared to when the expression was driven by omicron spike protein sequences that were designed using commercially available software that optimized only for a codon adaptation index of greater than or equal to 0.7 and where the expression was driven by UTR3, UTR4, UTR7, and Moderna UTRs in mRNA-1273 SARS-CoV-2 FDA-approved product (in KM61, KM62, KM63, and KM65 respectively).

FIGS. 32, 33A, and 33B show the geometric mean IgG titers on day 14, day 21, and day 35 from female mice treated on day 0 and day 21 of the study with 5 μg, 1 pg, or 0.2 pg of KM65 (SEQ ID NO: 44), KM68 (SEQ ID NO: 53), KM70 (SEQ ID NO: 59), or KM71 (SEQ ID NO: 62) RNA encapsulated in RV39 LNPs. The individual points are the IgG titers from each individual animal. FIG. 32 organizes the geometric means by RNA treatment, then by dose, then by day of serum extraction for IgG titers. FIGS. 33A and 33B organize the geometric means by day of serum extraction, then by dose, then by RNA treatment. Treatment with RNA encoded by CR2 optimization of SARS-CoV-2 omicron spike protein, which is driven by UTR4, elicited higher IgG titers at days 14 and 21 than treatment with KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. Treatment with RNA encoded by CR2 optimization of SARS-CoV-2 omicron spike protein, which is driven by UTR7, elicited higher IgG titers at days 14 and 21 at 5 μg doses and at day 35 at 5 μg and 0.2 μg than treatment with KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. Treatment with RNA encoded by CR1 optimization of SARS-CoV-2 omicron spike protein, which is driven by UTR7, elicited higher IgG titers at days 21 and 35 at 1 μg doses than treatment with KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product.

FIGS. 34, 35A, and 35B depict neutralizing antibody titers (NT50) against a pseudovirus comprising SARS-CoV-2 omicron spike protein on days 21 and 35 of the same animals treated in FIGS. 32, 33A, and 33B. At day 21 and a dose of 0.2 μg, administration of KM70 elicited higher neutralizing antibody titers than administration of KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. At day 23 and a dose of 1 μg, administration of KM71 or KM68 elicited higher neutralizing antibody titers than administration of KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. At day 23 and a dose of 0.2 μg, administration of KM68 (SEQ ID NO: 53), KM70 (SEQ ID NO: 59), or KM71 (SEQ ID NO: 62) elicited higher neutralizing antibody titers than administration of KM65 (SEQ ID NO: 44), RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product.

FIGS. 36 and 37 show neutralizing antibody titers (NT50) against a pseudovirus comprising SARS-CoV-2 delta spike protein on day 35 of the same animals treated in FIGS. 32, 33A, and 33B. At every dose, administration with KM68 (SEQ ID NO: 53), KM70 (SEQ ID NO: 59), or KM71 (SEQ ID NO: 62) elicited higher neutralizing antibody titers than administration of KM65 (SEQ ID NO: 44), RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product.

FIGS. 38 and 39 depict neutralizing antibody titers (NT50) against live SARS-CoV-2 virus on day 35 of the same animals treated in FIGS. 32, 33A, and 33B. Administration of KM68 (SEQ ID NO: 53), KM70 (SEQ ID NO: 59), or KM71 (SEQ ID NO: 62) elicited neutralizing antibodies (NT50) against live SARS-CoV-2 omicron virus at the same levels as those elicited by administration of KM65 (SEQ ID NO: 44), RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product.

FIGS. 40 and 41 show the dose response curve and area under the curve, respectively, of the HEK293 cell membrane surface expression of SARS-CoV-2 omicron spike protein encoded by 12.5 ng, 25 ng, 50 ng, and 100 ng of KM70 (SEQ ID NO: 59), where the length of the 3′ poly(A) tail consisted of 40 As, 60 As, 80 As, 100 As, 120 As, or 150 As.

FIGS. 42-45 depict the dose response curve of HEK293 cell membrane surface expression of SARS-CoV-2 omicron spike protein encoded by 12.5 ng, 25 ng, 50 ng, and 100 ng of KM65 (SEQ ID NO: 44), KM68 (SEQ ID NO: 53), KM70 (SEQ ID NO: 59), or KM71 (SEQ ID NO: 62) RNA, respectively, where the length of the 3′ poly(A) tail consisted of around 70 As (solid lines) or exactly 100 As (dotted lines).

FIG. 46 depicts the dose response curve of expression of spike protein from SARS-CoV-2 BA.1 omicron strain encoded by 10, 7, 3.5, 2.5, 1.75, 1.25, and 0.8765 ng/well the SARS-COV-2 BA.1 omicron spike protein mRNA sequences, optimized by codeRNA (CR) 2-7. Each construct incorporated the identical mRNA sequence for UTR4, differing only in the sequence encoding the BA.1 immunogen.

FIG. 47 depicts the dose response curve of expression of spike protein from SARS-CoV-2 BA.1 omicron strain encoded by 6.25, 12.5, 25, 50, 100, or 200 ng/well the SARS-COV-2 BA.1 omicron spike protein mRNA sequences, optimized by codeRNA (CR) 2-7. Each construct incorporated the identical mRNA sequence for UTR4, differing only in the sequence encoding the BA.1 immunogen.

FIG. 48 depicts the area under the curve (AUC) of spike protein from SARS-CoV-2 BA.1 omicron strain encoded by 6.25, 25, 50, 100, or 200 ng/well the SARS-COV-2 BA.1 omicron spike protein mRNA sequences, optimized by codeRNA (CR) 2-7 and with UTR 4 or UTR 7. Each construct incorporated the identical mRNA sequence for UTR4, differing only in the sequence encoding the BA.1 immunogen.

FIGS. 49 and 50 show statistically insignificant differences in serum levels of total IgG against anti-SARS-CoV2 BA.1 spike protein at day 14 (FIGS. 49 and 50) and at day 34 (FIG. 50) after the first administration of one of RNA constructs KM70, LL34, LL35, LL36, LL37, and LL38 formulated in RV39 at doses of 0.5 μg, 1 μg, or 5 μg to mice. A second administration of the same doses occurred at day 21 (21 days after the first dose (day 0)). Statistical significance was found in measures between saline treated and construct treated animals.

FIG. 51 shows statistically insignificant differences in serum levels of pseudovirus neutralization antibody titers at days 14 and 34, in the same mice treated with RNA constructs as in FIGS. 49 and 50. Statistical significance was found in measures between saline-treated and construct-treated animals.

FIG. 52. shows statistically insignificant differences in live virus neutralization antibody titers at day 34, in the serum of the same mice treated with RNA constructs as in FIGS. 49 and 50. Statistical significance was found in measures between saline-treated and construct-treated animals.

FIG. 53 shows the gating strategy for flow cytometry for cytokine analysis of T cells from a representative sample obtained from in vivo. Cytokines were gated on time/live/lymphocytes/singlets/CD3 CD4/CD44, or CD8/CD44. Phenotypic subsets were determined by the Boolean combination gate tool.

FIG. 54 depicts the flow cytometry gating scheme from a representative sample obtained from in vivo to identify follicular helper T cells (Tfh) cells. To measure total Tfh, cells were gated on time/live/lymphocytes/singlets/CD3/CD4/CD44/CXCR5+PD1+.

FIG. 55 shows statistically insignificant differences in SARS-COV-2 spike protein specific CD4-positive T cell responses at day 34 isolated from the spleens of the same mice treated with RNA constructs as in FIGS. 49 and 50. Statistical significance was found in measures between saline-treated and construct-treated animals.

FIG. 56 shows statistically insignificant differences in SARS-COV-2 spike protein specific CD8-positive T cell responses at day 34 isolated from the spleens of the same mice treated with RNA constructs as in FIGS. 49 and 50. Statistical significance was found in measures between saline-treated and construct-treated animals.

FIGS. 57A, 57B, 57C, 58A, and 58B show that HEK293 cells administered with LL59, LL60, LL61, LL62, LL66, or LL67 RNA construct had comparable whole cell spike protein expression 24 hrs (FIGS. 57A, 57B, 57C, 58A, and 58B) and 48 hrs (FIGS. 58A and 58B) after administration as measured by area under the curve of the dose response of 25, 50, 75, and 100 ng of RNA construct per well.

FIG. 59 show statistically insignificant differences in serum levels of total IgG against anti-SARS-CoV2 BA.5 spike protein at day 21 after the first administration of one of RNA constructs LL59 (a.k.a. KM70 prime), LL60, LL61, LL62, LL66, and LL67 formulated in RV39 at doses of 0.5 μg, 1 μg, or 5 μg to mice. A second administration of the same doses occurred at day 21 (21 days after the first dose (day 0)). Statistical significance was found in measures between saline treated and construct treated animals.

FIG. 60 shows that adding 5′ UTR7 and 3′ UTR4 into a construct, SP035, results in 2-2.45 higher area under the curve as obtained with LL59, which has 5′ UTR4 and 3′ UTR4. In both constructs, the 3′ poly(A) tail had 30 As, a linker, then 87 As.

### DETAILED DESCRIPTION OF THE INVENTION

The preferred materials and methods are described herein; any methods and materials similar or equivalent to those described herein can be used in the practice of or testing of the invention.

### Definitions

Unless defined otherwise, all technical and scientific terms herein have the same meaning as commonly understood by one of ordinary skill in the art. The following terminology will be used. It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element and more than one element.

“Or” supports, contemplates, and when recited in the claims, claims “one or a combination of” as in “one or a combination of A, B, or C.” To illustrate, “A, B, or C” means A alone, B alone, C alone, the combination of A and B, the combination of A and C, the combination of B and C, and the combination of A, B, and C, unless otherwise illustrated. That is, “or” supports and contemplates “and” as in “and/or” wherein “and/or” includes any combinations within the list of alternatives without being limited solely to the combination of all alternatives in a list (i.e. “A, B, or C” includes “A and B” and is not limited to “A, B, and C”).

Furthermore, the recitation of a list of alternatives, which may be conjoined by “and” and from which at least one alternative is selected (e.g. Markush language), further contemplates and supports all combinations within the list of alternatives. For example, “X is selected from the group of: A, B, and C” contemplates and supports “X is selected from the group of: A, B, C, and combinations thereof,” “X is selected from at least one of the group of: A, B, and C,” and “X is selected from one or more of the group of: A, B, and C.” For further example, “X is selected from the group consisting of A, B, and C” contemplates and supports “X is selected from the group consisting of A, B, C, and combinations thereof,” “X is selected from at least one of the group consisting of A, B, and C,” or “X is selected from one or more of the group consisting of A, B, and C.”

Each of the following contemplates and supports any of the others: “comprises,” “consists of,” “consists essentially of,” “is/are/being,” “is selected from,” “is at least selected from,” “is selected from the group of,” “is selected from the group consisting of,” “is at least selected from the group consisting of,” “is from at least one of the group consisting of,” and “is from one or more of the group consisting of.” For example and in consideration of the above regarding combinations of listed elements, recitation of “X comprises an A, a B, or a C” in the specification contemplates and supports embodiments wherein “X consists of an A, a B, or a C,” “X consists of an A, a B, a C, or combinations thereof,” “X consists of one or more of an A, a B, or a C,” “X is one or more of an A, a B, or a C,” “X is an A, a B, a C, or combinations thereof,” “X is selected from an A, a B, or a C,” “X is selected from an A, a B, a C, or combinations thereof,” “X is selected from the group consisting of an A, a B, a C, and combinations thereof,” “X is selected from at least one of the group consisting of an A, a B, and a C,” or “X is selected from one or more of the group consisting of an A, a B, and a C.”

When a specific component of an embodiment is listed—e.g. “X comprises A, B, or C”-then also supported and contemplated are any embodiments which specifically exclude any individual or combinations of components—e.g. “X comprises A, but not B or C” or “X comprises A but does not comprise B or C.”

“About” as used herein when referring to a measurable value such as an amount, a temporal duration, a quantum of measurement, and the like, is meant to encompass variations of .+−.20% or .+−0.10%, more preferably .+−.5%, even more preferably .+−.1%, and still more preferably .+−.0.1% from the specified value, as such variations are appropriate to perform the disclosed methods.

The term “antibody,” as used herein, refers to an immunoglobulin molecule, which comprises three heavy-chain complementary-determining regions and three light-chain complementary-determining regions (collectively an antigen-determining region), and therefrom specifically binds with an antigen. An antibody can comprise the quintessential “Y” shaped immunoglobulin which comprises two arms and a stem and which comprises two heavy-chains and two light-chains. Each arm comprises a variable region which comprises said light-chain and heavy-chain complementary-determining regions, and each arm comprising a light-chain and a portion (C_{H}1 region) of the heavy-chain. Each stem comprises two portions of a heavy-chain, each portion comprising a C_{H}2 region and a C_{H}3 region. That is, antibodies can be intact immunoglobulins derived from natural sources or from recombinant sources and can be immunoreactive portions of intact immunoglobulins. Antibodies can also be fragments of said intact antibody, wherein the fragment comprises said three heavy-chain complementary-determining regions and said three light-chain complementary-determining regions (i.e. said antigen-determining regions), and therefrom specifically binds with an antigen. The antibodies may exist in a variety of forms including, for example, Fv, Fab, F(ab)₂, linear antibodies, and single chain antibodies (scFv). Antibodies can include polyclonal antibodies, monoclonal antibodies, humanized antibodies, human antibodies, bispecific antibodies, and multi-specific antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “conservative sequence modifications” within the context of amino acid sequences refers to amino acid modifications that do not significantly affect or alter the binding characteristics of the antigen, immunogen, protein, antibody, or antibody fragment containing the amino acid sequence. Such conservative modifications include amino acid substitutions, additions, and deletions. Modifications can be introduced into an antigen, an immunogen, a protein, an antibody, or an antibody fragment by, for example, site-directed mutagenesis and PCR-mediated mutagenesis. Conservative amino acid substitutions are ones in which the amino acid residue is replaced with an amino acid residue having a similar side chain. Similar side chains include categorization by substitution of and with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine), and aromatic side chains (e.g., tyrosine, phenylalanine, tryptophan, histidine).

“Sequence,” “segment,” “nucleic acid,” or “region” as used within the context of a nucleic acid includes sense (i.e. positive) and anti-sense (i.e. negative, e.g. reverse complementary) sequences of the same nucleic acid. A “segment,” “sequence,” “nucleic acid,” or “region” that “encodes” a coding sequence, wherein the coding sequence is transcribed and/or translated, includes sense and antisense (e.g. reverse complementary) sequences of the same nucleic acid. In some embodiments, the coding sequences encode a heterologous polypeptide. In some embodiments, the heterologous polypeptide comprises an immunogen (a.k.a. antigens). In some embodiments, the coding sequence encodes an antibody. In some embodiments, the antibody is an antibody against the immunogen or antigen. In some embodiments, the immunogen or antigen comprises a venom, a poison, an allergen, a cancer antigen, a bacterial antigen, a viral antigen, a fungal antigen, a parasite antigen, or a fragment thereof. That is, in some embodiments, the antibody against an immunogen or an antigen is an antibody against a venom, a poison, an allergen, a cancer antigen, a bacterial antigen, a viral antigen, a fungal antigen, a parasite antigen, or a fragment thereof. And in some embodiments, the coding sequence encodes an immunogen or an antigen, which is a venom, a poison, an allergen, a cancer antigen, a bacterial antigen, a viral antigen, a fungal antigen, a parasite antigen, or a fragment thereof.

In some embodiments, the coding sequence can encode a heterologous polypeptide, which has a therapeutic use. For example, a subject can be deficient in absolute or relative activity of the protein by, for example, having a defective coding region, regulatory region, or regulatory elements for the gene encoding the protein. The administered RNA can provide the coding sequences or regulatory elements therefor for said protein to compensate for said deficiency. In some embodiments, the heterologous polypeptide can comprise an immunotherapeutic molecule or an enzyme. In some embodiments, the enzyme comprises a galactose-1-phosphate uridylyltransferase (GALT) or acid sphingomyelinase, which would be administered to a subject having reduced or deficient activity for the native enzyme, i.e. someone having galactosemia or Neiman-Pick disease respectively.

In some embodiments, a “segment,” “sequence,” “nucleic acid,” or “region” that encodes a coding sequence, is a segment, sequence, or region that encodes an immunogen (a.k.a. antigen) or encodes a heterologous polypeptide. In some embodiments, the heterologous polypeptide includes an antibody. In this regard, to provide for such recombination, the terms “sequence that encodes an immunogen,” “sequence that encodes an antigen,” and “sequence that encodes a heterologous polypeptide” specifically must include a gene sequence other than: for SEQ ID NOs: 1 and 2, albumin gene sequence from any species; for SEQ ID NOs: 3 and 4, an adenosine triphosphate (ATP) synthase beta subunit (ATPSB) gene sequence from any species; for SEQ ID NOs: 5 and 6, a fibroblast activation protein alpha gene sequence from any species; for SEQ ID NOs: 7 and 8, a histone H4 gene sequence from any species, including H4 clustered histone 14 and 15; for SEQ ID NOs: 9 and 10, a glyceraldehyde-3-phosphate dehydrogenase (GAPDH) gene sequence from any species; for SEQ ID NOs: 11 and 12, a heat shock protein family A (Hsp70) member 8 (HSPA8) gene sequence from any species; and for SEQ ID NOs: 13 and 14, an interleukin-2 gene sequence from any species. As noted above, the recombinant RNA molecules can comprise a sequence that encodes a heterologous polypeptide. In this regard, the terms “recombination” and “heterologous” are at least provided by the inclusion of a gene sequence encoding a polypeptide in addition to and other than the above-noted gene sequences for the above-noted SEQ ID NOs: 1-14.

Likewise, a “segment,” “sequence,” “nucleic acid,” or “region” that “encodes” a non-coding sequence, such as miRNA or a promoter, also contemplates and supports sense and antisense (e.g. reverse complementary) sequences of the same nucleic acid. This contemplation and support of both sense and antisense strands for the segment, sequence, nucleic acid, or region is due to the property of a nucleic acid to undergo semi-conservative replication whereby genetic information is retained. In semi-conservative replication, the two strands of double-stranded nucleic acid are separated (i.e. melt or are separated by a helicase), and each of the two strands are used as a template from which a newly synthesized, reverse complementary strand is formed. That is, in semi-conservative replication, the genetic information, whether as sense or antisense is preserved, and a protein, immunogen, miRNA, or promoter, for example, may be produced from the initial strand or strands synthesized therefrom regardless of whether the initial strand is sense or antisense.

In some embodiments, the recombinant RNA can be a self-replicating RNA or a plurality of auto-amplifying RNA. Within the context of self-amplifying RNA and plurality of auto-amplifying RNA, semi-conservative replication provides for the propagation of, for example, the protein, immunogen, miRNA, or promoter regardless of whether the segment encoding the protein, immunogen, miRNA, or promoter was sense or antisense. In this regard, it is understood that the a self-replicating RNA or a plurality of auto-amplifying RNA can comprise one or more segments or sequences that encode one or more proteins necessary for replicating the a self-replicating RNA or a plurality of auto-amplifying RNA in an intracellular environment, and that these segments or sequences that encode the one or more proteins necessary for replicating the a self-replicating RNA or a plurality of auto-amplifying RNA in an intracellular environment are encoded in a sense or positive strand orientation. It is further understood that the a self-replicating RNA or a plurality of auto-amplifying RNA can further comprise a first RNA, wherein the first RNA comprises a heterologous nucleic acid, which in some embodiments can encode a heterologous polypeptide, and wherein the heterologous nucleic acid which in some overlapping embodiments comprises an inhibitory RNA, and it is further understood that the heterologous nucleic acid may be transcribed and translated from the antisense or negative strand of the a self-replicating RNA or a plurality of auto-amplifying RNA.

In this regard, the above-noted support and contemplation of sense and antisense strands applies not only to a self-replicating RNA or a plurality of auto-amplifying RNA, but also to the making and use of a conventional RNA, which is often transcribed from DNA and plasmids, which may encode sense or antisense information, depending upon the step of in the manufacture of the conventional RNA. In this regard, support and contemplation of sense and antisense strands applies not only to the manufacturing process, but also the conventional RNA itself by, for example, supporting and contemplating RNA encoding a therapeutic that can suppress gene expression such as by, for example, antisense RNA, siRNA, RNAi, miRNA, etc.

To illustrate how “sequence,” “segment,” “nucleic acid,” or “region” as used within the context of a nucleic acid includes sense (i.e. positive) and anti-sense, if a specific sequence, called “A”, is listed as having the sequence of 5′-ATGG-3′ in the sense strand (i.e. positive strand) then it is contemplated, supported, and when listed in the claims, claimed that A also has the sequence of 3′-TACC-5′ in the antisense strand (i.e. negative strand) or complementary strand (i.e. A comprises 5′-ATGG-3′ or 3′-TACC-5′).

As statements above provide, “sequence,” “region,” or “segment” as used herein, unless otherwise specified, also contemplates and supports sequences incorporating different forms of nucleic acids, i.e. RNA and DNA, of the same information, or sequences incorporating differing nucleotides found in the different forms of the nucleic acids (i.e. uridines in RNA and thymidines in DNA), as well as sense and anti-sense (e.g. reverse complementary) information therein. For example, RNA may be produced from plasmids of DNA, and thereby the sequence of the plasmid contemplates and supports the sequence of the RNA and vice versa. Since the substitution standard nucleotides with modified nucleotides are contemplated herein (i.e. the substitution of uridines with N1-methylpseudouridines (N1ψ) is contemplated herein), a sequence, region, or segment herein also contemplates and supports sequences incorporating analogs of the standard nucleotides (i.e. uridines) herein. To illustrate, if A in RNA (sense) is 5′-AUGG-3′, A also comprises 5′-ATGG-3′, being the sense DNA, and 3′-TACC-5′ being the anti-sense DNA, as well as 3′-UACC-5′, being the antisense RNA. To also illustrate, 5′-AUGG-3′ also supports and contemplates the sequence of 5′-A(N1ψ)GG-3′, as well as 3′-(N1ψ)ACC-5.′ To distinguish between sense and anti-sense (e.g. complementary) sequences, a prime symbol (′) may be used, i.e. for ease of tracking original genomic material, transcripts, first strand synthesis, second strand synthesis, sense, and antisense strands. To further illustrate, if a first single-stranded region comprises SEQ ID NO: 114, 5′-AATGATACGGCGACCACCGA-3′, then that first single-stranded region also supports and comprises SEQ ID NO: 115, 5′-TCGGTGGTCGCCGTATCATT-3′.

In some embodiments, a “first RNA segment” and a “second RNA segment” are provided. It is to be understood that the second RNA segment is not necessarily downstream (3′) of the first RNA segment or that it is not necessarily upstream (5′) of the first RNA segment either. “First” or “second” with regard to an “RNA segment” is not meant to connote the order along a stand-alone molecule, but rather “first” and “second” are used for nominative convenience. In some embodiments, “first” and “second” lipids are provided. In this regard, a “first” or “second” or any numbered thing is to be understood to use such numbering as to differentiate between said things. For example, a first and second mole percentage may be used to distinguish between the mole percentage of something in a molecule (i.e. the mole percentage of N1-methylpseudouridines to the total of the N1-methylpseudouridines and the uridines in a SAM RNA molecule) and the mole percentage of something in an admixture (i.e. the mole percentage of N1-methylpseudouridines to the total of the N1-methylpseudouridines and the uridines in an in vitro transcription reaction). This thereby avoids the use of determinative language in each and every instance of a term.

“Identity,” “homology,” and within this context, “percent identity” or “percent homology” take the following definitions depending upon whether it is an amino acid sequence or a nucleic acid sequence. “Identity” or “homology” (i.e. percent identity) with respect to an amino acid sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

“Identity” or “homology” (i.e. percent identity) with respect to a nucleic acid sequence is defined herein as the percentage of nucleotides in the candidate sequence that are identical with the reference nucleic acid sequence after aligning the sequences and introducing gaps, if necessary, to achieve the maximum percent sequence identity. The exchange of uridine and thymidine shall be equivalent (i.e. not taken into account) for calculating percent identity. The substitution of uridine or thymidine with a uridine- or thymidine-substitutable modified nucleotide (i.e. U substituted with N1ψ) shall be equivalent (i.e. not taken into account) for calculating percent identity. The substitution of adenosine with an adenosine-substitutable modified nucleotide shall be equivalent (i.e. not taken into account) for calculating percent identity. The substitution of guanosine with a guanosine-substitutable modified nucleotide shall be equivalent (i.e. not taken into account) for calculating percent identity. The substitution of cytosine with a cytosine-substitutable modified nucleotide shall be equivalent (i.e. not taken into account) for calculating percent identity. Where the present disclosure refers to a sequence by reference to a UniProt or Genbank accession code, the sequence referred to is the current version at the filing date of the earliest effective filing date.

“Nucleic acid,” “polynucleotide,” and “oligonucleotide” as used herein all have the same meaning and they are inherently composed of a sequence of nucleotides, each nucleotide comprising a phosphate and a nucleoside, a nucleoside comprising a pentose sugar (e.g. deoxyribose and ribose) and a nucleobase (e.g. a purine comprising adenine or guanine and a pyrimidine comprising cytosine, uracil, N1-methyluracil, and thymine). In some embodiments, the nucleoside (i.e. sugar and nucleobase) can be standard nucleosides (i.e. adenosine, deoxyadenosine, guanosine, deoxyguanosine, thymidine (a.k.a. deoxythymidine), uridine, deoxyuridine, cytidine, or deoxycytidine, or methylates thereof (i.e. 5′-methyluridine) or they may be modified nucleosides (i.e. pseudouridine (a.k.a. 5-(β-D-Ribofuranosyl)pyrimidine-2,4(1H,3H)-dione or 5-[(2S,3R,4S,5R)-3,4-Dihydroxy-5-(hydroxymethyl)oxolan-2-yl]pyrimidine-2,4(1H,3H)-dione, CAS No. 1445-07-4, PubChem CID 15047), N1-methyluridine, N1-methylpseudouridine (a.k.a. 5-[(2S,3R,4S,5R)-3,4-Dihydroxy-5-(hydroxymethyl)oxolan-2-yl]-1-methylpyrimidine-2,4-dione, CAS No. 13860-38-3, PubChem CID 99543), or deoxyribose-containing or ribose-containing forms thereof). In some embodiments, the modified nucleotides comprise hypoxanthine, inosine, 8-oxo-adenine, 7-substituted derivatives thereof, dihydrouracil, pseudouridine, N1-methylpseudouridine, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxy cytosine, 5-(C1-C6)-alkylcytosine, 5-methylcytosine, 5-(C2-C6)-alkenylcytosine, 5-(C2-C6)alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, or hydrogen (abasic residue). A “nucleic acid,” “polynucleotide,” and “oligonucleotide” can be a stand-alone molecule (i.e. an RNA molecule) or they may be “region,” “sequence,” or “segment” therein, and in this regard, the use of “region,” “sequence,” or “segment” is used to distinguish between such and a stand-alone molecule.

The term “heterologous polypeptide” includes a heterologous glycoprotein of the heterologous polypeptide, a heterologous lipo-protein of the heterologous polypeptide, a heterologous lipo-glyco-protein of the heterologous polypeptide, and other terms for the protein that is the result of the post-translational modification of the heterologous polypeptide as translated. In this regard, the heterologous polypeptide may be translated from the segment that encodes the heterologous polypeptide once the recombinant RNA is in a cell. The translated heterologous polypeptide, as encoded by the segment that encodes the heterologous polypeptide, may have moieties to which post-translational modification adds the sugars, lipids, lipids and sugars, and other post-translational modifications. Accordingly, “the segment encodes a heterologous polypeptide” means and refers to a segment that encodes a heterologous polypeptide and post-translational products thereof, including glycoproteins thereof, lipoproteins thereof, lipo-glycoproteins thereof, and post-translational products thereof. The term “heterologous polypeptide” includes a pre-form and/or a pro-form of a biologically active molecule of the heterologous polypeptide and combinations thereof with the aforementioned post-translationally modified heterologous polypeptide, including heterologous glycoprotein of the heterologous polypeptide, a heterologous lipo-protein of the heterologous polypeptide, a heterologous lipo-glyco-protein of the heterologous polypeptide (e.g. pre-pro-insulin and respiratory syncytial virus pre-fusion glycoprotein). The term “heterologous polypeptide” specifically must include a polypeptide other than: for SEQ ID NOs: 1 and 2, albumin from any species; for SEQ ID NOs: 3 and 4, adenosine triphosphate (ATP) synthase beta subunit (ATPSB) from any species; for SEQ ID NOs: 5 and 6, fibroblast activation protein alpha from any species; for SEQ ID NOs: 7 and 8, histone H4 from any species, including H4 clustered histone 14 and 15; for SEQ ID NOs: 9 and 10, glyceraldehyde-3-phosphate dehydrogenase (GAPDH) from any species; for SEQ ID NOs: 11 and 12, heat shock protein family A (Hsp70) member 8 (HSPA8) from any species; and for SEQ ID NOs: 13 and 14, interleukin-2 from any species. As noted above, the recombinant RNA molecules can comprise a sequence that encodes a heterologous polypeptide. Thereby, the terms “recombination” and “heterologous” are at least provided for by the inclusion of a polypeptide other than the above-noted proteins for the above-noted SEQ ID NOs.

The term “immunogen” or also known as an “antigen” (Ag) refers to a molecule that provokes an immune response and can be bound to a protein comprising complementary-determining regions such as an antibody or a T-cell receptor. An antibody includes a B-cell receptor (i.e. an antibody complexed with CD79A and CD79B). As noted above, the recombinant RNA molecules can comprise a sequence that encodes an immunogen. In this regard, to provide for such recombination, the terms “immunogen” and “antigen” specifically must include a protein other than: for SEQ ID NOs: 1 and 2, albumin from any species; for SEQ ID NOs: 3 and 4, adenosine triphosphate (ATP) synthase beta subunit (ATPSB) from any species; for SEQ ID NOs: 5 and 6, fibroblast activation protein alpha from any species; for SEQ ID NOs: 7 and 8, histone H4 from any species, including H4 clustered histone 14 and 15; for SEQ ID NOs: 9 and 10, glyceraldehyde-3-phosphate dehydrogenase (GAPDH) from any species; for SEQ ID NOs: 11 and 12, heat shock protein family A (Hsp70) member 8 (HSPA8) from any species; and for SEQ ID NOs: 13 and 14, interleukin-2 from any species.

The above-noted immune response against the antigen may involve either antibody production against the antigen (i.e. antibody-antigen binding), or the activation of specific immunologically-competent cells to the antigen (i.e. T-cell receptor binding to the antigen), or both. Any macromolecule, including virtually all proteins or peptides, and further including all proteins and peptides comprising post-translational modifications such as the additions of sugars, lipids, and combinations thereof, can serve as an antigen. Antigens can be derived from recombinant or genomic nucleic acids. Any nucleic acid, which comprises a nucleotide sequence or a partial nucleotide sequence encoding a protein that elicits an immune response thereby encodes an “antigen” or “immunogen.” In some embodiments, an antigen or immunogen is encoded by a full-length nucleotide sequence of a gene. In some embodiments, antigens or immunogens are encoded by a partial nucleotide sequence and partial nucleotide sequences of more than one gene. In this regard, in some embodiments, the antigen or immunogen is the full-length native protein or proteins, and in some embodiments, the antigen or immunogen is a truncated portion of the full-length native protein or proteins. In some embodiments, these nucleotide sequences are arranged in various combinations to encode polypeptides that elicit the desired immune response. In this regard, an antigen need not be encoded by a “gene” at all. An antigen can be generated, synthesized, or derived from a biological sample and the amino acid sequence of the protein antigen might be reverse translated or codon optimized to generate a polynucleotide sequence that then encodes the antigen. With regard to the antigen being a full-length or truncated protein and with regard to the antigen activating a T-cell, it is understood that the cells expressing the RNA will express and/or process the antigen in the intracellular environment by truncating the antigen into lengths that can be expressed on by a major histocompatibility receptor so that the T-cell receptor may recognize the MHC-presented antigen.

Such a biological sample can include, but is not limited to a pathogen, a tissue sample, a tumor sample, a cell, or a fluid with other biological components. The pathogen can include a bacteria or a virus. In some embodiments, the immunogen or antigen comprises a venom, a poison, an allergen, a cancer antigen, a bacterial antigen, a viral antigen, a fungal antigen, a parasite antigen, or a fragment thereof.

The terms “protein,” “polypeptide,” and “peptide” are used interchangeably herein and refer to any peptide-linked chain of amino acids, regardless of length or post-translational modification (e.g. phosphorylation or addition of sugars, lipids, or combinations thereof).

The term “post-translational” used herein refers to events that occur after the translation of a nucleotide triplet into an amino acid and the formation of a peptide bond to the proceeding amino acid in the sequence. Such post-translational events may occur after the entire polypeptide was formed or already during the translation process on those parts of the polypeptide that have already been translated. Post-translational events typically alter or modify the chemical or structural properties of the resultant polypeptide. Examples of post-translational events include the addition of sugars, lipids, phospho-groups, cleavage of the peptide chain, or restructuring of folding by, for example, heat shock proteins.

The term “co-translational” used herein refers to events that occur during the translation process of a nucleotide triplet into an amino acid chain. Those events typically alter or modify the chemical or structural properties of the resultant amino acid chain. Examples of co-translational events include but are not limited to events that may stop the translation process entirely or interrupted the peptide bond formation resulting in two discreet translation products.

As used herein, the terms “polyprotein” or “artificial polyprotein” refer to an amino acid chain that comprises, or essentially consists of or consists of two amino acid chains that are not naturally connected to each other. The polyprotein may comprise one or more further amino acid chains. Each amino acid chain is preferably a complete protein, i.e. spanning an entire ORF, or a fragment, domain or epitope thereof. The individual parts of a polyprotein may either be permanently or temporarily connected to each other. Parts of a polyprotein that are permanently connected are translated from a single ORF and are not later separated co- or post-translationally. Parts of polyproteins that are connected temporarily may also derive from a single ORF but are divided co-translationally due to separation during the translation process or post-translationally due to cleavage of the peptide chain, e.g. by an endopeptidase. Additionally or alternatively, parts of a polyprotein may also be derived from two different ORF and are connected post-translationally, for instance through covalent bonds.

An “epitope”, also known as antigenic determinant, is the segment of a macromolecule that is recognized by the immune system, specifically by antibodies or TCRs, (e.g. by B cells, or T cells). Such epitope is that part or segment of a macromolecule capable of binding to an antibody or antigen-binding fragment thereof. In this context, the term “binding” preferably relates to a specific binding. It is preferred that the term “epitope” refers to the segment of protein or polyprotein that is recognized by the immune system. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

In “polyethylene glycol-2000,” “PEG-2000,” “polyethylene glycol-X,” “PEG-X,” “polyethylene of X Da,” or “PEG of X Da” where “X” is a molecular weight, the “2000” or “X” represents the nearest theoretical molecular weight, which is rounding off to the nearest integer of the number-average of monomers in the measured number-average molecular weight in Daltons of the PEG. For example, the monomer in a PEG-conjugated lipid is ethylene oxide (—CH₂—CH₂—O—), which has a molecular weight of 44.04 Da. Assume that the sample has a number-averaged molecular weight of 1990.608 Da, which has a number-average of 45.2 ethylene oxides (1990.608 Da/(44.04 Da per ethylene oxide)=45.2 number-average ethylene oxides). 45 ethylene oxides would be the nearest integer to a number average of 45.2 ethylene oxides in the number-average molecular weight of 1990.608 Da. 45 monomers would also result in a PEG with a theoretical molecular weight of 1981.8 Da (45 ethylene oxides×44.04 Da per ethylene oxide=1981.8 Da). Thus, the theoretical molecular weight is 1981.8 Da of a PEG lipid having a number-average of 45.2 ethylene oxides in a number-average molecular weight of 1990.608 Da. The molecule having the theoretical molecular weight of the PEG of 1981.8 would be the “PEG-2000” as it is the closest theoretical weight to 2000 Da from a sample having a number average molecular weight of 1990.608 Da.

“Protect” or “protection” in the context of protecting against infection, diseases, or conditions caused by a pathogen in a subject means to produce either directly (i.e. by encoding an antibody) or indirectly (i.e. by encoding an antigen to which the immune system responds by producing anti-antigen antibodies or anti-antigen TCR-mediated immune responses) or to elicit an immune response that decreases the likelihood of: 1) the host's body being a reservoir for the replication of the pathogen, and/or to such a level of replication that it can pass from that host to another, or 2) the likelihood or severity of a symptom or a reduction in the number of symptoms of infection by said pathogen. In some embodiments, protection reduces the incidence of an infection, disease, or condition caused by pathogen (i.e. whether symptomatic and asymptomatic) possibly leading to the control of the disease associated with said-pathogen (i.e. sudden acute respiratory syndrome coronavirus-2 (SARS-CoV-2) causing the disease known as coronavirus virus disease 2019 (COVID-19) and/or to the control of associated adverse health outcomes caused by the pathogen.

“Treat” or “treatment” in the context of infection, diseases, or conditions caused by a pathogen means to treat via administration, post-infection any pathogen-causing symptom, effect, or phenotype. Treatment may mean to decrease the severity or frequency of symptoms of the condition or disease in a subject, slow, or eliminate the progression of the condition, totally or partially eliminate the symptoms of the disease or condition in the subject, or reduce or eliminate the number of pathogens in the subject. Treatment of an infection, disease, or condition caused by a pathogen includes ameliorating, stabilizing, reducing, or eliminating the symptoms, effects, or phenotypes caused by the pathogen.

### Recombinant Ribonucleic Acid Molecules Comprising UTRS

In one aspect, provided is a recombinant ribonucleic acid (RNA) molecule comprising an optional 5′ cap structure (i.e. the 5′ optional cap structure comprising a 5′ cap nucleoside (i.e. a 7-methylguanosine), an optional linker, an optional first 5′ ribonucleoside), a 5′ untranslated region (5′ UTR), a segment that encodes a heterologous polypeptide, a 3′ untranslated region (3′ UTR), and an optional 3′ poly(adenosine monophosphate) (poly(A)) tail; wherein:
the 5′ UTR is 5′ of the segment that encodes the heterologous polypeptide;
the 3′ UTR is 3′ of the segment that encodes the heterologous polypeptide;
the optional 3′ poly(A) is 3′ of the 3′ UTR;
the 5′ UTR comprises a sequence that:
   (1) has at least 70% sequence identity to SEQ ID NO: 1, SEQ ID NO: 3, SEQ ID NO: 5, SEQ ID NO: 7, SEQ ID NO: 9, SEQ ID NO: 11, or SEQ ID NO: 13;
   (2) has at least or no more than 1, 2, 3, 4, 5, or 6 additions, deletions, or substitutions to SEQ ID NO: 1, SEQ ID NO: 3, SEQ ID NO: 5, SEQ ID NO: 7, SEQ ID NO: 9, SEQ ID NO: 11, or SEQ ID NO: 13; or
   (3) both (1) and (2).

In another, provided is a recombinant ribonucleic acid (RNA) molecule comprising an optional 5′ cap structure (i.e. the 5′ optional cap structure comprising a 5′ cap nucleoside (i.e. a 7-methylguanosine), an optional linker, an optional first 5′ ribonucleoside), a 5′ untranslated region (5′ UTR), a segment that encodes a heterologous polypeptide, a 3′ untranslated region (3′ UTR), and an optional 3′ poly(adenosine monophosphate) (poly(A)) tail; wherein:
the 5′ UTR is 5′ of the segment that encodes the heterologous polypeptide;
the 3′ UTR is 3′ of the segment that encodes the heterologous polypeptide;
the optional 3′ poly(A) is 3′ of the 3′ UTR; and
the 3′ UTR comprises a sequence that:
   (1) has at least 70% sequence identity to SEQ ID NO: 2, SEQ ID NO: 4, SEQ ID NO: 6, SEQ ID NO: 8, SEQ ID NO: 10, SEQ ID NO: 12, or SEQ ID NO: 14;
   (2) has 0, 1, 2, 3, 4, 5, or 6 additions, deletions, or substitutions to SEQ ID NO: 2, SEQ ID NO: 4, SEQ ID NO: 6, SEQ ID NO: 8, SEQ ID NO: 10, SEQ ID NO: 12, or SEQ ID NO: 14; or
   (3) has both (1) and (2).

In one aspect, provided is a recombinant ribonucleic acid (RNA) molecule comprising an optional 5′ 7-methylguanosine, an optional linker, an optional first 5′ ribonucleoside, a 5′ untranslated region (5′ UTR), a segment that encodes a heterologous polypeptide, a 3′ untranslated region (3′ UTR), and an optional 3′ poly(adenosine monophosphate) (poly(A)) tail; wherein:
the 5′ UTR is 5′ of the segment that encodes the heterologous polypeptide;
the 3′ UTR is 3′ of the segment that encodes the heterologous polypeptide;
the optional 3′ poly(A) is 3′ of the 3′ UTR;
the 5′ UTR comprises a sequence that has at least 70% sequence identity to SEQ ID NO: 1, SEQ ID NO: 3, SEQ ID NO: 5, SEQ ID NO: 7, SEQ ID NO: 9, SEQ ID NO: 11, or SEQ ID NO: 13; and
the 3′ UTR comprises a sequence that has at least 70% sequence identity to SEQ ID NO: 2, SEQ ID NO: 4, SEQ ID NO: 6, SEQ ID NO: 8, SEQ ID NO: 10, SEQ ID NO: 12, or SEQ ID NO: 14.

### UTR

In some embodiments, the 5′ UTR comprises, consists of, or is a sequence that has at least or no more than 1, 2, 3, 4, 5, or 6 additions, deletions, or substitutions to SEQ ID NO: 1, SEQ ID NO: 3, SEQ ID NO: 5, SEQ ID NO: 7, SEQ ID NO: 9, SEQ ID NO: 11, or SEQ ID NO: 13. In some embodiments, the 5′ UTR comprises, consists of, or is a sequence that has 0, 1, 2, 3, 4, 5, or 6 additions, deletions, or substitutions to SEQ ID NO: 1, SEQ ID NO: 3, SEQ ID NO: 5, SEQ ID NO: 7, SEQ ID NO: 9, SEQ ID NO: 11, or SEQ ID NO: 13.

In some embodiments, the 3′ UTR comprises, consists of, or is a sequence that has at least or no more than 1, 2, 3, 4, 5, or 6 additions, deletions, or substitutions to SEQ ID NO: 2, SEQ ID NO: 4, SEQ ID NO: 6, SEQ ID NO: 8, SEQ ID NO: 10, SEQ ID NO: 12, or SEQ ID NO: 14.

In some embodiments, the 3′ UTR comprises, consists of, or is a sequence that has 0, 1, 2, 3, 4, 5, or 6 additions, deletions, or substitutions to SEQ ID NO: 2, SEQ ID NO: 4, SEQ ID NO: 6, SEQ ID NO: 8, SEQ ID NO: 10, SEQ ID NO: 12, or SEQ ID NO: 14.

In some embodiments, the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1, SEQ ID NO: 3, SEQ ID NO: 5, SEQ ID NO: 7, SEQ ID NO: 9, SEQ ID NO: 11, or SEQ ID NO: 13; (2) has 0, 1, 2, 3, 4, 5, or 6 additions, deletions, or substitutions to SEQ ID NO: 1, SEQ ID NO: 3, SEQ ID NO: 5, SEQ ID NO: 7, SEQ ID NO: 9, SEQ ID NO: 11, or SEQ ID NO: 13; or (3) has both (1) and (2).

In some embodiments, the 3′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 2, SEQ ID NO: 4, SEQ ID NO: 6, SEQ ID NO: 8, SEQ ID NO: 10, SEQ ID NO: 12, or SEQ ID NO: 14; (2) has 0, 1, 2, 3, 4, 5, or 6 additions, deletions, or substitutions to SEQ ID NO: 2, SEQ ID NO: 4, SEQ ID NO: 6, SEQ ID NO: 8, SEQ ID NO: 10, SEQ ID NO: 12, or SEQ ID NO: 14; or (3) has both (1) and (2).

In some embodiments, when the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1; (2) has 0, 1, 2, 3, 4, 5, or 6 additions, deletions, or substitutions to SEQ ID NO: 1; or (3) has both (1) and (2), the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 2; (5) has 0, 1, 2, 3, 4, 5, or 6 additions, deletions, or substitutions to SEQ ID NO: 2; or (6) has both (4) and (5.

In some embodiments, when the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 3; (2) has 0, 1, 2, 3, 4, 5, or 6 additions, deletions, or substitutions to SEQ ID NO: 3; or (3) has both (1) and (2), the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 4; (5) has 0, 1, 2, 3, 4, 5, or 6 additions, deletions, or substitutions to SEQ ID NO: 4; or (6) has both (4) and (5).

In some embodiments, when the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 5; (2) has 0, 1, 2, 3, 4, 5, or 6 additions, deletions, or substitutions to SEQ ID NO: 5; or (3) has both (1) and (2), the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 6; (5) has 0, 1, 2, 3, 4, 5, or 6 additions, deletions, or substitutions to SEQ ID NO: 6; or (6) has both (4) and (5).

In some embodiments, when the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 7; (2) has 0, 1, 2, 3, 4, 5, or 6 additions, deletions, or substitutions to SEQ ID NO: 7; or (3) has both (1) and (2), the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 8; (5) has 0, 1, 2, 3, 4, 5, or 6 additions, deletions, or substitutions to SEQ ID NO: 8; or (6) has both (4) and (5).

In some embodiments, when the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 9; (2) has 0, 1, 2, 3, 4, 5, or 6 additions, deletions, or substitutions to SEQ ID NO: 9; or (3) has both (1) and (2), the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 10; (5) has 0, 1, 2, 3, 4, 5, or 6 additions, deletions, or substitutions to SEQ ID NO: 10; or (6) has both (4) and (5).

In some embodiments, when the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 11; (2) has 0, 1, 2, 3, 4, 5, or 6 additions, deletions, or substitutions to SEQ ID NO: 11; or (3) has both (1) and (2), the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 12; (5) has 0, 1, 2, 3, 4, 5, or 6 additions, deletions, or substitutions to SEQ ID NO: 12; or (6) has both (4) and (5).

In some embodiments, when the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 13; (2) has 0, 1, 2, 3, 4, 5, or 6 additions, deletions, or substitutions to SEQ ID NO: 13; or (3) has both (1) and (2), the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 14; (5) has 0, 1, 2, 3, 4, 5, or 6 additions, deletions, or substitutions to SEQ ID NO: 14; or (6) has both (4) and (5).

In some embodiments, the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 7; (2) has 0, 1, 2, 3, 4, 5, or 6 additions, deletions, or substitutions to SEQ ID NO: 7; or (3) has both (1) and (2); and the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 14; (5) has 0, 1, 2, 3, 4, 5, or 6 additions, deletions, or substitutions to SEQ ID NO: 14; or (6) has both (4) and (5).

In some embodiments, the 5′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 13; (2) has 0, 1, 2, 3, 4, 5, or 6 additions, deletions, or substitutions to SEQ ID NO: 13; or (3) has both (1) and (2); and the 3′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 8; (5) has 0, 1, 2, 3, 4, 5, or 6 additions, deletions, or substitutions to SEQ ID NO: 8; or (6) has both (4) and (5).

In some embodiments, when the 3′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 2; (2) has 0, 1, 2, 3, 4, 5, or 6 additions, deletions, or substitutions to SEQ ID NO: 2; or (3) has both (1) and (2); the 5′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1; (5) has 0, 1, 2, 3, 4, 5, or 6 additions, deletions, or substitutions to SEQ ID NO: 1; or (6) has both (4) and (5).

In some embodiments, when the 3′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 4; (2) has 0, 1, 2, 3, 4, 5, or 6 additions, deletions, or substitutions to SEQ ID NO: 4; or (3) has both (1) and (2); the 5′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 3; (5) has 0, 1, 2, 3, 4, 5, or 6 additions, deletions, or substitutions to SEQ ID NO: 3; or (6) has both (4) and (5).

In some embodiments, when the 3′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 6; (2) has 0, 1, 2, 3, 4, 5, or 6 additions, deletions, or substitutions to SEQ ID NO: 6; or (3) has both (1) and (2); the 5′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 5; (5) has 0, 1, 2, 3, 4, 5, or 6 additions, deletions, or substitutions to SEQ ID NO: 5; or (6) has both (4) and (5).

In some embodiments, when the 3′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 8; (2) has 0, 1, 2, 3, 4, 5, or 6 additions, deletions, or substitutions to SEQ ID NO: 8; or (3) has both (1) and (2); the 5′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 7; (5) has 0, 1, 2, 3, 4, 5, or 6 additions, deletions, or substitutions to SEQ ID NO: 7; or (6) has both (4) and (5).

In some embodiments, when the 3′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 10; (2) has 0, 1, 2, 3, 4, 5, or 6 additions, deletions, or substitutions to SEQ ID NO: 10; or (3) has both (1) and (2); the 5′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 9; (5) has 0, 1, 2, 3, 4, 5, or 6 additions, deletions, or substitutions to SEQ ID NO: 9; or (6) has both (4) and (5).

In some embodiments, when the 3′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 12; (2) has 0, 1, 2, 3, 4, 5, or 6 additions, deletions, or substitutions to SEQ ID NO: 12; or (3) has both (1) and (2); the 5′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 11; (5) has 0, 1, 2, 3, 4, 5, or 6 additions, deletions, or substitutions to SEQ ID NO: 11; or (6) has both (4) and (5).

In some embodiments, when the 3′ UTR comprises, consists of, or is a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 14; (2) has 0, 1, 2, 3, 4, 5, or 6 additions, deletions, or substitutions to SEQ ID NO: 14; or (3) has both (1) and (2); the 5′ UTR comprises, consists of, or is a sequence that: (4) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 13; (5) has 0, 1, 2, 3, 4, 5, or 6 additions, deletions, or substitutions to SEQ ID NO: 13; or (6) has both (4) and (5).

Segment or Sequence that Encodes a Heterologous Polypeptide

In some embodiments, the segment or sequence that encodes a heterologous polypeptide comprises or has one, two, three, four, five, or six stop codons at its 3′ end. In some embodiments, the segment or sequence that encodes a heterologous polypeptide comprises or has a translation initiation codon (i.e. “AUG”) at its 5′ end. In some embodiments, the segment or sequence that encodes a heterologous polypeptide comprises, consists of, consists essentially of, is, or has a segment or sequence that encodes: an immunogen, an antibody, an enzyme, a hormone, or a structural protein. In some embodiments, the heterologous polypeptide includes a heterologous glycoprotein of the heterologous polypeptide, a heterologous lipo-protein of the heterologous polypeptide, a heterologous lipo-glyco-protein of the heterologous polypeptide, and post-translational products of the heterologous polypeptide that was translated from the segment that encodes the heterologous polypeptide. In some embodiments, the heterologous polypeptide comprises an amino acid moiety to which post-translational modification adds the sugars, lipids, lipids and sugars, and post-translational modifications of the heterologous polypeptide. In some embodiments, of the methods disclosed below, the method comprises translation of the heterologous polypeptide from the segment comprising the heterologous polypeptide in the subject and post-translational modification of the amino acid moiety to which the post-translational modification adds a modification comprising a sugar, a lipid, thereby obtaining a cell expressing a post-translationally modified form of the heterologous amino acid.

In some embodiments, the heterologous polypeptide comprises pre-form or pro-form of biologically active proteins. In some embodiments, the immunogen, the antibody, the enzyme, the hormone, or the structural protein is a pre-form or pro-form of the biologically active immunogen, the biologically active antibody, the biologically active enzyme, the biologically active hormone, or the biologically active structural protein. In some embodiments, the immunogen, the antibody, the enzyme, the hormone, or the structural protein is the biologically active immunogen, the biologically active antibody, the biologically active enzyme, the biologically active hormone, or the biologically active structural protein. For example, respiratory syncytial virus (RSV) immunogens include RSV fusion protein/glycoprotein (RSV—F) and its prefusion form, RSV pre-F.

In some embodiments, the segment that encodes a heterologous polypeptide comprises a sequence that has at least 70% identity to at least one of SEQ ID NOs: 29-31 and 123-133. In some embodiments, the segment that encodes a heterologous polypeptide (e.g. the antigen-encoding sequence) comprises a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to at least one of SEQ ID NOs: 29-31 and 123-133; (2) has at least, no more than, or exactly 0, 1, 2, 3, 4, 5, 6, 7, 8, 9, or 10 additions, deletions, or substitutions to at least one of SEQ ID NOs: 29-31 and 123-133; or (3) has both (1) and (2).

In some embodiments, the recombinant RNA molecule comprises a sequence that: (1) has at least 70% identity to at least one of SEQ ID NOs: 32-40, 45-62, 69-77, 82-90, 134-173, and 178-197; (2) has at least, no more than, or exactly 0, 1, 2, 3, 4, 5, 6, 7, 8, 9, or 10 additions, deletions, or substitutions to at least one of SEQ ID NOs: 32-40, 45-62, 69-77, 82-90, 134-173, and 178-197; or (3) has both (1) and (2). In some embodiments, the recombinant RNA molecule comprises a sequence that: (1) has at least 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to at least one of SEQ ID NOs: 32-40, 45-62, 69-77, 82-90, 134-173, and 178-197; (2) has at least, no more than, or exactly 0, 1, 2, 3, 4, 5, 6, 7, 8, 9, or 10 additions, deletions, or substitutions to at least one of SEQ ID NOs: 32-40, 45-62, 69-77, 82-90, 134-173, and 178-197; or (3) has both (1) and (2).

### Immunogens

In some embodiments, the immunogen elicits an immune response against at least one of the following bacteria, is an immunogen from at least one of the following bacteria, is an immunogen derived from at least one of the following bacteria (i.e. by being derived, it may not be the exact immunogen polypeptide, but may have modifications which may stabilize the protein for, for example, expression in a cell or presentation by an immune response), or is an immunogen mimotope of a polypeptide from at least one of the following bacteria: Neisseria meningitidis including, but are not limited to, membrane proteins such as adhesins, autotransporters, toxins, iron acquisition proteins, and factor H binding protein; Streptococcus pneumoniae including, but are not limited to, the RrgB pilus subunit, the beta-N-acetyl-hexosaminidase precursor (spr0057), spr0096, general stress protein GSP-781 (spr2021, SP2216), serine/threonine kinase StkP (SP1732), and pneumococcal surface adhesin PsaA; Streptococcus pyogenes; Moraxella catarrhalis; Bordetella pertussis including but are not limited to, pertussis toxin or toxoid (PT), filamentous haemagglutinin (FHA), pertactin, and agglutinogens 2 and 3; Staphylococcus aureus including a hemolysin, esxA, esxB, ferrichrome-binding protein (sta006), and the sta011 lipoprotein; Clostridium tetani including tetanus toxoid immunogen; Cornynebacterium diphtheriae including diphtheria toxoid immunogen; Haemophilus influenzae; Pseudomonas aeruginosa; Streptococcus agalactiae; Chlamydia trachomatis including, but are not limited to, PepA, LcrE, ArtJ, DnaK, CT398, OmpH-like, L7/L12, OmcA, AtoS, CT547, Eno, HtrA, and MurG; Chlamydia pneumoniae; Helicobacter pylori; Escherichia coli, including the following pathological subforms enterotoxigenic E. coli (ETEC), enteroaggregative E. coli (EAggEC), diffusely adhering E. coli (DAEC), enteropathogenic E. coli (EPEC), enterohemorrhagic E. coli (EHEC), and extraintestinal pathogenic E. coli (ExPEC), including therein uropathogenic E. coli (UPEC) and meningitis/sepsis-associated E. coli (MNEC); Bacillus anthracis; Yersinia pestis; Staphylococcus epidermis; Clostridium difficile; Clostridium perfringens; Clostridium botulinums; Legionella pneumophila; Coxiella burnetiid; Brucella, including B. abortus, B. canis, B. melitensis, B. neotomae, B. ovis, B. suis, B. pinnipediae; Francisella, including F. novicida, F. philomiragia, F. tularensis. Neisseria gonorrhoeae, including polypeptides of the outer membrane vesicles; Treponema pallidum; Haemophilus ducreyi; Enterococcus faecalis; Enterococcus faecium; Staphylococcus saprophyticus; Yersinia enterocolitica; Mycobacterium tuberculosis; Rickettsia; Listeria monocytogenes; Vibrio cholerae; Salmonella including Salmonella typhii; Borrelia burgdorferi; Porphyromonas gingivalis; and Klebsiella. In some embodiments, Clostridium difficile immunogens comprise Toxin A and Toxin B (also known as TcdA and TcdB respectively), fragments thereof, detoxifying mutations thereof, and combinations thereof. In some embodiments, the detoxifying mutations of TcdA, TcdB, and fragments thereof comprise include insertions, deletions, and point-mutations. In some embodiments, the detoxifying mutations and fragments of TcdA and TcdB can be those in WO2014/197651, WO2015/123767, and WO2019/64115.

In some embodiments, the immunogen elicits an immune response against at least one of the following viruses, is an immunogen from at least one of the following viruses, is an immunogen derived from at least one of the following viruses (i.e. by being derived, it may not be the exact immunogen polypeptide, but may have modifications which may stabilize the polypeptide for, for example, expression in a cell or presentation by an immune response), or is an immunogen mimotope of a polypeptide from at least one of the following viruses: Orthomyxovirus including influenza A, B, or C virus, including from influenza A virus subtypes H1, H2, H3, H4, H5, H6, H7, H8, H9, H10, H11, H12, H13, H14, H15, and H16 and including the immunogens of neuraminidase matrix M2 proteins, and hemagglutinin; Paramyxoviridae viruses including, but are not limited to, those derived from Pneumoviruses including respiratory syncytial virus (RSV), Rubulaviruses including mumps virus, Paramyxoviruses including parainfluenza virus, Metapneumoviruses, Morbilliviruses including measles, including the spike-like proteins immunogens thereof, membrane fusion proteins, (i.e. encoded as proteins by the RNA but becoming glycoproteins once translated by the mammalian cell expressing said protein; e.g. RSV F and RSV pre-F) and attachment proteins (i.e. encoded as proteins by the RNA but becoming glycoproteins once translated by the mammalian cell expressing said protein; e.g. RSV G); Poxviridae: Viral immunogens include, but are not limited to, those derived from Orthopoxvirus such as Variola vera, including but not limited to, Variola major and Variola minor; Picornavirus including Rhinoviruses, Heparnavirus, Cardioviruses, Aphthoviruses, and Enteroviruses including EV71 enterovirus, coxsackie A virus, coxsackie B virus, type 1 poliovirus, type 2 poliovirusand type 3 poliovirus; Bunyavirus including Orthobunyavirus such as California encephalitis virus, a Phlebovirus such as Rift Valley Fever virus, and a Nairovirus such as Crimean-Congo hemorrhagic fever virus; Heparnavirus including hepatitis A virus (HAV); Filovirus including Marburg virus and Ebolavirus including Zaire ebolavirus, Tai Forest ebolavirus (nee Ivory Coast ebolavirus), Sudan ebolavirus, Reston ebolavirus, Bundibugyo ebolavirus, and Bombali ebolavirus; Togavirus including, Alphavirus, Arterivirus, and Rubivirus including Rubivirus rubella, Rubivirus ruteetense, and Rubivirus strelense; Flavivirus including Tick-borne encephalitis (TBE) virus, Dengue virus (e.g. types 1, 2, 3 or 4), Yellow Fever virus, Japanese encephalitis virus, Kyasanur Forest Virus, West Nile encephalitis virus, St. Louis encephalitis virus, Russian spring-summer encephalitis virus, Powassan encephalitis virus, and Zikavirus; Pestivirus including Bovine viral diarrhea (BVDV), Classical swine fever (CSFV), and Border disease (BDV); Hepadnavirus including Hepatitis B virus, such as hepatitis B virus surface antigen (HBsAg); other hepatitis viruses, including hepatitis C virus, delta hepatitis virus, hepatitis E virus, and hepatitis G virus; Rhabdovirus including alpharahabdovirinae, Almendravirus, Alphanemrhavirus, Alphapaprhavirus, Alpharicinrhavirus, Arurhavirus, Barhavirus, Caligrhavirus, Curiovirus, Ephemerovirus, Hapavirus, Ledantevirus, Lostrhavirus, Lyssavirus such as rabies virus, Merhavirus, Mousrhavirus, Ohlsrhavirus, Perhabdovirus, Sawgrhavirus, Sigmavirus, Sprivivirus, Sripuvirus, Sunrhavirus, Tibrovirus, Tupavirus, Vesiculovirus, Zarhavirus, Betarhabdovirinae, Alphanucleorhabdovirus, Betanucleorhabdovirus, Cytorhabdovirus, Dichorhavirus, Gammanucleorhabdovirus, Varicosavirus, Gammarhabdovirinae, Novirhabdovirus, Alphacrustrhavirus, Alphadrosrhavirus, Alphahymrhavirus, Betahymrhavirus, Betanemrhavirus, Betapaprhavirus, Betaricinrhavirus; Caliciviridae including Norwalk virus (Norovirus) and Norwalk-like Viruses, such as Hawaii Virus and Snow Mountain Virus; Coronavirus including alpha-, beta-, gamma-, and delta-coronaviruses, including severe acute respiratory syndrome (SARS) coronavirus, SARS-CoV-2, Middle East respiratory syndrome coronavirus, human coronavirus HKU1, coronavirus avian infectious bronchitis (IBV), Mouse hepatitis virus (MHV), and Porcine transmissible gastroenteritis virus (TGEV), including a spike polypeptide immunogen, nucleoprotein immunogen, membrane protein immunogen, and ORF3a protein immunogen; Retrovirus including Oncovirus, Lentivirus, such as human immunodeficiency virus 1 and 2, and Spumavirus; Reovirus including Orthoreovirus, Rotavirus, Orbivirus, and Coltivirus; Parvovirus including Parvovirus B19; Herpesvirus including a human herpesvirus including Herpes Simplex Viruses (HSV), such as HSV types 1 and 2, Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8), including such immunogens as gB, gEv(e.g. VZV gE), gH (e.g. CMV gH), gl, gL (e.g. CMV gL), gO; gM, gN; UL128, UL130, and UL131A (e.g. CMV UL128, UL130, and UL131A); Papovaviruses including Polyomaviruses and Papillomaviruses, including 1, 2, 4, 5, 6, 8, 11, 13, 16, 18, 31, 33, 35, 39, 41, 42, 47, 51, 57, 58, 63, and 65 thereof; and Adenovirus including Adenovirus A such as adenoviruses 12, 18, 31, Adenovirus B such as adenoviruses 3, 7, 11, 14, 16, 21, 34, 35, 50, and 55, Adenovirus C such as adenoviruses 1, 2, 5, 6, and 57, Adenovirus D such as adenoviruses 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56, 58, 59, 60, 62, 63, 64, 65, 67, 69, 70, 71, 72, 73, 74, and 75, Adenovirus E such as adenovirus 4, Adenovirus F such as adenoviruses 40 and 41, and Adenovirus G such as adenovirus 52.

In some embodiments, the immunogen elicits an immune response against at least one of the following fungi, is an immunogen from at least one of the following fungi, is an immunogen derived from at least one of the following fungi (i.e. by being derived, it may not be the exact immunogen polypeptide, but may have modifications which may stabilize the polypeptide for, for example, expression in a cell or presentation by an immune response), or is an immunogen mimotope of a polypeptide from at least one of the following fungi: Dermatophytres, Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis; Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

In some embodiments, the immunogen elicits an immune response against at least one of the following parasites, is an immunogen from at least one of the following parasites, is an immunogen derived from at least one of the following parasites (i.e. by being derived, it may not be the exact immunogen polypeptide, but may have modifications which may stabilize the polypeptide for, for example, expression in a cell or presentation by an immune response), or is an immunogen mimotope of a polypeptide from at least one of the following parasites: Plasmodium, such as P. falciparum, P. vivax, P. malariae, and P. ovale and Caligidae family, particularly those from the Lepeophtheirus and Caligus genera, such as sea lice such as Lepeophtheirus salmonis or Caligus rogercresseyi.

In some embodiments, the immunogen elicits an immune response against: pollen allergens (tree, herb, weed, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g.

Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments, the immunogen is a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH—P1, PSM-P1, PSM-P2, associated with e.g., prostate cancer; (f) immunoglobulin idiotypes (associated with myeloma and B cell lymphomas, for example). In certain embodiments, tumor immunogens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29BCAA), CA 195, CA 242, CA-50, CAM43, CD68KP1, CO-029, FGF-5, Ga733 (EpCAM), HTgp-175, M344, MA-50, MG7-Ag, MOV18, NB/70K, NY—CO-1, RCAS1, SDCCAG16, TA-90 (Mac-2 binding protein/cyclophilin C-associated protein), TAAL6, TAG72, TLP, TPS, and the like.

In some embodiments, the segment that encodes an immunogen comprises a sequence that has at least 70% identity to at least one of SEQ ID NOs: 29-31 and 123-133. In some embodiments, the segment that encodes an immunogen comprises a sequence that: (1) has at least 70%, 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to at least one of SEQ ID NOs: 29-31 and 123-133; (2) has at least, no more than, or exactly 0, 1, 2, 3, 4, 5, 6, 7, 8, 9, or 10 additions, deletions, or substitutions to at least one of SEQ ID NOs: 29-31 and 123-133; or (3) has both (1) and (2).

RECOMBINANT RIBONUCLEIC ACID MOLECULES COMPRISING SEQUENCE THAT ENCODE SARS-COV-2 OMICRON STRAIN-SPECIFIC SPIKE PROTEINS.

In another aspect, provided is a recombinant RNA molecule comprising a sequence (e.g. the antigen-encoding sequence) that: (1) has at least 70% identity to at least one of SEQ ID NOs: 29, 30, and 123-133; (2) has 0, 1, 2, 3, 4, 5, 6, 7, 8, 9, or 10 additions, deletions, or substitutions to at least one of SEQ ID NOs: 29, 30, and 123-133; or (3) has both (1) and (2); and optionally further comprising an optional 5′ cap structure (the optional 5′ cap structure comprising a 5′ cap nucleoside (i.e. an optional 5′ 7-methylguanosine), an linker, an optional first 5′ ribonucleoside), an optional 5′ UTR, an optional 3′ UTR, and an optional 3′ poly(A) tail.

In some embodiments, the sequence (e.g. the antigen-encoding sequence) that: (1) has at least 70% identity to at least one of SEQ ID NOs: 29, 30, and 123-133; (2) has 0, 1, 2, 3, 4, 5, 6, 7, 8, 9, or 10 additions, deletions, or substitutions to at least one of SEQ ID NOs: 29, 30, and 123-133; or (3) has both (1) and (2) has: (1) at least 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to at least one of SEQ ID NOs: 29, 30, and 123-133, (2) has 0, 1, 2, 3, 4, 5, 6, 7, 8, 9, or 10 additions, deletions, or substitutions to at least one of SEQ ID NOs: 29, 30, and 123-133; or (3) has both (1) and (2).

In some embodiments, the recombinant RNA molecule comprises a sequence that: (1) has at least 70% identity to at least one of SEQ ID NOs: 45-62, 134-173, and 178-197; (2) has at least, no more than, or exactly 0, 1, 2, 3, 4, 5, 6, 7, 8, 9, or 10 additions, deletions, or substitutions to at least one of SEQ ID NOs: 45-62, 134-173, and 178-197; or (3) has both (1) and (2). In some embodiments, the recombinant RNA molecule comprises a sequence that has at least 71%, 72%, 73%, 74%, 75%, 76%, 77%, 78%, 79%, 80%, 81%, 82%, 83%, 84%, 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to at least one of SEQ ID NOs: 45-62; (2) has at least, no more than, or exactly 0, 1, 2, 3, 4, 5, 6, 7, 8, 9, or 10 additions, deletions, or substitutions to at least one of SEQ ID NOs: 45-62, 134-173, and 178-197; or (3) has both (1) and (2).

In some embodiments, the recombinant RNA molecule further comprises segments that encode other immunogens, such as those noted above. In some embodiments, the recombinant RNA molecule is in a composition that comprises a RNA molecule that encodes an immunogen other than a SARS-CoV-2 omicron strain-specific spike protein or an RNA molecule that does not encode at least one of SEQ ID NOs: 29, 30, and 123-133.

### 3′ Poly(Adenosine Monophosphate) Tail (3′ Poly(A) Tail)

In some embodiments, the recombinant RNA comprises a 3′ poly(adenosine monophosphate) (poly(A)) tail. In some embodiments, the 3′ poly(A) tail is 3′ from the 3′ UTR. In some embodiments, the 3′ poly(A)) tail at the 3′ end of the recombinant RNA.

In some embodiments, the poly(A) tail comprises, consists of, consists essentially of, has, or is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 adenosine monophosphate residues. In some embodiments, the poly(A) tail comprises, consists of, consists essentially of, has, or is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105,106, 107,108,109, 110, 111, 112, 113, 114, 115, 116, 117, 118, 119, or 120 consecutive adenosine monophosphate residues.

In some embodiments, the poly(A) tail comprises, consists of, consists essentially of, has, or is no more than: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113,114,115,116,117,118,119, or 120 adenosine monophosphate residues. In some embodiments, the poly(A) tail comprises, consists of, consists essentially of, has, or is no more than 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104,105,106,107,108,109,110,111,112, 113, 114, 115, 116, 117, 118, 119, or 120 consecutive adenosine monophosphate residues.

Taking the above-noted embodiments into account, it is contemplated and supported that any of the above-noted “adenosine monophosphate residues” of “at least” and any of the above-noted “adenosine monophosphate residues” of “no more than” may be combined to provide an enclosed range (i.e. the 3′ poly(A) tail comprising from 60 to 90 consecutive adenosine monophosphate residues).

In some embodiments, the poly(A) tail comprises, consists of, consists essentially of, has, or is from 20 to: 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 21 to: 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 22 to: 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 23 to: 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 24 to: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 25 to: 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 26 to: 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 27 to: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 28 to: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 29 to: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 30 to: 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 31 to: 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 32 to: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 33 to: 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 34 to: 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 35 to: 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 36 to: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 37 to: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 38 to: 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 39 to: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 40 to: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 41 to: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 42 to: 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 43 to: 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 44 to: 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 45 to: 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 46 to: 47,48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 47 to: 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 48 to: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 49 to: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 50 to: 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 51 to: 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 52 to: 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 53 to: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 54 to: 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 55 to: 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 56 to: 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 57 to: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 58 to: 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 59 to: 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 60 to: 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 61 to: 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 62 to: 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 63 to: 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 64 to: 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 65 to: 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 66 to: 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 67 to: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 68 to: 69% 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 69 to: 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 70 to: 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 71 to: 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 72 to: 73, 74, 75, 76, 77, 78, 79, 80, 81, 82, 83, 84, 85, 86, 87, 88, 89, 90, 91, 92, 93, 94, 95, 96, 97, 98, 99, or 100; from 73 to: 74, 75, 76, 77, 78, 79, 80, 81, 82, 83, 84, 85, 86, 87, 88, 89, 90, 91, 92, 93, 94, 95, 96, 97, 98, 99, or 100; from 74 to: 75, 76, 77, 78, 79, 80, 81, 82, 83, 84, 85, 86, 87, 88, 89, 90, 91, 92, 93, 94, 95, 96, 97, 98, 99, or 100; or from 75 to: 76, 77, 78, 79, 80, 81, 82, 83, 84, 85, 86, 87, 88, 89, 90, 91, 92, 93, 94, 95, 96, 97, 98, 99, or 100 adenosine monophosphate residues.

In some embodiments, the poly(A) tail comprises, consists of, consists essentially of, has, or is from 20 to: 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 21 to: 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 22 to: 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 23 to: 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 24 to: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 25 to: 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 26 to: 27, 28, 29, 30, 31, 32, 33, 34, 35, 36, 37, 38, 39, 40, 41, 42, 43, 44, 45, 46, 47, 48,49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 27 to: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 28 to: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 29 to: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 30 to: 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 31 to: 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 32 to: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 33 to: 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 34 to: 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 35 to: 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 36 to: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 37 to: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 38 to: 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 39 to: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 40 to: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 41 to: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 42 to: 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 43 to: 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 44 to: 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 45 to: 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 46 to: 47,48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 47 to: 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 48 to: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 49 to: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 50 to: 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 51 to: 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 52 to: 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 53 to: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 54 to: 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 55 to: 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 56 to: 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 57 to: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 58 to: 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 59 to: 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 60 to: 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 61 to: 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 62 to: 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 63 to: 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 64 to: 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 65 to: 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 66 to: 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 67 to: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 68 to: 69% 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 69 to: 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 70 to: 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 71 to: 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 72 to: 73, 74, 75, 76, 77, 78, 79, 80, 81, 82, 83, 84, 85, 86, 87, 88, 89, 90, 91, 92, 93, 94, 95, 96, 97, 98, 99, or 100; from 73 to: 74, 75, 76, 77, 78, 79, 80, 81, 82, 83, 84, 85, 86, 87, 88, 89, 90, 91, 92, 93, 94, 95, 96, 97, 98, 99, or 100; from 74 to: 75, 76, 77, 78, 79, 80, 81, 82, 83, 84, 85, 86, 87, 88, 89, 90, 91, 92, 93, 94, 95, 96, 97, 98, 99, or 100; or from 75 to: 76, 77, 78, 79, 80, 81, 82, 83, 84, 85, 86, 87, 88, 89, 90, 91, 92, 93, 94, 95, 96, 97, 98, 99, or 100 consecutive adenosine monophosphate residues.

Also contemplated and supported are combinations of “adenosine monophosphate residues” of “at least” and any of the above-noted “adenosine monophosphate residues” of “no more than” with the number of adenosine monophosphate residues provided in an Example of this document. Also contemplated and supported are combinations of the adenosine monophosphate residues of the Example section of this document to provide a range (i.e. the number of adenosine monophosphate residues from [the number of adenosine monophosphate residues in RNA X₁ in the Examples] to [the number of adenosine monophosphate residues to [the number of adenosine monophosphate residues in RNA X₂ in the Examples] wherein X₁ and X₂ represent any two exemplary RNA molecules of the Examples.

In some embodiments, the RNA comprises a 3′ segmented poly(A) tail comprising a first segment of consecutive adenosine monophosphate residues, a spacer, and a second segment of consecutive adenosine monophosphate residues. In some embodiments, the 3′ segmented poly(A) tail is ordered from 5′ to 3′ as the first segment of consecutive adenosine monophosphate residues, the spacer, and the second segment of consecutive adenosine monophosphate residues. In some embodiments, the 3′ segmented poly(A) tail is 3′ from the 3′ UTR. In some embodiments, the 3′ segmented poly(A)) tail at the 3′ end of the recombinant RNA.

In some embodiments, the first segment of consecutive adenosine monophosphate residues comprises, consists of, consists essentially of, has, or is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105,106,107, 108, 109, 110, 111, 112, 113, 114, 115, 116, 117, 118, 119, or 120 adenosine monophosphate residues.

In some embodiments, the first segment of consecutive adenosine monophosphate residues comprises, consists of, consists essentially of, has, or is no more than: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103,104,105,106,107,108, 109, 110, 111, 112, 113, 114, 115, 116, 117, 118, 119, or 120 adenosine monophosphate residues.

Taking the above-noted embodiments into account, it is contemplated and supported that any of the above-noted “adenosine monophosphate residues” of “at least” and any of the above-noted “adenosine monophosphate residues” of “no more than” may be combined to provide an enclosed range (i.e. the first segment of consecutive adenosine monophosphate residues comprising from 60 to 90 consecutive adenosine monophosphate residues).

In some embodiments, the first segment of consecutive adenosine monophosphate residues comprises, consists of, consists essentially of, has, or is from 20 to: 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 21 to: 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 22 to: 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 23 to: 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 24 to: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 25 to: 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 26 to: 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 27 to: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 28 to: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 29 to: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 30 to: 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 31 to: 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 32 to: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 33 to: 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 34 to: 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 35 to: 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 36 to: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 37 to: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 38 to: 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 39 to: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 40 to: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 41 to: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 42 to: 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 43 to: 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 44 to: 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 45 to: 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 46 to: 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 47 to: 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 48 to: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 49 to: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 50 to: 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 51 to: 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 52 to: 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 53 to: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 54 to: 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 55 to: 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 56 to: 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 57 to: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 58 to: 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 59 to: 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 60 to: 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 61 to: 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 62 to: 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 63 to: 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 64 to: 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 65 to: 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 66 to: 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 67 to: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 68 to: 69% 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 69 to: 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 70 to: 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 71 to: 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 72 to: 73, 74, 75, 76, 77, 78, 79, 80, 81, 82, 83, 84, 85, 86, 87, 88, 89, 90, 91, 92, 93, 94, 95, 96, 97, 98, 99, or 100; from 73 to: 74, 75, 76, 77, 78, 79, 80, 81, 82, 83, 84, 85, 86, 87, 88, 89, 90, 91, 92, 93, 94, 95, 96, 97, 98, 99, or 100; from 74 to: 75, 76, 77, 78, 79, 80, 81, 82, 83, 84, 85, 86, 87, 88, 89, 90, 91, 92, 93, 94, 95, 96, 97, 98, 99, or 100; or from 75 to: 76, 77, 78, 79, 80, 81, 82, 83, 84, 85, 86, 87, 88, 89, 90, 91, 92, 93, 94, 95, 96, 97, 98, 99, or 100 adenosine monophosphate residues.

Also contemplated and supported are combinations of “first segment of consecutive adenosine monophosphate residues” of “at least” and any of the above-noted “first segment of consecutive adenosine monophosphate residues” of “no more than” with the number of adenosine monophosphate residues of first segment of consecutive adenosine monophosphate residues provided in an Example of this document. Also contemplated and supported are combinations of the number of adenosine monophosphate residues of first segment of consecutive adenosine monophosphate residues of the Example section of this document to provide a range (i.e. the number of adenosine monophosphate residues of first segment of consecutive adenosine monophosphate residues of [the number of adenosine monophosphate residues of the first segment of consecutive adenosine monophosphate residues in RNA X₁ in the Examples] to [the number of adenosine monophosphate residues of the first segment of consecutive adenosine monophosphate residues in RNA X₂ in the Examples] wherein X, and X₂ represent any two exemplary RNA molecules of the Examples.

In some embodiments, the second segment of consecutive adenosine monophosphate residues comprises, consists of, consists essentially of, has, or is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103,104,105,106,107, 108, 109, 110, 111, 112, 113, 114, 115, 116, 117, 118, 119, or 120 adenosine monophosphate residues.

In some embodiments, the second segment of consecutive adenosine monophosphate residues comprises, consists of, consists essentially of, has, or is no more than: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104,105,106,107,108,109,110,111,112,113, 114, 115, 116, 117, 118, 119, or 120 adenosine monophosphate residues.

Taking the above-noted embodiments into account, it is contemplated and supported that any of the above-noted “adenosine monophosphate residues” of “at least” and any of the above-noted “adenosine monophosphate residues” of “no more than” may be combined to provide an enclosed range (i.e. the second segment of consecutive adenosine monophosphate residues comprising from 60 to 90 consecutive adenosine monophosphate residues).

In some embodiments, the second segment of consecutive adenosine monophosphate residues comprises, consists of, consists essentially of, has, or is from 20 to: 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 21 to: 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 22 to: 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 23 to: 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 24 to: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 25 to: 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 26 to: 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 27 to: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 28 to: 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 29 to: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 30 to: 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 31 to: 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 32 to: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 33 to: 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 34 to: 35, 36, 37, 38, 39,40, 41,42, 43, 44,45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 35 to: 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 36 to: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 37 to: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 38 to: 39,40, 41,42, 43, 44,45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 39 to: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 40 to: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 41 to: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 42 to: 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 43 to: 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 44 to: 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 45 to: 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 46 to: 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 47 to: 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 48 to: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 49 to: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 50 to: 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 51 to: 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 52 to: 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 53 to: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 54 to: 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 55 to: 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 56 to: 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 57 to: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 58 to: 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 59 to: 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 60 to: 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 61 to: 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 62 to: 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 63 to: 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 64 to: 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 65 to: 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 66 to: 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 67 to: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 68 to: 69% 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 69 to: 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 70 to: 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 71 to: 72, 73, 74, 75, 76, 77, 78, 79, 80, 81, 82, 83, 84, 85, 86, 87, 88, 89, 90, 91, 92, 93, 94, 95, 96, 97, 98, 99, or 100; from 72 to: 73, 74, 75, 76, 77, 78, 79, 80, 81, 82, 83, 84, 85, 86, 87, 88, 89, 90, 91, 92, 93, 94, 95, 96, 97, 98, 99, or 100; from 73 to: 74, 75, 76, 77, 78, 79, 80, 81, 82, 83, 84, 85, 86, 87, 88, 89, 90, 91, 92, 93, 94, 95, 96, 97, 98, 99, or 100; from 74 to: 75, 76, 77, 78, 79, 80, 81, 82, 83, 84, 85, 86, 87, 88, 89, 90, 91, 92, 93, 94, 95, 96, 97, 98, 99, or 100; or from 75 to: 76, 77, 78, 79, 80, 81, 82, 83, 84, 85, 86, 87, 88, 89, 90, 91, 92, 93, 94, 95, 96, 97, 98, 99, or 100 adenosine monophosphate residues.

### 5′ Capping

In some embodiments, the RNA further comprises a 5′ cap. In some embodiments, the RNA further comprises a 5′ nucleoside (e.g. a 7-methylguanosine or a modified 7-methylguanosine (e.g. 3′-methyl, 7′-methylguanosine (a 3′-O-me, 7-me-G))), a 5′ first ribonucleoside, an optional 5′ second ribonucleoside, and an optional tri-phosphate bridge. In some embodiments, the 5′ nucleoside (e.g. a 7-methylguanosine or a modified 7-methylguanosine (e.g. 3′-methyl, 7′-methylguanosine (a 3′-O-me, 7-me-G))) is linked directly or indirectly 5′-to-5′ to the 5′ first ribonucleoside. In some embodiments, the 5′ nucleoside (e.g. a 7-methylguanosine or a modified 7-methylguanosine (e.g. 3′-methyl, 7′-methylguanosine (a 3′-O-me, 7-me-G))) is linked 5′-to-5′ to the 5′ first ribonucleoside by the triphosphate bridge. In some embodiments, the 5′ first ribonucleoside comprises a 2′-methylated ribose (2′-O-Me) (i.e. a cap-1 or cap-2). In some embodiments, the 5′ second ribonucleoside is bound to the 3′ end of the 5′ first ribonucleoside. In some embodiments, the 5′ second ribonucleoside comprises a 2′-methylated ribose (2′-O-Me) (i.e. a cap-2). In some embodiments, the 5′ first ribonucleoside comprises a 2′-methylated ribose (2′-O-Me) and the 5′ second ribonucleoside comprises a 2′-methylated ribose (2′-O-Me) (i.e. a cap-2). A 5′ cap comprises a guanosine connected to the RNA via a 5′ to 5′ triphosphate linkage by mRNA guanylyltransferase, and wherein the guanine of said guanosine is methylated at its 7 position. In this context and in some embodiments, a 5′ to 5′ triphosphate linkage occurs when the 5′ end of the ribose of said guanosine is linked to the 5′ end of the ribose of the RNA via a triphophosphate group by mRNA guanylyltransferase. In some embodiments, thereafter, the guanine of said guanosine is methylated at its 7 position by (guanine-N7-)-methyltransferase. In some embodiments, the addition of the 7-methylguanosine 5′-to-5′ to the 5′ first ribonucleoside occurs at once, without addition of the 7-guanosine and further methylation thereof to obtain 7-methylguanosine (i.e. CLEANCAP®). In some embodiments, the addition of the 7-methylguanosine 5′-to-5′ to the 5′ first ribonucleoside and the addition of the 5′ first ribonucleoside comprising a 2′-methlyated ribose or 5′ second ribonucleoside comprising a 2′-methylated ribose occurs at once (i.e. CLEANCAP®). In some embodiments, the cap structure is preformed (i.e. as cap-1, cap-2, or cap-0, with or without the addition of the 7 methyl-group on the 5′ guanosine/7-methylguanosine) and added to the recombinant RNA molecule (i.e. by ligation). In some embodiments, the preformed cap structure is added with a 5′-AG-3′ initiating sequence as described in the CLEANCAP® AG product insert (Trilink catalog number N-7113), which is incorporated by reference).

Without further methylation, a 7-methylguanosine bound 5′-to-5′ to the 5′ first ribonucleoside is known as cap-0 and is expressed as 5′(m7Gp)(ppN)[pN]_{N}, wherein the former “N” indicates the first (5′) nucleobase of the RNA, the “pN” indicates a further nucleotide in the RNA, and the addition of “[..]_{N}” in “[pN]_{N}” indicates the repeating polymeric structure of the RNA and thereby collectively each sequentially adjacent nucleotide in the RNA.

An additional oxygen-linked methylation by a 2′-O-methyltransferase to the 2′ carbon of the ribose of the nucleoside of the RNA immediately adjacent to said 7-methylguanosine (i.e. the 5′ first ribonucleoside) results in a cap-1 structure, which is expressed as 5′(m7Gp)(ppm2N)[pN]_{N}, wherein the addition of the “m2” indicates the oxygen-linked methylation of the 2′ carbon of the ribose of the nucleoside immediately adjacent (via the triphosphate linkage) to said 7-methylguanosine. And further still, an additional methylation to the 5′ second ribonucleoside (i.e the next (3′) nucleoside immediately adjacent to the 5′ first nucleoside methylated in cap-1) results in a cap-2 structure, which is expressed as 5′(m7Gp)(ppm2N)(m2pN)[pN]n, wherein the addition of the latter “m2” indicates the methylation of the nucleotide immediately adjacent to the nucleotide methylated in cap-1. This cap-2 methylation is also to the 2′ carbon of the ribose of that immediately adjacent nucleotide (i.e. 2′-O-Me). In some embodiments, the 5′ cap is a cap-0, a cap-1, or a cap-2. In some embodiments, the 5′ cap is a cap-0. In some embodiments, the 5′ cap is a cap-1. In some embodiments, the 5′ cap is a cap-2.

In some embodiments, the 5′ first ribonucleoside or the 5′ second ribonucleoside is exogenously added to the RNA. In some embodiments, the 5′ first ribonucleoside or the 5′ second ribonucleoside is native to the RNA (i.e. if the native sequence is 5′-UUAAT-3′, then the addition of the m7Gp would result in 5′-m7Gp(ppUUAAT-3′ when there is a triphosphate bride; if the native sequence is the same, then the cap-1 structure would result in 5′-(m7Gp)(ppm2U)UAAT-3′ and the cap-2 structure being 5′-(m7Gp)(ppm2U)(m2U)AAT-3′).

Kits providing all of the materials for a 5′ cap, whether it is cap-1 or cap-2, and supplemental kits adding cap-1 and cap-2 capacity to a cap-0 kit can be used. The methods for 5′ capping can be carried out according to the manufacturer's instructions.

### Modified Nucleotides

In some embodiments, the percentage of uridines or thymidines substituted with a uridine-substitutable modified nucleotide or a thymidine-substitutable modified nucleotide are at least: 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In some embodiments, the percentage of uridines or thymidines substituted with a uridine-substitutable modified nucleotide or a thymidine-substitutable modified nucleotide are no more than: 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, or 15%. Taking the above-noted embodiments into account, it is contemplated and supported that any of the above-noted “%” of “at least” and any of the above-noted “%” of “no more than” may be combined to provide an enclosed range (i.e. the percentage of uridines or thymidines substituted with a uridine-substitutable modified nucleotide or a thymidine-substitutable modified nucleotide are from 25% to 75%). In some embodiments, the percentage of uridines or thymidines substituted with a uridine-substitutable modified nucleotide or a thymidine-substitutable modified nucleotide are from 20% to: 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 21% to: 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 22% to: 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 23% to: 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 24% to: 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 25% to: 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 26% to: 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 27% to: 28%,29%,30%,31%,32%,33%,34%,35%,36%,37%,38%,39%,40%,41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 28% to: 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 29% to: 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 30% to: 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 31% to: 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 32% to: 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 33% to: 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 34% to: 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 35% to: 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 36% to: 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 37% to: 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 38% to: 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 39% to: 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 40% to: 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 41% to: 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 42% to: 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 43% to: 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 44% to: 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 45% to: 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 46% to: 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 47% to: 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 48% to: 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 49% to: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 50% to: 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 51% to: 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 52% to: 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 53% to: 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 54% to: 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 55% to: 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 56% to: 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 57% to: 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 58% to: 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 59% to: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 60% to: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 61% to: 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 62% to: 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 63% to: 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 64% to: 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 65% to: 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 66% to: 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 67% to: 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 68% to: 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; or from 69% to 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Also contemplated and supported are combinations of the above-noted “%” of “at least” or “%” of “no more than” with the percent substitution provided in the Examples section of this document. Also contemplated and supported are combinations of the percent substitution of the Example section of this document to provide a range (i.e. the percent substitution from [the percent substitution in RNA X₁ in the Examples] to [a percent substitution from [the percent substitution in RNA X₂ in the Examples] wherein X₁ and X₂ represent any two exemplary RNA molecules of the Examples.

In some embodiments, the percentage of cytosines substituted with a cytosine-substitutable modified nucleotide are at least: 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In some embodiments, the percentage of cytosines substituted with a cytosine-substitutable modified nucleotide are no more than: 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, or 15%. Taking the above-noted embodiments into account, it is contemplated and supported that any of the above-noted “%” of “at least” and any of the above-noted “%” of “no more than” may be combined to provide an enclosed range (i.e. the percentage of cytosines substituted with a cytosine-substitutable modified nucleotide are from 25% to 75%). In some embodiments, the percentage of cytosines substituted with a cytosine-substitutable modified nucleotide are from 20% to: 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 21% to: 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 22% to: 23%,24%,25%,26%,27%,28%,29%,30%,31%,32%,33%,34%,35%,36%,37%,38%,39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 23% to: 24%, 25%, 26%,27%,28%,29%,30%,31%, 32%, 33%, 34%, 35%, 36%, 37%, 38%, 39%, 40%, 41%,42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 24% to: 25%, 26%, 27%, 28%, 29%, 30%, 31%,32%,33%,34%,35%,36%,37%,38%,39%,40%,41%,42%,43%,44%,45%,46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 25% to: 26%, 27%, 28%, 29%, 30%, 31%, 32%, 33%, 34%, 35%, 36%,37%,38%,39%,40%,41%, 42%, 43%, 44%, 45%, 46%, 47%, 48%, 49%, 50%, 51%,52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 26% to: 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 27% to: 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 28% to: 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 29% to: 30%, 31%, 32%, 33%, 34%, 35%,36%,37%,38%,39%,40%,41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 30% to: 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 31% to: 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 32% to: 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 33% to: 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 34% to: 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 35% to: 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 36% to: 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 37% to: 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 38% to: 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 39% to: 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 40% to: 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 41% to: 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 42% to: 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 43% to: 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 44% to: 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 45% to: 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 46% to: 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 47% to: 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 48% to: 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 49% to: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 50% to: 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 51% to: 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 52% to: 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 53% to: 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 54% to: 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 55% to: 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 56% to: 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 57% to: 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 58% to: 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 59% to: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 60% to: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 61% to: 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 62% to: 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 63% to: 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 64% to: 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 65% to: 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 66% to: 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 67% to: 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 68% to: 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; or from 69% to 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Also contemplated and supported are combinations of the above-noted “%” of “at least” or “%” of “no more than” with the percent substitution provided in the Examples section of this document. Also contemplated and supported are combinations of the percent substitution of the Example section of this document to provide a range (i.e. the percent substitution from [the percent substitution in RNA X₁ in the Examples] to [a percent substitution from [the percent substitution in RNA X₂ in the Examples] wherein X₁ and X₂ represent any two exemplary RNA molecules of the Examples.

In some embodiments, the percentage of guanosines substituted with a guanosine-substitutable modified nucleotide are at least: 10%, 11%, 12%,13%,14%,15%,16%,17%,18%,19%,20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In some embodiments, the percentage of guanosines substituted with a guanosine-substitutable modified nucleotide are no more than: 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%,36%,35%,34%,33%,32%,31%,30%,29%,28%,27%,26%,25%,24%,23%,22%,21%, 20%, 19%, 18%, 17%, 16%, or 15%. Taking the above-noted embodiments into account, it is contemplated and supported that any of the above-noted “%” of “at least” and any of the above-noted “%” of “no more than” may be combined to provide an enclosed range (i.e. the percentage of guanosines substituted with a guanosine-substitutable modified nucleotide are from 25% to 75%). In some embodiments, the percentage of guanosines substituted with a guanosine-substitutable modified nucleotide are from 20% to: 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 21% to: 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 22% to: 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 23% to: 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 24% to: 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 25% to: 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 26% to: 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 27% to: 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 28% to: 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 29% to: 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 30% to: 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 31% to: 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 32% to: 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 33% to: 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 34% to: 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 35% to: 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 36% to: 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 37% to: 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 38% to: 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 39% to: 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 40% to: 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 41% to: 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 42% to: 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 43% to: 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 44% to: 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 45% to: 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 46% to: 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 47% to: 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 48% to: 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 49% to: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 50% to: 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 51% to: 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 52% to: 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 53% to: 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 54% to: 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 55% to: 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 56% to: 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 57% to: 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 58% to: 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 59% to: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 60% to: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 61% to: 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 62% to: 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 63% to: 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 64% to: 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 65% to: 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 66% to: 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 67% to: 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 68% to: 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; or from 69% to 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Also contemplated and supported are combinations of the above-noted “%” of “at least” or “%” of “no more than” with the percent substitution provided in the Examples section of this document. Also contemplated and supported are combinations of the percent substitution of the Example section of this document to provide a range (i.e. the percent substitution from [the percent substitution in RNA X₁ in the Examples] to [a percent substitution from [the percent substitution in RNA X₂ in the Examples] wherein X₁ and X₂ represent any two exemplary RNA molecules of the Examples.

In some embodiments, the percentage of adenosines substituted with a adenosine-substitutable modified nucleotide are at least: 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In some embodiments, the percentage of adenosines substituted with a adenosine-substitutable modified nucleotide are no more than: 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, or 15%. Taking the above-noted embodiments into account, it is contemplated and supported that any of the above-noted “%” of “at least” and any of the above-noted “%” of “no more than” may be combined to provide an enclosed range (i.e. the percentage of adenosines substituted with a adenosine-substitutable modified nucleotide are from 25% to 75%). In some embodiments, the percentage of adenosines substituted with a adenosine-substitutable modified nucleotide are from 20% to: 21%, 22%, 23%, 24%, 25%, 26%, 27%, 28%, 29%, 30%, 31%, 32%,33%,34%,35%,36%,37%,38%,39%,40%,41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 21% to: 22%, 23%, 24%, 25%, 26%, 27%, 28%, 29%, 30%, 31%, 32%, 33%,34%,35%,36%,37%,38%,39%,40%,41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 22% to: 23%, 24%, 25%, 26%, 27%, 28%, 29%,30%,31%,32%,33%,34%,35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 23% to: 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 24% to: 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 25% to: 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 26% to: 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 27% to: 28%, 29%,30%,31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 28% to: 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 29% to: 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 30% to: 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 31% to: 32%, 33%, 34%, 35%, 36%,37%,38%,39%,40%,41%,42%,43%,44%,45%,46%,47%,48%,49%,50%,51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 32% to: 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 33% to: 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 34% to: 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 35% to: 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 36% to: 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 37% to: 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 38% to: 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 39% to: 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 40% to: 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 41% to: 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 42% to: 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 43% to: 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 44% to: 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 45% to: 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 46% to: 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 47% to: 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 48% to: 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 49% to: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 50% to: 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 51% to: 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 52% to: 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 53% to: 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 54% to: 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 55% to: 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 56% to: 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 57% to: 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 58% to: 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 59% to: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 60% to: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 61% to: 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 62% to: 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 63% to: 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 64% to: 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 65% to: 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 66% to: 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 67% to: 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; from 68% to: 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%; or from 69% to 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Also contemplated and supported are combinations of the above-noted “%” of “at least” or “%” of “no more than” with the percent substitution provided in the Examples section of this document. Also contemplated and supported are combinations of the percent substitution of the Example section of this document to provide a range (i.e. the percent substitution from [the percent substitution in RNA X₁ in the Examples] to [a percent substitution from [the percent substitution in RNA X₂ in the Examples] wherein X₁ and X₂ represent any two exemplary RNA molecules of the Examples.

In some embodiments, the modified nucleotides comprise: pseudouridine; N1-methylpseudouridine; N1-ethylpseudouridine; 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deazaadenosine; N1-methyl-adenosine; N6,N6 (dimethyl)adenine; N6-cis-hydroxy-isopentenyladenosine; .alpha.-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; .alpha.-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine: 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine: 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; .alpha.-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester, 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridine; 5-carboxymethylaminomethyluridine; 5-Carbamoylmethyluridine TP; 5-methoxycaeoonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; .alpha.-thio-uridine; 1 (aminoalkylamino-carbonylethylenyl)-2(thio)-pseudouridine; 1 (aminoalkylaminocarbonylethylenyl)-2,4-(dithio)pseudouridine; 1 (aminoalkylaminocarbonylethylenyl)-4 (thio)pseudouridine; 1 (aminoalkylaminocarbonylethylenyl)-pseudouridine; 1 (aminocazbonylethylenyl)-2(thio)-pseudouridine; 1 (aminocarbonylethylenyl)-2,4-(dithio)pseudouridine; 1 (aminocarbonylethylenyl)-4 (thio)pseudouridine; 1 (aminocarbonylethylenyl)-pseudouridine; 1 substituted 2(thio)-pseudouridine; 1 substituted 2,4-(dithio)pseudouridine; 1 substituted 4 (thio)pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouridine; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouridine; 4-(thio)pseudouridine; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouridine; 5-(alkyl)-2,4 (dithio)pseudouridine; 5-(alkyl)-4 (thio)pseudouridine; 5-(alkyl)pseudouridine; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouridine; 5-(methyl)-2,4 (dithio)pseudouridine; 5-(methyl)-4 (thio)pseudouridine; 5-(methyl)pseudouridine; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouridine; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (.+−.)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1-(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminoethoxy)-ethoxy]-propionyl}pseudouridine TP; 1-Acetylpseudouridine TP; I-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; or N6-(19-Amino-pentaoxanonadecyl)adenosine TP.

In some embodiments, the adenosine-substitutable modified nucleotides comprise: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deazaadenosine; N1-methyl-adenosine; N6,N6 (dimethyl)adenine; N6-cis-hydroxy-isopentenyl-adenosine; .alpha.-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; or N6-(19-Amino-pentaoxanonadecyl)adenosine TP.

In some embodiments, the uridine-substitutable modified nucleotides or the thymidine-substitutable modified nucleotides comprise: pseudouridine; N1-methylpseudouridine; N1-ethylpseudouridine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester, 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridine; 5-carboxymethylaminomethyluridine; 5-Carbamoylmethyluridine TP; 5-methoxycaeoonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; .alpha.-thio-uridine; 1 (aminoalkylamino-carbonylethylenyl)-2(thio)-pseudouridine; 1 (aminoalkylaminocarbonylethylenyl)-2,4-(dithio)pseudouridine; 1 (aminoalkylaminocarbonylethylenyl)-4 (thio)pseudouridine; 1 (aminoalkylaminocarbonylethylenyl)-pseudouridine; 1 (aminocazbonylethylenyl)-2(thio)-pseudouridine; 1 (aminocarbonylethylenyl)-2,4-(dithio)pseudouridine; 1 (aminocarbonylethylenyl)-4 (thio)pseudouridine; 1 (aminocarbonylethylenyl)-pseudouridine; 1 substituted 2(thio)-pseudouridine; 1 substituted 2,4-(dithio)pseudouridine; 1 substituted 4 (thio)pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouridine; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouridine; 4-(thio)pseudouridine; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouridine; 5-(alkyl)-2,4 (dithio)pseudouridine; 5-(alkyl)-4 (thio)pseudouridine; 5-(alkyl)pseudouridine; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouridine; 5-(methyl)-2,4 (dithio)pseudouridine; 5-(methyl)-4 (thio)pseudouridine; 5-(methyl)pseudouridine; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouridine; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (.+−.)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1-(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminoethoxy)-ethoxy]-propionyl}pseudouridine TP; 1-Acetylpseudouridine TP; I-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-cytidine TP; 2′-OH-ara-uridine TP TP; or 5-(2-carbomethoxyvinyl)uridine TP.

In some embodiments, the cytosine-substitutable modified nucleotides comprise 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; .alpha.-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine: 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine: 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 2′fluoro-cytidine; or 2′-OH-ara-cytidine TP.

In some embodiments, the modified nucleotides comprise: 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; .alpha.-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; or 2′-OH-ara-guanosine TP.

### Lipid Carriers

As noted above, LNPs are listed as adjuvants, but in this regard, they also provide another function that is, in some embodiments, co-extensive with, and in some embodiments, independent of adjuvanticity. That is, RNA, by itself and unprotected, may be degraded by the subject's RNAases. LNPs provide a means to protect the RNA by encapsulating or comprising within them an amount of the RNA of the overall composition or formulation. The LNP's effects as being in some cases an adjuvant, in other cases a delivery vehicle, and in other cases both, can be cell-dependent. To illustrate but without being bound by a particular theory, the LNP may provide adjuvanticity to peripheral blood mononuclear cells in that the RNA activates the cells but may not be expressed therein, but the LNP may provide a delivery vehicle, but not adjuvanticity, to other somatic cell types, for arguendo example, skeletal muscle cells.

In some embodiments, the pharmaceutically acceptable delivery vehicle comprises a lipid nanoparticle (LNP). In some embodiments, the LNPs encapsulate, comprise within them, consist within them, consist essentially within them, or have within them at least: 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% of the RNA. In some embodiments, the LNPs encapsulate, comprise within them, consist within them, consist essentially within them, or have within them no more than: 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% of the RNA. In some embodiments, the LNPs encapsulate, comprise within them, consist within them, consist essentially within them, or have within them from 85% to: 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 86% to: 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 87% to 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 88% to: 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 89% to: 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 90% to: 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 91% to: 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 92% to: 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 93% to: 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 94% to: 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 95% to: 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 96% to: 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 97.0% to: 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 97.5% to: 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 98.0% to: 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 98.5% to: 98.6%, 98.7%, 98.8%, 98.9%, 99.0%, 99.1%, 99.2%, 99.3%, 99.4%, 99.5%, 99.6%, 99.7%, 99.8%, 99.9%, or 100%; or from: 99.0% to: 99.1%, 99.2%, 99.3%, 99.4%, 99.5%, 99.6%, 99.7%, 99.8%, 99.9%, or 100% of the RNA. In this regard, the foregoing percentages reference the number of RNA molecules encapsulated or comprised within the LNPs compared to the total number of RNA molecules in the composition and not the percentage of the length of any one RNA molecule being within and outside of the LNP. Also contemplated and supported are combinations of the above-noted percentages of “at least” and “no more than” with the percentages of encapsulation provided in the Examples section of this document. Also contemplated and supported are combinations of the percentages of encapsulation of this document to provide a range (i.e. the LNPs encapsulate, comprise within them, consist within them, consist essentially within them, or have within them from [the percent encapsulation of LNP formulation X₁ in the Examples] to [the percent encapsulation of LNP formulation X₂ in the Examples] wherein X₁ and X₂ represent any two exemplary LNP formulations of the Examples).

In some embodiments, 80% of the LNPs have a diameter of at least: 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm. In some embodiments, 80% of the LNPs have a diameter of no more than: 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

In some embodiments, the LNPs have a diameter of at least: 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm. In some embodiments, LNPs have a diameter of no more than: 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

In some embodiments, 80% of the LNPs have a diameter from 20 nm to: 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 25 nm to: 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 30 nm to: 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 35 nm to: 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from: 40 nm to: 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 45 nm to: 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 50 nm to: 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 55 nm to: 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 60 nm to: 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 65 nm to: 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 70 nm to: 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 75 nm to: 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 80 nm to: 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; or from 85 nm to: 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

In some embodiments, the LNPs have a diameter from 20 nm to: 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 25 nm to: 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 30 nm to: 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 35 nm to: 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from: 40 nm to: 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 45 nm to: 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 50 nm to: 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 55 nm to: 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 60 nm to: 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 65 nm to: 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 70 nm to: 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 75 nm to: 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 80 nm to: 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; or from 85 nm to: 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

Taking the above-noted embodiments into account, it is contemplated and supported that any of the above-noted “diameters of no more than” and “diameters of at least” may be combined to provide an enclosed range (i.e. 80% of the LNP have a diameter from 50 nm to 80 nm). Also contemplated and supported are combinations of the above-noted “diameter no more than” or “diameter of at least” with the diameters of the LNP formulations provided in the Examples section of this document. Also contemplated and supported are combinations of the diameters of the LNP formulation section of this document to provide a range (i.e. a cation-ionizable lipid having a diameter of from [the diameter of LNP formulation X₁ in the Examples] to [the LNP formulation X₂ in the Examples] wherein X₁ and X₂ represent any two exemplary LNP formulations of the Examples.

In some embodiments, LNP has a pKa of at least: 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10. In embodiments, the LNP has a pKa of no more than: 10, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, or 5.0. In some embodiments, LNP has a pKa from 5.0 to: 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.1 to: 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.3 to: 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.4 to: 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.5 to: 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.6 to: 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.7 to: 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from: 5.8 to: 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.9 to: 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.0 to: 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.1 to: 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from: 6.2 to: 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.3 to: 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.4 to: 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.5 to: 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.6 to: 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.7 to: 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.8 to: 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.9 to: 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.0 to: 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.1 to: 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.2 to: 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.3 to: 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.4 to: 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.5 to: 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.6 to: 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.7 to: 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.9 to: 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.0 to: 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.1 to: 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.2 to: 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.3 to: 8.4, 8.5, 8.6, 8.7, 8.8, 8.9, 9.0, 9.1, 9.2, 9.3, 9.4, 9.5, 9.6, 9.7, 9.8, 9.9, or 10; or from 8.4 to: 8.5, 8.6, 8.7, 8.8, 8.9, 9.0, 9.1, 9.2, 9.3, 9.4, 9.5, 9.6, 9.7, 9.8, 9.9, or 10. Taking the above-noted embodiments into account, it is contemplated and supported that any of the above-noted “pKa of at least” and “pKa of no more than” may be combined to provide an enclosed range (i.e. a pKa of from 5.0 to 7.6 or a pKa of from 5.5 to 7.0, etc.). Also contemplated and supported are combinations of the above-noted “pKa of at least” or “pKa of no more than” with the pKa of an LNP formulation provided in the Examples section of this document. Also contemplated and supported are combinations of the pKa of the Examples section of this document to provide a range (i.e. a LNP having a pKa of from [the pKa of LNP formulation X₁ in the Examples] to [the pKa of LNP formulation X₂ in the Examples] wherein X₁ and X₂ represent any two exemplary LNP formulations of the Examples. Such embodiments are distinguishable (and combinable) with embodiments wherein the first lipid or cation-ionizable lipid has a pKa by itself. In this regard, “the cation-ionizable lipid having a pKa” and “the LNP having a pKa” are distinguishable. The pKa of the LNP is influenced by the selection of cation-ionizable lipids and the mole percentages of the individual lipids in the overall formulation (i.e. the mole percentage of sterol, the mole percentage of a particular second lipid, the mole percentage of the particular first lipid) but to be clear the pKa of the LNP excludes the influence of the recombinant RNA molecules. That is, the term “pKa of the LNP” is defined to be measured from an LNP consisting of the lipids of the LNP formulation that comprises the lipids and the recombinant RNA molecules (i.e. a blank LNP wherein “blank” excludes recombinant RNA molecules and any other molecules that would be delivered by the LNP). The “pKa of the LNP” is measured by the following 6-(p-toluidino)-2-naphthalenesulfonic acid (TNS) assay. TNS was suspended at 300 μM in DMSO. Following Zhang et al. “Ionization behavior of amino lipids for siRNA delivery: determination of ionization constants, SAR, and the impact of lipid pKa on cationic lipid-biomembrane interactions, LANGMUIR 27: 1907-1914 (2001), the LNPs were admixed with the 300 μM TNS in DMSO and buffered solutions containing 20 mM boric acid, 10 mM imidazole, 10 mM sodium acetate, 10 mM glycylclycine, and 25 mM NaCl and an individual pH from 3 to 10, such that each final admixture had 75 μM of cation-ionizable lipids and 6 μM of TNS and the series of admixtures provided at least one admixture within each of one pH unit within the range of pH from 3 to 10 (i.e. an admixture with said buffer having a pH within 3 (i.e. from 3.0 to 3.99), an admixture with said buffer having a pH within 4 (i.e. from 4.0 to 4.99), an admixture with said buffer having a pH within 5 (i.e. from 5.0 to 5.99), an admixture with said buffer having a pH within 6 (i.e. from 6.0 to 6.99), an admixture with said buffer having a pH within 7 (i.e. from 7.0 to 7.99), an admixture with said buffer having a pH within 8 (i.e. from 8.0 to 8.99), and an admixture with said buffer having a pH within 9 (i.e. from 9.0 to 9.99)). The fluorescence of said mixtures are then determined with an excitation wavelength of 321 nm and an emission wavelength of 445 nm. The blank-subtracted fluorescence of each admixture is then determined by subtracting a blank solution's fluorescence from each admixture's emission fluorescence. The relative fluoresence is then determined by dividing the blank-subtracted fluorescence of each admixture from the relative fluorescence of the admixture that has the highest relative fluorescence. The relative fluorescences for all the admixtures versus the pHs of the respective buffers were then regressed following the Henderson-Hasselbalch equation to obtain a line of best fit. The pKa of the LNP was determined from the pH value that has a relative fluorescence of 0.5 on the line of best fit.

### First Lipids

In some embodiments, the LNP comprises lipids comprising: a first lipid (i.e. a cation-ionizable lipid), an optional sterol (e.g. cholesterol), an optional polymer-conjugated lipid, and an optional second lipid (i.e. an optional anionic lipid or an optional neutral lipid, including zwitterionic lipids). In some embodiments, the optional neutral lipid comprises a neutral lipid zwitterionic lipid. In some embodiments, the polymer-conjugated lipid comprises a polyethylene glycol-conjugated lipid. In some embodiments, the LNP comprises a lipid from WO2012/006376, WO2012/030901, WO2012/031046, WO2012/031043, WO2012/006378, WO2011/076807, WO2013/033563, WO2013/006825, WO2014/136086, WO2015/095340, WO2015/095346, WO2016/037053, WO2017/075531, WO2018/081480, WO2015/074085, WO2018/1703322, U.S. Patent Application Publication Nos.: 20220081392, 20220072155, 20220040285, 20210395188, 20210251898, 20210128488, 20210122703, 20210122702, 20210107861, 20200283372, 20200172472, 20200163878, 20200121809,20200046838,20190359556,20190314524,20190274968,20190270697, 20190022247, 20180185516, 20170283367,20170157268,20170119904,20160376224, 20160317676, or 20150376115, U.S. Application Nos. 61/905,724 or Ser. No. 15/614,499, or U.S. Pat. Nos. 8,802,863, 9,458,090, 9,593,077, 9,567,296, 9,604,908, 9,643,916, 9,669,097, 9,670,487, 9,737,619, 9,738,593, 9,725,720, 9,796,977,10,106,490, 10,166,298, 10,221,127, 10,723,692, 11,040,112, 11,168,051, or 11,285,222 (including ionizable and PEG-lipids, as referred to therein).

In some embodiments, the first lipid is a cation-ionizable lipid. In some embodiments, the cation-ionizable lipid has a pKa of at least: 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10. In embodiments, the cationic-ionizable lipid has a pKa of no more than: 10, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, or 5.0. In some embodiments, the cation-ionizable lipid has a pKa from 5.0 to: 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.1 to: 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.3 to: 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.4 to: 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.5 to: 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.6 to: 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.7 to: 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from: 5.8 to: 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.9 to: 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.0 to: 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.1 to: 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from: 6.2 to: 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.3 to: 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.4 to: 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.5 to: 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.6 to: 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.7 to: 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.8 to: 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.9 to: 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.0 to: 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.1 to: 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.2 to: 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.3 to: 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.4 to: 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.5 to: 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.6 to: 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.7 to: 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.9 to: 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.0 to: 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.1 to: 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.2 to: 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.3 to: 8.4, 8.5, 8.6, 8.7, 8.8, 8.9, 9.0, 9.1, 9.2, 9.3, 9.4, 9.5, 9.6, 9.7, 9.8, 9.9, or 10; or from 8.4 to: 8.5, 8.6, 8.7, 8.8, 8.9, 9.0, 9.1, 9.2, 9.3, 9.4, 9.5, 9.6, 9.7, 9.8, 9.9, or 10. Taking the above-noted embodiments into account, it is contemplated and supported that any of the above-noted “pKa of at least” and “pKa of no more than” may be combined to provide an enclosed range (i.e. a pKa of from 5.0 to 7.6 or a pKa of from 5.5 to 7.0, etc.). Also contemplated and supported are combinations of the above-noted “pKa of at least” or “pKa of no more than” with the pKa of a lipid provided in the Examples section of this document. Also contemplated and supported are combinations of the pKa of the lipids section of this document to provide a range (i.e. a cation-ionizable lipid having a pKa of from [the pKa of lipid X₁ in the Examples] to [the pKa of lipid X₂ in the Examples] wherein X₁ and X₂ represent any two exemplary lipids of the Examples. Such embodiments are distinguishable (and combinable) with embodiments wherein the LNP has a pKa as a whole (sometimes called “apparent pKa”). In this regard, “the cation-ionizable lipid having a pKa” and “the LNP having a pKa” are distinguishable. Within the meaning of “the cation-ionizable lipid having a pKa,” the pKa and the values of the cation-ionizable lipid is determined by (or the value is determined to fall within a pKa range of from value X to value Y, and thereby the values of X and Y are determined by):
1) admixing 400 μL of 2 mM of the first lipid that are in 100 volume % ethanol and 800 μL of 0.3 mM of fluorescent probe toluene nitrosulphonic acid (TNS), which is in 90 volume % ethanol and 10 volume % methanol, thereby obtaining a lipid/TNS mixture;
2) admixing 7.5 μL of the lipid/TNS mixture and 242.5 μL of a first buffer comprising a sodium salt buffer comprising 20 mM sodium phosphate, 25 mM sodium citrate, 20 mM sodium acetate, and 150 mM sodium chloride, wherein the first buffer has a first pH from 4.44 to 4.52, thereby obtaining a first mixture, and dispensing 100 μL of the first mixture in a first well of a 96-well plate, which has a clear bottom;
3) admixing 7.5 μL of the lipid/TNS mixture and 242.5 μL of a second buffer comprising the sodium salt buffer, wherein the second buffer has a second pH of 5.27, thereby obtaining a second mixture, and dispensing 100 μL of the second mixture in a second well of the 96-well plate;
4) admixing 7.5 μL of the lipid/TNS mixture and 242.5 μL of a third buffer comprising the sodium salt buffer, wherein the third buffer has a third pH of from 6.15 to 6.21, thereby obtaining a third mixture, and dispensing 100 μL of the third mixture in a third well of the 96-well plate;
5) admixing 7.5 μL of the lipid/TNS mixture and 242.5 μL of a fourth buffer comprising the sodium salt buffer, wherein the fourth buffer has a fourth pH of 6.57, thereby obtaining a fourth mixture, and dispensing 100 μL of the fourth mixture in a fourth well of the 96-well plate;
6) admixing 7.5 μL of the lipid/TNS mixture and 242.5 μL of a fifth buffer comprising the sodium salt buffer, wherein the fifth buffer has a fifth pH of from 7.10 to 7.20, thereby obtaining a fifth mixture, and dispensing 100 μL of the fifth mixture in a fifth well of the 96-well plate;
7) admixing 7.5 μL of the lipid/TNS mixture and 242.5 μL of a sixth comprising the sodium salt buffer, wherein the sixth buffer has a sixth pH of from 7.72 to 7.80, thereby obtaining a sixth mixture, and dispensing 100 μL of the sixth mixture in a sixth well of the 96-well plate;
8) admixing 7.5 μL of the lipid/TNS mixture and 242.5 μL of a seventh buffer comprising the sodium salt buffer, wherein the seventh buffer has a seventh pH of from 8.27 to 8.33, thereby obtaining a seventh mixture, and dispensing 100 μL of the seventh mixture in a seventh well of the 96-well plate;
9) admixing 7.5 μL of the lipid/TNS mixture and 242.5 μL of an eighth buffer comprising the sodium salt buffer, wherein the eighth buffer has a eighth pH of from 10.47 to 11.12, thereby obtaining an eighth mixture, and dispensing 100 μL of the eighth mixture in an eighth well of the 96-well plate;
10) measuring the absolute fluorescence at a wavelength of 431 nm with an excitation wavelength of 322 nm and a cut-off below 420 nm of each of the first through eighth wells and an empty well of the 96-well plate;
11) subtracting the absolute fluorescence of the empty well from each of the absolute fluorescences of the first through the eighth wells, thereby obtaining a blank-subtracted fluorescence for each of the first through eighth mixtures;
12) normalizing each of the blank-subtracted fluorescences of the first through eighth mixtures to the blank-subtracted fluorescence of the first mixture, thereby obtaining a relative fluorescence for each of the first through eighth mixtures, the relative fluorescence of the first mixture being 1;
13) regressing by the Henderson-Hasselbalch equation, the first through eighth pHs versus the respective relative fluorescences of the first through eighth mixtures thereby obtaining a line of best fit; and
14) determining the pKa as the pH at which a relative fluorescence of 0.5 is obtained on the line of best fit. (i.e. A line of best fit is a sigmoidal curve with four parameters.)

In some embodiments, the cation-ionizable lipid comprises an amine that can be a tertiary amine and become charged depending upon the pH of the solution that the cation-ionizable lipid is in when compared to the pKa of the cation-ionizable lipid. In some embodiments, at least half of the cation-ionizable lipids are neutrally charged and the amine is a tertiary amine when the pH of the solvent that the cation-ionizable lipids are in is above the pKa; and at least half of the cation-ionizable lipids are positively charged when the pH of the solvent that the cation-ionizable lipids are in is below the pKa. In this regard, in some embodiments, but without being limited to a particular theory, it is considered that the positive charge of the ionizable lipid is distributed on the (tertiary) amine. Since the amine can vary in charge depending upon the pH of the solution relative to the pKa of the cation-ionizable lipid and since, without being bound a particular theory, the amine can be neutrally or positively charged when tertiary, in some embodiments, the amine is an ionizable amine.

The cation-ionizable lipid will be further described when the amine is tertiary and when the cation-ionizable lipid is neutrally charged. That is, the lipid being in a tertiary amine state and having a neutral charged is hereby described, without having to describe the cation-ionizable lipid when the lipid becomes charged. In some embodiments, the cation-ionizable lipid further comprises, in addition to the above-noted ionizable amine, a headgroup (RH) and a fatty acid tail (R^{FA1} or R^{FA2}). In some embodiments, the cation-ionizable lipid further comprises (in addition to the above-noted ionizable amine) a headgroup and at least two fatty acid tails (R^{FA1} and R^{FA2}), such as in Formula I.

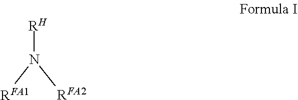

In some embodiments, the amine provides a branchpoint between the headgroup and a fatty acid tail. In some embodiments, the fatty acid tail (R^{FA}) or the at least two fatty acid tails (i.e. R^{FA1}R^{FA2} . . . ) are immediately off of the ionizable amine. In some embodiments, the fatty acid tail comprises, or the at least two fatty acid tails comprise, a biodegradeable group (i.e. R^{BD1} or R^{BD2}), and the at least two fatty acid tails are the same or independent of one another. In some embodiments, the at least two fatty acid tails each comprise a biodegradeable group, such as in Formula II, and the biodegradeable groups are the same or independent of one another. In some embodiments, the biodegradeable group comprises, in order from the ionizable amine, —O(C=O)—, —(C=O)O—, —C(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a}C(═O)O—, wherein X is 0, 1, or 2, and wherein R^{a} is a hydrogen or a C₁-C₁₂ alkyl. In some embodiments, the fatty acid comprises, or the at least two fatty acids comprise, a C₁-C₁₂ alkyl, a C₁-C₁₂ alkylene, or a C₁-C₁₂ alkenylene (i.e. R^{FC1} and R^{FC2}) between the amine branchpoint and the biodegradeable group), such as in Formula II. In some embodiments, the fatty acid comprises, or the two or more fatty acids comprise, distal to the ionizable amine and the biodegradeable group, a C₅-C₂₄ alkyl, a C₅-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈ alkyl, a C₁₂-C₁₈ alkylene, a C₁₃-C₁₇ alkyl, or a C₁₃-C₁₇ alkylene (i.e. R^{FC3} and R^{FC4}), such as in Formula II.

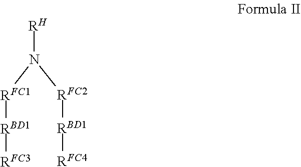

Wherein: R^{FC1} and R^{FC2} are each independently a C₁-C₁₂ alkyl, a C₁-C₁₂ alkylene, or a C₁-C₁₂ alkenylene;
R^{FC3} and R^{FC4} are each independently: a C₆-C₂₄ alkyl, a C₆-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈ alkyl, a C₁₂-C₁₈ alkylene, a C₁₃-C₁₇ alkyl, a C₁₃-C₁₇ alkylene;
R^{BD1} and R^{BD2} are each independently: —O(C=O)—, —(C=O)O—, —C(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a}C(═O)O—; wherein X is 0, 1, or 2, and wherein R^{a} is a hydrogen or a C₁-C₁₂ alkyl.

In some embodiments, the C₆-C₂₄ alkyl or the C₆-C₂₄ alkylene is connected to the biodegradeable group at C₆-C₁₂, C₇-C₁₁, C₅-C₁₀, or C₉ thereof. In some embodiments, the C₆-C₂₄ alkyl or the C₆-C₂₄ alkylene of each of the at least two fatty acid tails independently comprises:

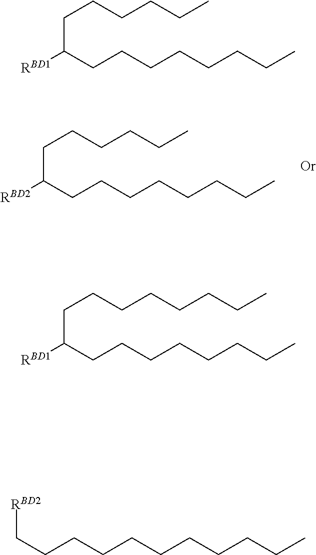

In some embodiments, the headgroup comprises, consists of, is, or has a first group (i.e. R^{H1}) and a second group (i.e. R^{H2}), wherein the first group is a C₁-C₂₄ alkyl, C₁-C₂₄ alkylene, a C₁-C₂₄ alkenylene, a C₃-C₈ cycloalkylene, or a C₃-C₈ cycloalkenylene, and the second group is H, —OH, CN, —C(═O)OR⁴, —OC(═O)OR⁴, —NR⁵C(═O)OR⁴, or OR⁵; wherein R⁴ is C₁-C₁₂ alkyl and R⁵ is H or C₁-C₆ alkyl. In some embodiments, the headgroup comprises a linear or branched form of: —(CH₂)₆OH, —(CH₂)₅OH, —(CH₂)OH, —(CH₂)₃OH, —(CH₂)₂OH, or —CH₂OH. In some embodiments, the cation-ionizable lipid comprises, consists of, or is 9-Heptadecanyl 8-{(2-hydroxyethyl)[6-oxo-6-(undecyloxy)hexyl]amino}octanoate.

In some embodiments, the cation-ionizable lipid is:

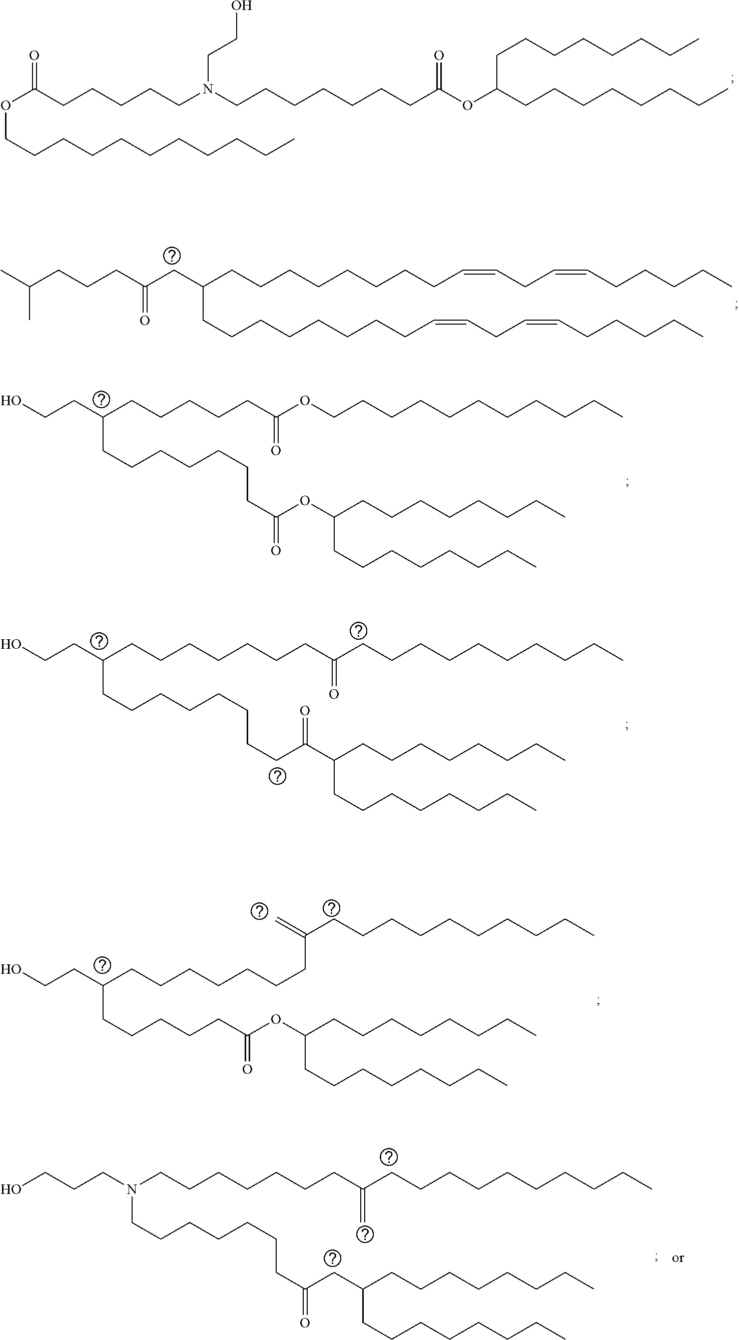

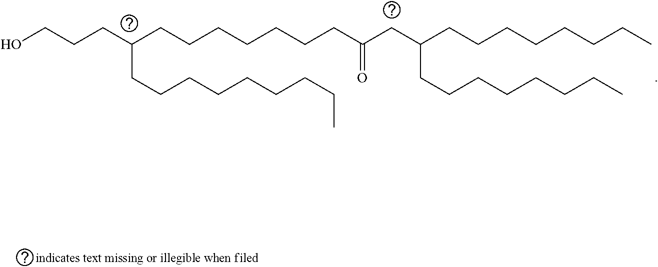 [SMILES: CCCCCCCCCC(CCCO)CCCCCCCC(=O)CC(CCCCCCCC)CCCCCCCC]

In an embodiment, the cation-ionizable lipid comprises, consists of, consists essentially of, or is RV28 having the following structure:

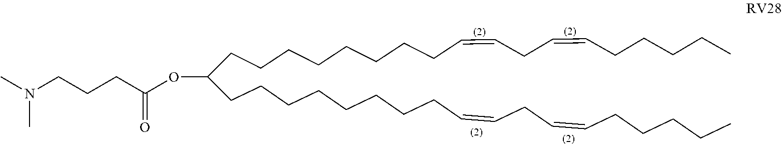 [SMILES: CCCCC/C=C\C/C=C\CCCCCCCCC(CCCCCCCC/C=C\C/C=C\CCCCC)OC(=O)CCCN(C)C]

In an embodiment, the cation-ionizable lipid comprises, consists of, consists essentially of, or is RV31 having the following structure:

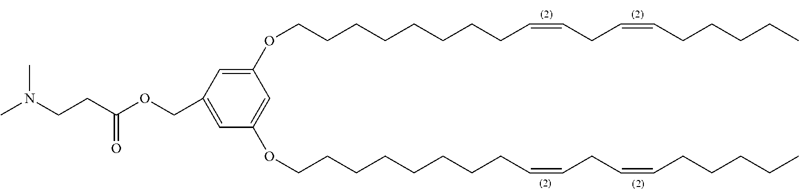 [SMILES: CCCCC/C=C\C/C=C\CCCCCCCCOc1cc(COC(=O)CCN(C)C)cc(OCCCCCCCC/C=C\C/C=C\CCCCC)c1]

In an embodiment, the cation-ionizable lipid comprises, consists of, consists essentially of, or is RV33 having the following structure:

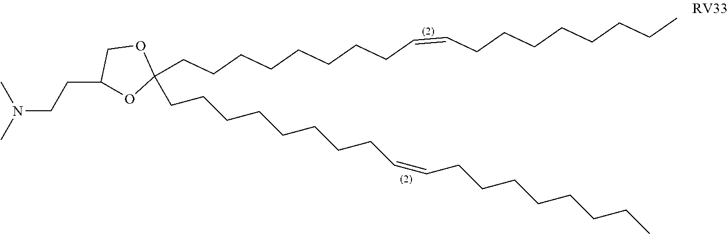 [SMILES: CCCCCCCC/C=C\CCCCCCCCC1(CCCCCCCC/C=C\CCCCCCCC)OCC(CCN(C)C)O1]

In an embodiment, the cation-ionizable lipid comprises, consists of, consists essentially of, or is RV37 having the following structure:

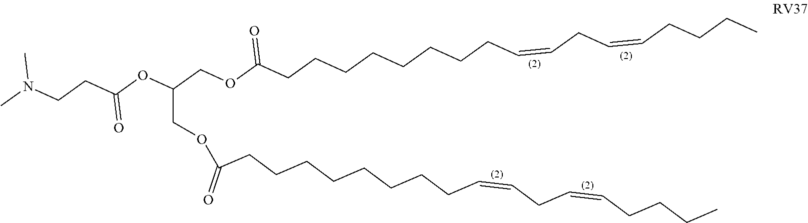 [SMILES: CCCC/C=C\C/C=C\CCCCCCCCC(=O)OCC(COC(=O)CCCCCCCC/C=C\C/C=C\CCCC)OC(=O)CCN(C)C]

In an embodiment, the cation-ionizable lipid comprises, consists of, consists essentially of, or is RV39, i.e., 2,5-bis((9Z,12Z)-octadeca-9,12-dien-1-yloxy)benzyl 4-(dimethylamino)butanoate):

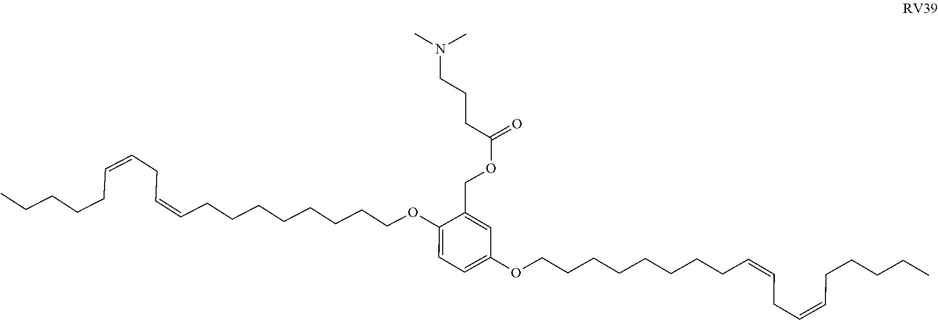 [SMILES: CCCCC/C=C\C/C=C\CCCCCCCCOc1ccc(OCCCCCCCC/C=C\C/C=C\CCCCC)c(COC(=O)CCCN(C)C)c1]

In an embodiment, the cation-ionizable lipid comprises, consists of, consists essentially of, or is RV42 having the following structure:

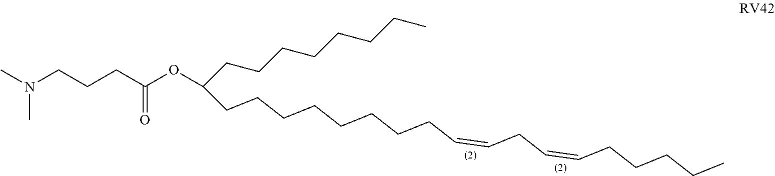 [SMILES: CCCCC/C=C\C/C=C\CCCCCCCCC(CCCCCCCC)OC(=O)CCCN(C)C]

In an embodiment, the cation-ionizable lipid comprises, consists of, consists essentially of, or is RV44 having the following structure:

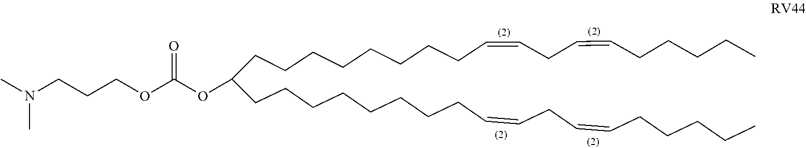 [SMILES: CCCCC/C=C\C/C=C\CCCCCCCCC(CCCCCCCC/C=C\C/C=C\CCCCC)OC(=O)OCCCN(C)C]

In an embodiment, the cation-ionizable lipid comprises, consists of, consists essentially of, or is RV73 having the following structure:

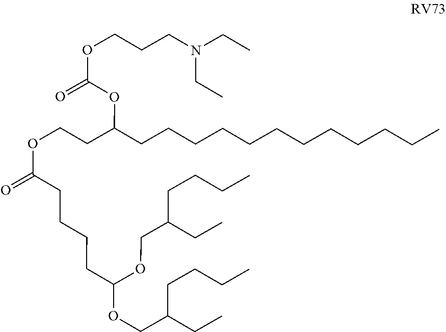 [SMILES: CCCCCCCCCCCCC(CCOC(=O)CCCCC(OCC(CC)CCCC)OCC(CC)CCCC)OC(=O)OCCCN(CC)CC]

In an embodiment, the cation-ionizable lipid comprises, consists of, consists essentially of, or is RV75 having the following structure:

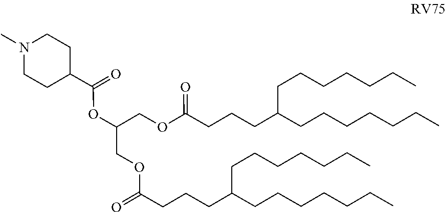 [SMILES: CCCCCCCC(CCCCCCC)CCCC(=O)OCC(COC(=O)CCCC(CCCCCCC)CCCCCCC)OC(=O)C1CCN(C)CC1]

In an embodiment, the cation-ionizable lipid comprises, consists of, consists essentially of, or is RV81 having the following structure:

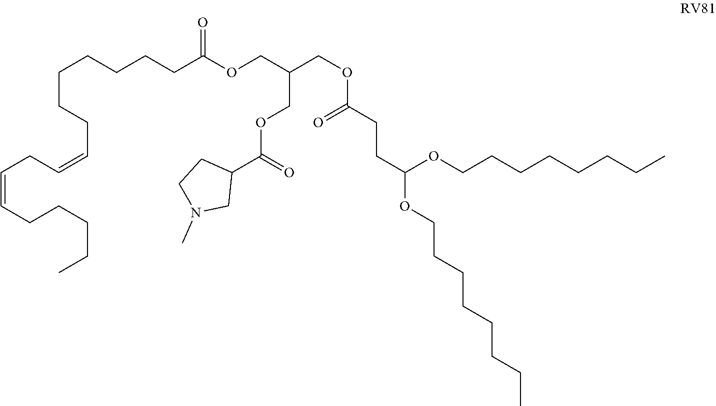 [SMILES: CCCCC/C=C\C/C=C\CCCCCCCC(=O)OCC(COC(=O)CCC(OCCCCCCCC)OCCCCCCCC)COC(=O)C1CCN(C)C1]

In an embodiment, the cation-ionizable lipid comprises, consists of, consists essentially of, or is RV84 having the following structure:

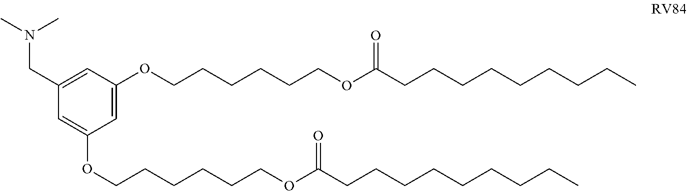 [SMILES: CCCCCCCCCC(=O)OCCCCCCOc1cc(CN(C)C)cc(OCCCCCCOC(=O)CCCCCCCCC)c1]

In an embodiment, the cation-ionizable lipid comprises, consists of, consists essentially of, or is RV85 having the following structure:

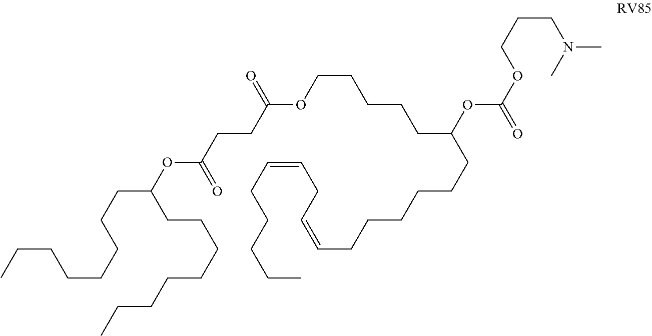 [SMILES: CCCCC/C=C\C/C=C\CCCCCCC(CCCCCOC(=O)CCC(=O)OC(CCCCCCCC)CCCCCCCC)OC(=O)OCCCN(C)C]

In an embodiment, the cation-ionizable lipid comprises, consists of, consists essentially of, or is RV86 having the following structure:

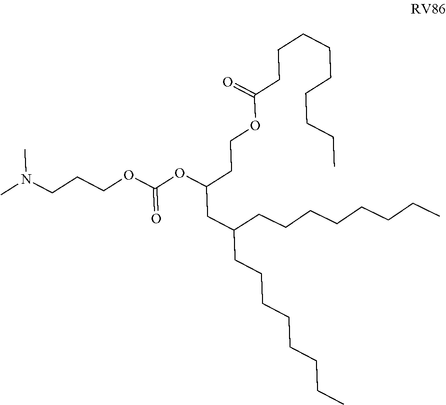 [SMILES: CCCCCCCCCC(=O)OCCC(CC(CCCCCCCC)CCCCCCCC)OC(=O)OCCCN(C)C]

In an embodiment, the cation-ionizable lipid comprises, consists of, consists essentially of, or is RV88 having the following structure:

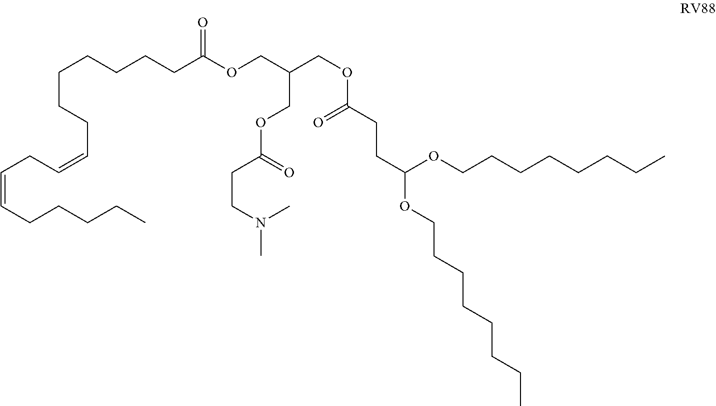 [SMILES: CCCCC/C=C\C/C=C\CCCCCCCC(=O)OCC(COC(=O)CCC(OCCCCCCCC)OCCCCCCCC)COC(=O)CCN(C)C]

In an embodiment, the cation-ionizable lipid comprises, consists of, consists essentially of, or is RV91 having the following structure:

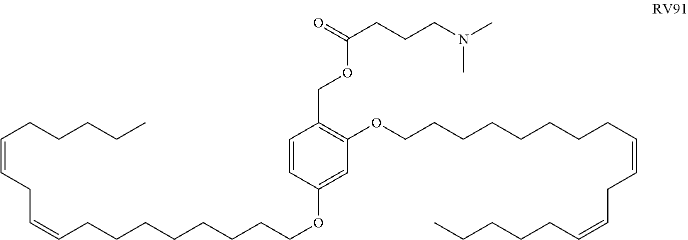 [SMILES: CCCCC/C=C\C/C=C\CCCCCCCCOc1ccc(COC(=O)CCCN(C)C)c(OCCCCCCCC/C=C\C/C=C\CCCCC)c1]

In an embodiment, the cation-ionizable lipid comprises, consists of, consists essentially of, or is RV92 having the following structure:

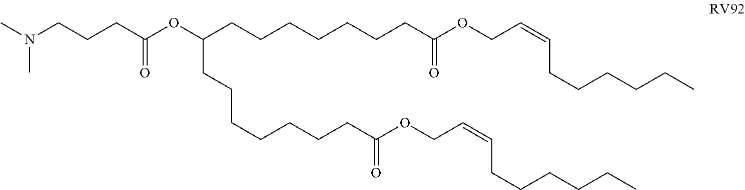 [SMILES: CCCCCC/C=C\COC(=O)CCCCCCCC(CCCCCCCC(=O)OC/C=C\CCCCCC)OC(=O)CCCN(C)C]

In an embodiment, the cation-ionizable lipid comprises, consists of, consists essentially of, or is RV93 having the following structure:

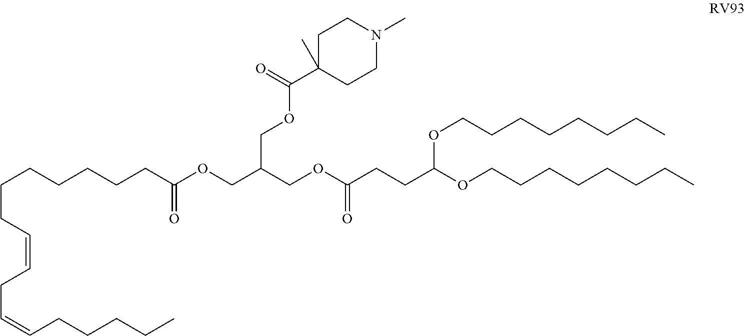 [SMILES: CCCCC/C=C\C/C=C\CCCCCCCC(=O)OCC(COC(=O)CCC(OCCCCCCCC)OCCCCCCCC)COC(=O)C1(C)CCN(C)CC1]

In an embodiment, the cation-ionizable lipid comprises, consists of, consists essentially of, or is 2-(5-((4-((1,4-dimethylpiperidine-4-carbonyl)oxy)hexadecyl)oxy)-5-oxopentyl)propane-1,3-diyl dioctanoate (RV94), having the following structure:

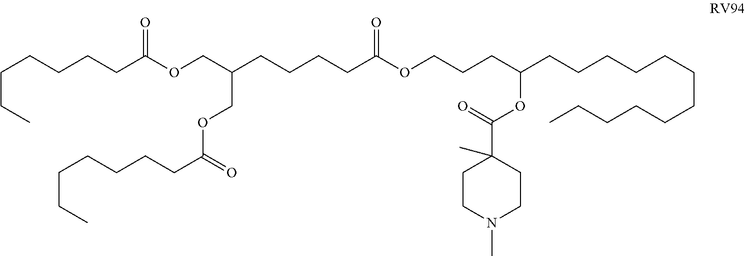 [SMILES: CCCCCCCCCCCCC(CCCOC(=O)CCCCC(COC(=O)CCCCCCC)COC(=O)CCCCCCC)OC(=O)C1(C)CCN(C)CC1]

In an embodiment, the cation-ionizable lipid comprises, consists of, consists essentially of, or is RV95 having the following structure:

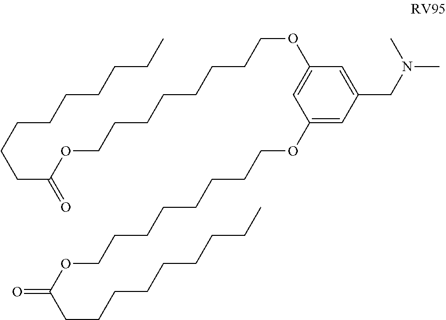 [SMILES: CCCCCCCCCC(=O)OCCCCCCCCOc1cc(CN(C)C)cc(OCCCCCCCCOC(=O)CCCCCCCCC)c1]

In an embodiment, the cation-ionizable lipid comprises, consists of, consists essentially of, or is RV96 having the following structure:

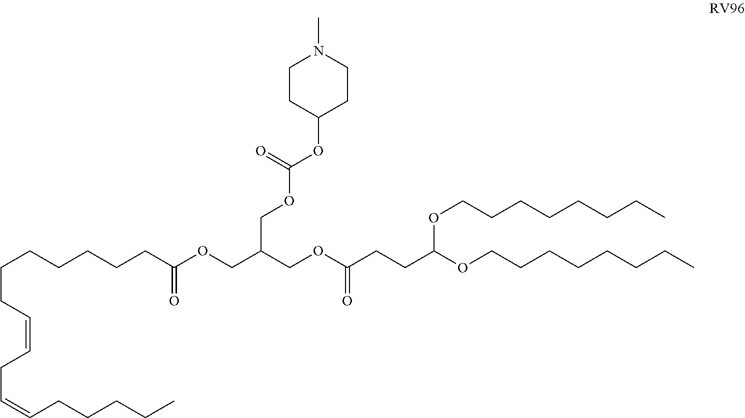 [SMILES: CCCCC/C=C\C/C=C\CCCCCCCC(=O)OCC(COC(=O)CCC(OCCCCCCCC)OCCCCCCCC)COC(=O)OC1CCN(C)CC1]

In an embodiment, the cation-ionizable lipid comprises, consists of, consists essentially of, or is RV97 having the following structure:

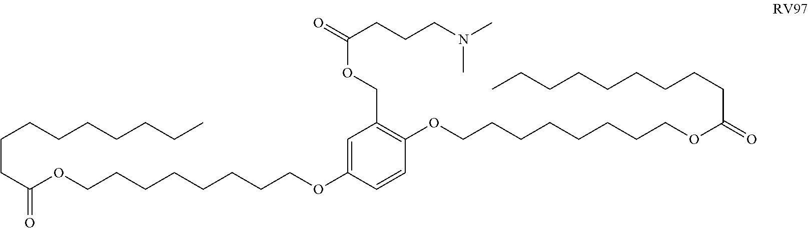 [SMILES: CCCCCCCCCC(=O)OCCCCCCCCOc1ccc(OCCCCCCCCOC(=O)CCCCCCCCC)c(COC(=O)CCCN(C)C)c1]

In an embodiment, the cation-ionizable lipid comprises, consists of, consists essentially of, or is RV99 having the following structure:

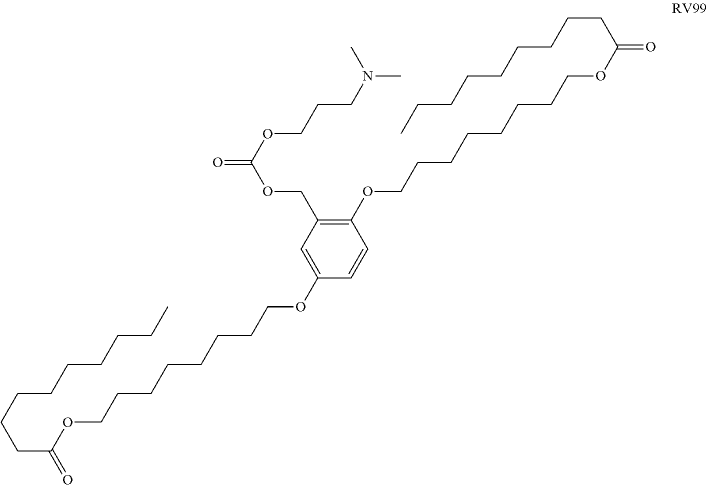 [SMILES: CCCCCCCCCC(=O)OCCCCCCCCOc1ccc(OCCCCCCCCOC(=O)CCCCCCCCC)c(COC(=O)OCCCN(C)C)c1]

In an embodiment, the cation-ionizable lipid comprises, consists of, consists essentially of, or is RV101 having the following structure:

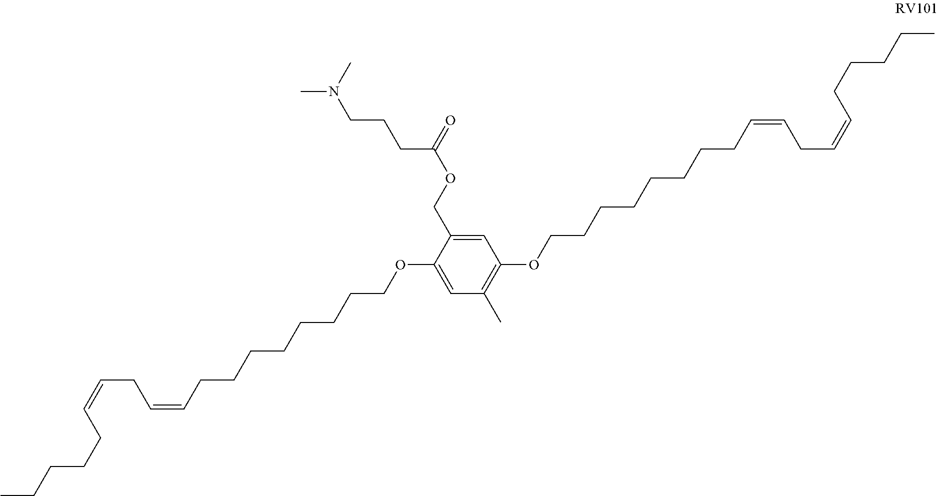 [SMILES: CCCCC/C=C\C/C=C\CCCCCCCCOc1cc(COC(=O)CCCN(C)C)c(OCCCCCCCC/C=C\C/C=C\CCCCC)cc1C]

In some embodiments, the cation-ionizable lipid comprise, consist of, consist essentially of, or are a lipid having the structure of Formula III:

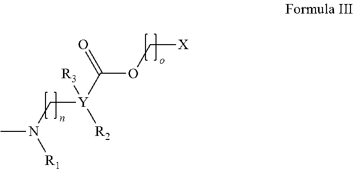

wherein n=an integer from 1 to 3 and
(i) R₁ is CH₃, R₂ and R₃ are both H, and Y is C; or
(ii) R₁and R₂ are collectively CH₂—CH₂ and together with the nitrogen form a five-, six-, or seven-membered heterocycloalkyl, R₃ is CH₃, and Y is C; or
(iii) R₁ is CH₃, R₂ and R₃ are both absent, and Y is O;
wherein o is 0 or 1;
wherein X is:
(i)

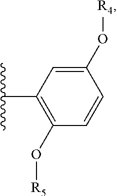

wherein R₄ and R₅ are independently a C₁₀₋₂₀ hydrocarbon chain having one or two cis alkene groups at either or both of the omega 6 and 9 positions; or
(ii) —CH(—R₆)—R₇, wherein
(1) R₆ is —(CH₂)p-O—C(O)—R₈ or —Cₚ—R₈;
(2) R₇ is —(CH₂)_{p′}—O—C(O)—R₈′ or —C_{p′}—R₈′,
(3) p and p′ are independently 0, 1, 2, 3 or 4; and
(4) R₈ and R_{8′} are independently a
(A) —C₈₋₂₀ hydrocarbon chain having one or two cis alkene groups at either or both of the omega 6 and 9 positions;
(B) —C₁₋₃—C(—O—C₆₋₁₂)—O—C₆₋₁₂ saturated or unsaturated hydrocarbon chain;
(C) —C₆₋₁₆ saturated hydrocarbon chain;
(D) —C(—C₆₋₁₆)—C₆₋₁₆ saturated or unsaturated hydrocarbon chain;
(E) —C[—C—O—C(O)—C₄₋₁₂]—C—O—C(O)—C₄₋₁₂ saturated or unsaturated hydrocarbon chain; and
(F) —C₆₋₁₆ saturated or unsaturated hydrocarbon chain.

In an embodiment, R₁ is CH₃, R₂ and R₃ are both H, and Y is C. In some embodiments, R₁ and R₂ are collectively CH₂—CH₂ and together with the nitrogen form a five-, six-, or seven-membered heterocycloalkyl, R₃ is CH₃, and Y is C. In some embodiments, R₁ is CH₃, R₂ and R₃ are both absent, and Y is O.

In an embodiment, X is

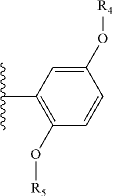

wherein R₄ and R₅ are independently a C₁₀₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇—C_{p′}—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)_{p′}—O—C(O)—R₈, R₇ is —C_{p′}—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C(—C₆₋₁₆)—C₆16 saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is C_{p′}—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a-C₁₋₃—C(—O—C₆₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₁₂ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; R₈ is a-C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C. 3-C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇—C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈″, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In some embodiments, the cation-ionizable lipid comprises a cationic lipid from WO2012/006376, WO2012/030901, WO2012/031046, WO2012/031043, WO2012/006378, WO2011/076807, WO2013/033563, WO2013/006825, WO2014/136086, WO2015/095340, WO2015/095346, WO2016/037053, WO2017/075531, WO2018/081480, WO2015/074085, WO2018/1703322, U.S. Patent Application Publication Nos.: 20220081392, 20220072155, 20220040285, 20210395188, 20210251898, 20210128488, 20210122703, 20210122702, 20210107861, 20200283372, 20200172472, 20200163878, 20200121809, 20200046838, 20190359556, 20190314524, 20190274968, 20190270697, 20190022247, 20180185516, 20170283367, 20170157268, 20170119904, 20160376224, 20160317676, or 20150376115, U.S. Application Nos. 61/905,724 or Ser. No. 15/614,499, or U.S. Pat. Nos. 8,802,863, 9,458,090, 9,593,077, 9,567,296, 9,604,908, 9,643,916, 9,669,097, 9,670,487, 9,737,619, 9,738,593, 9,725,720, 9,796,977,10,106,490, 10,166,298, 10,221,127, 10,723,692, 11,040,112, 11,168,051, 11,246,933, or 11,285,222 (called ionizable lipids therein).

In some embodiments, the cation-ionizable lipid comprises a first group and two biodegradable hydrophobic tails. In some embodiments, the first group comprises a central moiety and a head group, wherein the first group is capable of being positively charged. In some embodiments, the central moiety is directly bonded to each of the two biodegradable groups. In some embodiments, the central moiety is directly bonded to the head group. In some embodiments, the central moiety is selected from a central carbon atom, a central nitrogen atom, a central heteroaryl group, and a central heterocyclic group. In some embodiments, one of the two biodegradable hydrophobic tails, or each of the two biodegradable hydrophobic tails, has the formula of: -(a C₁-C₁₂ alkyl, a C₁-C₁₂ alkylene, or a C₁-C₁₂ alkenylene)-(the biodegradable group)-(a C₆-C₂₄ alkyl, a C₆-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈alkyl, a C₁₂-C₁₈ alkylene, a C₁₃-C₁₇ alkyl, a C₁₃-C₁₇ alkylene). In some embodiments, the each biodegradable group in the two biodegradable hydrophobic tails is independently selected from: —O(C=O)—, —(C=O)O—, —C(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a}C(═O)O—; wherein X is 0, 1, or 2, and wherein R^{a} is a hydrogen or a C₁-C₁₂ alkyl. In some embodiments, in one of the two biodegradable tails, or each of the two biodegradable tails: 1) has a terminal hydrophobic chain, which is a branched alkyl group, and a terminus, 2) the branching of the branched alkyl group has an alpha-position relative to the biodegradable group, 3) 6 to 12 carbon atoms of the biodegradable hydrophobic tail separate the terminus from the biodegradable group.

In some embodiments, the cation-ionizable lipid comprises bis(2-methacryloyl)oxyethyl disulfide (DSDMA, CAS No. 36837-97-5), N,N-dioleyl-N,N-dimethylammonium chloride (DODAC), N,N-distearyl-N,N-dimethylammonium bromide (DDAB), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N, N-dimethylaminopropane (DLenDMA), 1,2-di-y-linolenyloxy-N,N-dimethylaminopropane (y-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)b-utanoate (MC3), ALNY-100 ((3aR,5s,6aS)—N,N-dimethyl-2,2-di((9Z,12Z)-octadeca-9,12-dienyl)tetrahydr-o-3aH-cyclopenta[d][1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N1,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethy- lpropan-1-amine (MC3 Ether), 4-((6Z,9Z,28Z,31Z)-heptatriaconta-6,9,28,31-tetraen-1, 9-yloxy)-N,N-dimethy-lbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethyl-ammonium trifluoroacetate (DOSPA), (DOPE), from GIBCO/BRL); or TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US Patent Application Publication No. 20150140070A1).

Representative cation-ionizable lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl[1,3]-dioxolane (DLin-K-DMA), 2,2-dilinoleyl-4-(2-dimethylaminoethyl)[1,3]-dioxolane (DLin-KC2-DMA); dilinoleyl-methyl-4-dimethylaminobutyrate (DLin-MC3-DMA); MC3 (US Patent Publication No. 20100324120).

In some embodiments, the LNP can comprise multilamellar vesicles (MLV); small uniflagellar vesicles (SUV); or large unilamellar vesicles (LUV). MLVs have multiple bilayers in each vesicle, forming several separate aqueous compartments. SUVs and LUVs have a single bilayer encapsulating an aqueous core. For compositions comprising LNPs with different diameters, in some embodiments: (i) at least 80% by number should have diameters in the range of 20-220 nm, (ii) the average (median) diameter (Zav, by intensity) of the population is ideally in the range of 40-200 nm, or (iii) the diameters should have a polydispersity index <0.2.

Various amphiphilic lipids can form bilayers in an aqueous environment to encapsulate a RNA-containing aqueous core as a LNP. These lipids can have an anionic, cationic, or zwitterionic hydrophilic head group. Some phospholipids are anionic whereas other are zwitterionic and others are cationic. Suitable classes of phospholipid include, but are not limited to, phosphatidylethanolamines, phosphatidylcholines, phosphatidylserines, and phosphatidyl-glycerols, and some useful phospholipids are listed in Table 1. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), and 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are 1,2-dipalmitoyl-sn-glycero-3-phosphocholine (DPPC), 1,2-dioleoyl-sn-glycero-3-phosphocholine (DOPC), and dodecylphosphocholine. The lipids can be saturated or unsaturated. The use of at least one unsaturated lipid for preparing liposomes is preferred. If an unsaturated lipid has two tails, both tails can be unsaturated, or it can have one saturated tail and one unsaturated tail.

Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

### Polyethylene Glycol-Conjugated Lipids

In some embodiments, the LNP comprises a polyethylene glycol-conjugated (PEG-conjugated) lipid. In some embodiments, the PEG-conjugated lipid comprises a polyethylene glycol (PEG) having various lengths and molecular weights. In some embodiments, 80% of the PEGs in the PEG-conjugated lipids have a molecular weight from: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa. In some embodiments, 80% of the PEGs in the PEG-conjugated lipids have a molecular weight to: 8 kDa, 7.9 kDa, 7.8 kDa, 7.7 kDa, 7.6 kDa, 7.5 kDa, 7.4 kDa, 7.3 kDa, 7.2 kDa, 7.1 kDa, 7 kDa, 6.9 kDa, 6.8 kDa, 6.7 kDa, 6.6 kDa, 6.5 kDa, 6.4 kDa, 6.3 kDa, 6.2 kDa, 6.1 kDa, 6 kDa, 5.9 kDa, 5.8 kDa, 5.7 kDa, 5.6 kDa, 5.5 kDa, 5.4 kDa, 5.3 kDa, 5.2 kDa, 5.1 kDa, 5 kDa, 4.9 kDa, 4.8 kDa, 4.7 kDa, 4.6 kDa, 4.5 kDa, 4.4 kDa, 4.3 kDa, 4.2 kDa, 4.1 kDa, 4 kDa, 3.9 kDa, 3.8 kDa, 3.7 kDa, 3.6 kDa, 3.5 kDa, 3.4 kDa, 3.3 kDa, 3.2 kDa, 3.1 kDa, 3 kDa, 2.9 kDa, 2.8 kDa, 2.7 kDa, 2.6 kDa, 2.5 kDa, 2.4 kDa, 2.3 kDa, 2.2 kDa, 2.1 kDa, 2 kDa, 1.9 kDa, 1.8 kDa, 1.7 kDa, 1.6 kDa, 1.5 kDa, 1.4 kDa, 1.3 kDa, 1.2 kDa, or 1.1 kDa. In some embodiments, 80% of the PEGs in the PEG-conjugated lipids have a molecular weight from 0.5 kDa to: 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.6 kDa to: 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.7 kDa to: 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.8 kDa to: 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.9 kDa to: 0.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.0 kDa to: 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.1 kDa to: 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.2 kDa to: 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.3 kDa to: 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.4 kDa to: 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.5 kDa to: 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.6 kDa to: 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.7 kDa to: 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.8 kDa to: 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.9 kDa to: 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.0 kDa to: 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.1 kDa to: 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.2 kDa to: 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.3 kDa to: 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.5 to: 2.6 kDa, 2.7 kDa, 2.8 kDa, 2.9 kDa, 3.0 kDa, 3.1 kDa, 3.2 kDa, 3.3 kDa, or 3.4 kDa; from 2.6 kDa to: 2.7 kDa, 2.8 kDa, 2.9 kDa, 3.0 kDa, 3.1 kDa, 3.2 kDa, 3.3 kDa, or 3.4 kDa; from 2.7 kDa to: 2.8 kDa, 2.9 kDa, 3.0 kDa, 3.1 kDa, 3.2 kDa, 3.3 kDa, or 3.4 kDa; from 2.8 kDa to: 2.9 kDa, 3.0 kDa, 3.1 kDa, 3.2 kDa, 3.3 kDa, or 3.4 kDa; from 2.9 kDa to: 3.0 kDa, 3.1 kDa, 3.2 kDa, 3.3 kDa, or 3.4 kDa; or from 3.0 kDa to: 3.1 kDa, 3.2 kDa, 3.3 kDa, or 3.4 kDa.

In some embodiments, the PEGs in the PEG-conjugated lipids have a molecular weight from: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa. In some embodiments, the PEGs in the PEG-conjugated lipids have a molecular weight to: 8 kDa, 7.9 kDa, 7.8 kDa, 7.7 kDa, 7.6 kDa, 7.5 kDa, 7.4 kDa, 7.3 kDa, 7.2 kDa, 7.1 kDa, 7 kDa, 6.9 kDa, 6.8 kDa, 6.7 kDa, 6.6 kDa, 6.5 kDa, 6.4 kDa, 6.3 kDa, 6.2 kDa, 6.1 kDa, 6 kDa, 5.9 kDa, 5.8 kDa, 5.7 kDa, 5.6 kDa, 5.5 kDa, 5.4 kDa, 5.3 kDa, 5.2 kDa, 5.1 kDa, 5 kDa, 4.9 kDa, 4.8 kDa, 4.7 kDa, 4.6 kDa, 4.5 kDa, 4.4 kDa, 4.3 kDa, 4.2 kDa, 4.1 kDa, 4 kDa, 3.9 kDa, 3.8 kDa, 3.7 kDa, 3.6 kDa, 3.5 kDa, 3.4 kDa, 3.3 kDa, 3.2 kDa, 3.1 kDa, 3 kDa, 2.9 kDa, 2.8 kDa, 2.7 kDa, 2.6 kDa, 2.5 kDa, 2.4 kDa, 2.3 kDa, 2.2 kDa, 2.1 kDa, 2 kDa, 1.9 kDa, 1.8 kDa, 1.7 kDa, 1.6 kDa, 1.5 kDa, 1.4 kDa, 1.3 kDa, 1.2 kDa, or 1.1 kDa. In some embodiments, the PEGs in the PEG-conjugated lipids have a molecular weight from 0.5 kDa to: 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.6 kDa to: 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.7 kDa to: 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.8 kDa to: 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.9 kDa to: 0.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.0 kDa to: 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.1 kDa to: 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.2 kDa to: 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.3 kDa to: 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.4 kDa to: 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.5 kDa to: 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.6 kDa to: 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.7 kDa to: 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.8 kDa to: 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.9 kDa to: 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.0 kDa to: 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.1 kDa to: 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.2 kDa to: 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.3 kDa to: 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.5 to: 2.6 kDa, 2.7 kDa, 2.8 kDa, 2.9 kDa, 3.0 kDa, 3.1 kDa, 3.2 kDa, 3.3 kDa, or 3.4 kDa; from 2.6 kDa to: 2.7 kDa, 2.8 kDa, 2.9 kDa, 3.0 kDa, 3.1 kDa, 3.2 kDa, 3.3 kDa, or 3.4 kDa; from 2.7 kDa to: 2.8 kDa, 2.9 kDa, 3.0 kDa, 3.1 kDa, 3.2 kDa, 3.3 kDa, or 3.4 kDa; from 2.8 kDa to: 2.9 kDa, 3.0 kDa, 3.1 kDa, 3.2 kDa, 3.3 kDa, or 3.4 kDa; from 2.9 kDa to: 3.0 kDa, 3.1 kDa, 3.2 kDa, 3.3 kDa, or 3.4 kDa; or from 3.0 kDa to: 3.1 kDa, 3.2 kDa, 3.3 kDa, or 3.4 kDa. Taking the above-noted embodiments into account, it is contemplated and supported that any of the above-noted “molecular weight from” and “molecular weight to” may be combined to provide an enclosed range (i.e. a molecular weight from 1.1 kDa to 2.4 kDa). Also contemplated and supported are combinations of the above-noted “molecular weight from” or “molecular weight to” with the molecular weight of the PEG provided in the Examples section of this document to provide an enclosed range. Also contemplated and supported are combinations of the molecular weight of the Example section of this document to provide a range (i.e. PEG having a molecular weight of from [the PEG's molecular weight of lipid X₁ in the Examples] to [the PEG's molecular weight of lipid X₂ in the Examples] wherein X₁ and X₂ represent any two exemplary PEG-conjugated lipids of the Examples.

In some embodiments, the PEGs in the PEG-conjugated lipids have a median molecular weight (i.e. number-averaged molecular weight) from: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, 3.4 kDa, 3.5 kDa, 3.6 kDa, 3.7 kDa, 3.8 kDa, 3.9 kDa, 4.0 kDa, 4.1 kDa, 4.2 kDa, 4.3 kDa, 4.4 kDa, 4.5 kDa, 4.6 kDa, 4.7 kDa, 4.8 kDa, 4.9 kDa, 5.0 kDa, 5.1 kDa, 5.2 kDa, 5.3 kDa, 5.4 kDa, 5.5 kDa, 5.6 kDa, 5.7 kDa, 5.8 kDa, 5.9 kDa, or 6.0 kDa. In some embodiments, PEGs in the PEG-conjugated lipids have a median molecular weight (i.e. number-averaged molecular weight) to: 8 kDa, 7.9 kDa, 7.8 kDa, 7.7 kDa, 7.6 kDa, 7.5 kDa, 7.4 kDa, 7.3 kDa, 7.2 kDa, 7.1 kDa, 7 kDa, 6.9 kDa, 6.8 kDa, 6.7 kDa, 6.6 kDa, 6.5 kDa, 6.4 kDa, 6.3 kDa, 6.2 kDa, 6.1 kDa, 6 kDa, 5.9 kDa, 5.8 kDa, 5.7 kDa, 5.6 kDa, 5.5 kDa, 5.4 kDa, 5.3 kDa, 5.2 kDa, 5.1 kDa, 5 kDa, 4.9 kDa, 4.8 kDa, 4.7 kDa, 4.6 kDa, 4.5 kDa, 4.4 kDa, 4.3 kDa, 4.2 kDa, 4.1 kDa, 4 kDa, 3.9 kDa, 3.8 kDa, 3.7 kDa, 3.6 kDa, 3.5 kDa, 3.4 kDa, 3.3 kDa, 3.2 kDa, 3.1 kDa, 3 kDa, 2.9 kDa, 2.8 kDa, 2.7 kDa, 2.6 kDa, 2.5 kDa, 2.4 kDa, 2.3 kDa, 2.2 kDa, 2.1 kDa, 2 kDa, 1.9 kDa, 1.8 kDa, 1.7 kDa, 1.6 kDa, 1.5 kDa, 1.4 kDa, 1.3 kDa, 1.2 kDa, or 1.1 kDa. In some embodiments, the PEGs in the PEG-conjugated lipids have a median molecular weight (i.e. number-averaged molecular weight) from 0.5 kDa to: 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.6 kDa to: 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.7 kDa to: 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.8 kDa to: 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.9 kDa to: 0.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.0 kDa to: 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.1 kDa to: 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.2 kDa to: 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.3 kDa to: 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.4 kDa to: 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.5 kDa to: 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.6 kDa to: 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.7 kDa to: 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.8 kDa to: 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.9 kDa to: 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.0 kDa to: 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.1 kDa to: 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.2 kDa to: 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.3 kDa to: 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.5 to: 2.6 kDa, 2.7 kDa, 2.8 kDa, 2.9 kDa, 3.0 kDa, 3.1 kDa, 3.2 kDa, 3.3 kDa, or 3.4 kDa; from 2.6 kDa to: 2.7 kDa, 2.8 kDa, 2.9 kDa, 3.0 kDa, 3.1 kDa, 3.2 kDa, 3.3 kDa, or 3.4 kDa; from 2.7 kDa to: 2.8 kDa, 2.9 kDa, 3.0 kDa, 3.1 kDa, 3.2 kDa, 3.3 kDa, or 3.4 kDa; from 2.8 kDa to: 2.9 kDa, 3.0 kDa, 3.1 kDa, 3.2 kDa, 3.3 kDa, or 3.4 kDa; from 2.9 kDa to: 3.0 kDa, 3.1 kDa, 3.2 kDa, 3.3 kDa, or 3.4 kDa; or from 3.0 kDa to: 3.1 kDa, 3.2 kDa, 3.3 kDa, or 3.4 kDa. Taking the above-noted embodiments into account, it is contemplated and supported that any of the above-noted “median molecular weight (i.e. number-averaged molecular weight) from” and “median molecular weight (i.e. number-averaged molecular weight) to” may be combined to provide an enclosed range (i.e. a median molecular weight (i.e. number-averaged molecular weight) from 1.1 kDa to 2.4 kDa). Also contemplated and supported are combinations of the above-noted “median molecular weight (i.e. number-averaged molecular weight) from” or “median molecular weight (i.e. number-averaged molecular weight) to” with the median molecular weight of the PEG provided in the Examples section of this document to provide an enclosed range. Also contemplated and supported are combinations of the median molecular weight (i.e. number-averaged molecular weight) of the Example section of this document to provide a range (i.e. PEG having a median molecular weight (i.e. number-averaged molecular weight) of from [the PEG's median molecular weight (i.e. number-averaged molecular weight) of lipid X₁ in the Examples] to [the PEG's median molecular weight (i.e. number-averaged molecular weight) of lipid X₂ in the Examples] wherein X₁ and X₂ represent any two exemplary PEG-conjugated lipids of the Examples.

In some embodiments, the PEGs in the PEG-conjugated lipids have a median molecular weight (i.e. number-averaged molecular weight) of: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, 3.4 kDa, 3.5 kDa, 3.6 kDa, 3.7 kDa, 3.8 kDa, 3.9 kDa, 4.0 kDa, 4.1 kDa, 4.2 kDa, 4.3 kDa, 4.4 kDa, 4.5 kDa, 4.6 kDa, 4.7 kDa, 4.8 kDa, 4.9 kDa, 5.0 kDa, 5.1 kDa, 5.2 kDa, 5.3 kDa, 5.4 kDa, 5.5 kDa, 5.6 kDa, 5.7 kDa, 5.8 kDa, 5.9 kDa, or 6.0 kDa.

In some embodiments, the PEG-conjugated lipid comprises 1,2-dimyristoyl-rac-glycero-3-methoxypolyethylene glycol-2000. In 1,2-dimyristoyl-rac-glycero-3-methoxypolyethylene glycol-2000, the “2000” represents the median molecular weight in Daltons of the PEG. In some embodiments, the PEG-conjugated lipid comprises 1,2-dimyristoyl-sn-glycero-2-phosphoethanolamine-N-[methoxy(polyethylene glycol)]. In some embodiments, the PEG-conjugated lipid comprises 1,2-dimyristoyl-rac-glycerol-3-methoxypolyethylene glycol.

### Second Lipids

In some embodiments, the LNP further comprises a second lipid, which comprises an anionic lipid, a neutral lipid, or a zwitterionic lipid. In some embodiments, the neutral lipid comprises a neutral zwitterionic lipid. In some embodiments, the anionic lipid, a neutral lipid, or the zwitterionic lipid comprises a phospho-group (i.e. is a phospholipid), a choline, or a sphingolipid.

In some embodiments, the second lipid comprises 1,2-diheptadecanoyl-sn-glycero-3-phosphoethanolamine (17:0 PE), 1,2-dihexanoyl-sn-glycero-3-phosphoethanolamine (06:0 PE), 1,2-dioctanoyl-sn-glycero-3-phosphoethanolamine (08:0 PE), 1,2-didecanoyl-sn-glycero-3-phosphoethanolamine (10:0 PE), 1,2-dilauroyl-sn-glycero-3-phosphoethanolamine (12:0 PE), 1,2-dipentadecanoyl-sn-glycero-3-phosphoethanolamine (15:0 PE), 1,2-dipalmitoyl-sn-glycero-3-phosphoethanolamine (16:0 PE), 1,2-distearoyl-sn-glycero-3-phosphoethanolamine (18:0 PE), 1,2-dimyristoyl-sn-glycero-3-phosphoethanolamine (14:0 PE), 1,2-dipalmitoleoyl-sn-glycero-3-phosphoethanolamine (16:1 PE), 1,2-dioleoyl-sn-glycero-3-phosphoethanolamine (DOPE), 1,2-dielaidoyl-sn-glycero-3-phosphoethanolamine (18:1 (A9-Trans) PE), 1,2-dilinoleoyl-sn-glycero-3-phosphoethanolamine (18:2 PE), 1,2-dilinolenoyl-sn-glycero-3-phosphoethanolamine (18:3 PE), 1,2-didocosahexaenoyl-sn-glycero-3-phosphoethanolamine (22:6 PE), 1,2-diarachidonoyl-sn-glycero-3-phosphoethanolamine (20:4 PE), 1-pentadecanoyl-2-oleoyl-sn-glycero-3-phosphoethanolamine (15:0-18:1 PE), 1-palmitoyl-2-linoleoyl-sn-glycero-3-phosphoethanolamine (16:0-18:2 PE), 1-palmitoyl-2-oleoyl-sn-glycero-3-phosphoethanolamine (16:0-18:1 PE), 1-stearoyl-2-oleoyl-sn-glycero-3-phosphoethanolamine (18:0-18:1 PE), 1-palmitoyl-2-arachidonoyl-sn-glycero-3-phosphoethanolamine (16:0-20:4 PE), 1-palmitoyl-2-docosahexaenoyl-sn-glycero-3-phosphoethanolamine (16:0-22:6 PE), 1-stearoyl-2-linoleoyl-sn-glycero-3-phosphoethanolamine (18:0-18:2 PE), 1-stearoyl-2-arachidonoyl-sn-glycero-3-phosphoethanolamine (18:0-20:4 PE), 1-stearoyl-2-docosahexaenoyl-sn-glycero-3-phosphoethanolamine (18:0-22:6 PE), 1-oleoyl-2-hydroxy-sn-glycero-3-phosphoethanolamine (18:1 Lyso PE), 1-hydroxy-2-oleoyl-sn-glycero-3-phosphoethanolamine (2-18:1 Lyso PE), 1-palmitoyl-2-hydroxy-sn-glycero-3-phosphoethanolamine (16:0 Lyso PE), 1-tridecanoyl-sn-glycero-3-phosphoethanolamine (13:0 Lyso PE), 1-(10Z-heptadecenoyl)-sn-glycero-3-phosphoethanolamine (17:1 Lyso PE), 1-stearoyl-2-hydroxy-sn-glycero-3-phosphoethanolamine (18:0 Lyso PE), 1-myristoyl-2-hydroxy-sn-glycero-3-phosphoethanolamine (14:0 Lyso PE), L-a-phosphatidylethanolamine, 1,2-dibutyryl-sn-glycero-3-phosphocholine (04:0 PC), 1,2-dihexanoyl-sn-glycero-3-phosphocholine (DHPC), 1,2-diheptanoyl-sn-glycero-3-phosphocholine (7:0 PC), 1,2-dioctanoyl-sn-glycero-3-phosphocholine (8:0 PC), 1,2-dinonanoyl-sn-glycero-3-phosphocholine (9:0 PC), 1,2-didecanoyl-sn-glycero-3-phosphocholine (10:0 PC), 1,2-diundecanoyl-sn-glycero-3-phosphocholine (11:0 PC), 1,3-dipalmitoyl-rac-glycero-2-phosphocholine (16:0 2—PC), 1,2-dilauroyl-sn-glycero-3-phosphocholine (DLPC), 1,2-ditridecanoyl-sn-glycero-3-phosphocholine (13:0 PC), 1,2-dimyristoyl-sn-glycero-3-phosphocholine (DMPC), 1,2-dipentadecanoyl-sn-glycero-3-phosphocholine (15:0 PC), 1,2-dipalmitoyl-sn-glycero-3-phosphocholine (DPPC), 1,2-diheptadecanoyl-sn-glycero-3-phosphocholine (17:0 PC), 1,2-distearoyl-sn-glycero-3-phosphocholine (DSPC), 1,2-distearoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1,2-divaccenoyl-sn-glycero-3-phosphocholine (18:1 (11-cis) PC), 1,2-di[(8Z)octadecenoyl]-sn-glycero-3-phosphocholine (18:1 (8-cis) PC), 1,2-dimyristoleoyl-sn-glycero-3-phosphocholine (14:1 (A9-cis) PC), 1,2-dipetroselenoyl-sn-glycero-3-phosphocholine (18:1 (A6-cis) PC), 1,2-dioleoyl-sn-glycero-3-phosphocholine (DOPC), 1,2-dielaidoyl-sn-glycero-3-phosphocholine (18:1 (A9-trans) PC), 1,2-diheptadecanoyl-sn-glycero-3-phospho-(1′-rac-glycerol), 1,2-dihexanoyl-sn-glycero-3-phospho-(1′-rac-glycerol), 1,2-dioctanoyl-sn-glycero-3-phospho-(1′-rac-glycerol), 1,2-didecanoyl-sn-glycero-3-phospho-(1′-rac-glycerol), 1,2-dilauroyl-sn-glycero-3-phospho-(1′-rac-glycerol), 1,2-dimyristoyl-sn-glycero-3-phospho-(1′-rac-glycerol), 1,2-dipentadecanoyl-sn-glycero-3-phospho-(1′-rac-glycerol), 1,2-dipalmitoyl-sn-glycero-3-phospho-(1′-rac-glycerol), 1,2-distearoyl-sn-glycero-3-phospho-(1′-rac-glycerol), 1,2-dioleoyl-sn-glycero-3-phospho-(1′-rac-glycerol), 1,2-dielaidoyl-sn-glycero-3-phospho-(1′-rac-glycerol), 1,2-dilinoleoyl-sn-glycero-3-phospho-(1′-rac-glycerol), 1,2-dilinolenoyl-sn-glycero-3-phospho-(1′-rac-glycerol), 1,2-diarachidonoyl-sn-glycero-3-[phospho-rac-(1-glycerol)], 1,2-didocosahexaenoyl-sn-glycero-3-[phospho-rac-(1-glycerol)], 1,2-dilinoleoyl-sn-glycero-3-phosphocholine (DLPC), 1,2-dilinolenoyl-sn-glycero-3-phosphocholine (18:3 (cis) PC), 1,2-dieicosenoyl-sn-glycero-3-phosphocholine (20:1 (cis) PC), 1,2-diarachidonoyl-sn-glycero-3-phosphocholine (20:4 (cis) PC), 1,2-dierucoyl-sn-glycero-3-phosphocholine (22:1 (cis) PC), 1,2-dinervonoyl-sn-glycero-3-phosphocholine (24:1 (cis) PC), 1,2-didocosahexaenoyl-sn-glycero-3-phosphocholine (22:6 (cis) PC), 1-pentadecanoyl-2-oleoyl-sn-glycero-3-phosphocholine (15:0-18:1 PC), 1-myristoyl-2-palmitoyl-sn-glycero-3-phosphocholine (14:0-16:0 PC), 1-myristoyl-2-stearoyl-sn-glycero-3-phosphocholine (14:0-18:0 PC), 1-palmitoyl-2-myristoyl-sn-glycero-3-phosphocholine (16:0-14:0 PC), 1-palmitoyl-2-stearoyl-sn-glycero-3-phosphocholine (16:0-18:0 PC), 1-palmitoyl-2-oleoyl-glycero-3-phosphocholine (POPC), 1-palmitoyl-2-linoleoyl-sn-glycero-3-phosphocholine (16:0-18:2 PC), 1-palmitoyl-2-arachidonoyl-sn-glycero-3-phosphocholine (16:0-20:4 PC), 1-palmitoyl-2-docosahexaenoyl-sn-glycero-3-phosphocholine (16:0-22:6 PC), 1-stearoyl-2-myristoyl-sn-glycero-3-phosphocholine (18:0-14:0 PC), 1-stearoyl-2-palmitoyl-sn-glycero-3-phosphocholine (18:0-16:0 PC), 1-stearoyl-2-oleoyl-sn-glycero-3-phosphocholine (18:0-18:1 PC), 1-stearoyl-2-linoleoyl-sn-glycero-3-phosphocholine (18:0-18:2 PC), 1-stearoyl-2-arachidonoyl-sn-glycero-3-phosphocholine (18:0-20:4 PC), 1-stearoyl-2-docosahexaenoyl-sn-glycero-3-phosphocholine (18:0-22:6 PC), 1-oleoyl-2-myristoyl-sn-glycero-3-phosphocholine (18:1-14:0 PC), 1-oleoyl-2-palmitoyl-sn-glycero-3-phosphocholine (18:1-16:0 PC), 1-oleoyl-2-stearoyl-sn-glycero-3-phosphocholine (18:1-18:0 PC), 1-(8Z-octadecenoyl)-2-palmitoyl-sn-glycero-3-phosphocholine (18:1(n10)-16:0 PC), 1-palmitoyl-2-acetyl-sn-glycero-3-phosphocholine (16:0-02:0 PC), 1-palmitoyl-2-[12′-(palmitoyloxy)octadecanoyl]-sn-glycero-3-phosphoholine (16:0-(12-PAHSA) PC), 1-oleoyl-2-hydroxy-sn-glycero-3-phosphocholine, 1-hexanoyl-2-hydroxy-sn-glycero-3-phosphocholine, 1-heptanoyl-2-hydroxy-sn-glycero-3-phosphocholine, 1-octanoyl-2-hydroxy-sn-glycero-3-phosphocholine, 1-nonanoyl-2-hydroxy-sn-glycero-3-phosphocholine, 1-decanoyl-2-hydroxy-sn-glycero-3-phosphocholine, 1-undecanoyl-2-hydroxy-sn-glycero-3-phosphocholine, 1-lauroyl-2-hydroxy-sn-glycero-3-phosphocholine, 1-tridecanoyl-2-hydroxy-sn-glycero-3-phosphocholine, 1-myristoyl-2-hydroxy-sn-glycero-3-phosphocholine, 1-pentadecanoyl-2-hydroxy-sn-glycero-3-phosphocholine, 1-hydroxy-2-palmitoyl-sn-glycero-3-phosphocholine, 1-palmitoyl-2-hydroxy-sn-glycero-3-phosphocholine, 1-heptadecanoyl-2-hydroxy-sn-glycero-3-phosphocholine, 1-(10Z-heptadecenoyl)-2-hydroxy-sn-glycero-3-phosphocholine, 1-hydroxy-2-oleoyl-sn-glycero-3-phosphocholine, 2-stearoyl-sn-glycero-3-phosphocholine, 1-stearoyl-2-hydroxy-sn-glycero-3-phosphocholine, 1-nonadecanoyl-2-hydroxy-sn-glycero-3-phosphocholine, 1-arachidoyl-2-hydroxy-sn-glycero-3-phosphocholine, 1-behenoyl-2-hydroxy-sn-glycero-3-phosphocholine, 1-lignoceroyl-2-hydroxy-sn-glycero-3-phosphocholine, 1-hexacosanoyl-2-hydroxy-sn-glycero-3-phosphocholine, 1,2-dimyristoyl-sn-glycero-phosphocholine (DMPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-didocosahexaenoyl-sn-glycero-3-phosphoethanolamine, 1,2-dioleoyl-sn-glycero-3-phospho-rac-(1-glycerol) sodium salt (DOPG), N-lignoceroyl-D-erythro-sphingosylphosphoethanolamine, or sphingomyelin.

### Sterols

In some embodiments, the lipid nanoparticles further comprise a sterol. In some embodiments, the sterol comprises cholesterol, cholesterol sulfate, desmosterol, stigmasterol, lanosterol, 7-dehydrocholesterol, dihydrolanosterol, symosterol, lathosteriol, 14-demethyl-lanosterol, 8(9)-dehydrocholesterol, 8(14)-dehydrocholesterol, 14-demethyl-14-dehydrolanosterol (FF-MAS), diosgenin, dehydroepiandrosterone sulfate (DHEA sulfate), dehydroepiandrosterone, sitosterol, lanosterol-95, 4,4-dimethyl(d6)-cholest-8(9), 14-dien-3β-ol (dihydro-FF-MAS-d6), 4,4-dimethyl(d6)-cholest-8(9)-en-3β-ol (dihydro T-MAS-d6), zymostenol, sitostanol, campestanol, camperstanol, 7-dehydrodesmosterol, pregnenolone, 4,4-dimethyl-cholest-8(9)-en-3β-ol (dihyrdro T-MAS), Δ5-avensterol, brassicasterol, dihydro FF-MAS, 24-methylene cholesterol, oxysterols, deuterated sterols, fluorinated sterols, sulfonated sterols, phosphorylated sterols, A-ring substituted sterols, cholest-5-ene-3ß,4ß-diol, 5α-cholestan-3ß-ol, 4-cholesten-3-one, cholesta-8(9),24-dien-3-one, cholesta-8(9),24-dien-3-one, 2,2,3,4,4-pentadeuterio-5α-cholestan-3ß-ol, cholesteryl phosphocholine, cholesteryl-d7 pentadecanoate, cholesteryl-d7 palmitate, B-ring substituted sterols, cholestanol, 5ß,6ß-epoxy-d7, 3ß-hydroxy-5-cholestene-7-one, 6α-hydroxy-5α-cholestane, cholestanol, 5α,6α-epoxy, cholest-5-en-3ß,7α-diol, cholest-5-en-33,713-diol, cholestanol, 5α,6α-epoxy-d7, Δ5,7-cholesterol, cholesta-5,8(9)-dien-3ß-ol, cholesta-5,8(14)-dien-3ß-ol, 7α-hydroxy-4-cholesten-3-one, zymostenol-d7, zymostenol, 7-dehydrodesmosterol, 3b,5α-dihydroxy-cholestan-6-one, D-ring substituted sterols, 3ß-hydroxy-5α-cholest-8(14)-en-15-one, 3ß-hydroxy-5α-cholestane-15-one, 5α-cholest-8(14)-ene-3ß,15α-diol, 5α-cholest-8(14)-ene-3ß,15f 3,-diol, lanosterol-95, 5α-7,24-cholestadiene, 14-dehydro zymostenol, ergosta-5,7,9(11),22-tetraen-3ß-ol, cholest-5-ene-3ß,25-diol, cholest-(25R)-5-ene-3ß,27-diol, 24(R/S),25-epoxycholesterol, 24(S),25-epoxycholesterol, 24(R/S),25-epoxycholesterol-d6, cholest-5-ene-3ß,22(S)-diol, cholest-5-ene-3ß,22(R)-diol, cholest-5-ene-3ß,24(S)-diol, cholest-5-ene-3ß,24(R)-diol, 27-hydroxy-4-cholesten-3-one, campestanol, N,N-dimethyl-3ß-hydroxycholenamide, 25,27-dihydroxycholesterol, N,N-dimethyl-3ß-hydroxycholenamide, 25,27-dihydroxycholesterol, 5-cholestene-3β,20α-diol, 24S,25-epoxy-5α-cholest-8(9)-en-3β-ol, 24(S/R),25-epoxylanost-8(9)-en-3β-ol, 7-keto-27-hydroxycholesterol, 7α,27-dihydroxy-4-cholesten-3-one, 7α,27-dihydroxycholesterol, 7ß,27-dihydroxycholesterol, 5α,6ß-dihydroxycholestanol, 7α,25-dihydroxycholesterol, 7ß,25-dihydroxycholesterol, 7α,24(S)-dihydroxycholesterol, 7α,24(S)-dihydroxy-4-cholesten-3-one, 7-keto-25-hydroxycholesterol, 7α,24S,27-trihydroxycholesterol, dihydrotestosterone, testosterone, estrone, estrogen, estradiol, corticosterone, cortisol, or 24S,27-dihydroxycholesterol.

### Mole Amounts of Lipids in the LNP

In some embodiments, the lipids in the LNP have, comprise, consist of, consist essentially of, or are at least 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole % of first lipid. In some embodiments, the lipids in the LNP have, comprise, consist of, consist essentially of, or are no more than 80 mole %, 79 mole %, 78 mole %, 77 mole %, 76 mole %, 75 mole %, 74 mole %, 73 mole %, 72 mole %, 71 mole %, 70 mole %, 69 mole %, 68 mole %, 67 mole %, 66 mole %, 65 mole %, 64 mole %, 63 mole %, 62 mole %, 61 mole %, 60 mole %, 59 mole %, 58 mole %, 57 mole %, 56 mole %, 55 mole %, 54 mole %, 53 mole %, 52 mole %, 51 mole %, 50 mole %, 49 mole %, 48 mole %, 47 mole %, 46 mole %, 45 mole %, 44 mole %, 43 mole %, 42 mole %, 41 mole %, 40 mole %, 39 mole %, 38 mole %, 37 mole %, 36 mole %, 35 mole %, 34 mole %, 33 mole %, 32 mole %, 31 mole %, 30 mole %, 29 mole %, 28 mole %, 27 mole %, 26 mole %, 25 mole %, 24 mole %, 23 mole %, 22 mole %, 21 mole %, or 20 mole % of first lipid. Taking the above-noted embodiments into account, it is contemplated and supported that any of the above-noted “mole %” of “at least” and any of the above-noted “mole %” of “no more than” may be combined to provide an enclosed range (i.e. the lipids comprise from 20 mole % to 60 mole % of the first lipid). In some embodiments, the lipids in the LNP have, comprise, consist of, consist essentially of, or are from 20 mole % to: 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 21 mole % to: 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 22 mole % to: 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 23 mole % to: 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 24 mole % to: 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 25 mole % to: 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 26 mole % to: 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 27 mole % to: 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 28 mole % to: 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 29 mole % to: 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 30 mole % to: 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 31 mole % to: 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 32 mole % to: 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 33 mole % to: 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 34 mole % to: 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 35 mole % to: 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 36 mole % to: 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 37 mole % to: 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 38 mole % to: 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 39 mole % to: 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 40 mole % to: 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 41 mole % to: 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 42 mole % to: 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 43 mole % to: 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 44 mole % to: 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 45 mole % to: 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 46 mole % to: 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 47 mole % to: 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 48 mole % to: 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 49 mole % to: 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 50 mole % to: 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 51 mole % to: 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 52 mole % to: 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 53 mole % to: 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 54 mole % to: 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 55 mole % to: 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 56 mole % to: 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 57 mole % to: 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 58 mole % to: 59 mole %, 60 mole %, 61 mole %, 62 mole %, 63 mole %, 64 mole %, 65 mole %, 66 mole %, 67 mole %, 68 mole %, 69 mole %, or 70 mole %; from 59 mole % to: 60 mole %, 61 mole %, 62 mole %, 63 mole %, 64 mole %, 65 mole %, 66 mole %, 67 mole %, 68 mole %, 69 mole %, or 70 mole %; from 60 mole % to: 61 mole %, 62 mole %, 63 mole %, 64 mole %, 65 mole %, 66 mole %, 67 mole %, 68 mole %, 69 mole %, or 70 mole %; from 61 mole % to: 62 mole %, 63 mole %, 64 mole %, 65 mole %, 66 mole %, 67 mole %, 68 mole %, 69 mole %, or 70 mole %; from 62 mole % to: 63 mole %, 64 mole %, 65 mole %, 66 mole %, 67 mole %, 68 mole %, 69 mole %, or 70 mole %; from 63 mole % to: 64 mole %, 65 mole %, 66 mole %, 67 mole %, 68 mole %, 69 mole %, or 70 mole %; from 64 mole % to: 65 mole %, 66 mole %, 67 mole %, 68 mole %, 69 mole %, or 70 mole %; from 65 mole % to: 66 mole %, 67 mole %, 68 mole %, 69 mole %, or 70 mole %; from 66 mole % to: 67 mole %, 68 mole %, 69 mole %, or 70 mole %; from 67 mole % to: 68 mole %, 69 mole %, or 70 mole %; from 68 mole % to: 69 mole % or 70 mole %; or from 69 mole % to 70 mole % of first lipid. Also contemplated and supported are combinations of the above-noted “mole %” of “at least” or “mole %” of “no more than” with the mole % of the first lipid provided in the Examples section of this document. Also contemplated and supported are combinations of the mole % of the Example section of this document to provide a range (i.e. mole % from [the mole % of the first lipid of the lipids of the LNP formulation X₁ in the Examples] to [the mole % of the first lipid of the lipids of the LNP formulation X₂ in the Examples] wherein X₁ and X₂ represent any two exemplary LNP formulations of the Examples.

In some embodiments, the lipids in the LNP have, comprise, consist of, consist essentially of, or are at least 10 mole %, 11 mole %, 12 mole %, 13 mole %, 14 mole %, 15 mole %, 16 mole %, 17 mole %, 18 mole %, 19 mole %, 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole % of sterol.

In some embodiments, the lipids in the LNP have, comprise, consist of, consist essentially of, or are no more than 80 mole %, 79 mole %, 78 mole %, 77 mole %, 76 mole %, 75 mole %, 74 mole %, 73 mole %, 72 mole %, 71 mole %, 70 mole %, 69 mole %, 68 mole %, 67 mole %, 66 mole %, 65 mole %, 64 mole %, 63 mole %, 62 mole %, 61 mole %, 60 mole %, 59 mole %, 58 mole %, 57 mole %, 56 mole %, 55 mole %, 54 mole %, 53 mole %, 52 mole %, 51 mole %, 50 mole %, 49 mole %, 48 mole %, 47 mole %, 46 mole %, 45 mole %, 44 mole %, 43 mole %, 42 mole %, 41 mole %, 40 mole %, 39 mole %, 38 mole %, 37 mole %, 36 mole %, 35 mole %, 34 mole %, 33 mole %, 32 mole %, 31 mole %, 30 mole %, 29 mole %, 28 mole %, 27 mole %, 26 mole %, 25 mole %, 24 mole %, 23 mole %, 22 mole %, 21 mole %, or 20 mole % of sterol. Taking the above-noted embodiments into account, it is contemplated and supported that any of the above-noted “mole %” of “at least” and any of the above-noted “mole %” of “no more than” may be combined to provide an enclosed range (i.e. the lipids comprise from 20 mole % to 60 mole % of sterol). In some embodiments, the lipids in the LNP have, comprise, consist of, consist essentially of, or are from 20 mole % to: 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 21 mole % to: 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 22 mole % to: 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 23 mole % to: 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 24 mole % to: 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 25 mole % to: 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 26 mole % to: 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 27 mole % to: 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 28 mole % to: 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 29 mole % to: 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 30 mole % to: 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 31 mole % to: 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 32 mole % to: 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 33 mole % to: 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 34 mole % to: 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 35 mole % to: 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 36 mole % to: 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 37 mole % to: 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 38 mole % to: 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 39 mole % to: 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 40 mole % to: 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 41 mole % to: 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 42 mole % to: 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 43 mole % to: 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 44 mole % to: 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 45 mole % to: 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 46 mole % to: 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 47 mole % to: 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 48 mole % to: 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 49 mole % to: 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 50 mole % to: 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 51 mole % to: 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 52 mole % to: 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 53 mole % to: 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 54 mole % to: 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 55 mole % to: 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 56 mole % to: 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 57 mole % to: 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 58 mole % to: 59 mole %, 60 mole %, 61 mole %, 62 mole %, 63 mole %, 64 mole %, 65 mole %, 66 mole %, 67 mole %, 68 mole %, 69 mole %, or 70 mole %; from 59 mole % to: 60 mole %, 61 mole %, 62 mole %, 63 mole %, 64 mole %, 65 mole %, 66 mole %, 67 mole %, 68 mole %, 69 mole %, or 70 mole %; from 60 mole % to: 61 mole %, 62 mole %, 63 mole %, 64 mole %, 65 mole %, 66 mole %, 67 mole %, 68 mole %, 69 mole %, or 70 mole %; from 61 mole % to: 62 mole %, 63 mole %, 64 mole %, 65 mole %, 66 mole %, 67 mole %, 68 mole %, 69 mole %, or 70 mole %; from 62 mole % to: 63 mole %, 64 mole %, 65 mole %, 66 mole %, 67 mole %, 68 mole %, 69 mole %, or 70 mole %; from 63 mole % to: 64 mole %, 65 mole %, 66 mole %, 67 mole %, 68 mole %, 69 mole %, or 70 mole %; from 64 mole % to: 65 mole %, 66 mole %, 67 mole %, 68 mole %, 69 mole %, or 70 mole %; from 65 mole % to: 66 mole %, 67 mole %, 68 mole %, 69 mole %, or 70 mole %; from 66 mole % to: 67 mole %, 68 mole %, 69 mole %, or 70 mole %; from 67 mole % to: 68 mole %, 69 mole %, or 70 mole %; from 68 mole % to: 69 mole % or 70 mole %; or from 69 mole % to 70 mole % of sterol. Also contemplated and supported are combinations of the above-noted “mole %” of “at least” or “mole %” of “no more than” with the mole % of sterol provided in the Examples section of this document. Also contemplated and supported are combinations of the mole % of the Example section of this document to provide a range (i.e. mole % from [the mole % of sterol of the lipids of the LNP formulation X₁ in the Examples] to [the mole % of sterol of the lipids of the LNP formulation X₂ in the Examples] wherein X, and X₂ represent any two exemplary LNP formulations of the Examples.

In some embodiments, the lipids in the LNP have, comprise, consist of, consist essentially of, or are at least 0.1 mole %, 0.2 mole %, 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole % of polymer-conjugated lipid. In some embodiments, the lipids in the LNP have, comprise, consist of, consist essentially of, or are no more than 8.0 mole %, 7.9 mole %, 7.8 mole %, 7.7 mole %, 7.6 mole %, 7.5 mole %, 7.4 mole %, 7.3 mole %, 7.2 mole %, 7.1 mole %, 7.0 mole %, 6.9 mole %, 6.8 mole %, 6.7 mole %, 6.6 mole %, 6.5 mole %, 6.4 mole %, 6.3 mole %, 6.2 mole %, 6.1 mole %, 6.0 mole %, 5.9 mole %, 5.8 mole %, 5.7 mole %, 5.6 mole %, 5.5 mole %, 5.4 mole %, 5.3 mole %, 5.2 mole %, 5.1 mole %, 5.0 mole %, 4.9 mole %, 4.8 mole %, 4.7 mole %, 4.6 mole %, 4.5 mole %, 4.4 mole %, 4.3 mole %, 4.2 mole %, 4.1 mole %, 4.0 mole %, 3.9 mole %, 3.8 mole %, 3.7 mole %, 3.6 mole %, 3.5 mole %, 3.4 mole %, 3.3 mole %, 3.2 mole %, 3.1 mole %, 3.0 mole %, 2.9 mole %, 2.8 mole %, 2.7 mole %, 2.6 mole %, 2.5 mole %, 2.4 mole %, 2.3 mole %, 2.2 mole %, 2.1 mole %, 2.0 mole %, 1.9 mole %, 1.8 mole %, 1.7 mole %, 1.6 mole %, 1.5 mole %, 1.4 mole %, 1.3 mole %, 1.2 mole %, 1.1 mole %, 1.0 mole %, 0.9 mole %, 0.8 mole %, 0.7 mole %, 0.6 mole %, 0.5 mole %, 0.4 mole %, 0.3 mole %, 0.2 mole %, 0.1 mole %, or no mole % of polymer-conjugated lipid. Taking the above-noted embodiments into account, it is contemplated and supported that any of the above-noted “mole %” of “at least” and any of the above-noted “mole %” of “no more than” may be combined to provide an enclosed range (i.e. the lipids comprise from 20 mole % to 60 mole % of polymer-conjugated lipid).

In some embodiments, the lipids in the LNP have, comprise, consist of, consist essentially of, or are from 0.1 mole % to: 0.2 mole %, 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.2 mole % to: 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.3 mole % to: 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.4 mole % to: 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.5 mole % to: 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.6 mole % to: 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.7 mole % to: 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.8 mole % to: 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.9 mole % to: 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.0 mole % to: 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.1 mole % to: 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.2 mole % to: 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.3 mole % to: 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.4 mole % to: 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.5 mole % to: 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.6 mole % to: 1.7 mole % to: 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.7 mole % to: 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.8 mole % to: 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.9 mole % to: 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.0 mole % to: 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.1 mole % to: 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.2 mole % to: 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.3 mole % to: 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.4 mole % to: 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.5 mole % to: 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.6 mole % to: 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.7 mole % to: 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.8 mole % to: 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.9 mole % to: 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.0 mole % to: 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.1 mole % to: 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.2 mole % to: 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.3 mole % to: 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.4 mole % to: 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.5 mole % to: 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.6 mole % to: 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.7 mole % to: 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.8 mole % to: 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.9 mole % to: 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.0 mole % to: 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.1 mole % to: 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.2 mole % to: 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.3 mole % to: 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.4 mole % to: 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.5 mole % to: 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.6 mole % to: 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.7 mole % to: 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.8 mole % to: 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.9 mole % to: 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.0 mole % to: 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.1 mole % to: 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.2 mole % to: 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.3 mole % to: 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.4 mole % to: 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.5 mole % to: 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.6 mole % to: 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.7 mole % to: 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.8 mole % to: 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.9 mole % to: 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.0 mole % to: 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.1 mole % to: 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.2 mole % to: 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.3 mole % to: 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.4 mole % to: 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.5 mole % to: 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.6 mole % to: 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.7 mole % to: 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.8 mole % to: 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.9 mole % to: 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 7.0 mole % to: 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 7.1 mole % to: 7.2 mole %, 7.3 mole %, 7.4 mole %, 7.5 mole %, 7.6 mole %, 7.7 mole %, 7.8 mole %, 7.9 mole %, or 8.0 mole %; from 7.2 mole % to: 7.3 mole %, 7.4 mole %, 7.5 mole %, 7.6 mole %, 7.7 mole %, 7.8 mole %, 7.9 mole %, or 8.0 mole %; from 7.3 mole % to: 7.4 mole %, 7.5 mole %, 7.6 mole %, 7.7 mole %, 7.8 mole %, 7.9 mole %, or 8.0 mole %; from 7.4 mole % to: 7.5 mole %, 7.6 mole %, 7.7 mole %, 7.8 mole %, 7.9 mole %, or 8.0 mole %; from 7.5 mole % to: 7.6 mole %, 7.7 mole %, 7.8 mole %, 7.9 mole %, or 8.0 mole %; from 7.6 mole % to: 7.7 mole %, 7.8 mole %, 7.9 mole %, or 8.0 mole %; from 7.7 mole % to: 7.8 mole %, 7.9 mole %, or 8.0 mole %; from 7.8 mole % to: 7.9 mole %, or 8.0 mole %; or from 7.9 mole % to 8.0 mole % of polymer-conjugated lipid. Also contemplated and supported are combinations of the above-noted “mole %” of “at least” or “mole %” of “no more than” with the mole % of polymer-conjugated lipid provided in the Examples section of this document. Also contemplated and supported are combinations of the mole % of the Example section of this document to provide a range (i.e. mole % from [the mole % of polymer-conjugated lipid of the lipids of the LNP formulation X₁ in the Examples] to [the mole % of polymer-conjugated lipid of the lipids of the LNP formulation X₂ in the Examples] wherein X₁ and X₂ represent any two exemplary LNP formulations of the Examples.

In some embodiments, the lipids in the LNP have, comprise, consist of, consist essentially of, or are at least 0.1 mole %, 0.2 mole %, 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole % of second lipid. In some embodiments, the lipids in the LNP have, comprise, consist of, consist essentially of, or are no more than 11.0 mole %, 10.9 mole %, 10.8 mole %, 10.7 mole %, 10.6 mole %, 10.5 mole %, 10.4 mole %, 10.3 mole %, 10.2 mole %, 10.1 mole %, 10.0 mole %, 9.9 mole %, 9.8 mole %, 9.7 mole %, 9.6 mole %, 9.5 mole %, 9.4 mole %, 9.3 mole %, 9.2 mole %, 9.1 mole %, 8.0 mole %, 7.9 mole %, 7.8 mole %, 7.7 mole %, 7.6 mole %, 7.5 mole %, 7.4 mole %, 7.3 mole %, 7.2 mole %, 7.1 mole %, 7.0 mole %, 6.9 mole %, 6.8 mole %, 6.7 mole %, 6.6 mole %, 6.5 mole %, 6.4 mole %, 6.3 mole %, 6.2 mole %, 6.1 mole %, 6.0 mole %, 5.9 mole %, 5.8 mole %, 5.7 mole %, 5.6 mole %, 5.5 mole %, 5.4 mole %, 5.3 mole %, 5.2 mole %, 5.1 mole %, 5.0 mole %, 4.9 mole %, 4.8 mole %, 4.7 mole %, 4.6 mole %, 4.5 mole %, 4.4 mole %, 4.3 mole %, 4.2 mole %, 4.1 mole %, 4.0 mole %, 3.9 mole %, 3.8 mole %, 3.7 mole %, 3.6 mole %, 3.5 mole %, 3.4 mole %, 3.3 mole %, 3.2 mole %, 3.1 mole %, 3.0 mole %, 2.9 mole %, 2.8 mole %, 2.7 mole %, 2.6 mole %, 2.5 mole %, 2.4 mole %, 2.3 mole %, 2.2 mole %, 2.1 mole %, 2.0 mole %, 1.9 mole %, 1.8 mole %, 1.7 mole %, 1.6 mole %, 1.5 mole %, 1.4 mole %, 1.3 mole %, 1.2 mole %, 1.1 mole %, 1.0 mole %, 0.9 mole %, 0.8 mole %, 0.7 mole %, 0.6 mole %, 0.5 mole %, 0.4 mole %, 0.3 mole %, 0.2 mole %, 0.1 mole %, or no mole % of second lipid. Taking the above-noted embodiments into account, it is contemplated and supported that any of the above-noted “mole %” of “at least” and any of the above-noted “mole %” of “no more than” may be combined to provide an enclosed range (i.e. the lipids comprise from 20 mole % to 60 mole % of second lipid). In some embodiments, the lipids in the LNP have, comprise, consist of, consist essentially of, or are from 0.1 mole % to: 0.2 mole %, 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.2 mole % to: 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.3 mole % to: 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.4 mole % to: 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.5 mole % to: 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.6 mole % to: 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.7 mole % to: 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.8 mole % to: 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.9 mole % to: 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.0 mole % to: 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.1 mole % to: 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.2 mole % to: 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.3 mole % to: 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.4 mole % to: 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.5 mole % to: 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.6 mole % to: 1.7 mole % to: 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.7 mole % to: 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.8 mole % to: 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.9 mole % to: 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.0 mole % to: 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.1 mole % to: 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.2 mole % to: 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.3 mole % to: 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.4 mole % to: 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.5 mole % to: 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.6 mole % to: 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.7 mole % to: 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.8 mole % to: 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.9 mole % to: 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.0 mole % to: 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.1 mole % to: 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.2 mole % to: 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.3 mole % to: 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.4 mole % to: 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.5 mole % to: 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.6 mole % to: 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.7 mole % to: 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.8 mole % to: 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.9 mole % to: 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.0 mole % to: 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.1 mole % to: 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.2 mole % to: 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.3 mole % to: 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.4 mole % to: 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.5 mole % to: 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.6 mole % to: 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.7 mole % to: 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.8 mole % to: 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.9 mole % to: 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.0 mole % to: 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.1 mole % to: 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.2 mole % to: 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.3 mole % to: 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.4 mole % to: 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.5 mole % to: 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.6 mole % to: 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.7 mole % to: 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.8 mole % to: 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.9 mole % to: 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.0 mole % to: 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.1 mole % to: 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.2 mole % to: 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.3 mole % to: 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.4 mole % to: 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.5 mole % to: 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.6 mole % to: 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.7 mole % to: 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.8 mole % to: 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.9 mole % to: 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.0 mole % to: 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.1 mole % to: 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.2 mole % to: 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.3 mole % to: 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.4 mole % to: 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.5 mole % to: 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.6 mole % to: 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.7 mole % to: 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.8 mole % to: 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.9 mole % to: 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.0 mole % to: 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.1 mole % to: 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.2 mole % to: 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.3 mole % to: 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.4 mole % to: 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.5 mole % to: 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.6 mole % to: 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.7 mole % to: 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.8 mole % to: 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.9 mole % to: 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.0 mole % to: 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.1 mole % to: 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.2 mole % to: 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.3 mole % to: 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.4 mole % to: 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.5 mole % to: 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.6 mole % to: 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.7 mole % to: 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.8 mole % to: 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.9 mole % to: 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.0 mole % to: 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.1 mole % to: 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.2 mole % to: 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.3 mole % to: 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.4 mole % to: 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.5 mole % to: 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.6 mole % to: 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.7 mole % to: 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.8 mole % to: 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.9 mole % to: 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.0 mole % to: 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.1 mole % to: 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.2 mole % to: 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.3 mole % to: 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.4 mole % to: 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.5 mole % to: 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.6 mole % to: 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.7 mole % to: 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.8 mole % to: 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; or from 11.9 mole % to: 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; or from 12.0 mole % to: 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole % of second lipid. Also contemplated and supported are combinations of the above-noted “mole %” of “at least” or “mole %” of “no more than” with the mole % of second lipid provided in the Examples section of this document. Also contemplated and supported are combinations of the mole % of the Example section of this document to provide a range (i.e. mole % from [the mole % of second lipid of the lipids of the LNP formulation X₁ in the Examples] to [the mole % of second lipid of the lipids of the LNP formulation X₂ in the Examples] wherein X₁ and X₂ represent any two exemplary LNP formulations of the Examples.

In some embodiments, the lipids in the LNP have, comprise, consist of, consist essentially of, or are at least a mole amount of the second lipid of 0.01-times, 0.02-times, 0.03-times, 0.04-times, 0.05-times, 0.06-times, 0.07-times, 0.08-times, 0.09-times, 0.10-times, 0.11-times, 0.12-times, 0.13-times, 0.14-times, 0.15-times, 0.16-times, 0.17-times, 0.18-times, 0.19-times, 0.20-times, 0.21-times, 0.22-times, 0.23-times, 0.24-times, 0.25-times, 0.26-times, 0.27-times, 0.28-times, 0.29-times, 0.30-times, 0.31-times, 0.32-times, 0.33-times, 0.34-times, 0.35-times, 0.36-times, 0.37-times, 0.38-times, 0.39-times, 0.40-times, 0.41-times, 0.42-times, 0.43-times, 0.44-times, 0.45-times, 0.46-times, 0.47-times, 0.48-times, 0.49-times, 0.50-times, 0.51-times, 0.52-times, 0.53-times, 0.54-times, 0.55-times, 0.56-times, 0.57-times, 0.58-times, 0.59-times, 0.60-times, 0.61-times, 0.62-times, 0.63-times, 0.64-times, 0.65-times, 0.66-times, 0.67-times, 0.68-times, 0.69-times, or 0.70-times the mole amount of the first lipid. In some embodiments, the lipids in the LNP have, comprise, consist of, consist essentially of, or are no more than a mole amount of the second lipid of 1.0-times, 0.99-times, 0.98-times, 0.97-times, 0.96-times, 0.95-times, 0.94-times, 0.93-times, 0.92-times, 0.91-times, 0.90-times, 0.89-times, 0.88-times, 0.87-times, 0.86-times, 0.85-times, 0.84-times, 0.83-times, 0.82-times, 0.81-times, 0.80-times, 0.79-times, 0.78-times, 0.77-times, 0.76-times, 0.75-times, 0.74-times, 0.73-times, 0.72-times, 0.71-times, 0.70-times, 0.69-times, 0.68-times, 0.67-times, 0.66-times, 0.65-times, 0.64-times, 0.63-times, 0.62-times, 0.61-times, 0.60-times, 0.59-times, 0.58-times, 0.57-times, 0.56-times, 0.55-times, 0.54-times, 0.53-times, 0.52-times, 0.51-times, 0.50-times, 0.49-times, 0.48-times, 0.47-times, 0.46-times, 0.45-times, 0.44-times, 0.43-times, 0.42-times, 0.41-times, 0.40-times, 0.39-times, 0.38-times, 0.37-times, 0.36-times, 0.35-times, 0.34-times, 0.33-times, 0.32-times, 0.31-times, 0.30-times, 0.29-times, 0.28-times, 0.27-times, 0.26-times, 0.25-times, 0.24-times, 0.23-times, 0.22-times, 0.21-times, 0.20-times, 0.19-times, 0.18-times, 0.17-times, 0.16-times, 0.15-times, 0.14-times, 0.13-times, 0.12-times, 0.11-times, 0.10-times, 0.09-times, 0.08-times, 0.07-times, 0.06-times, 0.05-times, 0.04-times, 0.03-times, 0.02-times, 0.01-times, or 0.0-times the mole amount of the first lipid. Taking the above-noted embodiments into account, it is contemplated and supported that any of the above-noted “at least a mole amount of the second lipid of X-times the mole amount of the first lipid” and “no more than a mole amount of the second lipid of Y-times the mole amount of the first lipid” may be combined to provide an enclosed range (i.e. the lipids in the LNP comprise a mole amount of the second lipid that is from 0.1-times to 0.2-times the amount of the first lipid). Also contemplated and supported are combinations of the above-noted “at least a mole amount of the second lipid of X-times the mole amount of the first lipid” and “no more than a mole amount of the second lipid of Y-times the mole amount of the first lipid” with that provided in the Examples section of this document. Also contemplated and supported are combinations of the “mole amount of the second lipid of X-times the mole amount of the first lipid” of the Example section of this document to provide a range (i.e. the lipids in the LNP comprise a mole amount of the second lipid that is from X₁-times to X₂-times the amount of the first lipid wherein X₁ and X₂ represent the mole amount of any second lipids relative to the mole amount of the first lipid of any two exemplary LNP formulations of the Examples.

In some embodiments, the lipids in the LNP have any of the following mole % in combination:

| | | | second | | | | second | | |
|---|---|---|---|---|---|---|---|---|---|
| | | polymer- | lipid | | | polymer- | lipid | | |
| first | | conjugated | (i.e | first | | conjugated | (i.e | first | |
| lipid | | lipid | anionic, | lipid | | lipid | anionic, | lipid | |
| (i.e. | | (i.e. | neutral, | (i.e. | | (i.e. | neutral, | (i.e. | |
| cation- | sterol | PEG- | or | cation- | sterol | PEG- | or | cation- | sterol |
| ionizable | (i.e. | conjugated | zwitterionic | ionizable | (i.e. | conjugated | zwitterionic | ionizable | (i.e. |
| lipid) | cholesterol | lipid) | lipid) | lipid | cholesterol | lipid) | lipid) | lipid | cholesterol |
| (mole %) | (mole %) | (mole %) | (mole %) | (mole %) | (mole %) | (mole %) | (mole %) | (mole %) | (mole %) |
| 60 | 39 | 1 | 0 | 60 | 38 | 1 | 1 | 60 | 37 |
| 59 | 40 | 1 | 0 | 59 | 39 | 1 | 1 | 59 | 38 |
| 58 | 41 | 1 | 0 | 58 | 40 | 1 | 1 | 58 | 39 |
| 57 | 42 | 1 | 0 | 57 | 41 | 1 | 1 | 57 | 40 |
| 56 | 43 | 1 | 0 | 56 | 42 | 1 | 1 | 56 | 41 |
| 55 | 44 | 1 | 0 | 55 | 43 | 1 | 1 | 55 | 42 |
| 54 | 45 | 1 | 0 | 54 | 44 | 1 | 1 | 54 | 43 |
| 53 | 46 | 1 | 0 | 53 | 45 | 1 | 1 | 53 | 44 |
| 52 | 47 | 1 | 0 | 52 | 46 | 1 | 1 | 52 | 45 |
| 51 | 48 | 1 | 0 | 51 | 47 | 1 | 1 | 51 | 46 |
| 50 | 49 | 1 | 0 | 50 | 48 | 1 | 1 | 50 | 47 |
| 49 | 50 | 1 | 0 | 49 | 49 | 1 | 1 | 49 | 48 |
| 48 | 51 | 1 | 0 | 48 | 50 | 1 | 1 | 48 | 49 |
| 47 | 52 | 1 | 0 | 47 | 51 | 1 | 1 | 47 | 50 |
| 46 | 53 | 1 | 0 | 46 | 52 | 1 | 1 | 46 | 51 |
| 45 | 54 | 1 | 0 | 45 | 53 | 1 | 1 | 45 | 52 |
| 44 | 55 | 1 | 0 | 44 | 54 | 1 | 1 | 44 | 53 |
| 43 | 56 | 1 | 0 | 43 | 55 | 1 | 1 | 43 | 54 |
| 42 | 57 | 1 | 0 | 42 | 56 | 1 | 1 | 42 | 55 |
| 41 | 58 | 1 | 0 | 41 | 57 | 1 | 1 | 41 | 56 |
| 40 | 59 | 1 | 0 | 40 | 58 | 1 | 1 | 40 | 57 |
| 39 | 60 | 1 | 0 | 39 | 59 | 1 | 1 | 39 | 58 |
| 38 | 61 | 1 | 0 | 38 | 60 | 1 | 1 | 38 | 59 |
| 37 | 62 | 1 | 0 | 37 | 61 | 1 | 1 | 37 | 60 |
| 36 | 63 | 1 | 0 | 36 | 62 | 1 | 1 | 36 | 61 |
| 35 | 64 | 1 | 0 | 35 | 63 | 1 | 1 | 35 | 62 |
| 34 | 65 | 1 | 0 | 34 | 64 | 1 | 1 | 34 | 63 |
| 33 | 66 | 1 | 0 | 33 | 65 | 1 | 1 | 33 | 64 |
| 32 | 67 | 1 | 0 | 32 | 66 | 1 | 1 | 32 | 65 |
| 31 | 68 | 1 | 0 | 31 | 67 | 1 | 1 | 31 | 66 |
| 30 | 69 | 1 | 0 | 30 | 68 | 1 | 1 | 30 | 67 |
| 60 | 38.8 | 1.2 | 0 | 60 | 37.8 | 1.2 | 1 | 60 | 36.8 |
| 59 | 39.8 | 1.2 | 0 | 59 | 38.8 | 1.2 | 1 | 59 | 37.8 |
| 58 | 40.8 | 1.2 | 0 | 58 | 39.8 | 1.2 | 1 | 58 | 38.8 |
| 57 | 41.8 | 1.2 | 0 | 57 | 40.8 | 1.2 | 1 | 57 | 39.8 |
| 56 | 42.8 | 1.2 | 0 | 56 | 41.8 | 1.2 | 1 | 56 | 40.8 |
| 55 | 43.8 | 1.2 | 0 | 55 | 42.8 | 1.2 | 1 | 55 | 41.8 |
| 54 | 44.8 | 1.2 | 0 | 54 | 43.8 | 1.2 | 1 | 54 | 42.8 |
| 53 | 45.8 | 1.2 | 0 | 53 | 44.8 | 1.2 | 1 | 53 | 43.8 |
| 52 | 46.8 | 1.2 | 0 | 52 | 45.8 | 1.2 | 1 | 52 | 44.8 |
| 51 | 47.8 | 1.2 | 0 | 51 | 46.8 | 1.2 | 1 | 51 | 45.8 |
| 50 | 48.8 | 1.2 | 0 | 50 | 47.8 | 1.2 | 1 | 50 | 46.8 |
| 49 | 49.8 | 1.2 | 0 | 49 | 48.8 | 1.2 | 1 | 49 | 47.8 |
| 48 | 50.8 | 1.2 | 0 | 48 | 49.8 | 1.2 | 1 | 48 | 48.8 |
| 47 | 51.8 | 1.2 | 0 | 47 | 50.8 | 1.2 | 1 | 47 | 49.8 |
| 46 | 52.8 | 1.2 | 0 | 46 | 51.8 | 1.2 | 1 | 46 | 50.8 |
| 45 | 53.8 | 1.2 | 0 | 45 | 52.8 | 1.2 | 1 | 45 | 51.8 |
| 44 | 54.8 | 1.2 | 0 | 44 | 53.8 | 1.2 | 1 | 44 | 52.8 |
| 43 | 55.8 | 1.2 | 0 | 43 | 54.8 | 1.2 | 1 | 43 | 53.8 |
| 42 | 56.8 | 1.2 | 0 | 42 | 55.8 | 1.2 | 1 | 42 | 54.8 |
| 41 | 57.8 | 1.2 | 0 | 41 | 56.8 | 1.2 | 1 | 41 | 55.8 |
| 40 | 58.8 | 1.2 | 0 | 40 | 57.8 | 1.2 | 1 | 40 | 56.8 |
| 39 | 59.8 | 1.2 | 0 | 39 | 58.8 | 1.2 | 1 | 39 | 57.8 |
| 38 | 60.8 | 1.2 | 0 | 38 | 59.8 | 1.2 | 1 | 38 | 58.8 |
| 37 | 61.8 | 1.2 | 0 | 37 | 60.8 | 1.2 | 1 | 37 | 59.8 |
| 36 | 62.8 | 1.2 | 0 | 36 | 61.8 | 1.2 | 1 | 36 | 60.8 |
| 35 | 63.8 | 1.2 | 0 | 35 | 62.8 | 1.2 | 1 | 35 | 61.8 |
| 34 | 64.8 | 1.2 | 0 | 34 | 63.8 | 1.2 | 1 | 34 | 62.8 |
| 33 | 65.8 | 1.2 | 0 | 33 | 64.8 | 1.2 | 1 | 33 | 63.8 |
| 32 | 66.8 | 1.2 | 0 | 32 | 65.8 | 1.2 | 1 | 32 | 64.8 |
| 31 | 67.8 | 1.2 | 0 | 31 | 66.8 | 1.2 | 1 | 31 | 65.8 |
| 30 | 68.8 | 1.2 | 0 | 30 | 67.8 | 1.2 | 1 | 30 | 66.8 |
| 60 | 38.6 | 1.4 | 0 | 60 | 37.6 | 1.4 | 1 | 60 | 36.6 |
| 59 | 39.6 | 1.4 | 0 | 59 | 38.6 | 1.4 | 1 | 59 | 37.6 |
| 58 | 40.6 | 1.4 | 0 | 58 | 39.6 | 1.4 | 1 | 58 | 38.6 |
| 57 | 41.6 | 1.4 | 0 | 57 | 40.6 | 1.4 | 1 | 57 | 39.6 |
| 56 | 42.6 | 1.4 | 0 | 56 | 41.6 | 1.4 | 1 | 56 | 40.6 |
| 55 | 43.6 | 1.4 | 0 | 55 | 42.6 | 1.4 | 1 | 55 | 41.6 |
| 54 | 44.6 | 1.4 | 0 | 54 | 43.6 | 1.4 | 1 | 54 | 42.6 |
| 53 | 45.6 | 1.4 | 0 | 53 | 44.6 | 1.4 | 1 | 53 | 43.6 |
| 52 | 46.6 | 1.4 | 0 | 52 | 45.6 | 1.4 | 1 | 52 | 44.6 |
| 51 | 47.6 | 1.4 | 0 | 51 | 46.6 | 1.4 | 1 | 51 | 45.6 |
| 50 | 48.6 | 1.4 | 0 | 50 | 47.6 | 1.4 | 1 | 50 | 46.6 |
| 49 | 49.6 | 1.4 | 0 | 49 | 48.6 | 1.4 | 1 | 49 | 47.6 |
| 48 | 50.6 | 1.4 | 0 | 48 | 49.6 | 1.4 | 1 | 48 | 48.6 |
| 47 | 51.6 | 1.4 | 0 | 47 | 50.6 | 1.4 | 1 | 47 | 49.6 |
| 46 | 52.6 | 1.4 | 0 | 46 | 51.6 | 1.4 | 1 | 46 | 50.6 |
| 45 | 53.6 | 1.4 | 0 | 45 | 52.6 | 1.4 | 1 | 45 | 51.6 |
| 44 | 54.6 | 1.4 | 0 | 44 | 53.6 | 1.4 | 1 | 44 | 52.6 |
| 43 | 55.6 | 1.4 | 0 | 43 | 54.6 | 1.4 | 1 | 43 | 53.6 |
| 42 | 56.6 | 1.4 | 0 | 42 | 55.6 | 1.4 | 1 | 42 | 54.6 |
| 41 | 57.6 | 1.4 | 0 | 41 | 56.6 | 1.4 | 1 | 41 | 55.6 |
| 40 | 58.6 | 1.4 | 0 | 40 | 57.6 | 1.4 | 1 | 40 | 56.6 |
| 39 | 59.6 | 1.4 | 0 | 39 | 58.6 | 1.4 | 1 | 39 | 57.6 |
| 38 | 60.6 | 1.4 | 0 | 38 | 59.6 | 1.4 | 1 | 38 | 58.6 |
| 37 | 61.6 | 1.4 | 0 | 37 | 60.6 | 1.4 | 1 | 37 | 59.6 |
| 36 | 62.6 | 1.4 | 0 | 36 | 61.6 | 1.4 | 1 | 36 | 60.6 |
| 35 | 63.6 | 1.4 | 0 | 35 | 62.6 | 1.4 | 1 | 35 | 61.6 |
| 34 | 64.6 | 1.4 | 0 | 34 | 63.6 | 1.4 | 1 | 34 | 62.6 |
| 33 | 65.6 | 1.4 | 0 | 33 | 64.6 | 1.4 | 1 | 33 | 63.6 |
| 32 | 66.6 | 1.4 | 0 | 32 | 65.6 | 1.4 | 1 | 32 | 64.6 |
| 31 | 67.6 | 1.4 | 0 | 31 | 66.6 | 1.4 | 1 | 31 | 65.6 |
| 30 | 68.6 | 1.4 | 0 | 30 | 67.6 | 1.4 | 1 | 30 | 66.6 |
| 60 | 38.4 | 1.6 | 0 | 60 | 37.4 | 1.6 | 1 | 60 | 36.4 |
| 59 | 39.4 | 1.6 | 0 | 59 | 38.4 | 1.6 | 1 | 59 | 37.4 |
| 58 | 40.4 | 1.6 | 0 | 58 | 39.4 | 1.6 | 1 | 58 | 38.4 |
| 57 | 41.4 | 1.6 | 0 | 57 | 40.4 | 1.6 | 1 | 57 | 39.4 |
| 56 | 42.4 | 1.6 | 0 | 56 | 41.4 | 1.6 | 1 | 56 | 40.4 |
| 55 | 43.4 | 1.6 | 0 | 55 | 42.4 | 1.6 | 1 | 55 | 41.4 |
| 54 | 44.4 | 1.6 | 0 | 54 | 43.4 | 1.6 | 1 | 54 | 42.4 |
| 53 | 45.4 | 1.6 | 0 | 53 | 44.4 | 1.6 | 1 | 53 | 43.4 |
| 52 | 46.4 | 1.6 | 0 | 52 | 45.4 | 1.6 | 1 | 52 | 44.4 |
| 51 | 47.4 | 1.6 | 0 | 51 | 46.4 | 1.6 | 1 | 51 | 45.4 |
| 50 | 48.4 | 1.6 | 0 | 50 | 47.4 | 1.6 | 1 | 50 | 46.4 |
| 49 | 49.4 | 1.6 | 0 | 49 | 48.4 | 1.6 | 1 | 49 | 47.4 |
| 48 | 50.4 | 1.6 | 0 | 48 | 49.4 | 1.6 | 1 | 48 | 48.4 |
| 47 | 51.4 | 1.6 | 0 | 47 | 50.4 | 1.6 | 1 | 47 | 49.4 |
| 46 | 52.4 | 1.6 | 0 | 46 | 51.4 | 1.6 | 1 | 46 | 50.4 |
| 45 | 53.4 | 1.6 | 0 | 45 | 52.4 | 1.6 | 1 | 45 | 51.4 |
| 44 | 54.4 | 1.6 | 0 | 44 | 53.4 | 1.6 | 1 | 44 | 52.4 |
| 43 | 55.4 | 1.6 | 0 | 43 | 54.4 | 1.6 | 1 | 43 | 53.4 |
| 42 | 56.4 | 1.6 | 0 | 42 | 55.4 | 1.6 | 1 | 42 | 54.4 |
| 41 | 57.4 | 1.6 | 0 | 41 | 56.4 | 1.6 | 1 | 41 | 55.4 |
| 40 | 58.4 | 1.6 | 0 | 40 | 57.4 | 1.6 | 1 | 40 | 56.4 |
| 39 | 59.4 | 1.6 | 0 | 39 | 58.4 | 1.6 | 1 | 39 | 57.4 |
| 38 | 60.4 | 1.6 | 0 | 38 | 59.4 | 1.6 | 1 | 38 | 58.4 |
| 37 | 61.4 | 1.6 | 0 | 37 | 60.4 | 1.6 | 1 | 37 | 59.4 |
| 36 | 62.4 | 1.6 | 0 | 36 | 61.4 | 1.6 | 1 | 36 | 60.4 |
| 35 | 63.4 | 1.6 | 0 | 35 | 62.4 | 1.6 | 1 | 35 | 61.4 |
| 34 | 64.4 | 1.6 | 0 | 34 | 63.4 | 1.6 | 1 | 34 | 62.4 |
| 33 | 65.4 | 1.6 | 0 | 33 | 64.4 | 1.6 | 1 | 33 | 63.4 |
| 32 | 66.4 | 1.6 | 0 | 32 | 65.4 | 1.6 | 1 | 32 | 64.4 |
| 31 | 67.4 | 1.6 | 0 | 31 | 66.4 | 1.6 | 1 | 31 | 65.4 |
| 30 | 68.4 | 1.6 | 0 | 30 | 67.4 | 1.6 | 1 | 30 | 66.4 |
| 60 | 38.2 | 1.8 | 0 | 60 | 37.2 | 1.8 | 1 | 60 | 36.2 |
| 59 | 39.2 | 1.8 | 0 | 59 | 38.2 | 1.8 | 1 | 59 | 37.2 |
| 58 | 40.2 | 1.8 | 0 | 58 | 39.2 | 1.8 | 1 | 58 | 38.2 |
| 57 | 41.2 | 1.8 | 0 | 57 | 40.2 | 1.8 | 1 | 57 | 39.2 |
| 56 | 42.2 | 1.8 | 0 | 56 | 41.2 | 1.8 | 1 | 56 | 40.2 |
| 55 | 43.2 | 1.8 | 0 | 55 | 42.2 | 1.8 | 1 | 55 | 41.2 |
| 54 | 44.2 | 1.8 | 0 | 54 | 43.2 | 1.8 | 1 | 54 | 42.2 |
| 53 | 45.2 | 1.8 | 0 | 53 | 44.2 | 1.8 | 1 | 53 | 43.2 |
| 52 | 46.2 | 1.8 | 0 | 52 | 45.2 | 1.8 | 1 | 52 | 44.2 |
| 51 | 47.2 | 1.8 | 0 | 51 | 46.2 | 1.8 | 1 | 51 | 45.2 |
| 50 | 48.2 | 1.8 | 0 | 50 | 47.2 | 1.8 | 1 | 50 | 46.2 |
| 49 | 49.2 | 1.8 | 0 | 49 | 48.2 | 1.8 | 1 | 49 | 47.2 |
| 48 | 50.2 | 1.8 | 0 | 48 | 49.2 | 1.8 | 1 | 48 | 48.2 |
| 47 | 51.2 | 1.8 | 0 | 47 | 50.2 | 1.8 | 1 | 47 | 49.2 |
| 46 | 52.2 | 1.8 | 0 | 46 | 51.2 | 1.8 | 1 | 46 | 50.2 |
| 45 | 53.2 | 1.8 | 0 | 45 | 52.2 | 1.8 | 1 | 45 | 51.2 |
| 44 | 54.2 | 1.8 | 0 | 44 | 53.2 | 1.8 | 1 | 44 | 52.2 |
| 43 | 55.2 | 1.8 | 0 | 43 | 54.2 | 1.8 | 1 | 43 | 53.2 |
| 42 | 56.2 | 1.8 | 0 | 42 | 55.2 | 1.8 | 1 | 42 | 54.2 |
| 41 | 57.2 | 1.8 | 0 | 41 | 56.2 | 1.8 | 1 | 41 | 55.2 |
| 40 | 58.2 | 1.8 | 0 | 40 | 57.2 | 1.8 | 1 | 40 | 56.2 |
| 39 | 59.2 | 1.8 | 0 | 39 | 58.2 | 1.8 | 1 | 39 | 57.2 |
| 38 | 60.2 | 1.8 | 0 | 38 | 59.2 | 1.8 | 1 | 38 | 58.2 |
| 37 | 61.2 | 1.8 | 0 | 37 | 60.2 | 1.8 | 1 | 37 | 59.2 |
| 36 | 62.2 | 1.8 | 0 | 36 | 61.2 | 1.8 | 1 | 36 | 60.2 |
| 35 | 63.2 | 1.8 | 0 | 35 | 62.2 | 1.8 | 1 | 35 | 61.2 |
| 34 | 64.2 | 1.8 | 0 | 34 | 63.2 | 1.8 | 1 | 34 | 62.2 |
| 33 | 65.2 | 1.8 | 0 | 33 | 64.2 | 1.8 | 1 | 33 | 63.2 |
| 32 | 66.2 | 1.8 | 0 | 32 | 65.2 | 1.8 | 1 | 32 | 64.2 |
| 31 | 67.2 | 1.8 | 0 | 31 | 66.2 | 1.8 | 1 | 31 | 65.2 |
| 30 | 68.2 | 1.8 | 0 | 30 | 67.2 | 1.8 | 1 | 30 | 66.2 |
| 60 | 38 | 2 | 0 | 60 | 37 | 2 | 1 | 60 | 36 |
| 59 | 39 | 2 | 0 | 59 | 38 | 2 | 1 | 59 | 37 |
| 58 | 40 | 2 | 0 | 58 | 39 | 2 | 1 | 58 | 38 |
| 57 | 41 | 2 | 0 | 57 | 40 | 2 | 1 | 57 | 39 |
| 56 | 42 | 2 | 0 | 56 | 41 | 2 | 1 | 56 | 40 |
| 55 | 43 | 2 | 0 | 55 | 42 | 2 | 1 | 55 | 41 |
| 54 | 44 | 2 | 0 | 54 | 43 | 2 | 1 | 54 | 42 |
| 53 | 45 | 2 | 0 | 53 | 44 | 2 | 1 | 53 | 43 |
| 52 | 46 | 2 | 0 | 52 | 45 | 2 | 1 | 52 | 44 |
| 51 | 47 | 2 | 0 | 51 | 46 | 2 | 1 | 51 | 45 |
| 50 | 48 | 2 | 0 | 50 | 47 | 2 | 1 | 50 | 46 |
| 49 | 49 | 2 | 0 | 49 | 48 | 2 | 1 | 49 | 47 |
| 48 | 50 | 2 | 0 | 48 | 49 | 2 | 1 | 48 | 48 |
| 47 | 51 | 2 | 0 | 47 | 50 | 2 | 1 | 47 | 49 |
| 46 | 52 | 2 | 0 | 46 | 51 | 2 | 1 | 46 | 50 |
| 45 | 53 | 2 | 0 | 45 | 52 | 2 | 1 | 45 | 51 |
| 44 | 54 | 2 | 0 | 44 | 53 | 2 | 1 | 44 | 52 |
| 43 | 55 | 2 | 0 | 43 | 54 | 2 | 1 | 43 | 53 |
| 42 | 56 | 2 | 0 | 42 | 55 | 2 | 1 | 42 | 54 |
| 41 | 57 | 2 | 0 | 41 | 56 | 2 | 1 | 41 | 55 |
| 40 | 58 | 2 | 0 | 40 | 57 | 2 | 1 | 40 | 56 |
| 39 | 59 | 2 | 0 | 39 | 58 | 2 | 1 | 39 | 57 |
| 38 | 60 | 2 | 0 | 38 | 59 | 2 | 1 | 38 | 58 |
| 37 | 61 | 2 | 0 | 37 | 60 | 2 | 1 | 37 | 59 |
| 36 | 62 | 2 | 0 | 36 | 61 | 2 | 1 | 36 | 60 |
| 35 | 63 | 2 | 0 | 35 | 62 | 2 | 1 | 35 | 61 |
| 34 | 64 | 2 | 0 | 34 | 63 | 2 | 1 | 34 | 62 |
| 33 | 65 | 2 | 0 | 33 | 64 | 2 | 1 | 33 | 63 |
| 32 | 66 | 2 | 0 | 32 | 65 | 2 | 1 | 32 | 64 |
| 31 | 67 | 2 | 0 | 31 | 66 | 2 | 1 | 31 | 65 |
| 30 | 68 | 2 | 0 | 30 | 67 | 2 | 1 | 30 | 66 |
| 60 | 37.8 | 2.2 | 0 | 60 | 36.8 | 2.2 | 1 | 60 | 35.8 |
| 59 | 38.8 | 2.2 | 0 | 59 | 37.8 | 2.2 | 1 | 59 | 36.8 |
| 58 | 39.8 | 2.2 | 0 | 58 | 38.8 | 2.2 | 1 | 58 | 37.8 |
| 57 | 40.8 | 2.2 | 0 | 57 | 39.8 | 2.2 | 1 | 57 | 38.8 |
| 56 | 41.8 | 2.2 | 0 | 56 | 40.8 | 2.2 | 1 | 56 | 39.8 |
| 55 | 42.8 | 2.2 | 0 | 55 | 41.8 | 2.2 | 1 | 55 | 40.8 |
| 54 | 43.8 | 2.2 | 0 | 54 | 42.8 | 2.2 | 1 | 54 | 41.8 |
| 53 | 44.8 | 2.2 | 0 | 53 | 43.8 | 2.2 | 1 | 53 | 42.8 |
| 52 | 45.8 | 2.2 | 0 | 52 | 44.8 | 2.2 | 1 | 52 | 43.8 |
| 51 | 46.8 | 2.2 | 0 | 51 | 45.8 | 2.2 | 1 | 51 | 44.8 |
| 50 | 47.8 | 2.2 | 0 | 50 | 46.8 | 2.2 | 1 | 50 | 45.8 |
| 49 | 48.8 | 2.2 | 0 | 49 | 47.8 | 2.2 | 1 | 49 | 46.8 |
| 48 | 49.8 | 2.2 | 0 | 48 | 48.8 | 2.2 | 1 | 48 | 47.8 |
| 47 | 50.8 | 2.2 | 0 | 47 | 49.8 | 2.2 | 1 | 47 | 48.8 |
| 46 | 51.8 | 2.2 | 0 | 46 | 50.8 | 2.2 | 1 | 46 | 49.8 |
| 45 | 52.8 | 2.2 | 0 | 45 | 51.8 | 2.2 | 1 | 45 | 50.8 |
| 44 | 53.8 | 2.2 | 0 | 44 | 52.8 | 2.2 | 1 | 44 | 51.8 |
| 43 | 54.8 | 2.2 | 0 | 43 | 53.8 | 2.2 | 1 | 43 | 52.8 |
| 42 | 55.8 | 2.2 | 0 | 42 | 54.8 | 2.2 | 1 | 42 | 53.8 |
| 41 | 56.8 | 2.2 | 0 | 41 | 55.8 | 2.2 | 1 | 41 | 54.8 |
| 40 | 57.8 | 2.2 | 0 | 40 | 56.8 | 2.2 | 1 | 40 | 55.8 |
| 39 | 58.8 | 2.2 | 0 | 39 | 57.8 | 2.2 | 1 | 39 | 56.8 |
| 38 | 59.8 | 2.2 | 0 | 38 | 58.8 | 2.2 | 1 | 38 | 57.8 |
| 37 | 60.8 | 2.2 | 0 | 37 | 59.8 | 2.2 | 1 | 37 | 58.8 |
| 36 | 61.8 | 2.2 | 0 | 36 | 60.8 | 2.2 | 1 | 36 | 59.8 |
| 35 | 62.8 | 2.2 | 0 | 35 | 61.8 | 2.2 | 1 | 35 | 60.8 |
| 34 | 63.8 | 2.2 | 0 | 34 | 62.8 | 2.2 | 1 | 34 | 61.8 |
| 33 | 64.8 | 2.2 | 0 | 33 | 63.8 | 2.2 | 1 | 33 | 62.8 |
| 32 | 65.8 | 2.2 | 0 | 32 | 64.8 | 2.2 | 1 | 32 | 63.8 |
| 31 | 66.8 | 2.2 | 0 | 31 | 65.8 | 2.2 | 1 | 31 | 64.8 |
| 30 | 67.8 | 2.2 | 0 | 30 | 66.8 | 2.2 | 1 | 30 | 65.8 |
| 60 | 37.6 | 2.4 | 0 | 60 | 36.6 | 2.4 | 1 | 60 | 35.6 |
| 59 | 38.6 | 2.4 | 0 | 59 | 37.6 | 2.4 | 1 | 59 | 36.6 |
| 58 | 39.6 | 2.4 | 0 | 58 | 38.6 | 2.4 | 1 | 58 | 37.6 |
| 57 | 40.6 | 2.4 | 0 | 57 | 39.6 | 2.4 | 1 | 57 | 38.6 |
| 56 | 41.6 | 2.4 | 0 | 56 | 40.6 | 2.4 | 1 | 56 | 39.6 |
| 55 | 42.6 | 2.4 | 0 | 55 | 41.6 | 2.4 | 1 | 55 | 40.6 |
| 54 | 43.6 | 2.4 | 0 | 54 | 42.6 | 2.4 | 1 | 54 | 41.6 |
| 53 | 44.6 | 2.4 | 0 | 53 | 43.6 | 2.4 | 1 | 53 | 42.6 |
| 52 | 45.6 | 2.4 | 0 | 52 | 44.6 | 2.4 | 1 | 52 | 43.6 |
| 51 | 46.6 | 2.4 | 0 | 51 | 45.6 | 2.4 | 1 | 51 | 44.6 |
| 50 | 47.6 | 2.4 | 0 | 50 | 46.6 | 2.4 | 1 | 50 | 45.6 |
| 49 | 48.6 | 2.4 | 0 | 49 | 47.6 | 2.4 | 1 | 49 | 46.6 |
| 48 | 49.6 | 2.4 | 0 | 48 | 48.6 | 2.4 | 1 | 48 | 47.6 |
| 47 | 50.6 | 2.4 | 0 | 47 | 49.6 | 2.4 | 1 | 47 | 48.6 |
| 46 | 51.6 | 2.4 | 0 | 46 | 50.6 | 2.4 | 1 | 46 | 49.6 |
| 45 | 52.6 | 2.4 | 0 | 45 | 51.6 | 2.4 | 1 | 45 | 50.6 |
| 44 | 53.6 | 2.4 | 0 | 44 | 52.6 | 2.4 | 1 | 44 | 51.6 |
| 43 | 54.6 | 2.4 | 0 | 43 | 53.6 | 2.4 | 1 | 43 | 52.6 |
| 42 | 55.6 | 2.4 | 0 | 42 | 54.6 | 2.4 | 1 | 42 | 53.6 |
| 41 | 56.6 | 2.4 | 0 | 41 | 55.6 | 2.4 | 1 | 41 | 54.6 |
| 40 | 57.6 | 2.4 | 0 | 40 | 56.6 | 2.4 | 1 | 40 | 55.6 |
| 39 | 58.6 | 2.4 | 0 | 39 | 57.6 | 2.4 | 1 | 39 | 56.6 |
| 38 | 59.6 | 2.4 | 0 | 38 | 58.6 | 2.4 | 1 | 38 | 57.6 |
| 37 | 60.6 | 2.4 | 0 | 37 | 59.6 | 2.4 | 1 | 37 | 58.6 |
| 36 | 61.6 | 2.4 | 0 | 36 | 60.6 | 2.4 | 1 | 36 | 59.6 |
| 35 | 62.6 | 2.4 | 0 | 35 | 61.6 | 2.4 | 1 | 35 | 60.6 |
| 34 | 63.6 | 2.4 | 0 | 34 | 62.6 | 2.4 | 1 | 34 | 61.6 |
| 33 | 64.6 | 2.4 | 0 | 33 | 63.6 | 2.4 | 1 | 33 | 62.6 |
| 32 | 65.6 | 2.4 | 0 | 32 | 64.6 | 2.4 | 1 | 32 | 63.6 |
| 31 | 66.6 | 2.4 | 0 | 31 | 65.6 | 2.4 | 1 | 31 | 64.6 |
| 30 | 67.6 | 2.4 | 0 | 30 | 66.6 | 2.4 | 1 | 30 | 65.6 |
| 60 | 37.4 | 2.6 | 0 | 60 | 36.4 | 2.6 | 1 | 60 | 35.4 |
| 59 | 38.4 | 2.6 | 0 | 59 | 37.4 | 2.6 | 1 | 59 | 36.4 |
| 58 | 39.4 | 2.6 | 0 | 58 | 38.4 | 2.6 | 1 | 58 | 37.4 |
| 57 | 40.4 | 2.6 | 0 | 57 | 39.4 | 2.6 | 1 | 57 | 38.4 |
| 56 | 41.4 | 2.6 | 0 | 56 | 40.4 | 2.6 | 1 | 56 | 39.4 |
| 55 | 42.4 | 2.6 | 0 | 55 | 41.4 | 2.6 | 1 | 55 | 40.4 |
| 54 | 43.4 | 2.6 | 0 | 54 | 42.4 | 2.6 | 1 | 54 | 41.4 |
| 53 | 44.4 | 2.6 | 0 | 53 | 43.4 | 2.6 | 1 | 53 | 42.4 |
| 52 | 45.4 | 2.6 | 0 | 52 | 44.4 | 2.6 | 1 | 52 | 43.4 |
| 51 | 46.4 | 2.6 | 0 | 51 | 45.4 | 2.6 | 1 | 51 | 44.4 |
| 50 | 47.4 | 2.6 | 0 | 50 | 46.4 | 2.6 | 1 | 50 | 45.4 |
| 49 | 48.4 | 2.6 | 0 | 49 | 47.4 | 2.6 | 1 | 49 | 46.4 |
| 48 | 49.4 | 2.6 | 0 | 48 | 48.4 | 2.6 | 1 | 48 | 47.4 |
| 47 | 50.4 | 2.6 | 0 | 47 | 49.4 | 2.6 | 1 | 47 | 48.4 |
| 46 | 51.4 | 2.6 | 0 | 46 | 50.4 | 2.6 | 1 | 46 | 49.4 |
| 45 | 52.4 | 2.6 | 0 | 45 | 51.4 | 2.6 | 1 | 45 | 50.4 |
| 44 | 53.4 | 2.6 | 0 | 44 | 52.4 | 2.6 | 1 | 44 | 51.4 |
| 43 | 54.4 | 2.6 | 0 | 43 | 53.4 | 2.6 | 1 | 43 | 52.4 |
| 42 | 55.4 | 2.6 | 0 | 42 | 54.4 | 2.6 | 1 | 42 | 53.4 |
| 41 | 56.4 | 2.6 | 0 | 41 | 55.4 | 2.6 | 1 | 41 | 54.4 |
| 40 | 57.4 | 2.6 | 0 | 40 | 56.4 | 2.6 | 1 | 40 | 55.4 |
| 39 | 58.4 | 2.6 | 0 | 39 | 57.4 | 2.6 | 1 | 39 | 56.4 |
| 38 | 59.4 | 2.6 | 0 | 38 | 58.4 | 2.6 | 1 | 38 | 57.4 |
| 37 | 60.4 | 2.6 | 0 | 37 | 59.4 | 2.6 | 1 | 37 | 58.4 |
| 36 | 61.4 | 2.6 | 0 | 36 | 60.4 | 2.6 | 1 | 36 | 59.4 |
| 35 | 62.4 | 2.6 | 0 | 35 | 61.4 | 2.6 | 1 | 35 | 60.4 |
| 34 | 63.4 | 2.6 | 0 | 34 | 62.4 | 2.6 | 1 | 34 | 61.4 |
| 33 | 64.4 | 2.6 | 0 | 33 | 63.4 | 2.6 | 1 | 33 | 62.4 |
| 32 | 65.4 | 2.6 | 0 | 32 | 64.4 | 2.6 | 1 | 32 | 63.4 |
| 31 | 66.4 | 2.6 | 0 | 31 | 65.4 | 2.6 | 1 | 31 | 64.4 |
| 30 | 67.4 | 2.6 | 0 | 30 | 66.4 | 2.6 | 1 | 30 | 65.4 |
| 60 | 37.2 | 2.8 | 0 | 60 | 36.2 | 2.8 | 1 | 60 | 35.2 |
| 59 | 38.2 | 2.8 | 0 | 59 | 37.2 | 2.8 | 1 | 59 | 36.2 |
| 58 | 39.2 | 2.8 | 0 | 58 | 38.2 | 2.8 | 1 | 58 | 37.2 |
| 57 | 40.2 | 2.8 | 0 | 57 | 39.2 | 2.8 | 1 | 57 | 38.2 |
| 56 | 41.2 | 2.8 | 0 | 56 | 40.2 | 2.8 | 1 | 56 | 39.2 |
| 55 | 42.2 | 2.8 | 0 | 55 | 41.2 | 2.8 | 1 | 55 | 40.2 |
| 54 | 43.2 | 2.8 | 0 | 54 | 42.2 | 2.8 | 1 | 54 | 41.2 |
| 53 | 44.2 | 2.8 | 0 | 53 | 43.2 | 2.8 | 1 | 53 | 42.2 |
| 52 | 45.2 | 2.8 | 0 | 52 | 44.2 | 2.8 | 1 | 52 | 43.2 |
| 51 | 46.2 | 2.8 | 0 | 51 | 45.2 | 2.8 | 1 | 51 | 44.2 |
| 50 | 47.2 | 2.8 | 0 | 50 | 46.2 | 2.8 | 1 | 50 | 45.2 |
| 49 | 48.2 | 2.8 | 0 | 49 | 47.2 | 2.8 | 1 | 49 | 46.2 |
| 48 | 49.2 | 2.8 | 0 | 48 | 48.2 | 2.8 | 1 | 48 | 47.2 |
| 47 | 50.2 | 2.8 | 0 | 47 | 49.2 | 2.8 | 1 | 47 | 48.2 |
| 46 | 51.2 | 2.8 | 0 | 46 | 50.2 | 2.8 | 1 | 46 | 49.2 |
| 45 | 52.2 | 2.8 | 0 | 45 | 51.2 | 2.8 | 1 | 45 | 50.2 |
| 44 | 53.2 | 2.8 | 0 | 44 | 52.2 | 2.8 | 1 | 44 | 51.2 |
| 43 | 54.2 | 2.8 | 0 | 43 | 53.2 | 2.8 | 1 | 43 | 52.2 |
| 42 | 55.2 | 2.8 | 0 | 42 | 54.2 | 2.8 | 1 | 42 | 53.2 |
| 41 | 56.2 | 2.8 | 0 | 41 | 55.2 | 2.8 | 1 | 41 | 54.2 |
| 40 | 57.2 | 2.8 | 0 | 40 | 56.2 | 2.8 | 1 | 40 | 55.2 |
| 39 | 58.2 | 2.8 | 0 | 39 | 57.2 | 2.8 | 1 | 39 | 56.2 |
| 38 | 59.2 | 2.8 | 0 | 38 | 58.2 | 2.8 | 1 | 38 | 57.2 |
| 37 | 60.2 | 2.8 | 0 | 37 | 59.2 | 2.8 | 1 | 37 | 58.2 |
| 36 | 61.2 | 2.8 | 0 | 36 | 60.2 | 2.8 | 1 | 36 | 59.2 |
| 35 | 62.2 | 2.8 | 0 | 35 | 61.2 | 2.8 | 1 | 35 | 60.2 |
| 34 | 63.2 | 2.8 | 0 | 34 | 62.2 | 2.8 | 1 | 34 | 61.2 |
| 33 | 64.2 | 2.8 | 0 | 33 | 63.2 | 2.8 | 1 | 33 | 62.2 |
| 32 | 65.2 | 2.8 | 0 | 32 | 64.2 | 2.8 | 1 | 32 | 63.2 |
| 31 | 66.2 | 2.8 | 0 | 31 | 65.2 | 2.8 | 1 | 31 | 64.2 |
| 30 | 67.2 | 2.8 | 0 | 30 | 66.2 | 2.8 | 1 | 30 | 65.2 |
| 60 | 37 | 3 | 0 | 60 | 36 | 3 | 1 | 60 | 35 |
| 59 | 38 | 3 | 0 | 59 | 37 | 3 | 1 | 59 | 36 |
| 58 | 39 | 3 | 0 | 58 | 38 | 3 | 1 | 58 | 37 |
| 57 | 40 | 3 | 0 | 57 | 39 | 3 | 1 | 57 | 38 |
| 56 | 41 | 3 | 0 | 56 | 40 | 3 | 1 | 56 | 39 |
| 55 | 42 | 3 | 0 | 55 | 41 | 3 | 1 | 55 | 40 |
| 54 | 43 | 3 | 0 | 54 | 42 | 3 | 1 | 54 | 41 |
| 53 | 44 | 3 | 0 | 53 | 43 | 3 | 1 | 53 | 42 |
| 52 | 45 | 3 | 0 | 52 | 44 | 3 | 1 | 52 | 43 |
| 51 | 46 | 3 | 0 | 51 | 45 | 3 | 1 | 51 | 44 |
| 50 | 47 | 3 | 0 | 50 | 46 | 3 | 1 | 50 | 45 |
| 49 | 48 | 3 | 0 | 49 | 47 | 3 | 1 | 49 | 46 |
| 48 | 49 | 3 | 0 | 48 | 48 | 3 | 1 | 48 | 47 |
| 47 | 50 | 3 | 0 | 47 | 49 | 3 | 1 | 47 | 48 |
| 46 | 51 | 3 | 0 | 46 | 50 | 3 | 1 | 46 | 49 |
| 45 | 52 | 3 | 0 | 45 | 51 | 3 | 1 | 45 | 50 |
| 44 | 53 | 3 | 0 | 44 | 52 | 3 | 1 | 44 | 51 |
| 43 | 54 | 3 | 0 | 43 | 53 | 3 | 1 | 43 | 52 |
| 42 | 55 | 3 | 0 | 42 | 54 | 3 | 1 | 42 | 53 |
| 41 | 56 | 3 | 0 | 41 | 55 | 3 | 1 | 41 | 54 |
| 40 | 57 | 3 | 0 | 40 | 56 | 3 | 1 | 40 | 55 |
| 39 | 58 | 3 | 0 | 39 | 57 | 3 | 1 | 39 | 56 |
| 38 | 59 | 3 | 0 | 38 | 58 | 3 | 1 | 38 | 57 |
| 37 | 60 | 3 | 0 | 37 | 59 | 3 | 1 | 37 | 58 |
| 36 | 61 | 3 | 0 | 36 | 60 | 3 | 1 | 36 | 59 |
| 35 | 62 | 3 | 0 | 35 | 61 | 3 | 1 | 35 | 60 |
| 34 | 63 | 3 | 0 | 34 | 62 | 3 | 1 | 34 | 61 |
| 33 | 64 | 3 | 0 | 33 | 63 | 3 | 1 | 33 | 62 |
| 32 | 65 | 3 | 0 | 32 | 64 | 3 | 1 | 32 | 63 |
| 31 | 66 | 3 | 0 | 31 | 65 | 3 | 1 | 31 | 64 |
| 30 | 67 | 3 | 0 | 30 | 66 | 3 | 1 | 30 | 65 |
| 60 | 35 | 1 | 4 | 60 | 34 | 1 | 5 | 60 | 33 |
| 59 | 36 | 1 | 4 | 59 | 35 | 1 | 5 | 59 | 34 |
| 58 | 37 | 1 | 4 | 58 | 36 | 1 | 5 | 58 | 35 |
| 57 | 38 | 1 | 4 | 57 | 37 | 1 | 5 | 57 | 36 |
| 56 | 39 | 1 | 4 | 56 | 38 | 1 | 5 | 56 | 37 |
| 55 | 40 | 1 | 4 | 55 | 39 | 1 | 5 | 55 | 38 |
| 54 | 41 | 1 | 4 | 54 | 40 | 1 | 5 | 54 | 39 |
| 53 | 42 | 1 | 4 | 53 | 41 | 1 | 5 | 53 | 40 |
| 52 | 43 | 1 | 4 | 52 | 42 | 1 | 5 | 52 | 41 |
| 51 | 44 | 1 | 4 | 51 | 43 | 1 | 5 | 51 | 42 |
| 50 | 45 | 1 | 4 | 50 | 44 | 1 | 5 | 50 | 43 |
| 49 | 46 | 1 | 4 | 49 | 45 | 1 | 5 | 49 | 44 |
| 48 | 47 | 1 | 4 | 48 | 46 | 1 | 5 | 48 | 45 |
| 47 | 48 | 1 | 4 | 47 | 47 | 1 | 5 | 47 | 46 |
| 46 | 49 | 1 | 4 | 46 | 48 | 1 | 5 | 46 | 47 |
| 45 | 50 | 1 | 4 | 45 | 49 | 1 | 5 | 45 | 48 |
| 44 | 51 | 1 | 4 | 44 | 50 | 1 | 5 | 44 | 49 |
| 43 | 52 | 1 | 4 | 43 | 51 | 1 | 5 | 43 | 50 |
| 42 | 53 | 1 | 4 | 42 | 52 | 1 | 5 | 42 | 51 |
| 41 | 54 | 1 | 4 | 41 | 53 | 1 | 5 | 41 | 52 |
| 40 | 55 | 1 | 4 | 40 | 54 | 1 | 5 | 40 | 53 |
| 39 | 56 | 1 | 4 | 39 | 55 | 1 | 5 | 39 | 54 |
| 38 | 57 | 1 | 4 | 38 | 56 | 1 | 5 | 38 | 55 |
| 37 | 58 | 1 | 4 | 37 | 57 | 1 | 5 | 37 | 56 |
| 36 | 59 | 1 | 4 | 36 | 58 | 1 | 5 | 36 | 57 |
| 35 | 60 | 1 | 4 | 35 | 59 | 1 | 5 | 35 | 58 |
| 34 | 61 | 1 | 4 | 34 | 60 | 1 | 5 | 34 | 59 |
| 33 | 62 | 1 | 4 | 33 | 61 | 1 | 5 | 33 | 60 |
| 32 | 63 | 1 | 4 | 32 | 62 | 1 | 5 | 32 | 61 |
| 31 | 64 | 1 | 4 | 31 | 63 | 1 | 5 | 31 | 62 |
| 30 | 65 | 1 | 4 | 30 | 64 | 1 | 5 | 30 | 63 |
| 60 | 34.8 | 1.2 | 4 | 60 | 33.8 | 1.2 | 5 | 60 | 32.8 |
| 59 | 35.8 | 1.2 | 4 | 59 | 34.8 | 1.2 | 5 | 59 | 33.8 |
| 58 | 36.8 | 1.2 | 4 | 58 | 35.8 | 1.2 | 5 | 58 | 34.8 |
| 57 | 37.8 | 1.2 | 4 | 57 | 36.8 | 1.2 | 5 | 57 | 35.8 |
| 56 | 38.8 | 1.2 | 4 | 56 | 37.8 | 1.2 | 5 | 56 | 36.8 |
| 55 | 39.8 | 1.2 | 4 | 55 | 38.8 | 1.2 | 5 | 55 | 37.8 |
| 54 | 40.8 | 1.2 | 4 | 54 | 39.8 | 1.2 | 5 | 54 | 38.8 |
| 53 | 41.8 | 1.2 | 4 | 53 | 40.8 | 1.2 | 5 | 53 | 39.8 |
| 52 | 42.8 | 1.2 | 4 | 52 | 41.8 | 1.2 | 5 | 52 | 40.8 |
| 51 | 43.8 | 1.2 | 4 | 51 | 42.8 | 1.2 | 5 | 51 | 41.8 |
| 50 | 44.8 | 1.2 | 4 | 50 | 43.8 | 1.2 | 5 | 50 | 42.8 |
| 49 | 45.8 | 1.2 | 4 | 49 | 44.8 | 1.2 | 5 | 49 | 43.8 |
| 48 | 46.8 | 1.2 | 4 | 48 | 45.8 | 1.2 | 5 | 48 | 44.8 |
| 47 | 47.8 | 1.2 | 4 | 47 | 46.8 | 1.2 | 5 | 47 | 45.8 |
| 46 | 48.8 | 1.2 | 4 | 46 | 47.8 | 1.2 | 5 | 46 | 46.8 |
| 45 | 49.8 | 1.2 | 4 | 45 | 48.8 | 1.2 | 5 | 45 | 47.8 |
| 44 | 50.8 | 1.2 | 4 | 44 | 49.8 | 1.2 | 5 | 44 | 48.8 |
| 43 | 51.8 | 1.2 | 4 | 43 | 50.8 | 1.2 | 5 | 43 | 49.8 |
| 42 | 52.8 | 1.2 | 4 | 42 | 51.8 | 1.2 | 5 | 42 | 50.8 |
| 41 | 53.8 | 1.2 | 4 | 41 | 52.8 | 1.2 | 5 | 41 | 51.8 |
| 40 | 54.8 | 1.2 | 4 | 40 | 53.8 | 1.2 | 5 | 40 | 52.8 |
| 39 | 55.8 | 1.2 | 4 | 39 | 54.8 | 1.2 | 5 | 39 | 53.8 |
| 38 | 56.8 | 1.2 | 4 | 38 | 55.8 | 1.2 | 5 | 38 | 54.8 |
| 37 | 57.8 | 1.2 | 4 | 37 | 56.8 | 1.2 | 5 | 37 | 55.8 |
| 36 | 58.8 | 1.2 | 4 | 36 | 57.8 | 1.2 | 5 | 36 | 56.8 |
| 35 | 59.8 | 1.2 | 4 | 35 | 58.8 | 1.2 | 5 | 35 | 57.8 |
| 34 | 60.8 | 1.2 | 4 | 34 | 59.8 | 1.2 | 5 | 34 | 58.8 |
| 33 | 61.8 | 1.2 | 4 | 33 | 60.8 | 1.2 | 5 | 33 | 59.8 |
| 32 | 62.8 | 1.2 | 4 | 32 | 61.8 | 1.2 | 5 | 32 | 60.8 |
| 31 | 63.8 | 1.2 | 4 | 31 | 62.8 | 1.2 | 5 | 31 | 61.8 |
| 30 | 64.8 | 1.2 | 4 | 30 | 63.8 | 1.2 | 5 | 30 | 62.8 |
| 60 | 34.6 | 1.4 | 4 | 60 | 33.6 | 1.4 | 5 | 60 | 32.6 |
| 59 | 35.6 | 1.4 | 4 | 59 | 34.6 | 1.4 | 5 | 59 | 33.6 |
| 58 | 36.6 | 1.4 | 4 | 58 | 35.6 | 1.4 | 5 | 58 | 34.6 |
| 57 | 37.6 | 1.4 | 4 | 57 | 36.6 | 1.4 | 5 | 57 | 35.6 |
| 56 | 38.6 | 1.4 | 4 | 56 | 37.6 | 1.4 | 5 | 56 | 36.6 |
| 55 | 39.6 | 1.4 | 4 | 55 | 38.6 | 1.4 | 5 | 55 | 37.6 |
| 54 | 40.6 | 1.4 | 4 | 54 | 39.6 | 1.4 | 5 | 54 | 38.6 |
| 53 | 41.6 | 1.4 | 4 | 53 | 40.6 | 1.4 | 5 | 53 | 39.6 |
| 52 | 42.6 | 1.4 | 4 | 52 | 41.6 | 1.4 | 5 | 52 | 40.6 |
| 51 | 43.6 | 1.4 | 4 | 51 | 42.6 | 1.4 | 5 | 51 | 41.6 |
| 50 | 44.6 | 1.4 | 4 | 50 | 43.6 | 1.4 | 5 | 50 | 42.6 |
| 49 | 45.6 | 1.4 | 4 | 49 | 44.6 | 1.4 | 5 | 49 | 43.6 |
| 48 | 46.6 | 1.4 | 4 | 48 | 45.6 | 1.4 | 5 | 48 | 44.6 |
| 47 | 47.6 | 1.4 | 4 | 47 | 46.6 | 1.4 | 5 | 47 | 45.6 |
| 46 | 48.6 | 1.4 | 4 | 46 | 47.6 | 1.4 | 5 | 46 | 46.6 |
| 45 | 49.6 | 1.4 | 4 | 45 | 48.6 | 1.4 | 5 | 45 | 47.6 |
| 44 | 50.6 | 1.4 | 4 | 44 | 49.6 | 1.4 | 5 | 44 | 48.6 |
| 43 | 51.6 | 1.4 | 4 | 43 | 50.6 | 1.4 | 5 | 43 | 49.6 |
| 42 | 52.6 | 1.4 | 4 | 42 | 51.6 | 1.4 | 5 | 42 | 50.6 |
| 41 | 53.6 | 1.4 | 4 | 41 | 52.6 | 1.4 | 5 | 41 | 51.6 |
| 40 | 54.6 | 1.4 | 4 | 40 | 53.6 | 1.4 | 5 | 40 | 52.6 |
| 39 | 55.6 | 1.4 | 4 | 39 | 54.6 | 1.4 | 5 | 39 | 53.6 |
| 38 | 56.6 | 1.4 | 4 | 38 | 55.6 | 1.4 | 5 | 38 | 54.6 |
| 37 | 57.6 | 1.4 | 4 | 37 | 56.6 | 1.4 | 5 | 37 | 55.6 |
| 36 | 58.6 | 1.4 | 4 | 36 | 57.6 | 1.4 | 5 | 36 | 56.6 |
| 35 | 59.6 | 1.4 | 4 | 35 | 58.6 | 1.4 | 5 | 35 | 57.6 |
| 34 | 60.6 | 1.4 | 4 | 34 | 59.6 | 1.4 | 5 | 34 | 58.6 |
| 33 | 61.6 | 1.4 | 4 | 33 | 60.6 | 1.4 | 5 | 33 | 59.6 |
| 32 | 62.6 | 1.4 | 4 | 32 | 61.6 | 1.4 | 5 | 32 | 60.6 |
| 31 | 63.6 | 1.4 | 4 | 31 | 62.6 | 1.4 | 5 | 31 | 61.6 |
| 30 | 64.6 | 1.4 | 4 | 30 | 63.6 | 1.4 | 5 | 30 | 62.6 |
| 60 | 34.4 | 1.6 | 4 | 60 | 33.4 | 1.6 | 5 | 60 | 32.4 |
| 59 | 35.4 | 1.6 | 4 | 59 | 34.4 | 1.6 | 5 | 59 | 33.4 |
| 58 | 36.4 | 1.6 | 4 | 58 | 35.4 | 1.6 | 5 | 58 | 34.4 |
| 57 | 37.4 | 1.6 | 4 | 57 | 36.4 | 1.6 | 5 | 57 | 35.4 |
| 56 | 38.4 | 1.6 | 4 | 56 | 37.4 | 1.6 | 5 | 56 | 36.4 |
| 55 | 39.4 | 1.6 | 4 | 55 | 38.4 | 1.6 | 5 | 55 | 37.4 |
| 54 | 40.4 | 1.6 | 4 | 54 | 39.4 | 1.6 | 5 | 54 | 38.4 |
| 53 | 41.4 | 1.6 | 4 | 53 | 40.4 | 1.6 | 5 | 53 | 39.4 |
| 52 | 42.4 | 1.6 | 4 | 52 | 41.4 | 1.6 | 5 | 52 | 40.4 |
| 51 | 43.4 | 1.6 | 4 | 51 | 42.4 | 1.6 | 5 | 51 | 41.4 |
| 50 | 44.4 | 1.6 | 4 | 50 | 43.4 | 1.6 | 5 | 50 | 42.4 |
| 49 | 45.4 | 1.6 | 4 | 49 | 44.4 | 1.6 | 5 | 49 | 43.4 |
| 48 | 46.4 | 1.6 | 4 | 48 | 45.4 | 1.6 | 5 | 48 | 44.4 |
| 47 | 47.4 | 1.6 | 4 | 47 | 46.4 | 1.6 | 5 | 47 | 45.4 |
| 46 | 48.4 | 1.6 | 4 | 46 | 47.4 | 1.6 | 5 | 46 | 46.4 |
| 45 | 49.4 | 1.6 | 4 | 45 | 48.4 | 1.6 | 5 | 45 | 47.4 |
| 44 | 50.4 | 1.6 | 4 | 44 | 49.4 | 1.6 | 5 | 44 | 48.4 |
| 43 | 51.4 | 1.6 | 4 | 43 | 50.4 | 1.6 | 5 | 43 | 49.4 |
| 42 | 52.4 | 1.6 | 4 | 42 | 51.4 | 1.6 | 5 | 42 | 50.4 |
| 41 | 53.4 | 1.6 | 4 | 41 | 52.4 | 1.6 | 5 | 41 | 51.4 |
| 40 | 54.4 | 1.6 | 4 | 40 | 53.4 | 1.6 | 5 | 40 | 52.4 |
| 39 | 55.4 | 1.6 | 4 | 39 | 54.4 | 1.6 | 5 | 39 | 53.4 |
| 38 | 56.4 | 1.6 | 4 | 38 | 55.4 | 1.6 | 5 | 38 | 54.4 |
| 37 | 57.4 | 1.6 | 4 | 37 | 56.4 | 1.6 | 5 | 37 | 55.4 |
| 36 | 58.4 | 1.6 | 4 | 36 | 57.4 | 1.6 | 5 | 36 | 56.4 |
| 35 | 59.4 | 1.6 | 4 | 35 | 58.4 | 1.6 | 5 | 35 | 57.4 |
| 34 | 60.4 | 1.6 | 4 | 34 | 59.4 | 1.6 | 5 | 34 | 58.4 |
| 33 | 61.4 | 1.6 | 4 | 33 | 60.4 | 1.6 | 5 | 33 | 59.4 |
| 32 | 62.4 | 1.6 | 4 | 32 | 61.4 | 1.6 | 5 | 32 | 60.4 |
| 31 | 63.4 | 1.6 | 4 | 31 | 62.4 | 1.6 | 5 | 31 | 61.4 |
| 30 | 64.4 | 1.6 | 4 | 30 | 63.4 | 1.6 | 5 | 30 | 62.4 |
| 60 | 34.2 | 1.8 | 4 | 60 | 33.2 | 1.8 | 5 | 60 | 32.2 |
| 59 | 35.2 | 1.8 | 4 | 59 | 34.2 | 1.8 | 5 | 59 | 33.2 |
| 58 | 36.2 | 1.8 | 4 | 58 | 35.2 | 1.8 | 5 | 58 | 34.2 |
| 57 | 37.2 | 1.8 | 4 | 57 | 36.2 | 1.8 | 5 | 57 | 35.2 |
| 56 | 38.2 | 1.8 | 4 | 56 | 37.2 | 1.8 | 5 | 56 | 36.2 |
| 55 | 39.2 | 1.8 | 4 | 55 | 38.2 | 1.8 | 5 | 55 | 37.2 |
| 54 | 40.2 | 1.8 | 4 | 54 | 39.2 | 1.8 | 5 | 54 | 38.2 |
| 53 | 41.2 | 1.8 | 4 | 53 | 40.2 | 1.8 | 5 | 53 | 39.2 |
| 52 | 42.2 | 1.8 | 4 | 52 | 41.2 | 1.8 | 5 | 52 | 40.2 |
| 51 | 43.2 | 1.8 | 4 | 51 | 42.2 | 1.8 | 5 | 51 | 41.2 |
| 50 | 44.2 | 1.8 | 4 | 50 | 43.2 | 1.8 | 5 | 50 | 42.2 |
| 49 | 45.2 | 1.8 | 4 | 49 | 44.2 | 1.8 | 5 | 49 | 43.2 |
| 48 | 46.2 | 1.8 | 4 | 48 | 45.2 | 1.8 | 5 | 48 | 44.2 |
| 47 | 47.2 | 1.8 | 4 | 47 | 46.2 | 1.8 | 5 | 47 | 45.2 |
| 46 | 48.2 | 1.8 | 4 | 46 | 47.2 | 1.8 | 5 | 46 | 46.2 |
| 45 | 49.2 | 1.8 | 4 | 45 | 48.2 | 1.8 | 5 | 45 | 47.2 |
| 44 | 50.2 | 1.8 | 4 | 44 | 49.2 | 1.8 | 5 | 44 | 48.2 |
| 43 | 51.2 | 1.8 | 4 | 43 | 50.2 | 1.8 | 5 | 43 | 49.2 |
| 42 | 52.2 | 1.8 | 4 | 42 | 51.2 | 1.8 | 5 | 42 | 50.2 |
| 41 | 53.2 | 1.8 | 4 | 41 | 52.2 | 1.8 | 5 | 41 | 51.2 |
| 40 | 54.2 | 1.8 | 4 | 40 | 53.2 | 1.8 | 5 | 40 | 52.2 |
| 39 | 55.2 | 1.8 | 4 | 39 | 54.2 | 1.8 | 5 | 39 | 53.2 |
| 38 | 56.2 | 1.8 | 4 | 38 | 55.2 | 1.8 | 5 | 38 | 54.2 |
| 37 | 57.2 | 1.8 | 4 | 37 | 56.2 | 1.8 | 5 | 37 | 55.2 |
| 36 | 58.2 | 1.8 | 4 | 36 | 57.2 | 1.8 | 5 | 36 | 56.2 |
| 35 | 59.2 | 1.8 | 4 | 35 | 58.2 | 1.8 | 5 | 35 | 57.2 |
| 34 | 60.2 | 1.8 | 4 | 34 | 59.2 | 1.8 | 5 | 34 | 58.2 |
| 33 | 61.2 | 1.8 | 4 | 33 | 60.2 | 1.8 | 5 | 33 | 59.2 |
| 32 | 62.2 | 1.8 | 4 | 32 | 61.2 | 1.8 | 5 | 32 | 60.2 |
| 31 | 63.2 | 1.8 | 4 | 31 | 62.2 | 1.8 | 5 | 31 | 61.2 |
| 30 | 64.2 | 1.8 | 4 | 30 | 63.2 | 1.8 | 5 | 30 | 62.2 |
| 60 | 34 | 2 | 4 | 60 | 33 | 2 | 5 | 60 | 32 |
| 59 | 35 | 2 | 4 | 59 | 34 | 2 | 5 | 59 | 33 |
| 58 | 36 | 2 | 4 | 58 | 35 | 2 | 5 | 58 | 34 |
| 57 | 37 | 2 | 4 | 57 | 36 | 2 | 5 | 57 | 35 |
| 56 | 38 | 2 | 4 | 56 | 37 | 2 | 5 | 56 | 36 |
| 55 | 39 | 2 | 4 | 55 | 38 | 2 | 5 | 55 | 37 |
| 54 | 40 | 2 | 4 | 54 | 39 | 2 | 5 | 54 | 38 |
| 53 | 41 | 2 | 4 | 53 | 40 | 2 | 5 | 53 | 39 |
| 52 | 42 | 2 | 4 | 52 | 41 | 2 | 5 | 52 | 40 |
| 51 | 43 | 2 | 4 | 51 | 42 | 2 | 5 | 51 | 41 |
| 50 | 44 | 2 | 4 | 50 | 43 | 2 | 5 | 50 | 42 |
| 49 | 45 | 2 | 4 | 49 | 44 | 2 | 5 | 49 | 43 |
| 48 | 46 | 2 | 4 | 48 | 45 | 2 | 5 | 48 | 44 |
| 47 | 47 | 2 | 4 | 47 | 46 | 2 | 5 | 47 | 45 |
| 46 | 48 | 2 | 4 | 46 | 47 | 2 | 5 | 46 | 46 |
| 45 | 49 | 2 | 4 | 45 | 48 | 2 | 5 | 45 | 47 |
| 44 | 50 | 2 | 4 | 44 | 49 | 2 | 5 | 44 | 48 |
| 43 | 51 | 2 | 4 | 43 | 50 | 2 | 5 | 43 | 49 |
| 42 | 52 | 2 | 4 | 42 | 51 | 2 | 5 | 42 | 50 |
| 41 | 53 | 2 | 4 | 41 | 52 | 2 | 5 | 41 | 51 |
| 40 | 54 | 2 | 4 | 40 | 53 | 2 | 5 | 40 | 52 |
| 39 | 55 | 2 | 4 | 39 | 54 | 2 | 5 | 39 | 53 |
| 38 | 56 | 2 | 4 | 38 | 55 | 2 | 5 | 38 | 54 |
| 37 | 57 | 2 | 4 | 37 | 56 | 2 | 5 | 37 | 55 |
| 36 | 58 | 2 | 4 | 36 | 57 | 2 | 5 | 36 | 56 |
| 35 | 59 | 2 | 4 | 35 | 58 | 2 | 5 | 35 | 57 |
| 34 | 60 | 2 | 4 | 34 | 59 | 2 | 5 | 34 | 58 |
| 33 | 61 | 2 | 4 | 33 | 60 | 2 | 5 | 33 | 59 |
| 32 | 62 | 2 | 4 | 32 | 61 | 2 | 5 | 32 | 60 |
| 31 | 63 | 2 | 4 | 31 | 62 | 2 | 5 | 31 | 61 |
| 30 | 64 | 2 | 4 | 30 | 63 | 2 | 5 | 30 | 62 |
| 60 | 33.8 | 2.2 | 4 | 60 | 32.8 | 2.2 | 5 | 60 | 31.8 |
| 59 | 34.8 | 2.2 | 4 | 59 | 33.8 | 2.2 | 5 | 59 | 32.8 |
| 58 | 35.8 | 2.2 | 4 | 58 | 34.8 | 2.2 | 5 | 58 | 33.8 |
| 57 | 36.8 | 2.2 | 4 | 57 | 35.8 | 2.2 | 5 | 57 | 34.8 |
| 56 | 37.8 | 2.2 | 4 | 56 | 36.8 | 2.2 | 5 | 56 | 35.8 |
| 55 | 38.8 | 2.2 | 4 | 55 | 37.8 | 2.2 | 5 | 55 | 36.8 |
| 54 | 39.8 | 2.2 | 4 | 54 | 38.8 | 2.2 | 5 | 54 | 37.8 |
| 53 | 40.8 | 2.2 | 4 | 53 | 39.8 | 2.2 | 5 | 53 | 38.8 |
| 52 | 41.8 | 2.2 | 4 | 52 | 40.8 | 2.2 | 5 | 52 | 39.8 |
| 51 | 42.8 | 2.2 | 4 | 51 | 41.8 | 2.2 | 5 | 51 | 40.8 |
| 50 | 43.8 | 2.2 | 4 | 50 | 42.8 | 2.2 | 5 | 50 | 41.8 |
| 49 | 44.8 | 2.2 | 4 | 49 | 43.8 | 2.2 | 5 | 49 | 42.8 |
| 48 | 45.8 | 2.2 | 4 | 48 | 44.8 | 2.2 | 5 | 48 | 43.8 |
| 47 | 46.8 | 2.2 | 4 | 47 | 45.8 | 2.2 | 5 | 47 | 44.8 |
| 46 | 47.8 | 2.2 | 4 | 46 | 46.8 | 2.2 | 5 | 46 | 45.8 |
| 45 | 48.8 | 2.2 | 4 | 45 | 47.8 | 2.2 | 5 | 45 | 46.8 |
| 44 | 49.8 | 2.2 | 4 | 44 | 48.8 | 2.2 | 5 | 44 | 47.8 |
| 43 | 50.8 | 2.2 | 4 | 43 | 49.8 | 2.2 | 5 | 43 | 48.8 |
| 42 | 51.8 | 2.2 | 4 | 42 | 50.8 | 2.2 | 5 | 42 | 49.8 |
| 41 | 52.8 | 2.2 | 4 | 41 | 51.8 | 2.2 | 5 | 41 | 50.8 |
| 40 | 53.8 | 2.2 | 4 | 40 | 52.8 | 2.2 | 5 | 40 | 51.8 |
| 39 | 54.8 | 2.2 | 4 | 39 | 53.8 | 2.2 | 5 | 39 | 52.8 |
| 38 | 55.8 | 2.2 | 4 | 38 | 54.8 | 2.2 | 5 | 38 | 53.8 |
| 37 | 56.8 | 2.2 | 4 | 37 | 55.8 | 2.2 | 5 | 37 | 54.8 |
| 36 | 57.8 | 2.2 | 4 | 36 | 56.8 | 2.2 | 5 | 36 | 55.8 |
| 35 | 58.8 | 2.2 | 4 | 35 | 57.8 | 2.2 | 5 | 35 | 56.8 |
| 34 | 59.8 | 2.2 | 4 | 34 | 58.8 | 2.2 | 5 | 34 | 57.8 |
| 33 | 60.8 | 2.2 | 4 | 33 | 59.8 | 2.2 | 5 | 33 | 58.8 |
| 32 | 61.8 | 2.2 | 4 | 32 | 60.8 | 2.2 | 5 | 32 | 59.8 |
| 31 | 62.8 | 2.2 | 4 | 31 | 61.8 | 2.2 | 5 | 31 | 60.8 |
| 30 | 63.8 | 2.2 | 4 | 30 | 62.8 | 2.2 | 5 | 30 | 61.8 |
| 60 | 33.6 | 2.4 | 4 | 60 | 32.6 | 2.4 | 5 | 60 | 31.6 |
| 59 | 34.6 | 2.4 | 4 | 59 | 33.6 | 2.4 | 5 | 59 | 32.6 |
| 58 | 35.6 | 2.4 | 4 | 58 | 34.6 | 2.4 | 5 | 58 | 33.6 |
| 57 | 36.6 | 2.4 | 4 | 57 | 35.6 | 2.4 | 5 | 57 | 34.6 |
| 56 | 37.6 | 2.4 | 4 | 56 | 36.6 | 2.4 | 5 | 56 | 35.6 |
| 55 | 38.6 | 2.4 | 4 | 55 | 37.6 | 2.4 | 5 | 55 | 36.6 |
| 54 | 39.6 | 2.4 | 4 | 54 | 38.6 | 2.4 | 5 | 54 | 37.6 |
| 53 | 40.6 | 2.4 | 4 | 53 | 39.6 | 2.4 | 5 | 53 | 38.6 |
| 52 | 41.6 | 2.4 | 4 | 52 | 40.6 | 2.4 | 5 | 52 | 39.6 |
| 51 | 42.6 | 2.4 | 4 | 51 | 41.6 | 2.4 | 5 | 51 | 40.6 |
| 50 | 43.6 | 2.4 | 4 | 50 | 42.6 | 2.4 | 5 | 50 | 41.6 |
| 49 | 44.6 | 2.4 | 4 | 49 | 43.6 | 2.4 | 5 | 49 | 42.6 |
| 48 | 45.6 | 2.4 | 4 | 48 | 44.6 | 2.4 | 5 | 48 | 43.6 |
| 47 | 46.6 | 2.4 | 4 | 47 | 45.6 | 2.4 | 5 | 47 | 44.6 |
| 46 | 47.6 | 2.4 | 4 | 46 | 46.6 | 2.4 | 5 | 46 | 45.6 |
| 45 | 48.6 | 2.4 | 4 | 45 | 47.6 | 2.4 | 5 | 45 | 46.6 |
| 44 | 49.6 | 2.4 | 4 | 44 | 48.6 | 2.4 | 5 | 44 | 47.6 |
| 43 | 50.6 | 2.4 | 4 | 43 | 49.6 | 2.4 | 5 | 43 | 48.6 |
| 42 | 51.6 | 2.4 | 4 | 42 | 50.6 | 2.4 | 5 | 42 | 49.6 |
| 41 | 52.6 | 2.4 | 4 | 41 | 51.6 | 2.4 | 5 | 41 | 50.6 |
| 40 | 53.6 | 2.4 | 4 | 40 | 52.6 | 2.4 | 5 | 40 | 51.6 |
| 39 | 54.6 | 2.4 | 4 | 39 | 53.6 | 2.4 | 5 | 39 | 52.6 |
| 38 | 55.6 | 2.4 | 4 | 38 | 54.6 | 2.4 | 5 | 38 | 53.6 |
| 37 | 56.6 | 2.4 | 4 | 37 | 55.6 | 2.4 | 5 | 37 | 54.6 |
| 36 | 57.6 | 2.4 | 4 | 36 | 56.6 | 2.4 | 5 | 36 | 55.6 |
| 35 | 58.6 | 2.4 | 4 | 35 | 57.6 | 2.4 | 5 | 35 | 56.6 |
| 34 | 59.6 | 2.4 | 4 | 34 | 58.6 | 2.4 | 5 | 34 | 57.6 |
| 33 | 60.6 | 2.4 | 4 | 33 | 59.6 | 2.4 | 5 | 33 | 58.6 |
| 32 | 61.6 | 2.4 | 4 | 32 | 60.6 | 2.4 | 5 | 32 | 59.6 |
| 31 | 62.6 | 2.4 | 4 | 31 | 61.6 | 2.4 | 5 | 31 | 60.6 |
| 30 | 63.6 | 2.4 | 4 | 30 | 62.6 | 2.4 | 5 | 30 | 61.6 |
| 60 | 33.4 | 2.6 | 4 | 60 | 32.4 | 2.6 | 5 | 60 | 31.4 |
| 59 | 34.4 | 2.6 | 4 | 59 | 33.4 | 2.6 | 5 | 59 | 32.4 |
| 58 | 35.4 | 2.6 | 4 | 58 | 34.4 | 2.6 | 5 | 58 | 33.4 |
| 57 | 36.4 | 2.6 | 4 | 57 | 35.4 | 2.6 | 5 | 57 | 34.4 |
| 56 | 37.4 | 2.6 | 4 | 56 | 36.4 | 2.6 | 5 | 56 | 35.4 |
| 55 | 38.4 | 2.6 | 4 | 55 | 37.4 | 2.6 | 5 | 55 | 36.4 |
| 54 | 39.4 | 2.6 | 4 | 54 | 38.4 | 2.6 | 5 | 54 | 37.4 |
| 53 | 40.4 | 2.6 | 4 | 53 | 39.4 | 2.6 | 5 | 53 | 38.4 |
| 52 | 41.4 | 2.6 | 4 | 52 | 40.4 | 2.6 | 5 | 52 | 39.4 |
| 51 | 42.4 | 2.6 | 4 | 51 | 41.4 | 2.6 | 5 | 51 | 40.4 |
| 50 | 43.4 | 2.6 | 4 | 50 | 42.4 | 2.6 | 5 | 50 | 41.4 |
| 49 | 44.4 | 2.6 | 4 | 49 | 43.4 | 2.6 | 5 | 49 | 42.4 |
| 48 | 45.4 | 2.6 | 4 | 48 | 44.4 | 2.6 | 5 | 48 | 43.4 |
| 47 | 46.4 | 2.6 | 4 | 47 | 45.4 | 2.6 | 5 | 47 | 44.4 |
| 46 | 47.4 | 2.6 | 4 | 46 | 46.4 | 2.6 | 5 | 46 | 45.4 |
| 45 | 48.4 | 2.6 | 4 | 45 | 47.4 | 2.6 | 5 | 45 | 46.4 |
| 44 | 49.4 | 2.6 | 4 | 44 | 48.4 | 2.6 | 5 | 44 | 47.4 |
| 43 | 50.4 | 2.6 | 4 | 43 | 49.4 | 2.6 | 5 | 43 | 48.4 |
| 42 | 51.4 | 2.6 | 4 | 42 | 50.4 | 2.6 | 5 | 42 | 49.4 |
| 41 | 52.4 | 2.6 | 4 | 41 | 51.4 | 2.6 | 5 | 41 | 50.4 |
| 40 | 53.4 | 2.6 | 4 | 40 | 52.4 | 2.6 | 5 | 40 | 51.4 |
| 39 | 54.4 | 2.6 | 4 | 39 | 53.4 | 2.6 | 5 | 39 | 52.4 |
| 38 | 55.4 | 2.6 | 4 | 38 | 54.4 | 2.6 | 5 | 38 | 53.4 |
| 37 | 56.4 | 2.6 | 4 | 37 | 55.4 | 2.6 | 5 | 37 | 54.4 |
| 36 | 57.4 | 2.6 | 4 | 36 | 56.4 | 2.6 | 5 | 36 | 55.4 |
| 35 | 58.4 | 2.6 | 4 | 35 | 57.4 | 2.6 | 5 | 35 | 56.4 |
| 34 | 59.4 | 2.6 | 4 | 34 | 58.4 | 2.6 | 5 | 34 | 57.4 |
| 33 | 60.4 | 2.6 | 4 | 33 | 59.4 | 2.6 | 5 | 33 | 58.4 |
| 32 | 61.4 | 2.6 | 4 | 32 | 60.4 | 2.6 | 5 | 32 | 59.4 |
| 31 | 62.4 | 2.6 | 4 | 31 | 61.4 | 2.6 | 5 | 31 | 60.4 |
| 30 | 63.4 | 2.6 | 4 | 30 | 62.4 | 2.6 | 5 | 30 | 61.4 |
| 60 | 33.2 | 2.8 | 4 | 60 | 32.2 | 2.8 | 5 | 60 | 31.2 |
| 59 | 34.2 | 2.8 | 4 | 59 | 33.2 | 2.8 | 5 | 59 | 32.2 |
| 58 | 35.2 | 2.8 | 4 | 58 | 34.2 | 2.8 | 5 | 58 | 33.2 |
| 57 | 36.2 | 2.8 | 4 | 57 | 35.2 | 2.8 | 5 | 57 | 34.2 |
| 56 | 37.2 | 2.8 | 4 | 56 | 36.2 | 2.8 | 5 | 56 | 35.2 |
| 55 | 38.2 | 2.8 | 4 | 55 | 37.2 | 2.8 | 5 | 55 | 36.2 |
| 54 | 39.2 | 2.8 | 4 | 54 | 38.2 | 2.8 | 5 | 54 | 37.2 |
| 53 | 40.2 | 2.8 | 4 | 53 | 39.2 | 2.8 | 5 | 53 | 38.2 |
| 52 | 41.2 | 2.8 | 4 | 52 | 40.2 | 2.8 | 5 | 52 | 39.2 |
| 51 | 42.2 | 2.8 | 4 | 51 | 41.2 | 2.8 | 5 | 51 | 40.2 |
| 50 | 43.2 | 2.8 | 4 | 50 | 42.2 | 2.8 | 5 | 50 | 41.2 |
| 49 | 44.2 | 2.8 | 4 | 49 | 43.2 | 2.8 | 5 | 49 | 42.2 |
| 48 | 45.2 | 2.8 | 4 | 48 | 44.2 | 2.8 | 5 | 48 | 43.2 |
| 47 | 46.2 | 2.8 | 4 | 47 | 45.2 | 2.8 | 5 | 47 | 44.2 |
| 46 | 47.2 | 2.8 | 4 | 46 | 46.2 | 2.8 | 5 | 46 | 45.2 |
| 45 | 48.2 | 2.8 | 4 | 45 | 47.2 | 2.8 | 5 | 45 | 46.2 |
| 44 | 49.2 | 2.8 | 4 | 44 | 48.2 | 2.8 | 5 | 44 | 47.2 |
| 43 | 50.2 | 2.8 | 4 | 43 | 49.2 | 2.8 | 5 | 43 | 48.2 |
| 42 | 51.2 | 2.8 | 4 | 42 | 50.2 | 2.8 | 5 | 42 | 49.2 |
| 41 | 52.2 | 2.8 | 4 | 41 | 51.2 | 2.8 | 5 | 41 | 50.2 |
| 40 | 53.2 | 2.8 | 4 | 40 | 52.2 | 2.8 | 5 | 40 | 51.2 |
| 39 | 54.2 | 2.8 | 4 | 39 | 53.2 | 2.8 | 5 | 39 | 52.2 |
| 38 | 55.2 | 2.8 | 4 | 38 | 54.2 | 2.8 | 5 | 38 | 53.2 |
| 37 | 56.2 | 2.8 | 4 | 37 | 55.2 | 2.8 | 5 | 37 | 54.2 |
| 36 | 57.2 | 2.8 | 4 | 36 | 56.2 | 2.8 | 5 | 36 | 55.2 |
| 35 | 58.2 | 2.8 | 4 | 35 | 57.2 | 2.8 | 5 | 35 | 56.2 |
| 34 | 59.2 | 2.8 | 4 | 34 | 58.2 | 2.8 | 5 | 34 | 57.2 |
| 33 | 60.2 | 2.8 | 4 | 33 | 59.2 | 2.8 | 5 | 33 | 58.2 |
| 32 | 61.2 | 2.8 | 4 | 32 | 60.2 | 2.8 | 5 | 32 | 59.2 |
| 31 | 62.2 | 2.8 | 4 | 31 | 61.2 | 2.8 | 5 | 31 | 60.2 |
| 30 | 63.2 | 2.8 | 4 | 30 | 62.2 | 2.8 | 5 | 30 | 61.2 |
| 60 | 33 | 3 | 4 | 60 | 32 | 3 | 5 | 60 | 31 |
| 59 | 34 | 3 | 4 | 59 | 33 | 3 | 5 | 59 | 32 |
| 58 | 35 | 3 | 4 | 58 | 34 | 3 | 5 | 58 | 33 |
| 57 | 36 | 3 | 4 | 57 | 35 | 3 | 5 | 57 | 34 |
| 56 | 37 | 3 | 4 | 56 | 36 | 3 | 5 | 56 | 35 |
| 55 | 38 | 3 | 4 | 55 | 37 | 3 | 5 | 55 | 36 |
| 54 | 39 | 3 | 4 | 54 | 38 | 3 | 5 | 54 | 37 |
| 53 | 40 | 3 | 4 | 53 | 39 | 3 | 5 | 53 | 38 |
| 52 | 41 | 3 | 4 | 52 | 40 | 3 | 5 | 52 | 39 |
| 51 | 42 | 3 | 4 | 51 | 41 | 3 | 5 | 51 | 40 |
| 50 | 43 | 3 | 4 | 50 | 42 | 3 | 5 | 50 | 41 |
| 49 | 44 | 3 | 4 | 49 | 43 | 3 | 5 | 49 | 42 |
| 48 | 45 | 3 | 4 | 48 | 44 | 3 | 5 | 48 | 43 |
| 47 | 46 | 3 | 4 | 47 | 45 | 3 | 5 | 47 | 44 |
| 46 | 47 | 3 | 4 | 46 | 46 | 3 | 5 | 46 | 45 |
| 45 | 48 | 3 | 4 | 45 | 47 | 3 | 5 | 45 | 46 |
| 44 | 49 | 3 | 4 | 44 | 48 | 3 | 5 | 44 | 47 |
| 43 | 50 | 3 | 4 | 43 | 49 | 3 | 5 | 43 | 48 |
| 42 | 51 | 3 | 4 | 42 | 50 | 3 | 5 | 42 | 49 |
| 41 | 52 | 3 | 4 | 41 | 51 | 3 | 5 | 41 | 50 |
| 40 | 53 | 3 | 4 | 40 | 52 | 3 | 5 | 40 | 51 |
| 39 | 54 | 3 | 4 | 39 | 53 | 3 | 5 | 39 | 52 |
| 38 | 55 | 3 | 4 | 38 | 54 | 3 | 5 | 38 | 53 |
| 37 | 56 | 3 | 4 | 37 | 55 | 3 | 5 | 37 | 54 |
| 36 | 57 | 3 | 4 | 36 | 56 | 3 | 5 | 36 | 55 |
| 35 | 58 | 3 | 4 | 35 | 57 | 3 | 5 | 35 | 56 |
| 34 | 59 | 3 | 4 | 34 | 58 | 3 | 5 | 34 | 57 |
| 33 | 60 | 3 | 4 | 33 | 59 | 3 | 5 | 33 | 58 |
| 32 | 61 | 3 | 4 | 32 | 60 | 3 | 5 | 32 | 59 |
| 31 | 62 | 3 | 4 | 31 | 61 | 3 | 5 | 31 | 60 |
| 30 | 63 | 3 | 4 | 30 | 62 | 3 | 5 | 30 | 61 |
| 60 | 31 | 1 | 8 | 60 | 30 | 1 | 9 | 60 | 29 |
| 59 | 32 | 1 | 8 | 59 | 31 | 1 | 9 | 59 | 30 |
| 58 | 33 | 1 | 8 | 58 | 32 | 1 | 9 | 58 | 31 |
| 57 | 34 | 1 | 8 | 57 | 33 | 1 | 9 | 57 | 32 |
| 56 | 35 | 1 | 8 | 56 | 34 | 1 | 9 | 56 | 33 |
| 55 | 36 | 1 | 8 | 55 | 35 | 1 | 9 | 55 | 34 |
| 54 | 37 | 1 | 8 | 54 | 36 | 1 | 9 | 54 | 35 |
| 53 | 38 | 1 | 8 | 53 | 37 | 1 | 9 | 53 | 36 |
| 52 | 39 | 1 | 8 | 52 | 38 | 1 | 9 | 52 | 37 |
| 51 | 40 | 1 | 8 | 51 | 39 | 1 | 9 | 51 | 38 |
| 50 | 41 | 1 | 8 | 50 | 40 | 1 | 9 | 50 | 39 |
| 49 | 42 | 1 | 8 | 49 | 41 | 1 | 9 | 49 | 40 |
| 48 | 43 | 1 | 8 | 48 | 42 | 1 | 9 | 48 | 41 |
| 47 | 44 | 1 | 8 | 47 | 43 | 1 | 9 | 47 | 42 |
| 46 | 45 | 1 | 8 | 46 | 44 | 1 | 9 | 46 | 43 |
| 45 | 46 | 1 | 8 | 45 | 45 | 1 | 9 | 45 | 44 |
| 44 | 47 | 1 | 8 | 44 | 46 | 1 | 9 | 44 | 45 |
| 43 | 48 | 1 | 8 | 43 | 47 | 1 | 9 | 43 | 46 |
| 42 | 49 | 1 | 8 | 42 | 48 | 1 | 9 | 42 | 47 |
| 41 | 50 | 1 | 8 | 41 | 49 | 1 | 9 | 41 | 48 |
| 40 | 51 | 1 | 8 | 40 | 50 | 1 | 9 | 40 | 49 |
| 39 | 52 | 1 | 8 | 39 | 51 | 1 | 9 | 39 | 50 |
| 38 | 53 | 1 | 8 | 38 | 52 | 1 | 9 | 38 | 51 |
| 37 | 54 | 1 | 8 | 37 | 53 | 1 | 9 | 37 | 52 |
| 36 | 55 | 1 | 8 | 36 | 54 | 1 | 9 | 36 | 53 |
| 35 | 56 | 1 | 8 | 35 | 55 | 1 | 9 | 35 | 54 |
| 34 | 57 | 1 | 8 | 34 | 56 | 1 | 9 | 34 | 55 |
| 33 | 58 | 1 | 8 | 33 | 57 | 1 | 9 | 33 | 56 |
| 32 | 59 | 1 | 8 | 32 | 58 | 1 | 9 | 32 | 57 |
| 31 | 60 | 1 | 8 | 31 | 59 | 1 | 9 | 31 | 58 |
| 30 | 61 | 1 | 8 | 30 | 60 | 1 | 9 | 30 | 59 |
| 60 | 30.8 | 1.2 | 8 | 60 | 29.8 | 1.2 | 9 | 60 | 28.8 |
| 59 | 31.8 | 1.2 | 8 | 59 | 30.8 | 1.2 | 9 | 59 | 29.8 |
| 58 | 32.8 | 1.2 | 8 | 58 | 31.8 | 1.2 | 9 | 58 | 30.8 |
| 57 | 33.8 | 1.2 | 8 | 57 | 32.8 | 1.2 | 9 | 57 | 31.8 |
| 56 | 34.8 | 1.2 | 8 | 56 | 33.8 | 1.2 | 9 | 56 | 32.8 |
| 55 | 35.8 | 1.2 | 8 | 55 | 34.8 | 1.2 | 9 | 55 | 33.8 |
| 54 | 36.8 | 1.2 | 8 | 54 | 35.8 | 1.2 | 9 | 54 | 34.8 |
| 53 | 37.8 | 1.2 | 8 | 53 | 36.8 | 1.2 | 9 | 53 | 35.8 |
| 52 | 38.8 | 1.2 | 8 | 52 | 37.8 | 1.2 | 9 | 52 | 36.8 |
| 51 | 39.8 | 1.2 | 8 | 51 | 38.8 | 1.2 | 9 | 51 | 37.8 |
| 50 | 40.8 | 1.2 | 8 | 50 | 39.8 | 1.2 | 9 | 50 | 38.8 |
| 49 | 41.8 | 1.2 | 8 | 49 | 40.8 | 1.2 | 9 | 49 | 39.8 |
| 48 | 42.8 | 1.2 | 8 | 48 | 41.8 | 1.2 | 9 | 48 | 40.8 |
| 47 | 43.8 | 1.2 | 8 | 47 | 42.8 | 1.2 | 9 | 47 | 41.8 |
| 46 | 44.8 | 1.2 | 8 | 46 | 43.8 | 1.2 | 9 | 46 | 42.8 |
| 45 | 45.8 | 1.2 | 8 | 45 | 44.8 | 1.2 | 9 | 45 | 43.8 |
| 44 | 46.8 | 1.2 | 8 | 44 | 45.8 | 1.2 | 9 | 44 | 44.8 |
| 43 | 47.8 | 1.2 | 8 | 43 | 46.8 | 1.2 | 9 | 43 | 45.8 |
| 42 | 48.8 | 1.2 | 8 | 42 | 47.8 | 1.2 | 9 | 42 | 46.8 |
| 41 | 49.8 | 1.2 | 8 | 41 | 48.8 | 1.2 | 9 | 41 | 47.8 |
| 40 | 50.8 | 1.2 | 8 | 40 | 49.8 | 1.2 | 9 | 40 | 48.8 |
| 39 | 51.8 | 1.2 | 8 | 39 | 50.8 | 1.2 | 9 | 39 | 49.8 |
| 38 | 52.8 | 1.2 | 8 | 38 | 51.8 | 1.2 | 9 | 38 | 50.8 |
| 37 | 53.8 | 1.2 | 8 | 37 | 52.8 | 1.2 | 9 | 37 | 51.8 |
| 36 | 54.8 | 1.2 | 8 | 36 | 53.8 | 1.2 | 9 | 36 | 52.8 |
| 35 | 55.8 | 1.2 | 8 | 35 | 54.8 | 1.2 | 9 | 35 | 53.8 |
| 34 | 56.8 | 1.2 | 8 | 34 | 55.8 | 1.2 | 9 | 34 | 54.8 |
| 33 | 57.8 | 1.2 | 8 | 33 | 56.8 | 1.2 | 9 | 33 | 55.8 |
| 32 | 58.8 | 1.2 | 8 | 32 | 57.8 | 1.2 | 9 | 32 | 56.8 |
| 31 | 59.8 | 1.2 | 8 | 31 | 58.8 | 1.2 | 9 | 31 | 57.8 |
| 30 | 60.8 | 1.2 | 8 | 30 | 59.8 | 1.2 | 9 | 30 | 58.8 |
| 60 | 30.6 | 1.4 | 8 | 60 | 29.6 | 1.4 | 9 | 60 | 28.6 |
| 59 | 31.6 | 1.4 | 8 | 59 | 30.6 | 1.4 | 9 | 59 | 29.6 |
| 58 | 32.6 | 1.4 | 8 | 58 | 31.6 | 1.4 | 9 | 58 | 30.6 |
| 57 | 33.6 | 1.4 | 8 | 57 | 32.6 | 1.4 | 9 | 57 | 31.6 |
| 56 | 34.6 | 1.4 | 8 | 56 | 33.6 | 1.4 | 9 | 56 | 32.6 |
| 55 | 35.6 | 1.4 | 8 | 55 | 34.6 | 1.4 | 9 | 55 | 33.6 |
| 54 | 36.6 | 1.4 | 8 | 54 | 35.6 | 1.4 | 9 | 54 | 34.6 |
| 53 | 37.6 | 1.4 | 8 | 53 | 36.6 | 1.4 | 9 | 53 | 35.6 |
| 52 | 38.6 | 1.4 | 8 | 52 | 37.6 | 1.4 | 9 | 52 | 36.6 |
| 51 | 39.6 | 1.4 | 8 | 51 | 38.6 | 1.4 | 9 | 51 | 37.6 |
| 50 | 40.6 | 1.4 | 8 | 50 | 39.6 | 1.4 | 9 | 50 | 38.6 |
| 49 | 41.6 | 1.4 | 8 | 49 | 40.6 | 1.4 | 9 | 49 | 39.6 |
| 48 | 42.6 | 1.4 | 8 | 48 | 41.6 | 1.4 | 9 | 48 | 40.6 |
| 47 | 43.6 | 1.4 | 8 | 47 | 42.6 | 1.4 | 9 | 47 | 41.6 |
| 46 | 44.6 | 1.4 | 8 | 46 | 43.6 | 1.4 | 9 | 46 | 42.6 |
| 45 | 45.6 | 1.4 | 8 | 45 | 44.6 | 1.4 | 9 | 45 | 43.6 |
| 44 | 46.6 | 1.4 | 8 | 44 | 45.6 | 1.4 | 9 | 44 | 44.6 |
| 43 | 47.6 | 1.4 | 8 | 43 | 46.6 | 1.4 | 9 | 43 | 45.6 |
| 42 | 48.6 | 1.4 | 8 | 42 | 47.6 | 1.4 | 9 | 42 | 46.6 |
| 41 | 49.6 | 1.4 | 8 | 41 | 48.6 | 1.4 | 9 | 41 | 47.6 |
| 40 | 50.6 | 1.4 | 8 | 40 | 49.6 | 1.4 | 9 | 40 | 48.6 |
| 39 | 51.6 | 1.4 | 8 | 39 | 50.6 | 1.4 | 9 | 39 | 49.6 |
| 38 | 52.6 | 1.4 | 8 | 38 | 51.6 | 1.4 | 9 | 38 | 50.6 |
| 37 | 53.6 | 1.4 | 8 | 37 | 52.6 | 1.4 | 9 | 37 | 51.6 |
| 36 | 54.6 | 1.4 | 8 | 36 | 53.6 | 1.4 | 9 | 36 | 52.6 |
| 35 | 55.6 | 1.4 | 8 | 35 | 54.6 | 1.4 | 9 | 35 | 53.6 |
| 34 | 56.6 | 1.4 | 8 | 34 | 55.6 | 1.4 | 9 | 34 | 54.6 |
| 33 | 57.6 | 1.4 | 8 | 33 | 56.6 | 1.4 | 9 | 33 | 55.6 |
| 32 | 58.6 | 1.4 | 8 | 32 | 57.6 | 1.4 | 9 | 32 | 56.6 |
| 31 | 59.6 | 1.4 | 8 | 31 | 58.6 | 1.4 | 9 | 31 | 57.6 |
| 30 | 60.6 | 1.4 | 8 | 30 | 59.6 | 1.4 | 9 | 30 | 58.6 |
| 60 | 30.4 | 1.6 | 8 | 60 | 29.4 | 1.6 | 9 | 60 | 28.4 |
| 59 | 31.4 | 1.6 | 8 | 59 | 30.4 | 1.6 | 9 | 59 | 29.4 |
| 58 | 32.4 | 1.6 | 8 | 58 | 31.4 | 1.6 | 9 | 58 | 30.4 |
| 57 | 33.4 | 1.6 | 8 | 57 | 32.4 | 1.6 | 9 | 57 | 31.4 |
| 56 | 34.4 | 1.6 | 8 | 56 | 33.4 | 1.6 | 9 | 56 | 32.4 |
| 55 | 35.4 | 1.6 | 8 | 55 | 34.4 | 1.6 | 9 | 55 | 33.4 |
| 54 | 36.4 | 1.6 | 8 | 54 | 35.4 | 1.6 | 9 | 54 | 34.4 |
| 53 | 37.4 | 1.6 | 8 | 53 | 36.4 | 1.6 | 9 | 53 | 35.4 |
| 52 | 38.4 | 1.6 | 8 | 52 | 37.4 | 1.6 | 9 | 52 | 36.4 |
| 51 | 39.4 | 1.6 | 8 | 51 | 38.4 | 1.6 | 9 | 51 | 37.4 |
| 50 | 40.4 | 1.6 | 8 | 50 | 39.4 | 1.6 | 9 | 50 | 38.4 |
| 49 | 41.4 | 1.6 | 8 | 49 | 40.4 | 1.6 | 9 | 49 | 39.4 |
| 48 | 42.4 | 1.6 | 8 | 48 | 41.4 | 1.6 | 9 | 48 | 40.4 |
| 47 | 43.4 | 1.6 | 8 | 47 | 42.4 | 1.6 | 9 | 47 | 41.4 |
| 46 | 44.4 | 1.6 | 8 | 46 | 43.4 | 1.6 | 9 | 46 | 42.4 |
| 45 | 45.4 | 1.6 | 8 | 45 | 44.4 | 1.6 | 9 | 45 | 43.4 |
| 44 | 46.4 | 1.6 | 8 | 44 | 45.4 | 1.6 | 9 | 44 | 44.4 |
| 43 | 47.4 | 1.6 | 8 | 43 | 46.4 | 1.6 | 9 | 43 | 45.4 |
| 42 | 48.4 | 1.6 | 8 | 42 | 47.4 | 1.6 | 9 | 42 | 46.4 |
| 41 | 49.4 | 1.6 | 8 | 41 | 48.4 | 1.6 | 9 | 41 | 47.4 |
| 40 | 50.4 | 1.6 | 8 | 40 | 49.4 | 1.6 | 9 | 40 | 48.4 |
| 39 | 51.4 | 1.6 | 8 | 39 | 50.4 | 1.6 | 9 | 39 | 49.4 |
| 38 | 52.4 | 1.6 | 8 | 38 | 51.4 | 1.6 | 9 | 38 | 50.4 |
| 37 | 53.4 | 1.6 | 8 | 37 | 52.4 | 1.6 | 9 | 37 | 51.4 |
| 36 | 54.4 | 1.6 | 8 | 36 | 53.4 | 1.6 | 9 | 36 | 52.4 |
| 35 | 55.4 | 1.6 | 8 | 35 | 54.4 | 1.6 | 9 | 35 | 53.4 |
| 34 | 56.4 | 1.6 | 8 | 34 | 55.4 | 1.6 | 9 | 34 | 54.4 |
| 33 | 57.4 | 1.6 | 8 | 33 | 56.4 | 1.6 | 9 | 33 | 55.4 |
| 32 | 58.4 | 1.6 | 8 | 32 | 57.4 | 1.6 | 9 | 32 | 56.4 |
| 31 | 59.4 | 1.6 | 8 | 31 | 58.4 | 1.6 | 9 | 31 | 57.4 |
| 30 | 60.4 | 1.6 | 8 | 30 | 59.4 | 1.6 | 9 | 30 | 58.4 |
| 60 | 30.2 | 1.8 | 8 | 60 | 29.2 | 1.8 | 9 | 60 | 28.2 |
| 59 | 31.2 | 1.8 | 8 | 59 | 30.2 | 1.8 | 9 | 59 | 29.2 |
| 58 | 32.2 | 1.8 | 8 | 58 | 31.2 | 1.8 | 9 | 58 | 30.2 |
| 57 | 33.2 | 1.8 | 8 | 57 | 32.2 | 1.8 | 9 | 57 | 31.2 |
| 56 | 34.2 | 1.8 | 8 | 56 | 33.2 | 1.8 | 9 | 56 | 32.2 |
| 55 | 35.2 | 1.8 | 8 | 55 | 34.2 | 1.8 | 9 | 55 | 33.2 |
| 54 | 36.2 | 1.8 | 8 | 54 | 35.2 | 1.8 | 9 | 54 | 34.2 |
| 53 | 37.2 | 1.8 | 8 | 53 | 36.2 | 1.8 | 9 | 53 | 35.2 |
| 52 | 38.2 | 1.8 | 8 | 52 | 37.2 | 1.8 | 9 | 52 | 36.2 |
| 51 | 39.2 | 1.8 | 8 | 51 | 38.2 | 1.8 | 9 | 51 | 37.2 |
| 50 | 40.2 | 1.8 | 8 | 50 | 39.2 | 1.8 | 9 | 50 | 38.2 |
| 49 | 41.2 | 1.8 | 8 | 49 | 40.2 | 1.8 | 9 | 49 | 39.2 |
| 48 | 42.2 | 1.8 | 8 | 48 | 41.2 | 1.8 | 9 | 48 | 40.2 |
| 47 | 43.2 | 1.8 | 8 | 47 | 42.2 | 1.8 | 9 | 47 | 41.2 |
| 46 | 44.2 | 1.8 | 8 | 46 | 43.2 | 1.8 | 9 | 46 | 42.2 |
| 45 | 45.2 | 1.8 | 8 | 45 | 44.2 | 1.8 | 9 | 45 | 43.2 |
| 44 | 46.2 | 1.8 | 8 | 44 | 45.2 | 1.8 | 9 | 44 | 44.2 |
| 43 | 47.2 | 1.8 | 8 | 43 | 46.2 | 1.8 | 9 | 43 | 45.2 |
| 42 | 48.2 | 1.8 | 8 | 42 | 47.2 | 1.8 | 9 | 42 | 46.2 |
| 41 | 49.2 | 1.8 | 8 | 41 | 48.2 | 1.8 | 9 | 41 | 47.2 |
| 40 | 50.2 | 1.8 | 8 | 40 | 49.2 | 1.8 | 9 | 40 | 48.2 |
| 39 | 51.2 | 1.8 | 8 | 39 | 50.2 | 1.8 | 9 | 39 | 49.2 |
| 38 | 52.2 | 1.8 | 8 | 38 | 51.2 | 1.8 | 9 | 38 | 50.2 |
| 37 | 53.2 | 1.8 | 8 | 37 | 52.2 | 1.8 | 9 | 37 | 51.2 |
| 36 | 54.2 | 1.8 | 8 | 36 | 53.2 | 1.8 | 9 | 36 | 52.2 |
| 35 | 55.2 | 1.8 | 8 | 35 | 54.2 | 1.8 | 9 | 35 | 53.2 |
| 34 | 56.2 | 1.8 | 8 | 34 | 55.2 | 1.8 | 9 | 34 | 54.2 |
| 33 | 57.2 | 1.8 | 8 | 33 | 56.2 | 1.8 | 9 | 33 | 55.2 |
| 32 | 58.2 | 1.8 | 8 | 32 | 57.2 | 1.8 | 9 | 32 | 56.2 |
| 31 | 59.2 | 1.8 | 8 | 31 | 58.2 | 1.8 | 9 | 31 | 57.2 |
| 30 | 60.2 | 1.8 | 8 | 30 | 59.2 | 1.8 | 9 | 30 | 58.2 |
| 60 | 30 | 2 | 8 | 60 | 29 | 2 | 9 | 60 | 28 |
| 59 | 31 | 2 | 8 | 59 | 30 | 2 | 9 | 59 | 29 |
| 58 | 32 | 2 | 8 | 58 | 31 | 2 | 9 | 58 | 30 |
| 57 | 33 | 2 | 8 | 57 | 32 | 2 | 9 | 57 | 31 |
| 56 | 34 | 2 | 8 | 56 | 33 | 2 | 9 | 56 | 32 |
| 55 | 35 | 2 | 8 | 55 | 34 | 2 | 9 | 55 | 33 |
| 54 | 36 | 2 | 8 | 54 | 35 | 2 | 9 | 54 | 34 |
| 53 | 37 | 2 | 8 | 53 | 36 | 2 | 9 | 53 | 35 |
| 52 | 38 | 2 | 8 | 52 | 37 | 2 | 9 | 52 | 36 |
| 51 | 39 | 2 | 8 | 51 | 38 | 2 | 9 | 51 | 37 |
| 50 | 40 | 2 | 8 | 50 | 39 | 2 | 9 | 50 | 38 |
| 49 | 41 | 2 | 8 | 49 | 40 | 2 | 9 | 49 | 39 |
| 48 | 42 | 2 | 8 | 48 | 41 | 2 | 9 | 48 | 40 |
| 47 | 43 | 2 | 8 | 47 | 42 | 2 | 9 | 47 | 41 |
| 46 | 44 | 2 | 8 | 46 | 43 | 2 | 9 | 46 | 42 |
| 45 | 45 | 2 | 8 | 45 | 44 | 2 | 9 | 45 | 43 |
| 44 | 46 | 2 | 8 | 44 | 45 | 2 | 9 | 44 | 44 |
| 43 | 47 | 2 | 8 | 43 | 46 | 2 | 9 | 43 | 45 |
| 42 | 48 | 2 | 8 | 42 | 47 | 2 | 9 | 42 | 46 |
| 41 | 49 | 2 | 8 | 41 | 48 | 2 | 9 | 41 | 47 |
| 40 | 50 | 2 | 8 | 40 | 49 | 2 | 9 | 40 | 48 |
| 39 | 51 | 2 | 8 | 39 | 50 | 2 | 9 | 39 | 49 |
| 38 | 52 | 2 | 8 | 38 | 51 | 2 | 9 | 38 | 50 |
| 37 | 53 | 2 | 8 | 37 | 52 | 2 | 9 | 37 | 51 |
| 36 | 54 | 2 | 8 | 36 | 53 | 2 | 9 | 36 | 52 |
| 35 | 55 | 2 | 8 | 35 | 54 | 2 | 9 | 35 | 53 |
| 34 | 56 | 2 | 8 | 34 | 55 | 2 | 9 | 34 | 54 |
| 33 | 57 | 2 | 8 | 33 | 56 | 2 | 9 | 33 | 55 |
| 32 | 58 | 2 | 8 | 32 | 57 | 2 | 9 | 32 | 56 |
| co | 59 | 2 | 8 | 31 | 58 | 2 | 9 | 31 | 57 |
| 30 | 60 | 2 | 8 | 30 | 59 | 2 | 9 | 30 | 58 |
| 60 | 29.8 | 2.2 | 8 | 60 | 28.8 | 2.2 | 9 | 60 | 27.8 |
| 59 | 30.8 | 2.2 | 8 | 59 | 29.8 | 2.2 | 9 | 59 | 28.8 |
| 58 | 31.8 | 2.2 | 8 | 58 | 30.8 | 2.2 | 9 | 58 | 29.8 |
| 57 | 32.8 | 2.2 | 8 | 57 | 31.8 | 2.2 | 9 | 57 | 30.8 |
| 56 | 33.8 | 2.2 | 8 | 56 | 32.8 | 2.2 | 9 | 56 | 31.8 |
| 55 | 34.8 | 2.2 | 8 | 55 | 33.8 | 2.2 | 9 | 55 | 32.8 |
| 54 | 35.8 | 2.2 | 8 | 54 | 34.8 | 2.2 | 9 | 54 | 33.8 |
| 53 | 36.8 | 2.2 | 8 | 53 | 35.8 | 2.2 | 9 | 53 | 34.8 |
| 52 | 37.8 | 2.2 | 8 | 52 | 36.8 | 2.2 | 9 | 52 | 35.8 |
| 51 | 38.8 | 2.2 | 8 | 51 | 37.8 | 2.2 | 9 | 51 | 36.8 |
| 50 | 39.8 | 2.2 | 8 | 50 | 38.8 | 2.2 | 9 | 50 | 37.8 |
| 49 | 40.8 | 2.2 | 8 | 49 | 39.8 | 2.2 | 9 | 49 | 38.8 |
| 48 | 41.8 | 2.2 | 8 | 48 | 40.8 | 2.2 | 9 | 48 | 39.8 |
| 47 | 42.8 | 2.2 | 8 | 47 | 41.8 | 2.2 | 9 | 47 | 40.8 |
| 46 | 43.8 | 2.2 | 8 | 46 | 42.8 | 2.2 | 9 | 46 | 41.8 |
| 45 | 44.8 | 2.2 | 8 | 45 | 43.8 | 2.2 | 9 | 45 | 42.8 |
| 44 | 45.8 | 2.2 | 8 | 44 | 44.8 | 2.2 | 9 | 44 | 43.8 |
| 43 | 46.8 | 2.2 | 8 | 43 | 45.8 | 2.2 | 9 | 43 | 44.8 |
| 42 | 47.8 | 2.2 | 8 | 42 | 46.8 | 2.2 | 9 | 42 | 45.8 |
| 41 | 48.8 | 2.2 | 8 | 41 | 47.8 | 2.2 | 9 | 41 | 46.8 |
| 40 | 49.8 | 2.2 | 8 | 40 | 48.8 | 2.2 | 9 | 40 | 47.8 |
| 39 | 50.8 | 2.2 | 8 | 39 | 49.8 | 2.2 | 9 | 39 | 48.8 |
| 38 | 51.8 | 2.2 | 8 | 38 | 50.8 | 2.2 | 9 | 38 | 49.8 |
| 37 | 52.8 | 2.2 | 8 | 37 | 51.8 | 2.2 | 9 | 37 | 50.8 |
| 36 | 53.8 | 2.2 | 8 | 36 | 52.8 | 2.2 | 9 | 36 | 51.8 |
| 35 | 54.8 | 2.2 | 8 | 35 | 53.8 | 2.2 | 9 | 35 | 52.8 |
| 34 | 55.8 | 2.2 | 8 | 34 | 54.8 | 2.2 | 9 | 34 | 53.8 |
| 33 | 56.8 | 2.2 | 8 | 33 | 55.8 | 2.2 | 9 | 33 | 54.8 |
| 32 | 57.8 | 2.2 | 8 | 32 | 56.8 | 2.2 | 9 | 32 | 55.8 |
| 31 | 58.8 | 2.2 | 8 | 31 | 57.8 | 2.2 | 9 | 31 | 56.8 |
| 30 | 59.8 | 2.2 | 8 | 30 | 58.8 | 2.2 | 9 | 30 | 57.8 |
| 60 | 29.6 | 2.4 | 8 | 60 | 28.6 | 2.4 | 9 | 60 | 27.6 |
| 59 | 30.6 | 2.4 | 8 | 59 | 29.6 | 2.4 | 9 | 59 | 28.6 |
| 58 | 31.6 | 2.4 | 8 | 58 | 30.6 | 2.4 | 9 | 58 | 29.6 |
| 57 | 32.6 | 2.4 | 8 | 57 | 31.6 | 2.4 | 9 | 57 | 30.6 |
| 56 | 33.6 | 2.4 | 8 | 56 | 32.6 | 2.4 | 9 | 56 | 31.6 |
| 55 | 34.6 | 2.4 | 8 | 55 | 33.6 | 2.4 | 9 | 55 | 32.6 |
| 54 | 35.6 | 2.4 | 8 | 54 | 34.6 | 2.4 | 9 | 54 | 33.6 |
| 53 | 36.6 | 2.4 | 8 | 53 | 35.6 | 2.4 | 9 | 53 | 34.6 |
| 52 | 37.6 | 2.4 | 8 | 52 | 36.6 | 2.4 | 9 | 52 | 35.6 |
| 51 | 38.6 | 2.4 | 8 | 51 | 37.6 | 2.4 | 9 | 51 | 36.6 |
| 50 | 39.6 | 2.4 | 8 | 50 | 38.6 | 2.4 | 9 | 50 | 37.6 |
| 49 | 40.6 | 2.4 | 8 | 49 | 39.6 | 2.4 | 9 | 49 | 38.6 |
| 48 | 41.6 | 2.4 | 8 | 48 | 40.6 | 2.4 | 9 | 48 | 39.6 |
| 47 | 42.6 | 2.4 | 8 | 47 | 41.6 | 2.4 | 9 | 47 | 40.6 |
| 46 | 43.6 | 2.4 | 8 | 46 | 42.6 | 2.4 | 9 | 46 | 41.6 |
| 45 | 44.6 | 2.4 | 8 | 45 | 43.6 | 2.4 | 9 | 45 | 42.6 |
| 44 | 45.6 | 2.4 | 8 | 44 | 44.6 | 2.4 | 9 | 44 | 43.6 |
| 43 | 46.6 | 2.4 | 8 | 43 | 45.6 | 2.4 | 9 | 43 | 44.6 |
| 42 | 47.6 | 2.4 | 8 | 42 | 46.6 | 2.4 | 9 | 42 | 45.6 |
| 41 | 48.6 | 2.4 | 8 | 41 | 47.6 | 2.4 | 9 | 41 | 46.6 |
| 40 | 49.6 | 2.4 | 8 | 40 | 48.6 | 2.4 | 9 | 40 | 47.6 |
| 39 | 50.6 | 2.4 | 8 | 39 | 49.6 | 2.4 | 9 | 39 | 48.6 |
| 38 | 51.6 | 2.4 | 8 | 38 | 50.6 | 2.4 | 9 | 38 | 49.6 |
| 37 | 52.6 | 2.4 | 8 | 37 | 51.6 | 2.4 | 9 | 37 | 50.6 |
| 36 | 53.6 | 2.4 | 8 | 36 | 52.6 | 2.4 | 9 | 36 | 51.6 |
| 35 | 54.6 | 2.4 | 8 | 35 | 53.6 | 2.4 | 9 | 35 | 52.6 |
| 34 | 55.6 | 2.4 | 8 | 34 | 54.6 | 2.4 | 9 | 34 | 53.6 |
| 33 | 56.6 | 2.4 | 8 | 33 | 55.6 | 2.4 | 9 | 33 | 54.6 |
| 32 | 57.6 | 2.4 | 8 | 32 | 56.6 | 2.4 | 9 | 32 | 55.6 |
| 31 | 58.6 | 2.4 | 8 | 31 | 57.6 | 2.4 | 9 | 31 | 56.6 |
| 30 | 59.6 | 2.4 | 8 | 30 | 58.6 | 2.4 | 9 | 30 | 57.6 |
| 60 | 29.4 | 2.6 | 8 | 60 | 28.4 | 2.6 | 9 | 60 | 27.4 |
| 59 | 30.4 | 2.6 | 8 | 59 | 29.4 | 2.6 | 9 | 59 | 28.4 |
| 58 | 31.4 | 2.6 | 8 | 58 | 30.4 | 2.6 | 9 | 58 | 29.4 |
| 57 | 32.4 | 2.6 | 8 | 57 | 31.4 | 2.6 | 9 | 57 | 30.4 |
| 56 | 33.4 | 2.6 | 8 | 56 | 32.4 | 2.6 | 9 | 56 | 31.4 |
| 55 | 34.4 | 2.6 | 8 | 55 | 33.4 | 2.6 | 9 | 55 | 32.4 |
| 54 | 35.4 | 2.6 | 8 | 54 | 34.4 | 2.6 | 9 | 54 | 33.4 |
| 53 | 36.4 | 2.6 | 8 | 53 | 35.4 | 2.6 | 9 | 53 | 34.4 |
| 52 | 37.4 | 2.6 | 8 | 52 | 36.4 | 2.6 | 9 | 52 | 35.4 |
| 51 | 38.4 | 2.6 | 8 | 51 | 37.4 | 2.6 | 9 | 51 | 36.4 |
| 50 | 39.4 | 2.6 | 8 | 50 | 38.4 | 2.6 | 9 | 50 | 37.4 |
| 49 | 40.4 | 2.6 | 8 | 49 | 39.4 | 2.6 | 9 | 49 | 38.4 |
| 48 | 41.4 | 2.6 | 8 | 48 | 40.4 | 2.6 | 9 | 48 | 39.4 |
| 47 | 42.4 | 2.6 | 8 | 47 | 41.4 | 2.6 | 9 | 47 | 40.4 |
| 46 | 43.4 | 2.6 | 8 | 46 | 42.4 | 2.6 | 9 | 46 | 41.4 |
| 45 | 44.4 | 2.6 | 8 | 45 | 43.4 | 2.6 | 9 | 45 | 42.4 |
| 44 | 45.4 | 2.6 | 8 | 44 | 44.4 | 2.6 | 9 | 44 | 43.4 |
| 43 | 46.4 | 2.6 | 8 | 43 | 45.4 | 2.6 | 9 | 43 | 44.4 |
| 42 | 47.4 | 2.6 | 8 | 42 | 46.4 | 2.6 | 9 | 42 | 45.4 |
| 41 | 48.4 | 2.6 | 8 | 41 | 47.4 | 2.6 | 9 | 41 | 46.4 |
| 40 | 49.4 | 2.6 | 8 | 40 | 48.4 | 2.6 | 9 | 40 | 47.4 |
| 39 | 50.4 | 2.6 | 8 | 39 | 49.4 | 2.6 | 9 | 39 | 48.4 |
| 38 | 51.4 | 2.6 | 8 | 38 | 50.4 | 2.6 | 9 | 38 | 49.4 |
| 37 | 52.4 | 2.6 | 8 | 37 | 51.4 | 2.6 | 9 | 37 | 50.4 |
| 36 | 53.4 | 2.6 | 8 | 36 | 52.4 | 2.6 | 9 | 36 | 51.4 |
| 35 | 54.4 | 2.6 | 8 | 35 | 53.4 | 2.6 | 9 | 35 | 52.4 |
| 34 | 55.4 | 2.6 | 8 | 34 | 54.4 | 2.6 | 9 | 34 | 53.4 |
| 33 | 56.4 | 2.6 | 8 | 33 | 55.4 | 2.6 | 9 | 33 | 54.4 |
| 32 | 57.4 | 2.6 | 8 | 32 | 56.4 | 2.6 | 9 | 32 | 55.4 |
| 31 | 58.4 | 2.6 | 8 | 31 | 57.4 | 2.6 | 9 | 31 | 56.4 |
| 30 | 59.4 | 2.6 | 8 | 30 | 58.4 | 2.6 | 9 | 30 | 57.4 |
| 60 | 29.2 | 2.8 | 8 | 60 | 28.2 | 2.8 | 9 | 60 | 27.2 |
| 59 | 30.2 | 2.8 | 8 | 59 | 29.2 | 2.8 | 9 | 59 | 28.2 |
| 58 | 31.2 | 2.8 | 8 | 58 | 30.2 | 2.8 | 9 | 58 | 29.2 |
| 57 | 32.2 | 2.8 | 8 | 57 | 31.2 | 2.8 | 9 | 57 | 30.2 |
| 56 | 33.2 | 2.8 | 8 | 56 | 32.2 | 2.8 | 9 | 56 | 31.2 |
| 55 | 34.2 | 2.8 | 8 | 55 | 33.2 | 2.8 | 9 | 55 | 32.2 |
| 54 | 35.2 | 2.8 | 8 | 54 | 34.2 | 2.8 | 9 | 54 | 33.2 |
| 53 | 36.2 | 2.8 | 8 | 53 | 35.2 | 2.8 | 9 | 53 | 34.2 |
| 52 | 37.2 | 2.8 | 8 | 52 | 36.2 | 2.8 | 9 | 52 | 35.2 |
| 51 | 38.2 | 2.8 | 8 | 51 | 37.2 | 2.8 | 9 | 51 | 36.2 |
| 50 | 39.2 | 2.8 | 8 | 50 | 38.2 | 2.8 | 9 | 50 | 37.2 |
| 49 | 40.2 | 2.8 | 8 | 49 | 39.2 | 2.8 | 9 | 49 | 38.2 |
| 48 | 41.2 | 2.8 | 8 | 48 | 40.2 | 2.8 | 9 | 48 | 39.2 |
| 47 | 42.2 | 2.8 | 8 | 47 | 41.2 | 2.8 | 9 | 47 | 40.2 |
| 46 | 43.2 | 2.8 | 8 | 46 | 42.2 | 2.8 | 9 | 46 | 41.2 |
| 45 | 44.2 | 2.8 | 8 | 45 | 43.2 | 2.8 | 9 | 45 | 42.2 |
| 44 | 45.2 | 2.8 | 8 | 44 | 44.2 | 2.8 | 9 | 44 | 43.2 |
| 43 | 46.2 | 2.8 | 8 | 43 | 45.2 | 2.8 | 9 | 43 | 44.2 |
| 42 | 47.2 | 2.8 | 8 | 42 | 46.2 | 2.8 | 9 | 42 | 45.2 |
| 41 | 48.2 | 2.8 | 8 | 41 | 47.2 | 2.8 | 9 | 41 | 46.2 |
| 40 | 49.2 | 2.8 | 8 | 40 | 48.2 | 2.8 | 9 | 40 | 47.2 |
| 39 | 50.2 | 2.8 | 8 | 39 | 49.2 | 2.8 | 9 | 39 | 48.2 |
| 38 | 51.2 | 2.8 | 8 | 38 | 50.2 | 2.8 | 9 | 38 | 49.2 |
| 37 | 52.2 | 2.8 | 8 | 37 | 51.2 | 2.8 | 9 | 37 | 50.2 |
| 36 | 53.2 | 2.8 | 8 | 36 | 52.2 | 2.8 | 9 | 36 | 51.2 |
| 35 | 54.2 | 2.8 | 8 | 35 | 53.2 | 2.8 | 9 | 35 | 52.2 |
| 34 | 55.2 | 2.8 | 8 | 34 | 54.2 | 2.8 | 9 | 34 | 53.2 |
| 33 | 56.2 | 2.8 | 8 | 33 | 55.2 | 2.8 | 9 | 33 | 54.2 |
| 32 | 57.2 | 2.8 | 8 | 32 | 56.2 | 2.8 | 9 | 32 | 55.2 |
| 31 | 58.2 | 2.8 | 8 | 31 | 57.2 | 2.8 | 9 | 31 | 56.2 |
| 30 | 59.2 | 2.8 | 8 | 30 | 58.2 | 2.8 | 9 | 30 | 57.2 |
| 60 | 29 | 3 | 8 | 60 | 28 | 3 | 9 | 60 | 27 |
| 59 | 30 | 3 | 8 | 59 | 29 | 3 | 9 | 59 | 28 |
| 58 | 31 | 3 | 8 | 58 | 30 | 3 | 9 | 58 | 29 |
| 57 | 32 | 3 | 8 | 57 | 31 | 3 | 9 | 57 | 30 |
| 56 | 33 | 3 | 8 | 56 | 32 | 3 | 9 | 56 | 31 |
| 55 | 34 | 3 | 8 | 55 | 33 | 3 | 9 | 55 | 32 |
| 54 | 35 | 3 | 8 | 54 | 34 | 3 | 9 | 54 | 33 |
| 53 | 36 | 3 | 8 | 53 | 35 | 3 | 9 | 53 | 34 |
| 52 | 37 | 3 | 8 | 52 | 36 | 3 | 9 | 52 | 35 |
| 51 | 38 | 3 | 8 | 51 | 37 | 3 | 9 | 51 | 36 |
| 50 | 39 | 3 | 8 | 50 | 38 | 3 | 9 | 50 | 37 |
| 49 | 40 | 3 | 8 | 49 | 39 | 3 | 9 | 49 | 38 |
| 48 | 41 | 3 | 8 | 48 | 40 | 3 | 9 | 48 | 39 |
| 47 | 42 | 3 | 8 | 47 | 41 | 3 | 9 | 47 | 40 |
| 46 | 43 | 3 | 8 | 46 | 42 | 3 | 9 | 46 | 41 |
| 45 | 44 | 3 | 8 | 45 | 43 | 3 | 9 | 45 | 42 |
| 44 | 45 | 3 | 8 | 44 | 44 | 3 | 9 | 44 | 43 |
| 43 | 46 | 3 | 8 | 43 | 45 | 3 | 9 | 43 | 44 |
| 42 | 47 | 3 | 8 | 42 | 46 | 3 | 9 | 42 | 45 |
| 41 | 48 | 3 | 8 | 41 | 47 | 3 | 9 | 41 | 46 |
| 40 | 49 | 3 | 8 | 40 | 48 | 3 | 9 | 40 | 47 |
| 39 | 50 | 3 | 8 | 39 | 49 | 3 | 9 | 39 | 48 |
| 38 | 51 | 3 | 8 | 38 | 50 | 3 | 9 | 38 | 49 |
| 37 | 52 | 3 | 8 | 37 | 51 | 3 | 9 | 37 | 50 |
| 36 | 53 | 3 | 8 | 36 | 52 | 3 | 9 | 36 | 51 |
| 35 | 54 | 3 | 8 | 35 | 53 | 3 | 9 | 35 | 52 |
| 34 | 55 | 3 | 8 | 34 | 54 | 3 | 9 | 34 | 53 |
| 33 | 56 | 3 | 8 | 33 | 55 | 3 | 9 | 33 | 54 |
| 32 | 57 | 3 | 8 | 32 | 56 | 3 | 9 | 32 | 55 |
| 31 | 58 | 3 | 8 | 31 | 57 | 3 | 9 | 31 | 56 |
| 30 | 59 | 3 | 8 | 30 | 58 | 3 | 9 | 30 | 57 |
| 60 | 27 | 1 | 12 | 60 | 26 | 1 | 13 | 60 | 25 |
| 59 | 28 | 1 | 12 | 59 | 27 | 1 | 13 | 59 | 26 |
| 58 | 29 | 1 | 12 | 58 | 28 | 1 | 13 | 58 | 27 |
| 57 | 30 | 1 | 12 | 57 | 29 | 1 | 13 | 57 | 28 |
| 56 | 31 | 1 | 12 | 56 | 30 | 1 | 13 | 56 | 29 |
| 55 | 32 | 1 | 12 | 55 | 31 | 1 | 13 | 55 | 30 |
| 54 | 33 | 1 | 12 | 54 | 32 | 1 | 13 | 54 | 31 |
| 53 | 34 | 1 | 12 | 53 | 33 | 1 | 13 | 53 | 32 |
| 52 | 35 | 1 | 12 | 52 | 34 | 1 | 13 | 52 | 33 |
| 51 | 36 | 1 | 12 | 51 | 35 | 1 | 13 | 51 | 34 |
| 50 | 37 | 1 | 12 | 50 | 36 | 1 | 13 | 50 | 35 |
| 49 | 38 | 1 | 12 | 49 | 37 | 1 | 13 | 49 | 36 |
| 48 | 39 | 1 | 12 | 48 | 38 | 1 | 13 | 48 | 37 |
| 47 | 40 | 1 | 12 | 47 | 39 | 1 | 13 | 47 | 38 |
| 46 | 41 | 1 | 12 | 46 | 40 | 1 | 13 | 46 | 39 |
| 45 | 42 | 1 | 12 | 45 | 41 | 1 | 13 | 45 | 40 |
| 44 | 43 | 1 | 12 | 44 | 42 | 1 | 13 | 44 | 41 |
| 43 | 44 | 1 | 12 | 43 | 43 | 1 | 13 | 43 | 42 |
| 42 | 45 | 1 | 12 | 42 | 44 | 1 | 13 | 42 | 43 |
| 41 | 46 | 1 | 12 | 41 | 45 | 1 | 13 | 41 | 44 |
| 40 | 47 | 1 | 12 | 40 | 46 | 1 | 13 | 40 | 45 |
| 39 | 48 | 1 | 12 | 39 | 47 | 1 | 13 | 39 | 46 |
| 38 | 49 | 1 | 12 | 38 | 48 | 1 | 13 | 38 | 47 |
| 37 | 50 | 1 | 12 | 37 | 49 | 1 | 13 | 37 | 48 |
| 36 | 51 | 1 | 12 | 36 | 50 | 1 | 13 | 36 | 49 |
| 35 | 52 | 1 | 12 | 35 | 51 | 1 | 13 | 35 | 50 |
| 34 | 53 | 1 | 12 | 34 | 52 | 1 | 13 | 34 | 51 |
| 33 | 54 | 1 | 12 | 33 | 53 | 1 | 13 | 33 | 52 |
| 32 | 55 | 1 | 12 | 32 | 54 | 1 | 13 | 32 | 53 |
| 31 | 56 | 1 | 12 | 31 | 55 | 1 | 13 | 31 | 54 |
| 30 | 57 | 1 | 12 | 30 | 56 | 1 | 13 | 30 | 55 |
| 60 | 26.8 | 1.2 | 12 | 60 | 25.8 | 1.2 | 13 | 60 | 24.8 |
| 59 | 27.8 | 1.2 | 12 | 59 | 26.8 | 1.2 | 13 | 59 | 25.8 |
| 58 | 28.8 | 1.2 | 12 | 58 | 27.8 | 1.2 | 13 | 58 | 26.8 |
| 57 | 29.8 | 1.2 | 12 | 57 | 28.8 | 1.2 | 13 | 57 | 27.8 |
| 56 | 30.8 | 1.2 | 12 | 56 | 29.8 | 1.2 | 13 | 56 | 28.8 |
| 55 | 31.8 | 1.2 | 12 | 55 | 30.8 | 1.2 | 13 | 55 | 29.8 |
| 54 | 32.8 | 1.2 | 12 | 54 | 31.8 | 1.2 | 13 | 54 | 30.8 |
| 53 | 33.8 | 1.2 | 12 | 53 | 32.8 | 1.2 | 13 | 53 | 31.8 |
| 52 | 34.8 | 1.2 | 12 | 52 | 33.8 | 1.2 | 13 | 52 | 32.8 |
| 51 | 35.8 | 1.2 | 12 | 51 | 34.8 | 1.2 | 13 | 51 | 33.8 |
| 50 | 36.8 | 1.2 | 12 | 50 | 35.8 | 1.2 | 13 | 50 | 34.8 |
| 49 | 37.8 | 1.2 | 12 | 49 | 36.8 | 1.2 | 13 | 49 | 35.8 |
| 48 | 38.8 | 1.2 | 12 | 48 | 37.8 | 1.2 | 13 | 48 | 36.8 |
| 47 | 39.8 | 1.2 | 12 | 47 | 38.8 | 1.2 | 13 | 47 | 37.8 |
| 46 | 40.8 | 1.2 | 12 | 46 | 39.8 | 1.2 | 13 | 46 | 38.8 |
| 45 | 41.8 | 1.2 | 12 | 45 | 40.8 | 1.2 | 13 | 45 | 39.8 |
| 44 | 42.8 | 1.2 | 12 | 44 | 41.8 | 1.2 | 13 | 44 | 40.8 |
| 43 | 43.8 | 1.2 | 12 | 43 | 42.8 | 1.2 | 13 | 43 | 41.8 |
| 42 | 44.8 | 1.2 | 12 | 42 | 43.8 | 1.2 | 13 | 42 | 42.8 |
| 41 | 45.8 | 1.2 | 12 | 41 | 44.8 | 1.2 | 13 | 41 | 43.8 |
| 40 | 46.8 | 1.2 | 12 | 40 | 45.8 | 1.2 | 13 | 40 | 44.8 |
| 39 | 47.8 | 1.2 | 12 | 39 | 46.8 | 1.2 | 13 | 39 | 45.8 |
| 38 | 48.8 | 1.2 | 12 | 38 | 47.8 | 1.2 | 13 | 38 | 46.8 |
| 37 | 49.8 | 1.2 | 12 | 37 | 48.8 | 1.2 | 13 | 37 | 47.8 |
| 36 | 50.8 | 1.2 | 12 | 36 | 49.8 | 1.2 | 13 | 36 | 48.8 |
| 35 | 51.8 | 1.2 | 12 | 35 | 50.8 | 1.2 | 13 | 35 | 49.8 |
| 34 | 52.8 | 1.2 | 12 | 34 | 51.8 | 1.2 | 13 | 34 | 50.8 |
| 33 | 53.8 | 1.2 | 12 | 33 | 52.8 | 1.2 | 13 | 33 | 51.8 |
| 32 | 54.8 | 1.2 | 12 | 32 | 53.8 | 1.2 | 13 | 32 | 52.8 |
| 31 | 55.8 | 1.2 | 12 | 31 | 54.8 | 1.2 | 13 | 31 | 53.8 |
| 30 | 56.8 | 1.2 | 12 | 30 | 55.8 | 1.2 | 13 | 30 | 54.8 |
| 60 | 26.6 | 1.4 | 12 | 60 | 25.6 | 1.4 | 13 | 60 | 24.6 |
| 59 | 27.6 | 1.4 | 12 | 59 | 26.6 | 1.4 | 13 | 59 | 25.6 |
| 58 | 28.6 | 1.4 | 12 | 58 | 27.6 | 1.4 | 13 | 58 | 26.6 |
| 57 | 29.6 | 1.4 | 12 | 57 | 28.6 | 1.4 | 13 | 57 | 27.6 |
| 56 | 30.6 | 1.4 | 12 | 56 | 29.6 | 1.4 | 13 | 56 | 28.6 |
| 55 | 31.6 | 1.4 | 12 | 55 | 30.6 | 1.4 | 13 | 55 | 29.6 |
| 54 | 32.6 | 1.4 | 12 | 54 | 31.6 | 1.4 | 13 | 54 | 30.6 |
| 53 | 33.6 | 1.4 | 12 | 53 | 32.6 | 1.4 | 13 | 53 | 31.6 |
| 52 | 34.6 | 1.4 | 12 | 52 | 33.6 | 1.4 | 13 | 52 | 32.6 |
| 51 | 35.6 | 1.4 | 12 | 51 | 34.6 | 1.4 | 13 | 51 | 33.6 |
| 50 | 36.6 | 1.4 | 12 | 50 | 35.6 | 1.4 | 13 | 50 | 34.6 |
| 49 | 37.6 | 1.4 | 12 | 49 | 36.6 | 1.4 | 13 | 49 | 35.6 |
| 48 | 38.6 | 1.4 | 12 | 48 | 37.6 | 1.4 | 13 | 48 | 36.6 |
| 47 | 39.6 | 1.4 | 12 | 47 | 38.6 | 1.4 | 13 | 47 | 37.6 |
| 46 | 40.6 | 1.4 | 12 | 46 | 39.6 | 1.4 | 13 | 46 | 38.6 |
| 45 | 41.6 | 1.4 | 12 | 45 | 40.6 | 1.4 | 13 | 45 | 39.6 |
| 44 | 42.6 | 1.4 | 12 | 44 | 41.6 | 1.4 | 13 | 44 | 40.6 |
| 43 | 43.6 | 1.4 | 12 | 43 | 42.6 | 1.4 | 13 | 43 | 41.6 |
| 42 | 44.6 | 1.4 | 12 | 42 | 43.6 | 1.4 | 13 | 42 | 42.6 |
| 41 | 45.6 | 1.4 | 12 | 41 | 44.6 | 1.4 | 13 | 41 | 43.6 |
| 40 | 46.6 | 1.4 | 12 | 40 | 45.6 | 1.4 | 13 | 40 | 44.6 |
| 39 | 47.6 | 1.4 | 12 | 39 | 46.6 | 1.4 | 13 | 39 | 45.6 |
| 38 | 48.6 | 1.4 | 12 | 38 | 47.6 | 1.4 | 13 | 38 | 46.6 |
| 37 | 49.6 | 1.4 | 12 | 37 | 48.6 | 1.4 | 13 | 37 | 47.6 |
| 36 | 50.6 | 1.4 | 12 | 36 | 49.6 | 1.4 | 13 | 36 | 48.6 |
| 35 | 51.6 | 1.4 | 12 | 35 | 50.6 | 1.4 | 13 | 35 | 49.6 |
| 34 | 52.6 | 1.4 | 12 | 34 | 51.6 | 1.4 | 13 | 34 | 50.6 |
| 33 | 53.6 | 1.4 | 12 | 33 | 52.6 | 1.4 | 13 | 33 | 51.6 |
| 32 | 54.6 | 1.4 | 12 | 32 | 53.6 | 1.4 | 13 | 32 | 52.6 |
| 31 | 55.6 | 1.4 | 12 | 31 | 54.6 | 1.4 | 13 | 31 | 53.6 |
| 30 | 56.6 | 1.4 | 12 | 30 | 55.6 | 1.4 | 13 | 30 | 54.6 |
| 60 | 26.4 | 1.6 | 12 | 60 | 25.4 | 1.6 | 13 | 60 | 24.4 |
| 59 | 27.4 | 1.6 | 12 | 59 | 26.4 | 1.6 | 13 | 59 | 25.4 |
| 58 | 28.4 | 1.6 | 12 | 58 | 27.4 | 1.6 | 13 | 58 | 26.4 |
| 57 | 29.4 | 1.6 | 12 | 57 | 28.4 | 1.6 | 13 | 57 | 27.4 |
| 56 | 30.4 | 1.6 | 12 | 56 | 29.4 | 1.6 | 13 | 56 | 28.4 |
| 55 | 31.4 | 1.6 | 12 | 55 | 30.4 | 1.6 | 13 | 55 | 29.4 |
| 54 | 32.4 | 1.6 | 12 | 54 | 31.4 | 1.6 | 13 | 54 | 30.4 |
| 53 | 33.4 | 1.6 | 12 | 53 | 32.4 | 1.6 | 13 | 53 | 31.4 |
| 52 | 34.4 | 1.6 | 12 | 52 | 33.4 | 1.6 | 13 | 52 | 32.4 |
| 51 | 35.4 | 1.6 | 12 | 51 | 34.4 | 1.6 | 13 | 51 | 33.4 |
| 50 | 36.4 | 1.6 | 12 | 50 | 35.4 | 1.6 | 13 | 50 | 34.4 |
| 49 | 37.4 | 1.6 | 12 | 49 | 36.4 | 1.6 | 13 | 49 | 35.4 |
| 48 | 38.4 | 1.6 | 12 | 48 | 37.4 | 1.6 | 13 | 48 | 36.4 |
| 47 | 39.4 | 1.6 | 12 | 47 | 38.4 | 1.6 | 13 | 47 | 37.4 |
| 46 | 40.4 | 1.6 | 12 | 46 | 39.4 | 1.6 | 13 | 46 | 38.4 |
| 45 | 41.4 | 1.6 | 12 | 45 | 40.4 | 1.6 | 13 | 45 | 39.4 |
| 44 | 42.4 | 1.6 | 12 | 44 | 41.4 | 1.6 | 13 | 44 | 40.4 |
| 43 | 43.4 | 1.6 | 12 | 43 | 42.4 | 1.6 | 13 | 43 | 41.4 |
| 42 | 44.4 | 1.6 | 12 | 42 | 43.4 | 1.6 | 13 | 42 | 42.4 |
| 41 | 45.4 | 1.6 | 12 | 41 | 44.4 | 1.6 | 13 | 41 | 43.4 |
| 40 | 46.4 | 1.6 | 12 | 40 | 45.4 | 1.6 | 13 | 40 | 44.4 |
| 39 | 47.4 | 1.6 | 12 | 39 | 46.4 | 1.6 | 13 | 39 | 45.4 |
| 38 | 48.4 | 1.6 | 12 | 38 | 47.4 | 1.6 | 13 | 38 | 46.4 |
| 37 | 49.4 | 1.6 | 12 | 37 | 48.4 | 1.6 | 13 | 37 | 47.4 |
| 36 | 50.4 | 1.6 | 12 | 36 | 49.4 | 1.6 | 13 | 36 | 48.4 |
| 35 | 51.4 | 1.6 | 12 | 35 | 50.4 | 1.6 | 13 | 35 | 49.4 |
| 34 | 52.4 | 1.6 | 12 | 34 | 51.4 | 1.6 | 13 | 34 | 50.4 |
| 33 | 53.4 | 1.6 | 12 | 33 | 52.4 | 1.6 | 13 | 33 | 51.4 |
| 32 | 54.4 | 1.6 | 12 | 32 | 53.4 | 1.6 | 13 | 32 | 52.4 |
| 31 | 55.4 | 1.6 | 12 | 31 | 54.4 | 1.6 | 13 | 31 | 53.4 |
| 30 | 56.4 | 1.6 | 12 | 30 | 55.4 | 1.6 | 13 | 30 | 54.4 |
| 60 | 26.2 | 1.8 | 12 | 60 | 25.2 | 1.8 | 13 | 60 | 24.2 |
| 59 | 27.2 | 1.8 | 12 | 59 | 26.2 | 1.8 | 13 | 59 | 25.2 |
| 58 | 28.2 | 1.8 | 12 | 58 | 27.2 | 1.8 | 13 | 58 | 26.2 |
| 57 | 29.2 | 1.8 | 12 | 57 | 28.2 | 1.8 | 13 | 57 | 27.2 |
| 56 | 30.2 | 1.8 | 12 | 56 | 29.2 | 1.8 | 13 | 56 | 28.2 |
| 55 | 31.2 | 1.8 | 12 | 55 | 30.2 | 1.8 | 13 | 55 | 29.2 |
| 54 | 32.2 | 1.8 | 12 | 54 | 31.2 | 1.8 | 13 | 54 | 30.2 |
| 53 | 33.2 | 1.8 | 12 | 53 | 32.2 | 1.8 | 13 | 53 | 31.2 |
| 52 | 34.2 | 1.8 | 12 | 52 | 33.2 | 1.8 | 13 | 52 | 32.2 |
| 51 | 35.2 | 1.8 | 12 | 51 | 34.2 | 1.8 | 13 | 51 | 33.2 |
| 50 | 36.2 | 1.8 | 12 | 50 | 35.2 | 1.8 | 13 | 50 | 34.2 |
| 49 | 37.2 | 1.8 | 12 | 49 | 36.2 | 1.8 | 13 | 49 | 35.2 |
| 48 | 38.2 | 1.8 | 12 | 48 | 37.2 | 1.8 | 13 | 48 | 36.2 |
| 47 | 39.2 | 1.8 | 12 | 47 | 38.2 | 1.8 | 13 | 47 | 37.2 |
| 46 | 40.2 | 1.8 | 12 | 46 | 39.2 | 1.8 | 13 | 46 | 38.2 |
| 45 | 41.2 | 1.8 | 12 | 45 | 40.2 | 1.8 | 13 | 45 | 39.2 |
| 44 | 42.2 | 1.8 | 12 | 44 | 41.2 | 1.8 | 13 | 44 | 40.2 |
| 43 | 43.2 | 1.8 | 12 | 43 | 42.2 | 1.8 | 13 | 43 | 41.2 |
| 42 | 44.2 | 1.8 | 12 | 42 | 43.2 | 1.8 | 13 | 42 | 42.2 |
| 41 | 45.2 | 1.8 | 12 | 41 | 44.2 | 1.8 | 13 | 41 | 43.2 |
| 40 | 46.2 | 1.8 | 12 | 40 | 45.2 | 1.8 | 13 | 40 | 44.2 |
| 39 | 47.2 | 1.8 | 12 | 39 | 46.2 | 1.8 | 13 | 39 | 45.2 |
| 38 | 48.2 | 1.8 | 12 | 38 | 47.2 | 1.8 | 13 | 38 | 46.2 |
| 37 | 49.2 | 1.8 | 12 | 37 | 48.2 | 1.8 | 13 | 37 | 47.2 |
| 36 | 50.2 | 1.8 | 12 | 36 | 49.2 | 1.8 | 13 | 36 | 48.2 |
| 35 | 51.2 | 1.8 | 12 | 35 | 50.2 | 1.8 | 13 | 35 | 49.2 |
| 34 | 52.2 | 1.8 | 12 | 34 | 51.2 | 1.8 | 13 | 34 | 50.2 |
| 33 | 53.2 | 1.8 | 12 | 33 | 52.2 | 1.8 | 13 | 33 | 51.2 |
| 32 | 54.2 | 1.8 | 12 | 32 | 53.2 | 1.8 | 13 | 32 | 52.2 |
| 31 | 55.2 | 1.8 | 12 | 31 | 54.2 | 1.8 | 13 | 31 | 53.2 |
| 30 | 56.2 | 1.8 | 12 | 30 | 55.2 | 1.8 | 13 | 30 | 54.2 |
| 60 | 26 | 2 | 12 | 60 | 25 | 2 | 13 | 60 | 24 |
| 59 | 27 | 2 | 12 | 59 | 26 | 2 | 13 | 59 | 25 |
| 58 | 28 | 2 | 12 | 58 | 27 | 2 | 13 | 58 | 26 |
| 57 | 29 | 2 | 12 | 57 | 28 | 2 | 13 | 57 | 27 |
| 56 | 30 | 2 | 12 | 56 | 29 | 2 | 13 | 56 | 28 |
| 55 | 31 | 2 | 12 | 55 | 30 | 2 | 13 | 55 | 29 |
| 54 | 32 | 2 | 12 | 54 | 31 | 2 | 13 | 54 | 30 |
| 53 | 33 | 2 | 12 | 53 | 32 | 2 | 13 | 53 | 31 |
| 52 | 34 | 2 | 12 | 52 | 33 | 2 | 13 | 52 | 32 |
| 51 | 35 | 2 | 12 | 51 | 34 | 2 | 13 | 51 | 33 |
| 50 | 36 | 2 | 12 | 50 | 35 | 2 | 13 | 50 | 34 |
| 49 | 37 | 2 | 12 | 49 | 36 | 2 | 13 | 49 | 35 |
| 48 | 38 | 2 | 12 | 48 | 37 | 2 | 13 | 48 | 36 |
| 47 | 39 | 2 | 12 | 47 | 38 | 2 | 13 | 47 | 37 |
| 46 | 40 | 2 | 12 | 46 | 39 | 2 | 13 | 46 | 38 |
| 45 | 41 | 2 | 12 | 45 | 40 | 2 | 13 | 45 | 39 |
| 44 | 42 | 2 | 12 | 44 | 41 | 2 | 13 | 44 | 40 |
| 43 | 43 | 2 | 12 | 43 | 42 | 2 | 13 | 43 | 41 |
| 42 | 44 | 2 | 12 | 42 | 43 | 2 | 13 | 42 | 42 |
| 41 | 45 | 2 | 12 | 41 | 44 | 2 | 13 | 41 | 43 |
| 40 | 46 | 2 | 12 | 40 | 45 | 2 | 13 | 40 | 44 |
| 39 | 47 | 2 | 12 | 39 | 46 | 2 | 13 | 39 | 45 |
| 38 | 48 | 2 | 12 | 38 | 47 | 2 | 13 | 38 | 46 |
| 37 | 49 | 2 | 12 | 37 | 48 | 2 | 13 | 37 | 47 |
| 36 | 50 | 2 | 12 | 36 | 49 | 2 | 13 | 36 | 48 |
| 35 | 51 | 2 | 12 | 35 | 50 | 2 | 13 | 35 | 49 |
| 34 | 52 | 2 | 12 | 34 | 51 | 2 | 13 | 34 | 50 |
| 33 | 53 | 2 | 12 | 33 | 52 | 2 | 13 | 33 | 51 |
| 32 | 54 | 2 | 12 | 32 | 53 | 2 | 13 | 32 | 52 |
| 31 | 55 | 2 | 12 | 31 | 54 | 2 | 13 | 31 | 53 |
| 30 | 56 | 2 | 12 | 30 | 55 | 2 | 13 | 30 | 54 |
| 60 | 25.8 | 2.2 | 12 | 60 | 24.8 | 2.2 | 13 | 60 | 23.8 |
| 59 | 26.8 | 2.2 | 12 | 59 | 25.8 | 2.2 | 13 | 59 | 24.8 |
| 58 | 27.8 | 2.2 | 12 | 58 | 26.8 | 2.2 | 13 | 58 | 25.8 |
| 57 | 28.8 | 2.2 | 12 | 57 | 27.8 | 2.2 | 13 | 57 | 26.8 |
| 56 | 29.8 | 2.2 | 12 | 56 | 28.8 | 2.2 | 13 | 56 | 27.8 |
| 55 | 30.8 | 2.2 | 12 | 55 | 29.8 | 2.2 | 13 | 55 | 28.8 |
| 54 | 31.8 | 2.2 | 12 | 54 | 30.8 | 2.2 | 13 | 54 | 29.8 |
| 53 | 32.8 | 2.2 | 12 | 53 | 31.8 | 2.2 | 13 | 53 | 30.8 |
| 52 | 33.8 | 2.2 | 12 | 52 | 32.8 | 2.2 | 13 | 52 | 31.8 |
| 51 | 34.8 | 2.2 | 12 | 51 | 33.8 | 2.2 | 13 | 51 | 32.8 |
| 50 | 35.8 | 2.2 | 12 | 50 | 34.8 | 2.2 | 13 | 50 | 33.8 |
| 49 | 36.8 | 2.2 | 12 | 49 | 35.8 | 2.2 | 13 | 49 | 34.8 |
| 48 | 37.8 | 2.2 | 12 | 48 | 36.8 | 2.2 | 13 | 48 | 35.8 |
| 47 | 38.8 | 2.2 | 12 | 47 | 37.8 | 2.2 | 13 | 47 | 36.8 |
| 46 | 39.8 | 2.2 | 12 | 46 | 38.8 | 2.2 | 13 | 46 | 37.8 |
| 45 | 40.8 | 2.2 | 12 | 45 | 39.8 | 2.2 | 13 | 45 | 38.8 |
| 44 | 41.8 | 2.2 | 12 | 44 | 40.8 | 2.2 | 13 | 44 | 39.8 |
| 43 | 42.8 | 2.2 | 12 | 43 | 41.8 | 2.2 | 13 | 43 | 40.8 |
| 42 | 43.8 | 2.2 | 12 | 42 | 42.8 | 2.2 | 13 | 42 | 41.8 |
| 41 | 44.8 | 2.2 | 12 | 41 | 43.8 | 2.2 | 13 | 41 | 42.8 |
| 40 | 45.8 | 2.2 | 12 | 40 | 44.8 | 2.2 | 13 | 40 | 43.8 |
| 39 | 46.8 | 2.2 | 12 | 39 | 45.8 | 2.2 | 13 | 39 | 44.8 |
| 38 | 47.8 | 2.2 | 12 | 38 | 46.8 | 2.2 | 13 | 38 | 45.8 |
| 37 | 48.8 | 2.2 | 12 | 37 | 47.8 | 2.2 | 13 | 37 | 46.8 |
| 36 | 49.8 | 2.2 | 12 | 36 | 48.8 | 2.2 | 13 | 36 | 47.8 |
| 35 | 50.8 | 2.2 | 12 | 35 | 49.8 | 2.2 | 13 | 35 | 48.8 |
| 34 | 51.8 | 2.2 | 12 | 34 | 50.8 | 2.2 | 13 | 34 | 49.8 |
| 33 | 52.8 | 2.2 | 12 | 33 | 51.8 | 2.2 | 13 | 33 | 50.8 |
| 32 | 53.8 | 2.2 | 12 | 32 | 52.8 | 2.2 | 13 | 32 | 51.8 |
| 31 | 54.8 | 2.2 | 12 | 31 | 53.8 | 2.2 | 13 | 31 | 52.8 |
| 30 | 55.8 | 2.2 | 12 | 30 | 54.8 | 2.2 | 13 | 30 | 53.8 |
| 60 | 25.6 | 2.4 | 12 | 60 | 24.6 | 2.4 | 13 | 60 | 23.6 |
| 59 | 26.6 | 2.4 | 12 | 59 | 25.6 | 2.4 | 13 | 59 | 24.6 |
| 58 | 27.6 | 2.4 | 12 | 58 | 26.6 | 2.4 | 13 | 58 | 25.6 |
| 57 | 28.6 | 2.4 | 12 | 57 | 27.6 | 2.4 | 13 | 57 | 26.6 |
| 56 | 29.6 | 2.4 | 12 | 56 | 28.6 | 2.4 | 13 | 56 | 27.6 |
| 55 | 30.6 | 2.4 | 12 | 55 | 29.6 | 2.4 | 13 | 55 | 28.6 |
| 54 | 31.6 | 2.4 | 12 | 54 | 30.6 | 2.4 | 13 | 54 | 29.6 |
| 53 | 32.6 | 2.4 | 12 | 53 | 31.6 | 2.4 | 13 | 53 | 30.6 |
| 52 | 33.6 | 2.4 | 12 | 52 | 32.6 | 2.4 | 13 | 52 | 31.6 |
| 51 | 34.6 | 2.4 | 12 | 51 | 33.6 | 2.4 | 13 | 51 | 32.6 |
| 50 | 35.6 | 2.4 | 12 | 50 | 34.6 | 2.4 | 13 | 50 | 33.6 |
| 49 | 36.6 | 2.4 | 12 | 49 | 35.6 | 2.4 | 13 | 49 | 34.6 |
| 48 | 37.6 | 2.4 | 12 | 48 | 36.6 | 2.4 | 13 | 48 | 35.6 |
| 47 | 38.6 | 2.4 | 12 | 47 | 37.6 | 2.4 | 13 | 47 | 36.6 |
| 46 | 39.6 | 2.4 | 12 | 46 | 38.6 | 2.4 | 13 | 46 | 37.6 |
| 45 | 40.6 | 2.4 | 12 | 45 | 39.6 | 2.4 | 13 | 45 | 38.6 |
| 44 | 41.6 | 2.4 | 12 | 44 | 40.6 | 2.4 | 13 | 44 | 39.6 |
| 43 | 42.6 | 2.4 | 12 | 43 | 41.6 | 2.4 | 13 | 43 | 40.6 |
| 42 | 43.6 | 2.4 | 12 | 42 | 42.6 | 2.4 | 13 | 42 | 41.6 |
| 41 | 44.6 | 2.4 | 12 | 41 | 43.6 | 2.4 | 13 | 41 | 42.6 |
| 40 | 45.6 | 2.4 | 12 | 40 | 44.6 | 2.4 | 13 | 40 | 43.6 |
| 39 | 46.6 | 2.4 | 12 | 39 | 45.6 | 2.4 | 13 | 39 | 44.6 |
| 38 | 47.6 | 2.4 | 12 | 38 | 46.6 | 2.4 | 13 | 38 | 45.6 |
| 37 | 48.6 | 2.4 | 12 | 37 | 47.6 | 2.4 | 13 | 37 | 46.6 |
| 36 | 49.6 | 2.4 | 12 | 36 | 48.6 | 2.4 | 13 | 36 | 47.6 |
| 35 | 50.6 | 2.4 | 12 | 35 | 49.6 | 2.4 | 13 | 35 | 48.6 |
| 34 | 51.6 | 2.4 | 12 | 34 | 50.6 | 2.4 | 13 | 34 | 49.6 |
| 33 | 52.6 | 2.4 | 12 | 33 | 51.6 | 2.4 | 13 | 33 | 50.6 |
| 32 | 53.6 | 2.4 | 12 | 32 | 52.6 | 2.4 | 13 | 32 | 51.6 |
| 31 | 54.6 | 2.4 | 12 | 31 | 53.6 | 2.4 | 13 | 31 | 52.6 |
| 30 | 55.6 | 2.4 | 12 | 30 | 54.6 | 2.4 | 13 | 30 | 53.6 |
| 60 | 25.4 | 2.6 | 12 | 60 | 24.4 | 2.6 | 13 | 60 | 23.4 |
| 59 | 26.4 | 2.6 | 12 | 59 | 25.4 | 2.6 | 13 | 59 | 24.4 |
| 58 | 27.4 | 2.6 | 12 | 58 | 26.4 | 2.6 | 13 | 58 | 25.4 |
| 57 | 28.4 | 2.6 | 12 | 57 | 27.4 | 2.6 | 13 | 57 | 26.4 |
| 56 | 29.4 | 2.6 | 12 | 56 | 28.4 | 2.6 | 13 | 56 | 27.4 |
| 55 | 30.4 | 2.6 | 12 | 55 | 29.4 | 2.6 | 13 | 55 | 28.4 |
| 54 | 31.4 | 2.6 | 12 | 54 | 30.4 | 2.6 | 13 | 54 | 29.4 |
| 53 | 32.4 | 2.6 | 12 | 53 | 31.4 | 2.6 | 13 | 53 | 30.4 |
| 52 | 33.4 | 2.6 | 12 | 52 | 32.4 | 2.6 | 13 | 52 | 31.4 |
| 51 | 34.4 | 2.6 | 12 | 51 | 33.4 | 2.6 | 13 | 51 | 32.4 |
| 50 | 35.4 | 2.6 | 12 | 50 | 34.4 | 2.6 | 13 | 50 | 33.4 |
| 49 | 36.4 | 2.6 | 12 | 49 | 35.4 | 2.6 | 13 | 49 | 34.4 |
| 48 | 37.4 | 2.6 | 12 | 48 | 36.4 | 2.6 | 13 | 48 | 35.4 |
| 47 | 38.4 | 2.6 | 12 | 47 | 37.4 | 2.6 | 13 | 47 | 36.4 |
| 46 | 39.4 | 2.6 | 12 | 46 | 38.4 | 2.6 | 13 | 46 | 37.4 |
| 45 | 40.4 | 2.6 | 12 | 45 | 39.4 | 2.6 | 13 | 45 | 38.4 |
| 44 | 41.4 | 2.6 | 12 | 44 | 40.4 | 2.6 | 13 | 44 | 39.4 |
| 43 | 42.4 | 2.6 | 12 | 43 | 41.4 | 2.6 | 13 | 43 | 40.4 |
| 42 | 43.4 | 2.6 | 12 | 42 | 42.4 | 2.6 | 13 | 42 | 41.4 |
| 41 | 44.4 | 2.6 | 12 | 41 | 43.4 | 2.6 | 13 | 41 | 42.4 |
| 40 | 45.4 | 2.6 | 12 | 40 | 44.4 | 2.6 | 13 | 40 | 43.4 |
| 39 | 46.4 | 2.6 | 12 | 39 | 45.4 | 2.6 | 13 | 39 | 44.4 |
| 38 | 47.4 | 2.6 | 12 | 38 | 46.4 | 2.6 | 13 | 38 | 45.4 |
| 37 | 48.4 | 2.6 | 12 | 37 | 47.4 | 2.6 | 13 | 37 | 46.4 |
| 36 | 49.4 | 2.6 | 12 | 36 | 48.4 | 2.6 | 13 | 36 | 47.4 |
| 35 | 50.4 | 2.6 | 12 | 35 | 49.4 | 2.6 | 13 | 35 | 48.4 |
| 34 | 51.4 | 2.6 | 12 | 34 | 50.4 | 2.6 | 13 | 34 | 49.4 |
| 33 | 52.4 | 2.6 | 12 | 33 | 51.4 | 2.6 | 13 | 33 | 50.4 |
| 32 | 53.4 | 2.6 | 12 | 32 | 52.4 | 2.6 | 13 | 32 | 51.4 |
| 31 | 54.4 | 2.6 | 12 | 31 | 53.4 | 2.6 | 13 | 31 | 52.4 |
| 30 | 55.4 | 2.6 | 12 | 30 | 54.4 | 2.6 | 13 | 30 | 53.4 |
| 60 | 25.2 | 2.8 | 12 | 60 | 24.2 | 2.8 | 13 | 60 | 23.2 |
| 59 | 26.2 | 2.8 | 12 | 59 | 25.2 | 2.8 | 13 | 59 | 24.2 |
| 58 | 27.2 | 2.8 | 12 | 58 | 26.2 | 2.8 | 13 | 58 | 25.2 |
| 57 | 28.2 | 2.8 | 12 | 57 | 27.2 | 2.8 | 13 | 57 | 26.2 |
| 56 | 29.2 | 2.8 | 12 | 56 | 28.2 | 2.8 | 13 | 56 | 27.2 |
| 55 | 30.2 | 2.8 | 12 | 55 | 29.2 | 2.8 | 13 | 55 | 28.2 |
| 54 | 31.2 | 2.8 | 12 | 54 | 30.2 | 2.8 | 13 | 54 | 29.2 |
| 53 | 32.2 | 2.8 | 12 | 53 | 31.2 | 2.8 | 13 | 53 | 30.2 |
| 52 | 33.2 | 2.8 | 12 | 52 | 32.2 | 2.8 | 13 | 52 | 31.2 |
| 51 | 34.2 | 2.8 | 12 | 51 | 33.2 | 2.8 | 13 | 51 | 32.2 |
| 50 | 35.2 | 2.8 | 12 | 50 | 34.2 | 2.8 | 13 | 50 | 33.2 |
| 49 | 36.2 | 2.8 | 12 | 49 | 35.2 | 2.8 | 13 | 49 | 34.2 |
| 48 | 37.2 | 2.8 | 12 | 48 | 36.2 | 2.8 | 13 | 48 | 35.2 |
| 47 | 38.2 | 2.8 | 12 | 47 | 37.2 | 2.8 | 13 | 47 | 36.2 |
| 46 | 39.2 | 2.8 | 12 | 46 | 38.2 | 2.8 | 13 | 46 | 37.2 |
| 45 | 40.2 | 2.8 | 12 | 45 | 39.2 | 2.8 | 13 | 45 | 38.2 |
| 44 | 41.2 | 2.8 | 12 | 44 | 40.2 | 2.8 | 13 | 44 | 39.2 |
| 43 | 42.2 | 2.8 | 12 | 43 | 41.2 | 2.8 | 13 | 43 | 40.2 |
| 42 | 43.2 | 2.8 | 12 | 42 | 42.2 | 2.8 | 13 | 42 | 41.2 |
| 41 | 44.2 | 2.8 | 12 | 41 | 43.2 | 2.8 | 13 | 41 | 42.2 |
| 40 | 45.2 | 2.8 | 12 | 40 | 44.2 | 2.8 | 13 | 40 | 43.2 |
| 39 | 46.2 | 2.8 | 12 | 39 | 45.2 | 2.8 | 13 | 39 | 44.2 |
| 38 | 47.2 | 2.8 | 12 | 38 | 46.2 | 2.8 | 13 | 38 | 45.2 |
| 37 | 48.2 | 2.8 | 12 | 37 | 47.2 | 2.8 | 13 | 37 | 46.2 |
| 36 | 49.2 | 2.8 | 12 | 36 | 48.2 | 2.8 | 13 | 36 | 47.2 |
| 35 | 50.2 | 2.8 | 12 | 35 | 49.2 | 2.8 | 13 | 35 | 48.2 |
| 34 | 51.2 | 2.8 | 12 | 34 | 50.2 | 2.8 | 13 | 34 | 49.2 |
| 33 | 52.2 | 2.8 | 12 | 33 | 51.2 | 2.8 | 13 | 33 | 50.2 |
| 32 | 53.2 | 2.8 | 12 | 32 | 52.2 | 2.8 | 13 | 32 | 51.2 |
| 31 | 54.2 | 2.8 | 12 | 31 | 53.2 | 2.8 | 13 | 31 | 52.2 |
| 30 | 55.2 | 2.8 | 12 | 30 | 54.2 | 2.8 | 13 | 30 | 53.2 |
| 60 | 25 | 3 | 12 | 60 | 24 | 3 | 13 | 60 | 23 |
| 59 | 26 | 3 | 12 | 59 | 25 | 3 | 13 | 59 | 24 |
| 58 | 27 | 3 | 12 | 58 | 26 | 3 | 13 | 58 | 25 |
| 57 | 28 | 3 | 12 | 57 | 27 | 3 | 13 | 57 | 26 |
| 56 | 29 | 3 | 12 | 56 | 28 | 3 | 13 | 56 | 27 |
| 55 | 30 | 3 | 12 | 55 | 29 | 3 | 13 | 55 | 28 |
| 54 | 31 | 3 | 12 | 54 | 30 | 3 | 13 | 54 | 29 |
| 53 | 32 | 3 | 12 | 53 | 31 | 3 | 13 | 53 | 30 |
| 52 | 33 | 3 | 12 | 52 | 32 | 3 | 13 | 52 | 31 |
| 51 | 34 | 3 | 12 | 51 | 33 | 3 | 13 | 51 | 32 |
| 50 | 35 | 3 | 12 | 50 | 34 | 3 | 13 | 50 | 33 |
| 49 | 36 | 3 | 12 | 49 | 35 | 3 | 13 | 49 | 34 |
| 48 | 37 | 3 | 12 | 48 | 36 | 3 | 13 | 48 | 35 |
| 47 | 38 | 3 | 12 | 47 | 37 | 3 | 13 | 47 | 36 |
| 46 | 39 | 3 | 12 | 46 | 38 | 3 | 13 | 46 | 37 |
| 45 | 40 | 3 | 12 | 45 | 39 | 3 | 13 | 45 | 38 |
| 44 | 41 | 3 | 12 | 44 | 40 | 3 | 13 | 44 | 39 |
| 43 | 42 | 3 | 12 | 43 | 41 | 3 | 13 | 43 | 40 |
| 42 | 43 | 3 | 12 | 42 | 42 | 3 | 13 | 42 | 41 |
| 41 | 44 | 3 | 12 | 41 | 43 | 3 | 13 | 41 | 42 |
| 40 | 45 | 3 | 12 | 40 | 44 | 3 | 13 | 40 | 43 |
| 39 | 46 | 3 | 12 | 39 | 45 | 3 | 13 | 39 | 44 |
| 38 | 47 | 3 | 12 | 38 | 46 | 3 | 13 | 38 | 45 |
| 37 | 48 | 3 | 12 | 37 | 47 | 3 | 13 | 37 | 46 |
| 36 | 49 | 3 | 12 | 36 | 48 | 3 | 13 | 36 | 47 |
| 35 | 50 | 3 | 12 | 35 | 49 | 3 | 13 | 35 | 48 |
| 34 | 51 | 3 | 12 | 34 | 50 | 3 | 13 | 34 | 49 |
| 33 | 52 | 3 | 12 | 33 | 51 | 3 | 13 | 33 | 50 |
| 32 | 53 | 3 | 12 | 32 | 52 | 3 | 13 | 32 | 51 |
| 31 | 54 | 3 | 12 | 31 | 53 | 3 | 13 | 31 | 52 |
| 30 | 55 | 3 | 12 | 30 | 54 | 3 | 13 | 30 | 53 |

| | second | | | | second |
|---|---|---|---|---|---|
| polymer- | lipid | | | polymer- | lipid |
| conjugated | (i.e | first | | conjugated | (i.e |
| lipid | anionic, | lipid | | lipid | anionic, |
| (i.e. | neutral, | (i.e. | | (i.e. | neutral, |
| PEG- | or | cation- | sterol | PEG- | or |
| conjugated | zwitterionic | ionizable | (i.e. | conjugated | zwitterionic |
| lipid) | lipid) | lipid | cholesterol | lipid) | lipid) |
| (mole %) | (mole %) | (mole %) | (mole %) | (mole %) | (mole %) |
| 1 | 2 | 60 | 36 | 1 | 3 |
| 1 | 2 | 59 | 37 | 1 | 3 |
| 1 | 2 | 58 | 38 | 1 | 3 |
| 1 | 2 | 57 | 39 | 1 | 3 |
| 1 | 2 | 56 | 40 | 1 | 3 |
| 1 | 2 | 55 | 41 | 1 | 3 |
| 1 | 2 | 54 | 42 | 1 | 3 |
| 1 | 2 | 53 | 43 | 1 | 3 |
| 1 | 2 | 52 | 44 | 1 | 3 |
| 1 | 2 | 51 | 45 | 1 | 3 |
| 1 | 2 | 50 | 46 | 1 | 3 |
| 1 | 2 | 49 | 47 | 1 | 3 |
| 1 | 2 | 48 | 48 | 1 | 3 |
| 1 | 2 | 47 | 49 | 1 | 3 |
| 1 | 2 | 46 | 50 | 1 | 3 |
| 1 | 2 | 45 | 51 | 1 | 3 |
| 1 | 2 | 44 | 52 | 1 | 3 |
| 1 | 2 | 43 | 53 | 1 | 3 |
| 1 | 2 | 42 | 54 | 1 | 3 |
| 1 | 2 | 41 | 55 | 1 | 3 |
| 1 | 2 | 40 | 56 | 1 | 3 |
| 1 | 2 | 39 | 57 | 1 | 3 |
| 1 | 2 | 38 | 58 | 1 | 3 |
| 1 | 2 | 37 | 59 | 1 | 3 |
| 1 | 2 | 36 | 60 | 1 | 3 |
| 1 | 2 | 35 | 61 | 1 | 3 |
| 1 | 2 | 34 | 62 | 1 | 3 |
| 1 | 2 | 33 | 63 | 1 | 3 |
| 1 | 2 | 32 | 64 | 1 | 3 |
| 1 | 2 | 31 | 65 | 1 | 3 |
| 1 | 2 | 30 | 66 | 1 | 3 |
| 1.2 | 2 | 60 | 35.8 | 1.2 | 3 |
| 1.2 | 2 | 59 | 36.8 | 1.2 | 3 |
| 1.2 | 2 | 58 | 37.8 | 1.2 | 3 |
| 1.2 | 2 | 57 | 38.8 | 1.2 | 3 |
| 1.2 | 2 | 56 | 39.8 | 1.2 | 3 |
| 1.2 | 2 | 55 | 40.8 | 1.2 | 3 |
| 1.2 | 2 | 54 | 41.8 | 1.2 | 3 |
| 1.2 | 2 | 53 | 42.8 | 1.2 | 3 |
| 1.2 | 2 | 52 | 43.8 | 1.2 | 3 |
| 1.2 | 2 | 51 | 44.8 | 1.2 | 3 |
| 1.2 | 2 | 50 | 45.8 | 1.2 | 3 |
| 1.2 | 2 | 49 | 46.8 | 1.2 | 3 |
| 1.2 | 2 | 48 | 47.8 | 1.2 | 3 |
| 1.2 | 2 | 47 | 48.8 | 1.2 | 3 |
| 1.2 | 2 | 46 | 49.8 | 1.2 | 3 |
| 1.2 | 2 | 45 | 50.8 | 1.2 | 3 |
| 1.2 | 2 | 44 | 51.8 | 1.2 | 3 |
| 1.2 | 2 | 43 | 52.8 | 1.2 | 3 |
| 1.2 | 2 | 42 | 53.8 | 1.2 | 3 |
| 1.2 | 2 | 41 | 54.8 | 1.2 | 3 |
| 1.2 | 2 | 40 | 55.8 | 1.2 | 3 |
| 1.2 | 2 | 39 | 56.8 | 1.2 | 3 |
| 1.2 | 2 | 38 | 57.8 | 1.2 | 3 |
| 1.2 | 2 | 37 | 58.8 | 1.2 | 3 |
| 1.2 | 2 | 36 | 59.8 | 1.2 | 3 |
| 1.2 | 2 | 35 | 60.8 | 1.2 | 3 |
| 1.2 | 2 | 34 | 61.8 | 1.2 | 3 |
| 1.2 | 2 | 33 | 62.8 | 1.2 | 3 |
| 1.2 | 2 | 32 | 63.8 | 1.2 | 3 |
| 1.2 | 2 | 31 | 64.8 | 1.2 | 3 |
| 1.2 | 2 | 30 | 65.8 | 1.2 | 3 |
| 1.4 | 2 | 60 | 35.6 | 1.4 | 3 |
| 1.4 | 2 | 59 | 36.6 | 1.4 | 3 |
| 1.4 | 2 | 58 | 37.6 | 1.4 | 3 |
| 1.4 | 2 | 57 | 38.6 | 1.4 | 3 |
| 1.4 | 2 | 56 | 39.6 | 1.4 | 3 |
| 1.4 | 2 | 55 | 40.6 | 1.4 | 3 |
| 1.4 | 2 | 54 | 41.6 | 1.4 | 3 |
| 1.4 | 2 | 53 | 42.6 | 1.4 | 3 |
| 1.4 | 2 | 52 | 43.6 | 1.4 | 3 |
| 1.4 | 2 | 51 | 44.6 | 1.4 | 3 |
| 1.4 | 2 | 50 | 45.6 | 1.4 | 3 |
| 1.4 | 2 | 49 | 46.6 | 1.4 | 3 |
| 1.4 | 2 | 48 | 47.6 | 1.4 | 3 |
| 1.4 | 2 | 47 | 48.6 | 1.4 | 3 |
| 1.4 | 2 | 46 | 49.6 | 1.4 | 3 |
| 1.4 | 2 | 45 | 50.6 | 1.4 | 3 |
| 1.4 | 2 | 44 | 51.6 | 1.4 | 3 |
| 1.4 | 2 | 43 | 52.6 | 1.4 | 3 |
| 1.4 | 2 | 42 | 53.6 | 1.4 | 3 |
| 1.4 | 2 | 41 | 54.6 | 1.4 | 3 |
| 1.4 | 2 | 40 | 55.6 | 1.4 | 3 |
| 1.4 | 2 | 39 | 56.6 | 1.4 | 3 |
| 1.4 | 2 | 38 | 57.6 | 1.4 | 3 |
| 1.4 | 2 | 37 | 58.6 | 1.4 | 3 |
| 1.4 | 2 | 36 | 59.6 | 1.4 | 3 |
| 1.4 | 2 | 35 | 60.6 | 1.4 | 3 |
| 1.4 | 2 | 34 | 61.6 | 1.4 | 3 |
| 1.4 | 2 | 33 | 62.6 | 1.4 | 3 |
| 1.4 | 2 | 32 | 63.6 | 1.4 | 3 |
| 1.4 | 2 | 31 | 64.6 | 1.4 | 3 |
| 1.4 | 2 | 30 | 65.6 | 1.4 | 3 |
| 1.6 | 2 | 60 | 35.4 | 1.6 | 3 |
| 1.6 | 2 | 59 | 36.4 | 1.6 | 3 |
| 1.6 | 2 | 58 | 37.4 | 1.6 | 3 |
| 1.6 | 2 | 57 | 38.4 | 1.6 | 3 |
| 1.6 | 2 | 56 | 39.4 | 1.6 | 3 |
| 1.6 | 2 | 55 | 40.4 | 1.6 | 3 |
| 1.6 | 2 | 54 | 41.4 | 1.6 | 3 |
| 1.6 | 2 | 53 | 42.4 | 1.6 | 3 |
| 1.6 | 2 | 52 | 43.4 | 1.6 | 3 |
| 1.6 | 2 | 51 | 44.4 | 1.6 | 3 |
| 1.6 | 2 | 50 | 45.4 | 1.6 | 3 |
| 1.6 | 2 | 49 | 46.4 | 1.6 | 3 |
| 1.6 | 2 | 48 | 47.4 | 1.6 | 3 |
| 1.6 | 2 | 47 | 48.4 | 1.6 | 3 |
| 1.6 | 2 | 46 | 49.4 | 1.6 | 3 |
| 1.6 | 2 | 45 | 50.4 | 1.6 | 3 |
| 1.6 | 2 | 44 | 51.4 | 1.6 | 3 |
| 1.6 | 2 | 43 | 52.4 | 1.6 | 3 |
| 1.6 | 2 | 42 | 53.4 | 1.6 | 3 |
| 1.6 | 2 | 41 | 54.4 | 1.6 | 3 |
| 1.6 | 2 | 40 | 55.4 | 1.6 | 3 |
| 1.6 | 2 | 39 | 56.4 | 1.6 | 3 |
| 1.6 | 2 | 38 | 57.4 | 1.6 | 3 |
| 1.6 | 2 | 37 | 58.4 | 1.6 | 3 |
| 1.6 | 2 | 36 | 59.4 | 1.6 | 3 |
| 1.6 | 2 | 35 | 60.4 | 1.6 | 3 |
| 1.6 | 2 | 34 | 61.4 | 1.6 | 3 |
| 1.6 | 2 | 33 | 62.4 | 1.6 | 3 |
| 1.6 | 2 | 32 | 63.4 | 1.6 | 3 |
| 1.6 | 2 | 31 | 64.4 | 1.6 | 3 |
| 1.6 | 2 | 30 | 65.4 | 1.6 | 3 |
| 1.8 | 2 | 60 | 35.2 | 1.8 | 3 |
| 1.8 | 2 | 59 | 36.2 | 1.8 | 3 |
| 1.8 | 2 | 58 | 37.2 | 1.8 | 3 |
| 1.8 | 2 | 57 | 38.2 | 1.8 | 3 |
| 1.8 | 2 | 56 | 39.2 | 1.8 | 3 |
| 1.8 | 2 | 55 | 40.2 | 1.8 | 3 |
| 1.8 | 2 | 54 | 41.2 | 1.8 | 3 |
| 1.8 | 2 | 53 | 42.2 | 1.8 | 3 |
| 1.8 | 2 | 52 | 43.2 | 1.8 | 3 |
| 1.8 | 2 | 51 | 44.2 | 1.8 | 3 |
| 1.8 | 2 | 50 | 45.2 | 1.8 | 3 |
| 1.8 | 2 | 49 | 46.2 | 1.8 | 3 |
| 1.8 | 2 | 48 | 47.2 | 1.8 | 3 |
| 1.8 | 2 | 47 | 48.2 | 1.8 | 3 |
| 1.8 | 2 | 46 | 49.2 | 1.8 | 3 |
| 1.8 | 2 | 45 | 50.2 | 1.8 | 3 |
| 1.8 | 2 | 44 | 51.2 | 1.8 | 3 |
| 1.8 | 2 | 43 | 52.2 | 1.8 | 3 |
| 1.8 | 2 | 42 | 53.2 | 1.8 | 3 |
| 1.8 | 2 | 41 | 54.2 | 1.8 | 3 |
| 1.8 | 2 | 40 | 55.2 | 1.8 | 3 |
| 1.8 | 2 | 39 | 56.2 | 1.8 | 3 |
| 1.8 | 2 | 38 | 57.2 | 1.8 | 3 |
| 1.8 | 2 | 37 | 58.2 | 1.8 | 3 |
| 1.8 | 2 | 36 | 59.2 | 1.8 | 3 |
| 1.8 | 2 | 35 | 60.2 | 1.8 | 3 |
| 1.8 | 2 | 34 | 61.2 | 1.8 | 3 |
| 1.8 | 2 | 33 | 62.2 | 1.8 | 3 |
| 1.8 | 2 | 32 | 63.2 | 1.8 | 3 |
| 1.8 | 2 | 31 | 64.2 | 1.8 | 3 |
| 1.8 | 2 | 30 | 65.2 | 1.8 | 3 |
| 2 | 2 | 60 | 35 | 2 | 3 |
| 2 | 2 | 59 | 36 | 2 | 3 |
| 2 | 2 | 58 | 37 | 2 | 3 |
| 2 | 2 | 57 | 38 | 2 | 3 |
| 2 | 2 | 56 | 39 | 2 | 3 |
| 2 | 2 | 55 | 40 | 2 | 3 |
| 2 | 2 | 54 | 41 | 2 | 3 |
| 2 | 2 | 53 | 42 | 2 | 3 |
| 2 | 2 | 52 | 43 | 2 | 3 |
| 2 | 2 | 51 | 44 | 2 | 3 |
| 2 | 2 | 50 | 45 | 2 | 3 |
| 2 | 2 | 49 | 46 | 2 | 3 |
| 2 | 2 | 48 | 47 | 2 | 3 |
| 2 | 2 | 47 | 48 | 2 | 3 |
| 2 | 2 | 46 | 49 | 2 | 3 |
| 2 | 2 | 45 | 50 | 2 | 3 |
| 2 | 2 | 44 | 51 | 2 | 3 |
| 2 | 2 | 43 | 52 | 2 | 3 |
| 2 | 2 | 42 | 53 | 2 | 3 |
| 2 | 2 | 41 | 54 | 2 | 3 |
| 2 | 2 | 40 | 55 | 2 | 3 |
| 2 | 2 | 39 | 56 | 2 | 3 |
| 2 | 2 | 38 | 57 | 2 | 3 |
| 2 | 2 | 37 | 58 | 2 | 3 |
| 2 | 2 | 36 | 59 | 2 | 3 |
| 2 | 2 | 35 | 60 | 2 | 3 |
| 2 | 2 | 34 | 61 | 2 | 3 |
| 2 | 2 | 33 | 62 | 2 | 3 |
| 2 | 2 | 32 | 63 | 2 | 3 |
| 2 | 2 | 31 | 64 | 2 | 3 |
| 2 | 2 | 30 | 65 | 2 | 3 |
| 2.2 | 2 | 60 | 34.8 | 2.2 | 3 |
| 2.2 | 2 | 59 | 35.8 | 2.2 | 3 |
| 2.2 | 2 | 58 | 36.8 | 2.2 | 3 |
| 2.2 | 2 | 57 | 37.8 | 2.2 | 3 |
| 2.2 | 2 | 56 | 38.8 | 2.2 | 3 |
| 2.2 | 2 | 55 | 39.8 | 2.2 | 3 |
| 2.2 | 2 | 54 | 40.8 | 2.2 | 3 |
| 2.2 | 2 | 53 | 41.8 | 2.2 | 3 |
| 2.2 | 2 | 52 | 42.8 | 2.2 | 3 |
| 2.2 | 2 | 51 | 43.8 | 2.2 | 3 |
| 2.2 | 2 | 50 | 44.8 | 2.2 | 3 |
| 2.2 | 2 | 49 | 45.8 | 2.2 | 3 |
| 2.2 | 2 | 48 | 46.8 | 2.2 | 3 |
| 2.2 | 2 | 47 | 47.8 | 2.2 | 3 |
| 2.2 | 2 | 46 | 48.8 | 2.2 | 3 |
| 2.2 | 2 | 45 | 49.8 | 2.2 | 3 |
| 2.2 | 2 | 44 | 50.8 | 2.2 | 3 |
| 2.2 | 2 | 43 | 51.8 | 2.2 | 3 |
| 2.2 | 2 | 42 | 52.8 | 2.2 | 3 |
| 2.2 | 2 | 41 | 53.8 | 2.2 | 3 |
| 2.2 | 2 | 40 | 54.8 | 2.2 | 3 |
| 2.2 | 2 | 39 | 55.8 | 2.2 | 3 |
| 2.2 | 2 | 38 | 56.8 | 2.2 | 3 |
| 2.2 | 2 | 37 | 57.8 | 2.2 | 3 |
| 2.2 | 2 | 36 | 58.8 | 2.2 | 3 |
| 2.2 | 2 | 35 | 59.8 | 2.2 | 3 |
| 2.2 | 2 | 34 | 60.8 | 2.2 | 3 |
| 2.2 | 2 | 33 | 61.8 | 2.2 | 3 |
| 2.2 | 2 | 32 | 62.8 | 2.2 | 3 |
| 2.2 | 2 | 31 | 63.8 | 2.2 | 3 |
| 2.2 | 2 | 30 | 64.8 | 2.2 | 3 |
| 2.4 | 2 | 60 | 34.6 | 2.4 | 3 |
| 2.4 | 2 | 59 | 35.6 | 2.4 | 3 |
| 2.4 | 2 | 58 | 36.6 | 2.4 | 3 |
| 2.4 | 2 | 57 | 37.6 | 2.4 | 3 |
| 2.4 | 2 | 56 | 38.6 | 2.4 | 3 |
| 2.4 | 2 | 55 | 39.6 | 2.4 | 3 |
| 2.4 | 2 | 54 | 40.6 | 2.4 | 3 |
| 2.4 | 2 | 53 | 41.6 | 2.4 | 3 |
| 2.4 | 2 | 52 | 42.6 | 2.4 | 3 |
| 2.4 | 2 | 51 | 43.6 | 2.4 | 3 |
| 2.4 | 2 | 50 | 44.6 | 2.4 | 3 |
| 2.4 | 2 | 49 | 45.6 | 2.4 | 3 |
| 2.4 | 2 | 48 | 46.6 | 2.4 | 3 |
| 2.4 | 2 | 47 | 47.6 | 2.4 | 3 |
| 2.4 | 2 | 46 | 48.6 | 2.4 | 3 |
| 2.4 | 2 | 45 | 49.6 | 2.4 | 3 |
| 2.4 | 2 | 44 | 50.6 | 2.4 | 3 |
| 2.4 | 2 | 43 | 51.6 | 2.4 | 3 |
| 2.4 | 2 | 42 | 52.6 | 2.4 | 3 |
| 2.4 | 2 | 41 | 53.6 | 2.4 | 3 |
| 2.4 | 2 | 40 | 54.6 | 2.4 | 3 |
| 2.4 | 2 | 39 | 55.6 | 2.4 | 3 |
| 2.4 | 2 | 38 | 56.6 | 2.4 | 3 |
| 2.4 | 2 | 37 | 57.6 | 2.4 | 3 |
| 2.4 | 2 | 36 | 58.6 | 2.4 | 3 |
| 2.4 | 2 | 35 | 59.6 | 2.4 | 3 |
| 2.4 | 2 | 34 | 60.6 | 2.4 | 3 |
| 2.4 | 2 | 33 | 61.6 | 2.4 | 3 |
| 2.4 | 2 | 32 | 62.6 | 2.4 | 3 |
| 2.4 | 2 | 31 | 63.6 | 2.4 | 3 |
| 2.4 | 2 | 30 | 64.6 | 2.4 | 3 |
| 2.6 | 2 | 60 | 34.4 | 2.6 | 3 |
| 2.6 | 2 | 59 | 35.4 | 2.6 | 3 |
| 2.6 | 2 | 58 | 36.4 | 2.6 | 3 |
| 2.6 | 2 | 57 | 37.4 | 2.6 | 3 |
| 2.6 | 2 | 56 | 38.4 | 2.6 | 3 |
| 2.6 | 2 | 55 | 39.4 | 2.6 | 3 |
| 2.6 | 2 | 54 | 40.4 | 2.6 | 3 |
| 2.6 | 2 | 53 | 41.4 | 2.6 | 3 |
| 2.6 | 2 | 52 | 42.4 | 2.6 | 3 |
| 2.6 | 2 | 51 | 43.4 | 2.6 | 3 |
| 2.6 | 2 | 50 | 44.4 | 2.6 | 3 |
| 2.6 | 2 | 49 | 45.4 | 2.6 | 3 |
| 2.6 | 2 | 48 | 46.4 | 2.6 | 3 |
| 2.6 | 2 | 47 | 47.4 | 2.6 | 3 |
| 2.6 | 2 | 46 | 48.4 | 2.6 | 3 |
| 2.6 | 2 | 45 | 49.4 | 2.6 | 3 |
| 2.6 | 2 | 44 | 50.4 | 2.6 | 3 |
| 2.6 | 2 | 43 | 51.4 | 2.6 | 3 |
| 2.6 | 2 | 42 | 52.4 | 2.6 | 3 |
| 2.6 | 2 | 41 | 53.4 | 2.6 | 3 |
| 2.6 | 2 | 40 | 54.4 | 2.6 | 3 |
| 2.6 | 2 | 39 | 55.4 | 2.6 | 3 |
| 2.6 | 2 | 38 | 56.4 | 2.6 | 3 |
| 2.6 | 2 | 37 | 57.4 | 2.6 | 3 |
| 2.6 | 2 | 36 | 58.4 | 2.6 | 3 |
| 2.6 | 2 | 35 | 59.4 | 2.6 | 3 |
| 2.6 | 2 | 34 | 60.4 | 2.6 | 3 |
| 2.6 | 2 | 33 | 61.4 | 2.6 | 3 |
| 2.6 | 2 | 32 | 62.4 | 2.6 | 3 |
| 2.6 | 2 | 31 | 63.4 | 2.6 | 3 |
| 2.6 | 2 | 30 | 64.4 | 2.6 | 3 |
| 2.8 | 2 | 60 | 34.2 | 2.8 | 3 |
| 2.8 | 2 | 59 | 35.2 | 2.8 | 3 |
| 2.8 | 2 | 58 | 36.2 | 2.8 | 3 |
| 2.8 | 2 | 57 | 37.2 | 2.8 | 3 |
| 2.8 | 2 | 56 | 38.2 | 2.8 | 3 |
| 2.8 | 2 | 55 | 39.2 | 2.8 | 3 |
| 2.8 | 2 | 54 | 40.2 | 2.8 | 3 |
| 2.8 | 2 | 53 | 41.2 | 2.8 | 3 |
| 2.8 | 2 | 52 | 42.2 | 2.8 | 3 |
| 2.8 | 2 | 51 | 43.2 | 2.8 | 3 |
| 2.8 | 2 | 50 | 44.2 | 2.8 | 3 |
| 2.8 | 2 | 49 | 45.2 | 2.8 | 3 |
| 2.8 | 2 | 48 | 46.2 | 2.8 | 3 |
| 2.8 | 2 | 47 | 47.2 | 2.8 | 3 |
| 2.8 | 2 | 46 | 48.2 | 2.8 | 3 |
| 2.8 | 2 | 45 | 49.2 | 2.8 | 3 |
| 2.8 | 2 | 44 | 50.2 | 2.8 | 3 |
| 2.8 | 2 | 43 | 51.2 | 2.8 | 3 |
| 2.8 | 2 | 42 | 52.2 | 2.8 | 3 |
| 2.8 | 2 | 41 | 53.2 | 2.8 | 3 |
| 2.8 | 2 | 40 | 54.2 | 2.8 | 3 |
| 2.8 | 2 | 39 | 55.2 | 2.8 | 3 |
| 2.8 | 2 | 38 | 56.2 | 2.8 | 3 |
| 2.8 | 2 | 37 | 57.2 | 2.8 | 3 |
| 2.8 | 2 | 36 | 58.2 | 2.8 | 3 |
| 2.8 | 2 | 35 | 59.2 | 2.8 | 3 |
| 2.8 | 2 | 34 | 60.2 | 2.8 | 3 |
| 2.8 | 2 | 33 | 61.2 | 2.8 | 3 |
| 2.8 | 2 | 32 | 62.2 | 2.8 | 3 |
| 2.8 | 2 | 31 | 63.2 | 2.8 | 3 |
| 2.8 | 2 | 30 | 64.2 | 2.8 | 3 |
| 3 | 2 | 60 | 34 | 3 | 3 |
| 3 | 2 | 59 | 35 | 3 | 3 |
| 3 | 2 | 58 | 36 | 3 | 3 |
| 3 | 2 | 57 | 37 | 3 | 3 |
| 3 | 2 | 56 | 38 | 3 | 3 |
| 3 | 2 | 55 | 39 | 3 | 3 |
| 3 | 2 | 54 | 40 | 3 | 3 |
| 3 | 2 | 53 | 41 | 3 | 3 |
| 3 | 2 | 52 | 42 | 3 | 3 |
| 3 | 2 | 51 | 43 | 3 | 3 |
| 3 | 2 | 50 | 44 | 3 | 3 |
| 3 | 2 | 49 | 45 | 3 | 3 |
| 3 | 2 | 48 | 46 | 3 | 3 |
| 3 | 2 | 47 | 47 | 3 | 3 |
| 3 | 2 | 46 | 48 | 3 | 3 |
| 3 | 2 | 45 | 49 | 3 | 3 |
| 3 | 2 | 44 | 50 | 3 | 3 |
| 3 | 2 | 43 | 51 | 3 | 3 |
| 3 | 2 | 42 | 52 | 3 | 3 |
| 3 | 2 | 41 | 53 | 3 | 3 |
| 3 | 2 | 40 | 54 | 3 | 3 |
| 3 | 2 | 39 | 55 | 3 | 3 |
| 3 | 2 | 38 | 56 | 3 | 3 |
| 3 | 2 | 37 | 57 | 3 | 3 |
| 3 | 2 | 36 | 58 | 3 | 3 |
| 3 | 2 | 35 | 59 | 3 | 3 |
| 3 | 2 | 34 | 60 | 3 | 3 |
| 3 | 2 | 33 | 61 | 3 | 3 |
| 3 | 2 | 32 | 62 | 3 | 3 |
| 3 | 2 | 31 | 63 | 3 | 3 |
| 3 | 2 | 30 | 64 | 3 | 3 |
| 1 | 2 | 60 | 32 | 1 | 7 |
| 1 | 2 | 59 | 33 | 1 | 7 |
| 1 | 2 | 58 | 34 | 1 | 7 |
| 1 | 2 | 57 | 35 | 1 | 7 |
| 1 | 2 | 56 | 36 | 1 | 7 |
| 1 | 2 | 55 | 37 | 1 | 7 |
| 1 | 2 | 54 | 38 | 1 | 7 |
| 1 | 2 | 53 | 39 | 1 | 7 |
| 1 | 2 | 52 | 40 | 1 | 7 |
| 1 | 2 | 51 | 41 | 1 | 7 |
| 1 | 2 | 50 | 42 | 1 | 7 |
| 1 | 2 | 49 | 43 | 1 | 7 |
| 1 | 2 | 48 | 44 | 1 | 7 |
| 1 | 2 | 47 | 45 | 1 | 7 |
| 1 | 2 | 46 | 46 | 1 | 7 |
| 1 | 2 | 45 | 47 | 1 | 7 |
| 1 | 6 | 44 | 48 | 1 | 7 |
| 1 | 6 | 43 | 49 | 1 | 7 |
| 1 | 6 | 42 | 50 | 1 | 7 |
| 1 | 6 | 41 | 51 | 1 | 7 |
| 1 | 6 | 40 | 52 | 1 | 7 |
| 1 | 6 | 39 | 53 | 1 | 7 |
| 1 | 6 | 38 | 54 | 1 | 7 |
| 1 | 6 | 37 | 55 | 1 | 7 |
| 1 | 6 | 36 | 56 | 1 | 7 |
| 1 | 6 | 35 | 57 | 1 | 7 |
| 1 | 6 | 34 | 58 | 1 | 7 |
| 1 | 6 | 33 | 59 | 1 | 7 |
| 1 | 6 | 32 | 60 | 1 | 7 |
| 1 | 6 | 31 | 61 | 1 | 7 |
| 1 | 6 | 30 | 62 | 1 | 7 |
| 1.2 | 6 | 60 | 31.8 | 1.2 | 7 |
| 1.2 | 6 | 59 | 32.8 | 1.2 | 7 |
| 1.2 | 6 | 58 | 33.8 | 1.2 | 7 |
| 1.2 | 6 | 57 | 34.8 | 1.2 | 7 |
| 1.2 | 6 | 56 | 35.8 | 1.2 | 7 |
| 1.2 | 6 | 55 | 36.8 | 1.2 | 7 |
| 1.2 | 6 | 54 | 37.8 | 1.2 | 7 |
| 1.2 | 6 | 53 | 38.8 | 1.2 | 7 |
| 1.2 | 6 | 52 | 39.8 | 1.2 | 7 |
| 1.2 | 6 | 51 | 40.8 | 1.2 | 7 |
| 1.2 | 6 | 50 | 41.8 | 1.2 | 7 |
| 1.2 | 6 | 49 | 42.8 | 1.2 | 7 |
| 1.2 | 6 | 48 | 43.8 | 1.2 | 7 |
| 1.2 | 6 | 47 | 44.8 | 1.2 | 7 |
| 1.2 | 6 | 46 | 45.8 | 1.2 | 7 |
| 1.2 | 6 | 45 | 46.8 | 1.2 | 7 |
| 1.2 | 6 | 44 | 47.8 | 1.2 | 7 |
| 1.2 | 6 | 43 | 48.8 | 1.2 | 7 |
| 1.2 | 6 | 42 | 49.8 | 1.2 | 7 |
| 1.2 | 6 | 41 | 50.8 | 1.2 | 7 |
| 1.2 | 6 | 40 | 51.8 | 1.2 | 7 |
| 1.2 | 6 | 39 | 52.8 | 1.2 | 7 |
| 1.2 | 6 | 38 | 53.8 | 1.2 | 7 |
| 1.2 | 6 | 37 | 54.8 | 1.2 | 7 |
| 1.2 | 6 | 36 | 55.8 | 1.2 | 7 |
| 1.2 | 6 | 35 | 56.8 | 1.2 | 7 |
| 1.2 | 6 | 34 | 57.8 | 1.2 | 7 |
| 1.2 | 6 | 33 | 58.8 | 1.2 | 7 |
| 1.2 | 6 | 32 | 59.8 | 1.2 | 7 |
| 1.2 | 6 | 31 | 60.8 | 1.2 | 7 |
| 1.2 | 6 | 30 | 61.8 | 1.2 | 7 |
| 1.4 | 6 | 60 | 31.6 | 1.4 | 7 |
| 1.4 | 6 | 59 | 32.6 | 1.4 | 7 |
| 1.4 | 6 | 58 | 33.6 | 1.4 | 7 |
| 1.4 | 6 | 57 | 34.6 | 1.4 | 7 |
| 1.4 | 6 | 56 | 35.6 | 1.4 | 7 |
| 1.4 | 6 | 55 | 36.6 | 1.4 | 7 |
| 1.4 | 6 | 54 | 37.6 | 1.4 | 7 |
| 1.4 | 6 | 53 | 38.6 | 1.4 | 7 |
| 1.4 | 6 | 52 | 39.6 | 1.4 | 7 |
| 1.4 | 6 | 51 | 40.6 | 1.4 | 7 |
| 1.4 | 6 | 50 | 41.6 | 1.4 | 7 |
| 1.4 | 6 | 49 | 42.6 | 1.4 | 7 |
| 1.4 | 6 | 48 | 43.6 | 1.4 | 7 |
| 1.4 | 6 | 47 | 44.6 | 1.4 | 7 |
| 1.4 | 6 | 46 | 45.6 | 1.4 | 7 |
| 1.4 | 6 | 45 | 46.6 | 1.4 | 7 |
| 1.4 | 6 | 44 | 47.6 | 1.4 | 7 |
| 1.4 | 6 | 43 | 48.6 | 1.4 | 7 |
| 1.4 | 6 | 42 | 49.6 | 1.4 | 7 |
| 1.4 | 6 | 41 | 50.6 | 1.4 | 7 |
| 1.4 | 6 | 40 | 51.6 | 1.4 | 7 |
| 1.4 | 6 | 39 | 52.6 | 1.4 | 7 |
| 1.4 | 6 | 38 | 53.6 | 1.4 | 7 |
| 1.4 | 6 | 37 | 54.6 | 1.4 | 7 |
| 1.4 | 6 | 36 | 55.6 | 1.4 | 7 |
| 1.4 | 6 | 35 | 56.6 | 1.4 | 7 |
| 1.4 | 6 | 34 | 57.6 | 1.4 | 7 |
| 1.4 | 6 | 33 | 58.6 | 1.4 | 7 |
| 1.4 | 6 | 32 | 59.6 | 1.4 | 7 |
| 1.4 | 6 | 31 | 60.6 | 1.4 | 7 |
| 1.4 | 6 | 30 | 61.6 | 1.4 | 7 |
| 1.6 | 6 | 60 | 31.4 | 1.6 | 7 |
| 1.6 | 6 | 59 | 32.4 | 1.6 | 7 |
| 1.6 | 6 | 58 | 33.4 | 1.6 | 7 |
| 1.6 | 6 | 57 | 34.4 | 1.6 | 7 |
| 1.6 | 6 | 56 | 35.4 | 1.6 | 7 |
| 1.6 | 6 | 55 | 36.4 | 1.6 | 7 |
| 1.6 | 6 | 54 | 37.4 | 1.6 | 7 |
| 1.6 | 6 | 53 | 38.4 | 1.6 | 7 |
| 1.6 | 6 | 52 | 39.4 | 1.6 | 7 |
| 1.6 | 6 | 51 | 40.4 | 1.6 | 7 |
| 1.6 | 6 | 50 | 41.4 | 1.6 | 7 |
| 1.6 | 6 | 49 | 42.4 | 1.6 | 7 |
| 1.6 | 6 | 48 | 43.4 | 1.6 | 7 |
| 1.6 | 6 | 47 | 44.4 | 1.6 | 7 |
| 1.6 | 6 | 46 | 45.4 | 1.6 | 7 |
| 1.6 | 6 | 45 | 46.4 | 1.6 | 7 |
| 1.6 | 6 | 44 | 47.4 | 1.6 | 7 |
| 1.6 | 6 | 43 | 48.4 | 1.6 | 7 |
| 1.6 | 6 | 42 | 49.4 | 1.6 | 7 |
| 1.6 | 6 | 41 | 50.4 | 1.6 | 7 |
| 1.6 | 6 | 40 | 51.4 | 1.6 | 7 |
| 1.6 | 6 | 39 | 52.4 | 1.6 | 7 |
| 1.6 | 6 | 38 | 53.4 | 1.6 | 7 |
| 1.6 | 6 | 37 | 54.4 | 1.6 | 7 |
| 1.6 | 6 | 36 | 55.4 | 1.6 | 7 |
| 1.6 | 6 | 35 | 56.4 | 1.6 | 7 |
| 1.6 | 6 | 34 | 57.4 | 1.6 | 7 |
| 1.6 | 6 | 33 | 58.4 | 1.6 | 7 |
| 1.6 | 6 | 32 | 59.4 | 1.6 | 7 |
| 1.6 | 6 | 31 | 60.4 | 1.6 | 7 |
| 1.6 | 6 | 30 | 61.4 | 1.6 | 7 |
| 1.8 | 6 | 60 | 31.2 | 1.8 | 7 |
| 1.8 | 6 | 59 | 32.2 | 1.8 | 7 |
| 1.8 | 6 | 58 | 33.2 | 1.8 | 7 |
| 1.8 | 6 | 57 | 34.2 | 1.8 | 7 |
| 1.8 | 6 | 56 | 35.2 | 1.8 | 7 |
| 1.8 | 6 | 55 | 36.2 | 1.8 | 7 |
| 1.8 | 6 | 54 | 37.2 | 1.8 | 7 |
| 1.8 | 6 | 53 | 38.2 | 1.8 | 7 |
| 1.8 | 6 | 52 | 39.2 | 1.8 | 7 |
| 1.8 | 6 | 51 | 40.2 | 1.8 | 7 |
| 1.8 | 6 | 50 | 41.2 | 1.8 | 7 |
| 1.8 | 6 | 49 | 42.2 | 1.8 | 7 |
| 1.8 | 6 | 48 | 43.2 | 1.8 | 7 |
| 1.8 | 6 | 47 | 44.2 | 1.8 | 7 |
| 1.8 | 6 | 46 | 45.2 | 1.8 | 7 |
| 1.8 | 6 | 45 | 46.2 | 1.8 | 7 |
| 1.8 | 6 | 44 | 47.2 | 1.8 | 7 |
| 1.8 | 6 | 43 | 48.2 | 1.8 | 7 |
| 1.8 | 6 | 42 | 49.2 | 1.8 | 7 |
| 1.8 | 6 | 41 | 50.2 | 1.8 | 7 |
| 1.8 | 6 | 40 | 51.2 | 1.8 | 7 |
| 1.8 | 6 | 39 | 52.2 | 1.8 | 7 |
| 1.8 | 6 | 38 | 53.2 | 1.8 | 7 |
| 1.8 | 6 | 37 | 54.2 | 1.8 | 7 |
| 1.8 | 6 | 36 | 55.2 | 1.8 | 7 |
| 1.8 | 6 | 35 | 56.2 | 1.8 | 7 |
| 1.8 | 6 | 34 | 57.2 | 1.8 | 7 |
| 1.8 | 6 | 33 | 58.2 | 1.8 | 7 |
| 1.8 | 6 | 32 | 59.2 | 1.8 | 7 |
| 1.8 | 6 | 31 | 60.2 | 1.8 | 7 |
| 1.8 | 6 | 30 | 61.2 | 1.8 | 7 |
| 2 | 6 | 60 | 31 | 2 | 7 |
| 2 | 6 | 59 | 32 | 2 | 7 |
| 2 | 6 | 58 | 33 | 2 | 7 |
| 2 | 6 | 57 | 34 | 2 | 7 |
| 2 | 6 | 56 | 35 | 2 | 7 |
| 2 | 6 | 55 | 36 | 2 | 7 |
| 2 | 6 | 54 | 37 | 2 | 7 |
| 2 | 6 | 53 | 38 | 2 | 7 |
| 2 | 6 | 52 | 39 | 2 | 7 |
| 2 | 6 | 51 | 40 | 2 | 7 |
| 2 | 6 | 50 | 41 | 2 | 7 |
| 2 | 6 | 49 | 42 | 2 | 7 |
| 2 | 6 | 48 | 43 | 2 | 7 |
| 2 | 6 | 47 | 44 | 2 | 7 |
| 2 | 6 | 46 | 45 | 2 | 7 |
| 2 | 6 | 45 | 46 | 2 | 7 |
| 2 | 6 | 44 | 47 | 2 | 7 |
| 2 | 6 | 43 | 48 | 2 | 7 |
| 2 | 6 | 42 | 49 | 2 | 7 |
| 2 | 6 | 41 | 50 | 2 | 7 |
| 2 | 6 | 40 | 51 | 2 | 7 |
| 2 | 6 | 39 | 52 | 2 | 7 |
| 2 | 6 | 38 | 53 | 2 | 7 |
| 2 | 6 | 37 | 54 | 2 | 7 |
| 2 | 6 | 36 | 55 | 2 | 7 |
| 2 | 6 | 35 | 56 | 2 | 7 |
| 2 | 6 | 34 | 57 | 2 | 7 |
| 2 | 6 | 33 | 58 | 2 | 7 |
| 2 | 6 | 32 | 59 | 2 | 7 |
| 2 | 6 | 31 | 60 | 2 | 7 |
| 2 | 6 | 30 | 61 | 2 | 7 |
| 2.2 | 6 | 60 | 30.8 | 2.2 | 7 |
| 2.2 | 6 | 59 | 31.8 | 2.2 | 7 |
| 2.2 | 6 | 58 | 32.8 | 2.2 | 7 |
| 2.2 | 6 | 57 | 33.8 | 2.2 | 7 |
| 2.2 | 6 | 56 | 34.8 | 2.2 | 7 |
| 2.2 | 6 | 55 | 35.8 | 2.2 | 7 |
| 2.2 | 6 | 54 | 36.8 | 2.2 | 7 |
| 2.2 | 6 | 53 | 37.8 | 2.2 | 7 |
| 2.2 | 6 | 52 | 38.8 | 2.2 | 7 |
| 2.2 | 6 | 51 | 39.8 | 2.2 | 7 |
| 2.2 | 6 | 50 | 40.8 | 2.2 | 7 |
| 2.2 | 6 | 49 | 41.8 | 2.2 | 7 |
| 2.2 | 6 | 48 | 42.8 | 2.2 | 7 |
| 2.2 | 6 | 47 | 43.8 | 2.2 | 7 |
| 2.2 | 6 | 46 | 44.8 | 2.2 | 7 |
| 2.2 | 6 | 45 | 45.8 | 2.2 | 7 |
| 2.2 | 6 | 44 | 46.8 | 2.2 | 7 |
| 2.2 | 6 | 43 | 47.8 | 2.2 | 7 |
| 2.2 | 6 | 42 | 48.8 | 2.2 | 7 |
| 2.2 | 6 | 41 | 49.8 | 2.2 | 7 |
| 2.2 | 6 | 40 | 50.8 | 2.2 | 7 |
| 2.2 | 6 | 39 | 51.8 | 2.2 | 7 |
| 2.2 | 6 | 38 | 52.8 | 2.2 | 7 |
| 2.2 | 6 | 37 | 53.8 | 2.2 | 7 |
| 2.2 | 6 | 36 | 54.8 | 2.2 | 7 |
| 2.2 | 6 | 35 | 55.8 | 2.2 | 7 |
| 2.2 | 6 | 34 | 56.8 | 2.2 | 7 |
| 2.2 | 6 | 33 | 57.8 | 2.2 | 7 |
| 2.2 | 6 | 32 | 58.8 | 2.2 | 7 |
| 2.2 | 6 | 31 | 59.8 | 2.2 | 7 |
| 2.2 | 6 | 30 | 60.8 | 2.2 | 7 |
| 2.4 | 6 | 60 | 30.6 | 2.4 | 7 |
| 2.4 | 6 | 59 | 31.6 | 2.4 | 7 |
| 2.4 | 6 | 58 | 32.6 | 2.4 | 7 |
| 2.4 | 6 | 57 | 33.6 | 2.4 | 7 |
| 2.4 | 6 | 56 | 34.6 | 2.4 | 7 |
| 2.4 | 6 | 55 | 35.6 | 2.4 | 7 |
| 2.4 | 6 | 54 | 36.6 | 2.4 | 7 |
| 2.4 | 6 | 53 | 37.6 | 2.4 | 7 |
| 2.4 | 6 | 52 | 38.6 | 2.4 | 7 |
| 2.4 | 6 | 51 | 39.6 | 2.4 | 7 |
| 2.4 | 6 | 50 | 40.6 | 2.4 | 7 |
| 2.4 | 6 | 49 | 41.6 | 2.4 | 7 |
| 2.4 | 6 | 48 | 42.6 | 2.4 | 7 |
| 2.4 | 6 | 47 | 43.6 | 2.4 | 7 |
| 2.4 | 6 | 46 | 44.6 | 2.4 | 7 |
| 2.4 | 6 | 45 | 45.6 | 2.4 | 7 |
| 2.4 | 6 | 44 | 46.6 | 2.4 | 7 |
| 2.4 | 6 | 43 | 47.6 | 2.4 | 7 |
| 2.4 | 6 | 42 | 48.6 | 2.4 | 7 |
| 2.4 | 6 | 41 | 49.6 | 2.4 | 7 |
| 2.4 | 6 | 40 | 50.6 | 2.4 | 7 |
| 2.4 | 6 | 39 | 51.6 | 2.4 | 7 |
| 2.4 | 6 | 38 | 52.6 | 2.4 | 7 |
| 2.4 | 6 | 37 | 53.6 | 2.4 | 7 |
| 2.4 | 6 | 36 | 54.6 | 2.4 | 7 |
| 2.4 | 6 | 35 | 55.6 | 2.4 | 7 |
| 2.4 | 6 | 34 | 56.6 | 2.4 | 7 |
| 2.4 | 6 | 33 | 57.6 | 2.4 | 7 |
| 2.4 | 6 | 32 | 58.6 | 2.4 | 7 |
| 2.4 | 6 | 31 | 59.6 | 2.4 | 7 |
| 2.4 | 6 | 30 | 60.6 | 2.4 | 7 |
| 2.6 | 6 | 60 | 30.4 | 2.6 | 7 |
| 2.6 | 6 | 59 | 31.4 | 2.6 | 7 |
| 2.6 | 6 | 58 | 32.4 | 2.6 | 7 |
| 2.6 | 6 | 57 | 33.4 | 2.6 | 7 |
| 2.6 | 6 | 56 | 34.4 | 2.6 | 7 |
| 2.6 | 6 | 55 | 35.4 | 2.6 | 7 |
| 2.6 | 6 | 54 | 36.4 | 2.6 | 7 |
| 2.6 | 6 | 53 | 37.4 | 2.6 | 7 |
| 2.6 | 6 | 52 | 38.4 | 2.6 | 7 |
| 2.6 | 6 | 51 | 39.4 | 2.6 | 7 |
| 2.6 | 6 | 50 | 40.4 | 2.6 | 7 |
| 2.6 | 6 | 49 | 41.4 | 2.6 | 7 |
| 2.6 | 6 | 48 | 42.4 | 2.6 | 7 |
| 2.6 | 6 | 47 | 43.4 | 2.6 | 7 |
| 2.6 | 6 | 46 | 44.4 | 2.6 | 7 |
| 2.6 | 6 | 45 | 45.4 | 2.6 | 7 |
| 2.6 | 6 | 44 | 46.4 | 2.6 | 7 |
| 2.6 | 6 | 43 | 47.4 | 2.6 | 7 |
| 2.6 | 6 | 42 | 48.4 | 2.6 | 7 |
| 2.6 | 6 | 41 | 49.4 | 2.6 | 7 |
| 2.6 | 6 | 40 | 50.4 | 2.6 | 7 |
| 2.6 | 6 | 39 | 51.4 | 2.6 | 7 |
| 2.6 | 6 | 38 | 52.4 | 2.6 | 7 |
| 2.6 | 6 | 37 | 53.4 | 2.6 | 7 |
| 2.6 | 6 | 36 | 54.4 | 2.6 | 7 |
| 2.6 | 6 | 35 | 55.4 | 2.6 | 7 |
| 2.6 | 6 | 34 | 56.4 | 2.6 | 7 |
| 2.6 | 6 | 33 | 57.4 | 2.6 | 7 |
| 2.6 | 6 | 32 | 58.4 | 2.6 | 7 |
| 2.6 | 6 | 31 | 59.4 | 2.6 | 7 |
| 2.6 | 6 | 30 | 60.4 | 2.6 | 7 |
| 2.8 | 6 | 60 | 30.2 | 2.8 | 7 |
| 2.8 | 6 | 59 | 31.2 | 2.8 | 7 |
| 2.8 | 6 | 58 | 32.2 | 2.8 | 7 |
| 2.8 | 6 | 57 | 33.2 | 2.8 | 7 |
| 2.8 | 6 | 56 | 34.2 | 2.8 | 7 |
| 2.8 | 6 | 55 | 35.2 | 2.8 | 7 |
| 2.8 | 6 | 54 | 36.2 | 2.8 | 7 |
| 2.8 | 6 | 53 | 37.2 | 2.8 | 7 |
| 2.8 | 6 | 52 | 38.2 | 2.8 | 7 |
| 2.8 | 6 | 51 | 39.2 | 2.8 | 7 |
| 2.8 | 6 | 50 | 40.2 | 2.8 | 7 |
| 2.8 | 6 | 49 | 41.2 | 2.8 | 7 |
| 2.8 | 6 | 48 | 42.2 | 2.8 | 7 |
| 2.8 | 6 | 47 | 43.2 | 2.8 | 7 |
| 2.8 | 6 | 46 | 44.2 | 2.8 | 7 |
| 2.8 | 6 | 45 | 45.2 | 2.8 | 7 |
| 2.8 | 6 | 44 | 46.2 | 2.8 | 7 |
| 2.8 | 6 | 43 | 47.2 | 2.8 | 7 |
| 2.8 | 6 | 42 | 48.2 | 2.8 | 7 |
| 2.8 | 6 | 41 | 49.2 | 2.8 | 7 |
| 2.8 | 6 | 40 | 50.2 | 2.8 | 7 |
| 2.8 | 6 | 39 | 51.2 | 2.8 | 7 |
| 2.8 | 6 | 38 | 52.2 | 2.8 | 7 |
| 2.8 | 6 | 37 | 53.2 | 2.8 | 7 |
| 2.8 | 6 | 36 | 54.2 | 2.8 | 7 |
| 2.8 | 6 | 35 | 55.2 | 2.8 | 7 |
| 2.8 | 6 | 34 | 56.2 | 2.8 | 7 |
| 2.8 | 6 | 33 | 57.2 | 2.8 | 7 |
| 2.8 | 6 | 32 | 58.2 | 2.8 | 7 |
| 2.8 | 6 | 31 | 59.2 | 2.8 | 7 |
| 2.8 | 6 | 30 | 60.2 | 2.8 | 7 |
| 3 | 6 | 60 | 30 | 3 | 7 |
| 3 | 6 | 59 | 31 | 3 | 7 |
| 3 | 6 | 58 | 32 | 3 | 7 |
| 3 | 6 | 57 | 33 | 3 | 7 |
| 3 | 6 | 56 | 34 | 3 | 7 |
| 3 | 6 | 55 | 35 | 3 | 7 |
| 3 | 6 | 54 | 36 | 3 | 7 |
| 3 | 6 | 53 | 37 | 3 | 7 |
| 3 | 6 | 52 | 38 | 3 | 7 |
| 3 | 6 | 51 | 39 | 3 | 7 |
| 3 | 6 | 50 | 40 | 3 | 7 |
| 3 | 6 | 49 | 41 | 3 | 7 |
| 3 | 6 | 48 | 42 | 3 | 7 |
| 3 | 6 | 47 | 43 | 3 | 7 |
| 3 | 6 | 46 | 44 | 3 | 7 |
| 3 | 6 | 45 | 45 | 3 | 7 |
| 3 | 6 | 44 | 46 | 3 | 7 |
| 3 | 6 | 43 | 47 | 3 | 7 |
| 3 | 6 | 42 | 48 | 3 | 7 |
| 3 | 6 | 41 | 49 | 3 | 7 |
| 3 | 6 | 40 | 50 | 3 | 7 |
| 3 | 6 | 39 | 51 | 3 | 7 |
| 3 | 6 | 38 | 52 | 3 | 7 |
| 3 | 6 | 37 | 53 | 3 | 7 |
| 3 | 6 | 36 | 54 | 3 | 7 |
| 3 | 6 | 35 | 55 | 3 | 7 |
| 3 | 6 | 34 | 56 | 3 | 7 |
| 3 | 6 | 33 | 57 | 3 | 7 |
| 3 | 6 | 32 | 58 | 3 | 7 |
| 3 | 6 | 31 | 59 | 3 | 7 |
| 3 | 6 | 30 | 60 | 3 | 7 |
| 1 | 10 | 60 | 28 | 1 | 11 |
| 1 | 10 | 59 | 29 | 1 | 11 |
| 1 | 10 | 58 | 30 | 1 | 11 |
| 1 | 10 | 57 | 31 | 1 | 11 |
| 1 | 10 | 56 | 32 | 1 | 11 |
| 1 | 10 | 55 | 33 | 1 | 11 |
| 1 | 10 | 54 | 34 | 1 | 11 |
| 1 | 10 | 53 | 35 | 1 | 11 |
| 1 | 10 | 52 | 36 | 1 | 11 |
| 1 | 10 | 51 | 37 | 1 | 11 |
| 1 | 10 | 50 | 38 | 1 | 11 |
| 1 | 10 | 49 | 39 | 1 | 11 |
| 1 | 10 | 48 | 40 | 1 | 11 |
| 1 | 10 | 47 | 41 | 1 | 11 |
| 1 | 10 | 46 | 42 | 1 | 11 |
| 1 | 10 | 45 | 43 | 1 | 11 |
| 1 | 10 | 44 | 44 | 1 | 11 |
| 1 | 10 | 43 | 45 | 1 | 11 |
| 1 | 10 | 42 | 46 | 1 | 11 |
| 1 | 10 | 41 | 47 | 1 | 11 |
| 1 | 10 | 40 | 48 | 1 | 11 |
| 1 | 10 | 39 | 49 | 1 | 11 |
| 1 | 10 | 38 | 50 | 1 | 11 |
| 1 | 10 | 37 | 51 | 1 | 11 |
| 1 | 10 | 36 | 52 | 1 | 11 |
| 1 | 10 | 35 | 53 | 1 | 11 |
| 1 | 10 | 34 | 54 | 1 | 11 |
| 1 | 10 | 33 | 55 | 1 | 11 |
| 1 | 10 | 32 | 56 | 1 | 11 |
| 1 | 10 | 31 | 57 | 1 | 11 |
| 1 | 10 | 30 | 58 | 1 | 11 |
| 1.2 | 10 | 60 | 27.8 | 1.2 | 11 |
| 1.2 | 10 | 59 | 28.8 | 1.2 | 11 |
| 1.2 | 10 | 58 | 29.8 | 1.2 | 11 |
| 1.2 | 10 | 57 | 30.8 | 1.2 | 11 |
| 1.2 | 10 | 56 | 31.8 | 1.2 | 11 |
| 1.2 | 10 | 55 | 32.8 | 1.2 | 11 |
| 1.2 | 10 | 54 | 33.8 | 1.2 | 11 |
| 1.2 | 10 | 53 | 34.8 | 1.2 | 11 |
| 1.2 | 10 | 52 | 35.8 | 1.2 | 11 |
| 1.2 | 10 | 51 | 36.8 | 1.2 | 11 |
| 1.2 | 10 | 50 | 37.8 | 1.2 | 11 |
| 1.2 | 10 | 49 | 38.8 | 1.2 | 11 |
| 1.2 | 10 | 48 | 39.8 | 1.2 | 11 |
| 1.2 | 10 | 47 | 40.8 | 1.2 | 11 |
| 1.2 | 10 | 46 | 41.8 | 1.2 | 11 |
| 1.2 | 10 | 45 | 42.8 | 1.2 | 11 |
| 1.2 | 10 | 44 | 43.8 | 1.2 | 11 |
| 1.2 | 10 | 43 | 44.8 | 1.2 | 11 |
| 1.2 | 10 | 42 | 45.8 | 1.2 | 11 |
| 1.2 | 10 | 41 | 46.8 | 1.2 | 11 |
| 1.2 | 10 | 40 | 47.8 | 1.2 | 11 |
| 1.2 | 10 | 39 | 48.8 | 1.2 | 11 |
| 1.2 | 10 | 38 | 49.8 | 1.2 | 11 |
| 1.2 | 10 | 37 | 50.8 | 1.2 | 11 |
| 1.2 | 10 | 36 | 51.8 | 1.2 | 11 |
| 1.2 | 10 | 35 | 52.8 | 1.2 | 11 |
| 1.2 | 10 | 34 | 53.8 | 1.2 | 11 |
| 1.2 | 10 | 33 | 54.8 | 1.2 | 11 |
| 1.2 | 10 | 32 | 55.8 | 1.2 | 11 |
| 1.2 | 10 | 31 | 56.8 | 1.2 | 11 |
| 1.2 | 10 | 30 | 57.8 | 1.2 | 11 |
| 1.4 | 10 | 60 | 27.6 | 1.4 | 11 |
| 1.4 | 10 | 59 | 28.6 | 1.4 | 11 |
| 1.4 | 10 | 58 | 29.6 | 1.4 | 11 |
| 1.4 | 10 | 57 | 30.6 | 1.4 | 11 |
| 1.4 | 10 | 56 | 31.6 | 1.4 | 11 |
| 1.4 | 10 | 55 | 32.6 | 1.4 | 11 |
| 1.4 | 10 | 54 | 33.6 | 1.4 | 11 |
| 1.4 | 10 | 53 | 34.6 | 1.4 | 11 |
| 1.4 | 10 | 52 | 35.6 | 1.4 | 11 |
| 1.4 | 10 | 51 | 36.6 | 1.4 | 11 |
| 1.4 | 10 | 50 | 37.6 | 1.4 | 11 |
| 1.4 | 10 | 49 | 38.6 | 1.4 | 11 |
| 1.4 | 10 | 48 | 39.6 | 1.4 | 11 |
| 1.4 | 10 | 47 | 40.6 | 1.4 | 11 |
| 1.4 | 10 | 46 | 41.6 | 1.4 | 11 |
| 1.4 | 10 | 45 | 42.6 | 1.4 | 11 |
| 1.4 | 10 | 44 | 43.6 | 1.4 | 11 |
| 1.4 | 10 | 43 | 44.6 | 1.4 | 11 |
| 1.4 | 10 | 42 | 45.6 | 1.4 | 11 |
| 1.4 | 10 | 41 | 46.6 | 1.4 | 11 |
| 1.4 | 10 | 40 | 47.6 | 1.4 | 11 |
| 1.4 | 10 | 39 | 48.6 | 1.4 | 11 |
| 1.4 | 10 | 38 | 49.6 | 1.4 | 11 |
| 1.4 | 10 | 37 | 50.6 | 1.4 | 11 |
| 1.4 | 10 | 36 | 51.6 | 1.4 | 11 |
| 1.4 | 10 | 35 | 52.6 | 1.4 | 11 |
| 1.4 | 10 | 34 | 53.6 | 1.4 | 11 |
| 1.4 | 10 | 33 | 54.6 | 1.4 | 11 |
| 1.4 | 10 | 32 | 55.6 | 1.4 | 11 |
| 1.4 | 10 | 31 | 56.6 | 1.4 | 11 |
| 1.4 | 10 | 30 | 57.6 | 1.4 | 11 |
| 1.6 | 10 | 60 | 27.4 | 1.6 | 11 |
| 1.6 | 10 | 59 | 28.4 | 1.6 | 11 |
| 1.6 | 10 | 58 | 29.4 | 1.6 | 11 |
| 1.6 | 10 | 57 | 30.4 | 1.6 | 11 |
| 1.6 | 10 | 56 | 31.4 | 1.6 | 11 |
| 1.6 | 10 | 55 | 32.4 | 1.6 | 11 |
| 1.6 | 10 | 54 | 33.4 | 1.6 | 11 |
| 1.6 | 10 | 53 | 34.4 | 1.6 | 11 |
| 1.6 | 10 | 52 | 35.4 | 1.6 | 11 |
| 1.6 | 10 | 51 | 36.4 | 1.6 | 11 |
| 1.6 | 10 | 50 | 37.4 | 1.6 | 11 |
| 1.6 | 10 | 49 | 38.4 | 1.6 | 11 |
| 1.6 | 10 | 48 | 39.4 | 1.6 | 11 |
| 1.6 | 10 | 47 | 40.4 | 1.6 | 11 |
| 1.6 | 10 | 46 | 41.4 | 1.6 | 11 |
| 1.6 | 10 | 45 | 42.4 | 1.6 | 11 |
| 1.6 | 10 | 44 | 43.4 | 1.6 | 11 |
| 1.6 | 10 | 43 | 44.4 | 1.6 | 11 |
| 1.6 | 10 | 42 | 45.4 | 1.6 | 11 |
| 1.6 | 10 | 41 | 46.4 | 1.6 | 11 |
| 1.6 | 10 | 40 | 47.4 | 1.6 | 11 |
| 1.6 | 10 | 39 | 48.4 | 1.6 | 11 |
| 1.6 | 10 | 38 | 49.4 | 1.6 | 11 |
| 1.6 | 10 | 37 | 50.4 | 1.6 | 11 |
| 1.6 | 10 | 36 | 51.4 | 1.6 | 11 |
| 1.6 | 10 | 35 | 52.4 | 1.6 | 11 |
| 1.6 | 10 | 34 | 53.4 | 1.6 | 11 |
| 1.6 | 10 | 33 | 54.4 | 1.6 | 11 |
| 1.6 | 10 | 32 | 55.4 | 1.6 | 11 |
| 1.6 | 10 | 31 | 56.4 | 1.6 | 11 |
| 1.6 | 10 | 30 | 57.4 | 1.6 | 11 |
| 1.8 | 10 | 60 | 27.2 | 1.8 | 11 |
| 1.8 | 10 | 59 | 28.2 | 1.8 | 11 |
| 1.8 | 10 | 58 | 29.2 | 1.8 | 11 |
| 1.8 | 10 | 57 | 30.2 | 1.8 | 11 |
| 1.8 | 10 | 56 | 31.2 | 1.8 | 11 |
| 1.8 | 10 | 55 | 32.2 | 1.8 | 11 |
| 1.8 | 10 | 54 | 33.2 | 1.8 | 11 |
| 1.8 | 10 | 53 | 34.2 | 1.8 | 11 |
| 1.8 | 10 | 52 | 35.2 | 1.8 | 11 |
| 1.8 | 10 | 51 | 36.2 | 1.8 | 11 |
| 1.8 | 10 | 50 | 37.2 | 1.8 | 11 |
| 1.8 | 10 | 49 | 38.2 | 1.8 | 11 |
| 1.8 | 10 | 48 | 39.2 | 1.8 | 11 |
| 1.8 | 10 | 47 | 40.2 | 1.8 | 11 |
| 1.8 | 10 | 46 | 41.2 | 1.8 | 11 |
| 1.8 | 10 | 45 | 42.2 | 1.8 | 11 |
| 1.8 | 10 | 44 | 43.2 | 1.8 | 11 |
| 1.8 | 10 | 43 | 44.2 | 1.8 | 11 |
| 1.8 | 10 | 42 | 45.2 | 1.8 | 11 |
| 1.8 | 10 | 41 | 46.2 | 1.8 | 11 |
| 1.8 | 10 | 40 | 47.2 | 1.8 | 11 |
| 1.8 | 10 | 39 | 48.2 | 1.8 | 11 |
| 1.8 | 10 | 38 | 49.2 | 1.8 | 11 |
| 1.8 | 10 | 37 | 50.2 | 1.8 | 11 |
| 1.8 | 10 | 36 | 51.2 | 1.8 | 11 |
| 1.8 | 10 | 35 | 52.2 | 1.8 | 11 |
| 1.8 | 10 | 34 | 53.2 | 1.8 | 11 |
| 1.8 | 10 | 33 | 54.2 | 1.8 | 11 |
| 1.8 | 10 | 32 | 55.2 | 1.8 | 11 |
| 1.8 | 10 | 31 | 56.2 | 1.8 | 11 |
| 1.8 | 10 | 30 | 57.2 | 1.8 | 11 |
| 2 | 10 | 60 | 27 | 2 | 11 |
| 2 | 10 | 59 | 28 | 2 | 11 |
| 2 | 10 | 58 | 29 | 2 | 11 |
| 2 | 10 | 57 | 30 | 2 | 11 |
| 2 | 10 | 56 | 31 | 2 | 11 |
| 2 | 10 | 55 | 32 | 2 | 11 |
| 2 | 10 | 54 | 33 | 2 | 11 |
| 2 | 10 | 53 | 34 | 2 | 11 |
| 2 | 10 | 52 | 35 | 2 | 11 |
| 2 | 10 | 51 | 36 | 2 | 11 |
| 2 | 10 | 50 | 37 | 2 | 11 |
| 2 | 10 | 49 | 38 | 2 | 11 |
| 2 | 10 | 48 | 39 | 2 | 11 |
| 2 | 10 | 47 | 40 | 2 | 11 |
| 2 | 10 | 46 | 41 | 2 | 11 |
| 2 | 10 | 45 | 42 | 2 | 11 |
| 2 | 10 | 44 | 43 | 2 | 11 |
| 2 | 10 | 43 | 44 | 2 | 11 |
| 2 | 10 | 42 | 45 | 2 | 11 |
| 2 | 10 | 41 | 46 | 2 | 11 |
| 2 | 10 | 40 | 47 | 2 | 11 |
| 2 | 10 | 39 | 48 | 2 | 11 |
| 2 | 10 | 38 | 49 | 2 | 11 |
| 2 | 10 | 37 | 50 | 2 | 11 |
| 2 | 10 | 36 | 51 | 2 | 11 |
| 2 | 10 | 35 | 52 | 2 | 11 |
| 2 | 10 | 34 | 53 | 2 | 11 |
| 2 | 10 | 33 | 54 | 2 | 11 |
| 2 | 10 | 32 | 55 | 2 | 11 |
| 2 | 10 | 31 | 56 | 2 | 11 |
| 2 | 10 | 30 | 57 | 2 | 11 |
| 2.2 | 10 | 60 | 26.8 | 2.2 | 11 |
| 2.2 | 10 | 59 | 27.8 | 2.2 | 11 |
| 2.2 | 10 | 58 | 28.8 | 2.2 | 11 |
| 2.2 | 10 | 57 | 29.8 | 2.2 | 11 |
| 2.2 | 10 | 56 | 30.8 | 2.2 | 11 |
| 2.2 | 10 | 55 | 31.8 | 2.2 | 11 |
| 2.2 | 10 | 54 | 32.8 | 2.2 | 11 |
| 2.2 | 10 | 53 | 33.8 | 2.2 | 11 |
| 2.2 | 10 | 52 | 34.8 | 2.2 | 11 |
| 2.2 | 10 | 51 | 35.8 | 2.2 | 11 |
| 2.2 | 10 | 50 | 36.8 | 2.2 | 11 |
| 2.2 | 10 | 49 | 37.8 | 2.2 | 11 |
| 2.2 | 10 | 48 | 38.8 | 2.2 | 11 |
| 2.2 | 10 | 47 | 39.8 | 2.2 | 11 |
| 2.2 | 10 | 46 | 40.8 | 2.2 | 11 |
| 2.2 | 10 | 45 | 41.8 | 2.2 | 11 |
| 2.2 | 10 | 44 | 42.8 | 2.2 | 11 |
| 2.2 | 10 | 43 | 43.8 | 2.2 | 11 |
| 2.2 | 10 | 42 | 44.8 | 2.2 | 11 |
| 2.2 | 10 | 41 | 45.8 | 2.2 | 11 |
| 2.2 | 10 | 40 | 46.8 | 2.2 | 11 |
| 2.2 | 10 | 39 | 47.8 | 2.2 | 11 |
| 2.2 | 10 | 38 | 48.8 | 2.2 | 11 |
| 2.2 | 10 | 37 | 49.8 | 2.2 | 11 |
| 2.2 | 10 | 36 | 50.8 | 2.2 | 11 |
| 2.2 | 10 | 35 | 51.8 | 2.2 | 11 |
| 2.2 | 10 | 34 | 52.8 | 2.2 | 11 |
| 2.2 | 10 | 33 | 53.8 | 2.2 | 11 |
| 2.2 | 10 | 32 | 54.8 | 2.2 | 11 |
| 2.2 | 10 | 31 | 55.8 | 2.2 | 11 |
| 2.2 | 10 | 30 | 56.8 | 2.2 | 11 |
| 2.4 | 10 | 60 | 26.6 | 2.4 | 11 |
| 2.4 | 10 | 59 | 27.6 | 2.4 | 11 |
| 2.4 | 10 | 58 | 28.6 | 2.4 | 11 |
| 2.4 | 10 | 57 | 29.6 | 2.4 | 11 |
| 2.4 | 10 | 56 | 30.6 | 2.4 | 11 |
| 2.4 | 10 | 55 | 31.6 | 2.4 | 11 |
| 2.4 | 10 | 54 | 32.6 | 2.4 | 11 |
| 2.4 | 10 | 53 | 33.6 | 2.4 | 11 |
| 2.4 | 10 | 52 | 34.6 | 2.4 | 11 |
| 2.4 | 10 | 51 | 35.6 | 2.4 | 11 |
| 2.4 | 10 | 50 | 36.6 | 2.4 | 11 |
| 2.4 | 10 | 49 | 37.6 | 2.4 | 11 |
| 2.4 | 10 | 48 | 38.6 | 2.4 | 11 |
| 2.4 | 10 | 47 | 39.6 | 2.4 | 11 |
| 2.4 | 10 | 46 | 40.6 | 2.4 | 11 |
| 2.4 | 10 | 45 | 41.6 | 2.4 | 11 |
| 2.4 | 10 | 44 | 42.6 | 2.4 | 11 |
| 2.4 | 10 | 43 | 43.6 | 2.4 | 11 |
| 2.4 | 10 | 42 | 44.6 | 2.4 | 11 |
| 2.4 | 10 | 41 | 45.6 | 2.4 | 11 |
| 2.4 | 10 | 40 | 46.6 | 2.4 | 11 |
| 2.4 | 10 | 39 | 47.6 | 2.4 | 11 |
| 2.4 | 10 | 38 | 48.6 | 2.4 | 11 |
| 2.4 | 10 | 37 | 49.6 | 2.4 | 11 |
| 2.4 | 10 | 36 | 50.6 | 2.4 | 11 |
| 2.4 | 10 | 35 | 51.6 | 2.4 | 11 |
| 2.4 | 10 | 34 | 52.6 | 2.4 | 11 |
| 2.4 | 10 | 33 | 53.6 | 2.4 | 11 |
| 2.4 | 10 | 32 | 54.6 | 2.4 | 11 |
| 2.4 | 10 | 31 | 55.6 | 2.4 | 11 |
| 2.4 | 10 | 30 | 56.6 | 2.4 | 11 |
| 2.6 | 10 | 60 | 26.4 | 2.6 | 11 |
| 2.6 | 10 | 59 | 27.4 | 2.6 | 11 |
| 2.6 | 10 | 58 | 28.4 | 2.6 | 11 |
| 2.6 | 10 | 57 | 29.4 | 2.6 | 11 |
| 2.6 | 10 | 56 | 30.4 | 2.6 | 11 |
| 2.6 | 10 | 55 | 31.4 | 2.6 | 11 |
| 2.6 | 10 | 54 | 32.4 | 2.6 | 11 |
| 2.6 | 10 | 53 | 33.4 | 2.6 | 11 |
| 2.6 | 10 | 52 | 34.4 | 2.6 | 11 |
| 2.6 | 10 | 51 | 35.4 | 2.6 | 11 |
| 2.6 | 10 | 50 | 36.4 | 2.6 | 11 |
| 2.6 | 10 | 49 | 37.4 | 2.6 | 11 |
| 2.6 | 10 | 48 | 38.4 | 2.6 | 11 |
| 2.6 | 10 | 47 | 39.4 | 2.6 | 11 |
| 2.6 | 10 | 46 | 40.4 | 2.6 | 11 |
| 2.6 | 10 | 45 | 41.4 | 2.6 | 11 |
| 2.6 | 10 | 44 | 42.4 | 2.6 | 11 |
| 2.6 | 10 | 43 | 43.4 | 2.6 | 11 |
| 2.6 | 10 | 42 | 44.4 | 2.6 | 11 |
| 2.6 | 10 | 41 | 45.4 | 2.6 | 11 |
| 2.6 | 10 | 40 | 46.4 | 2.6 | 11 |
| 2.6 | 10 | 39 | 47.4 | 2.6 | 11 |
| 2.6 | 10 | 38 | 48.4 | 2.6 | 11 |
| 2.6 | 10 | 37 | 49.4 | 2.6 | 11 |
| 2.6 | 10 | 36 | 50.4 | 2.6 | 11 |
| 2.6 | 10 | 35 | 51.4 | 2.6 | 11 |
| 2.6 | 10 | 34 | 52.4 | 2.6 | 11 |
| 2.6 | 10 | 33 | 53.4 | 2.6 | 11 |
| 2.6 | 10 | 32 | 54.4 | 2.6 | 11 |
| 2.6 | 10 | 31 | 55.4 | 2.6 | 11 |
| 2.6 | 10 | 30 | 56.4 | 2.6 | 11 |
| 2.8 | 10 | 60 | 26.2 | 2.8 | 11 |
| 2.8 | 10 | 59 | 27.2 | 2.8 | 11 |
| 2.8 | 10 | 58 | 28.2 | 2.8 | 11 |
| 2.8 | 10 | 57 | 29.2 | 2.8 | 11 |
| 2.8 | 10 | 56 | 30.2 | 2.8 | 11 |
| 2.8 | 10 | 55 | 31.2 | 2.8 | 11 |
| 2.8 | 10 | 54 | 32.2 | 2.8 | 11 |
| 2.8 | 10 | 53 | 33.2 | 2.8 | 11 |
| 2.8 | 10 | 52 | 34.2 | 2.8 | 11 |
| 2.8 | 10 | 51 | 35.2 | 2.8 | 11 |
| 2.8 | 10 | 50 | 36.2 | 2.8 | 11 |
| 2.8 | 10 | 49 | 37.2 | 2.8 | 11 |
| 2.8 | 10 | 48 | 38.2 | 2.8 | 11 |
| 2.8 | 10 | 47 | 39.2 | 2.8 | 11 |
| 2.8 | 10 | 46 | 40.2 | 2.8 | 11 |
| 2.8 | 10 | 45 | 41.2 | 2.8 | 11 |
| 2.8 | 10 | 44 | 42.2 | 2.8 | 11 |
| 2.8 | 10 | 43 | 43.2 | 2.8 | 11 |
| 2.8 | 10 | 42 | 44.2 | 2.8 | 11 |
| 2.8 | 10 | 41 | 45.2 | 2.8 | 11 |
| 2.8 | 10 | 40 | 46.2 | 2.8 | 11 |
| 2.8 | 10 | 39 | 47.2 | 2.8 | 11 |
| 2.8 | 10 | 38 | 48.2 | 2.8 | 11 |
| 2.8 | 10 | 37 | 49.2 | 2.8 | 11 |
| 2.8 | 10 | 36 | 50.2 | 2.8 | 11 |
| 2.8 | 10 | 35 | 51.2 | 2.8 | 11 |
| 2.8 | 10 | 34 | 52.2 | 2.8 | 11 |
| 2.8 | 10 | 33 | 53.2 | 2.8 | 11 |
| 2.8 | 10 | 32 | 54.2 | 2.8 | 11 |
| 2.8 | 10 | 31 | 55.2 | 2.8 | 11 |
| 2.8 | 10 | 30 | 56.2 | 2.8 | 11 |
| 3 | 10 | 60 | 26 | 3 | 11 |
| 3 | 10 | 59 | 27 | 3 | 11 |
| 3 | 10 | 58 | 28 | 3 | 11 |
| 3 | 10 | 57 | 29 | 3 | 11 |
| 3 | 10 | 56 | 30 | 3 | 11 |
| 3 | 10 | 55 | 31 | 3 | 11 |
| 3 | 10 | 54 | 32 | 3 | 11 |
| 3 | 10 | 53 | 33 | 3 | 11 |
| 3 | 10 | 52 | 34 | 3 | 11 |
| 3 | 10 | 51 | 35 | 3 | 11 |
| 3 | 10 | 50 | 36 | 3 | 11 |
| 3 | 10 | 49 | 37 | 3 | 11 |
| 3 | 10 | 48 | 38 | 3 | 11 |
| 3 | 10 | 47 | 39 | 3 | 11 |
| 3 | 10 | 46 | 40 | 3 | 11 |
| 3 | 10 | 45 | 41 | 3 | 11 |
| 3 | 10 | 44 | 42 | 3 | 11 |
| 3 | 10 | 43 | 43 | 3 | 11 |
| 3 | 10 | 42 | 44 | 3 | 11 |
| 3 | 10 | 41 | 45 | 3 | 11 |
| 3 | 10 | 40 | 46 | 3 | 11 |
| 3 | 10 | 39 | 47 | 3 | 11 |
| 3 | 10 | 38 | 48 | 3 | 11 |
| 3 | 10 | 37 | 49 | 3 | 11 |
| 3 | 10 | 36 | 50 | 3 | 11 |
| 3 | 10 | 35 | 51 | 3 | 11 |
| 3 | 10 | 34 | 52 | 3 | 11 |
| 3 | 10 | 33 | 53 | 3 | 11 |
| 3 | 10 | 32 | 54 | 3 | 11 |
| 3 | 10 | 31 | 55 | 3 | 11 |
| 3 | 10 | 30 | 56 | 3 | 11 |
| 1 | 14 | 60 | 24 | 1 | 15 |
| 1 | 14 | 59 | 25 | 1 | 15 |
| 1 | 14 | 58 | 26 | 1 | 15 |
| 1 | 14 | 57 | 27 | 1 | 15 |
| 1 | 14 | 56 | 28 | 1 | 15 |
| 1 | 14 | 55 | 29 | 1 | 15 |
| 1 | 14 | 54 | 30 | 1 | 15 |
| 1 | 14 | 53 | 31 | 1 | 15 |
| 1 | 14 | 52 | 32 | 1 | 15 |
| 1 | 14 | 51 | 33 | 1 | 15 |
| 1 | 14 | 50 | 34 | 1 | 15 |
| 1 | 14 | 49 | 35 | 1 | 15 |
| 1 | 14 | 48 | 36 | 1 | 15 |
| 1 | 14 | 47 | 37 | 1 | 15 |
| 1 | 14 | 46 | 38 | 1 | 15 |
| 1 | 14 | 45 | 39 | 1 | 15 |
| 1 | 14 | 44 | 40 | 1 | 15 |
| 1 | 14 | 43 | 41 | 1 | 15 |
| 1 | 14 | 42 | 42 | 1 | 15 |
| 1 | 14 | 41 | 43 | 1 | 15 |
| 1 | 14 | 40 | 44 | 1 | 15 |
| 1 | 14 | 39 | 45 | 1 | 15 |
| 1 | 14 | 38 | 46 | 1 | 15 |
| 1 | 14 | 37 | 47 | 1 | 15 |
| 1 | 14 | 36 | 48 | 1 | 15 |
| 1 | 14 | 35 | 49 | 1 | 15 |
| 1 | 14 | 34 | 50 | 1 | 15 |
| 1 | 14 | 33 | 51 | 1 | 15 |
| 1 | 14 | 32 | 52 | 1 | 15 |
| 1 | 14 | 31 | 53 | 1 | 15 |
| 1 | 14 | 30 | 54 | 1 | 15 |
| 1.2 | 14 | 60 | 23.8 | 1.2 | 15 |
| 1.2 | 14 | 59 | 24.8 | 1.2 | 15 |
| 1.2 | 14 | 58 | 25.8 | 1.2 | 15 |
| 1.2 | 14 | 57 | 26.8 | 1.2 | 15 |
| 1.2 | 14 | 56 | 27.8 | 1.2 | 15 |
| 1.2 | 14 | 55 | 28.8 | 1.2 | 15 |
| 1.2 | 14 | 54 | 29.8 | 1.2 | 15 |
| 1.2 | 14 | 53 | 30.8 | 1.2 | 15 |
| 1.2 | 14 | 52 | 31.8 | 1.2 | 15 |
| 1.2 | 14 | 51 | 32.8 | 1.2 | 15 |
| 1.2 | 14 | 50 | 33.8 | 1.2 | 15 |
| 1.2 | 14 | 49 | 34.8 | 1.2 | 15 |
| 1.2 | 14 | 48 | 35.8 | 1.2 | 15 |
| 1.2 | 14 | 47 | 36.8 | 1.2 | 15 |
| 1.2 | 14 | 46 | 37.8 | 1.2 | 15 |
| 1.2 | 14 | 45 | 38.8 | 1.2 | 15 |
| 1.2 | 14 | 44 | 39.8 | 1.2 | 15 |
| 1.2 | 14 | 43 | 40.8 | 1.2 | 15 |
| 1.2 | 14 | 42 | 41.8 | 1.2 | 15 |
| 1.2 | 14 | 41 | 42.8 | 1.2 | 15 |
| 1.2 | 14 | 40 | 43.8 | 1.2 | 15 |
| 1.2 | 14 | 39 | 44.8 | 1.2 | 15 |
| 1.2 | 14 | 38 | 45.8 | 1.2 | 15 |
| 1.2 | 14 | 37 | 46.8 | 1.2 | 15 |
| 1.2 | 14 | 36 | 47.8 | 1.2 | 15 |
| 1.2 | 14 | 35 | 48.8 | 1.2 | 15 |
| 1.2 | 14 | 34 | 49.8 | 1.2 | 15 |
| 1.2 | 14 | 33 | 50.8 | 1.2 | 15 |
| 1.2 | 14 | 32 | 51.8 | 1.2 | 15 |
| 1.2 | 14 | 31 | 52.8 | 1.2 | 15 |
| 1.2 | 14 | 30 | 53.8 | 1.2 | 15 |
| 1.4 | 14 | 60 | 23.6 | 1.4 | 15 |
| 1.4 | 14 | 59 | 24.6 | 1.4 | 15 |
| 1.4 | 14 | 58 | 25.6 | 1.4 | 15 |
| 1.4 | 14 | 57 | 26.6 | 1.4 | 15 |
| 1.4 | 14 | 56 | 27.6 | 1.4 | 15 |
| 1.4 | 14 | 55 | 28.6 | 1.4 | 15 |
| 1.4 | 14 | 54 | 29.6 | 1.4 | 15 |
| 1.4 | 14 | 53 | 30.6 | 1.4 | 15 |
| 1.4 | 14 | 52 | 31.6 | 1.4 | 15 |
| 1.4 | 14 | 51 | 32.6 | 1.4 | 15 |
| 1.4 | 14 | 50 | 33.6 | 1.4 | 15 |
| 1.4 | 14 | 49 | 34.6 | 1.4 | 15 |
| 1.4 | 14 | 48 | 35.6 | 1.4 | 15 |
| 1.4 | 14 | 47 | 36.6 | 1.4 | 15 |
| 1.4 | 14 | 46 | 37.6 | 1.4 | 15 |
| 1.4 | 14 | 45 | 38.6 | 1.4 | 15 |
| 1.4 | 14 | 44 | 39.6 | 1.4 | 15 |
| 1.4 | 14 | 43 | 40.6 | 1.4 | 15 |
| 1.4 | 14 | 42 | 41.6 | 1.4 | 15 |
| 1.4 | 14 | 41 | 42.6 | 1.4 | 15 |
| 1.4 | 14 | 40 | 43.6 | 1.4 | 15 |
| 1.4 | 14 | 39 | 44.6 | 1.4 | 15 |
| 1.4 | 14 | 38 | 45.6 | 1.4 | 15 |
| 1.4 | 14 | 37 | 46.6 | 1.4 | 15 |
| 1.4 | 14 | 36 | 47.6 | 1.4 | 15 |
| 1.4 | 14 | 35 | 48.6 | 1.4 | 15 |
| 1.4 | 14 | 34 | 49.6 | 1.4 | 15 |
| 1.4 | 14 | 33 | 50.6 | 1.4 | 15 |
| 1.4 | 14 | 32 | 51.6 | 1.4 | 15 |
| 1.4 | 14 | 31 | 52.6 | 1.4 | 15 |
| 1.4 | 14 | 30 | 53.6 | 1.4 | 15 |
| 1.6 | 14 | 60 | 23.4 | 1.6 | 15 |
| 1.6 | 14 | 59 | 24.4 | 1.6 | 15 |
| 1.6 | 14 | 58 | 25.4 | 1.6 | 15 |
| 1.6 | 14 | 57 | 26.4 | 1.6 | 15 |
| 1.6 | 14 | 56 | 27.4 | 1.6 | 15 |
| 1.6 | 14 | 55 | 28.4 | 1.6 | 15 |
| 1.6 | 14 | 54 | 29.4 | 1.6 | 15 |
| 1.6 | 14 | 53 | 30.4 | 1.6 | 15 |
| 1.6 | 14 | 52 | 31.4 | 1.6 | 15 |
| 1.6 | 14 | 51 | 32.4 | 1.6 | 15 |
| 1.6 | 14 | 50 | 33.4 | 1.6 | 15 |
| 1.6 | 14 | 49 | 34.4 | 1.6 | 15 |
| 1.6 | 14 | 48 | 35.4 | 1.6 | 15 |
| 1.6 | 14 | 47 | 36.4 | 1.6 | 15 |
| 1.6 | 14 | 46 | 37.4 | 1.6 | 15 |
| 1.6 | 14 | 45 | 38.4 | 1.6 | 15 |
| 1.6 | 14 | 44 | 39.4 | 1.6 | 15 |
| 1.6 | 14 | 43 | 40.4 | 1.6 | 15 |
| 1.6 | 14 | 42 | 41.4 | 1.6 | 15 |
| 1.6 | 14 | 41 | 42.4 | 1.6 | 15 |
| 1.6 | 14 | 40 | 43.4 | 1.6 | 15 |
| 1.6 | 14 | 39 | 44.4 | 1.6 | 15 |
| 1.6 | 14 | 38 | 45.4 | 1.6 | 15 |
| 1.6 | 14 | 37 | 46.4 | 1.6 | 15 |
| 1.6 | 14 | 36 | 47.4 | 1.6 | 15 |
| 1.6 | 14 | 35 | 48.4 | 1.6 | 15 |
| 1.6 | 14 | 34 | 49.4 | 1.6 | 15 |
| 1.6 | 14 | 33 | 50.4 | 1.6 | 15 |
| 1.6 | 14 | 32 | 51.4 | 1.6 | 15 |
| 1.6 | 14 | 31 | 52.4 | 1.6 | 15 |
| 1.6 | 14 | 30 | 53.4 | 1.6 | 15 |
| 1.8 | 14 | 60 | 23.2 | 1.8 | 15 |
| 1.8 | 14 | 59 | 24.2 | 1.8 | 15 |
| 1.8 | 14 | 58 | 25.2 | 1.8 | 15 |
| 1.8 | 14 | 57 | 26.2 | 1.8 | 15 |
| 1.8 | 14 | 56 | 27.2 | 1.8 | 15 |
| 1.8 | 14 | 55 | 28.2 | 1.8 | 15 |
| 1.8 | 14 | 54 | 29.2 | 1.8 | 15 |
| 1.8 | 14 | 53 | 30.2 | 1.8 | 15 |
| 1.8 | 14 | 52 | 31.2 | 1.8 | 15 |
| 1.8 | 14 | 51 | 32.2 | 1.8 | 15 |
| 1.8 | 14 | 50 | 33.2 | 1.8 | 15 |
| 1.8 | 14 | 49 | 34.2 | 1.8 | 15 |
| 1.8 | 14 | 48 | 35.2 | 1.8 | 15 |
| 1.8 | 14 | 47 | 36.2 | 1.8 | 15 |
| 1.8 | 14 | 46 | 37.2 | 1.8 | 15 |
| 1.8 | 14 | 45 | 38.2 | 1.8 | 15 |
| 1.8 | 14 | 44 | 39.2 | 1.8 | 15 |
| 1.8 | 14 | 43 | 40.2 | 1.8 | 15 |
| 1.8 | 14 | 42 | 41.2 | 1.8 | 15 |
| 1.8 | 14 | 41 | 42.2 | 1.8 | 15 |
| 1.8 | 14 | 40 | 43.2 | 1.8 | 15 |
| 1.8 | 14 | 39 | 44.2 | 1.8 | 15 |
| 1.8 | 14 | 38 | 45.2 | 1.8 | 15 |
| 1.8 | 14 | 37 | 46.2 | 1.8 | 15 |
| 1.8 | 14 | 36 | 47.2 | 1.8 | 15 |
| 1.8 | 14 | 35 | 48.2 | 1.8 | 15 |
| 1.8 | 14 | 34 | 49.2 | 1.8 | 15 |
| 1.8 | 14 | 33 | 50.2 | 1.8 | 15 |
| 1.8 | 14 | 32 | 51.2 | 1.8 | 15 |
| 1.8 | 14 | 31 | 52.2 | 1.8 | 15 |
| 2 | 14 | 30 | 53.2 | 2 | 15 |
| 2 | 14 | 60 | 23 | 2 | 15 |
| 2 | 14 | 59 | 24 | 2 | 15 |
| 2 | 14 | 58 | 25 | 2 | 15 |
| 2 | 14 | 57 | 26 | 2 | 15 |
| 2 | 14 | 56 | 27 | 2 | 15 |
| 2 | 14 | 55 | 28 | 2 | 15 |
| 2 | 14 | 54 | 29 | 2 | 15 |
| 2 | 14 | 53 | 30 | 2 | 15 |
| 2 | 14 | 52 | 31 | 2 | 15 |
| 2 | 14 | 51 | 32 | 2 | 15 |
| 2 | 14 | 50 | 33 | 2 | 15 |
| 2 | 14 | 49 | 34 | 2 | 15 |
| 2 | 14 | 48 | 35 | 2 | 15 |
| 2 | 14 | 47 | 36 | 2 | 15 |
| 2 | 14 | 46 | 37 | 2 | 15 |
| 2 | 14 | 45 | 38 | 2 | 15 |
| 2 | 14 | 44 | 39 | 2 | 15 |
| 2 | 14 | 43 | 40 | 2 | 15 |
| 2 | 14 | 42 | 41 | 2 | 15 |
| 2 | 14 | 41 | 42 | 2 | 15 |
| 2 | 14 | 40 | 43 | 2 | 15 |
| 2 | 14 | 39 | 44 | 2 | 15 |
| 2 | 14 | 38 | 45 | 2 | 15 |
| 2 | 14 | 37 | 46 | 2 | 15 |
| 2 | 14 | 36 | 47 | 2 | 15 |
| 2 | 14 | 35 | 48 | 2 | 15 |
| 2 | 14 | 34 | 49 | 2 | 15 |
| 2 | 14 | 33 | 50 | 2 | 15 |
| 2 | 14 | 32 | 51 | 2 | 15 |
| 2 | 14 | 31 | 52 | 2 | 15 |
| 2 | 14 | 30 | 53 | 2 | 15 |
| 2.2 | 14 | 60 | 22.8 | 2.2 | 15 |
| 2.2 | 14 | 59 | 23.8 | 2.2 | 15 |
| 2.2 | 14 | 58 | 24.8 | 2.2 | 15 |
| 2.2 | 14 | 57 | 25.8 | 2.2 | 15 |
| 2.2 | 14 | 56 | 26.8 | 2.2 | 15 |
| 2.2 | 14 | 55 | 27.8 | 2.2 | 15 |
| 2.2 | 14 | 54 | 28.8 | 2.2 | 15 |
| 2.2 | 14 | 53 | 29.8 | 2.2 | 15 |
| 2.2 | 14 | 52 | 30.8 | 2.2 | 15 |
| 2.2 | 14 | 51 | 31.8 | 2.2 | 15 |
| 2.2 | 14 | 50 | 32.8 | 2.2 | 15 |
| 2.2 | 14 | 49 | 33.8 | 2.2 | 15 |
| 2.2 | 14 | 48 | 34.8 | 2.2 | 15 |
| 2.2 | 14 | 47 | 35.8 | 2.2 | 15 |
| 2.2 | 14 | 46 | 36.8 | 2.2 | 15 |
| 2.2 | 14 | 45 | 37.8 | 2.2 | 15 |
| 2.2 | 14 | 44 | 38.8 | 2.2 | 15 |
| 2.2 | 14 | 43 | 39.8 | 2.2 | 15 |
| 2.2 | 14 | 42 | 40.8 | 2.2 | 15 |
| 2.2 | 14 | 41 | 41.8 | 2.2 | 15 |
| 2.2 | 14 | 40 | 42.8 | 2.2 | 15 |
| 2.2 | 14 | 39 | 43.8 | 2.2 | 15 |
| 2.2 | 14 | 38 | 44.8 | 2.2 | 15 |
| 2.2 | 14 | 37 | 45.8 | 2.2 | 15 |
| 2.2 | 14 | 36 | 46.8 | 2.2 | 15 |
| 2.2 | 14 | 35 | 47.8 | 2.2 | 15 |
| 2.2 | 14 | 34 | 48.8 | 2.2 | 15 |
| 2.2 | 14 | 33 | 49.8 | 2.2 | 15 |
| 2.2 | 14 | 32 | 50.8 | 2.2 | 15 |
| 2.2 | 14 | 31 | 51.8 | 2.2 | 15 |
| 2.2 | 14 | 30 | 52.8 | 2.2 | 15 |
| 2.4 | 14 | 60 | 22.6 | 2.4 | 15 |
| 2.4 | 14 | 59 | 23.6 | 2.4 | 15 |
| 2.4 | 14 | 58 | 24.6 | 2.4 | 15 |
| 2.4 | 14 | 57 | 25.6 | 2.4 | 15 |
| 2.4 | 14 | 56 | 26.6 | 2.4 | 15 |
| 2.4 | 14 | 55 | 27.6 | 2.4 | 15 |
| 2.4 | 14 | 54 | 28.6 | 2.4 | 15 |
| 2.4 | 14 | 53 | 29.6 | 2.4 | 15 |
| 2.4 | 14 | 52 | 30.6 | 2.4 | 15 |
| 2.4 | 14 | 51 | 31.6 | 2.4 | 15 |
| 2.4 | 14 | 50 | 32.6 | 2.4 | 15 |
| 2.4 | 14 | 49 | 33.6 | 2.4 | 15 |
| 2.4 | 14 | 48 | 34.6 | 2.4 | 15 |
| 2.4 | 14 | 47 | 35.6 | 2.4 | 15 |
| 2.4 | 14 | 46 | 36.6 | 2.4 | 15 |
| 2.4 | 14 | 45 | 37.6 | 2.4 | 15 |
| 2.4 | 14 | 44 | 38.6 | 2.4 | 15 |
| 2.4 | 14 | 43 | 39.6 | 2.4 | 15 |
| 2.4 | 14 | 42 | 40.6 | 2.4 | 15 |
| 2.4 | 14 | 41 | 41.6 | 2.4 | 15 |
| 2.4 | 14 | 40 | 42.6 | 2.4 | 15 |
| 2.4 | 14 | 39 | 43.6 | 2.4 | 15 |
| 2.4 | 14 | 38 | 44.6 | 2.4 | 15 |
| 2.4 | 14 | 37 | 45.6 | 2.4 | 15 |
| 2.4 | 14 | 36 | 46.6 | 2.4 | 15 |
| 2.4 | 14 | 35 | 47.6 | 2.4 | 15 |
| 2.4 | 14 | 34 | 48.6 | 2.4 | 15 |
| 2.4 | 14 | 33 | 49.6 | 2.4 | 15 |
| 2.4 | 14 | 32 | 50.6 | 2.4 | 15 |
| 2.4 | 14 | 31 | 51.6 | 2.4 | 15 |
| 2.4 | 14 | 30 | 52.6 | 2.4 | 15 |
| 2.6 | 14 | 60 | 22.4 | 2.6 | 15 |
| 2.6 | 14 | 59 | 23.4 | 2.6 | 15 |
| 2.6 | 14 | 58 | 24.4 | 2.6 | 15 |
| 2.6 | 14 | 57 | 25.4 | 2.6 | 15 |
| 2.6 | 14 | 56 | 26.4 | 2.6 | 15 |
| 2.6 | 14 | 55 | 27.4 | 2.6 | 15 |
| 2.6 | 14 | 54 | 28.4 | 2.6 | 15 |
| 2.6 | 14 | 53 | 29.4 | 2.6 | 15 |
| 2.6 | 14 | 52 | 30.4 | 2.6 | 15 |
| 2.6 | 14 | 51 | 31.4 | 2.6 | 15 |
| 2.6 | 14 | 50 | 32.4 | 2.6 | 15 |
| 2.6 | 14 | 49 | 33.4 | 2.6 | 15 |
| 2.6 | 14 | 48 | 34.4 | 2.6 | 15 |
| 2.6 | 14 | 47 | 35.4 | 2.6 | 15 |
| 2.6 | 14 | 46 | 36.4 | 2.6 | 15 |
| 2.6 | 14 | 45 | 37.4 | 2.6 | 15 |
| 2.6 | 14 | 44 | 38.4 | 2.6 | 15 |
| 2.6 | 14 | 43 | 39.4 | 2.6 | 15 |
| 2.6 | 14 | 42 | 40.4 | 2.6 | 15 |
| 2.6 | 14 | 41 | 41.4 | 2.6 | 15 |
| 2.6 | 14 | 40 | 42.4 | 2.6 | 15 |
| 2.6 | 14 | 39 | 43.4 | 2.6 | 15 |
| 2.6 | 14 | 38 | 44.4 | 2.6 | 15 |
| 2.6 | 14 | 37 | 45.4 | 2.6 | 15 |
| 2.6 | 14 | 36 | 46.4 | 2.6 | 15 |
| 2.6 | 14 | 35 | 47.4 | 2.6 | 15 |
| 2.6 | 14 | 34 | 48.4 | 2.6 | 15 |
| 2.6 | 14 | 33 | 49.4 | 2.6 | 15 |
| 2.6 | 14 | 32 | 50.4 | 2.6 | 15 |
| 2.6 | 14 | 31 | 51.4 | 2.6 | 15 |
| 2.6 | 14 | 30 | 52.4 | 2.6 | 15 |
| 2.8 | 14 | 60 | 22.2 | 2.8 | 15 |
| 2.8 | 14 | 59 | 23.2 | 2.8 | 15 |
| 2.8 | 14 | 58 | 24.2 | 2.8 | 15 |
| 2.8 | 14 | 57 | 25.2 | 2.8 | 15 |
| 2.8 | 14 | 56 | 26.2 | 2.8 | 15 |
| 2.8 | 14 | 55 | 27.2 | 2.8 | 15 |
| 2.8 | 14 | 54 | 28.2 | 2.8 | 15 |
| 2.8 | 14 | 53 | 29.2 | 2.8 | 15 |
| 2.8 | 14 | 52 | 30.2 | 2.8 | 15 |
| 2.8 | 14 | 51 | 31.2 | 2.8 | 15 |
| 2.8 | 14 | 50 | 32.2 | 2.8 | 15 |
| 2.8 | 14 | 49 | 33.2 | 2.8 | 15 |
| 2.8 | 14 | 48 | 34.2 | 2.8 | 15 |
| 2.8 | 14 | 47 | 35.2 | 2.8 | 15 |
| 2.8 | 14 | 46 | 36.2 | 2.8 | 15 |
| 2.8 | 14 | 45 | 37.2 | 2.8 | 15 |
| 2.8 | 14 | 44 | 38.2 | 2.8 | 15 |
| 2.8 | 14 | 43 | 39.2 | 2.8 | 15 |
| 2.8 | 14 | 42 | 40.2 | 2.8 | 15 |
| 2.8 | 14 | 41 | 41.2 | 2.8 | 15 |
| 2.8 | 14 | 40 | 42.2 | 2.8 | 15 |
| 2.8 | 14 | 39 | 43.2 | 2.8 | 15 |
| 2.8 | 14 | 38 | 44.2 | 2.8 | 15 |
| 2.8 | 14 | 37 | 45.2 | 2.8 | 15 |
| 2.8 | 14 | 36 | 46.2 | 2.8 | 15 |
| 2.8 | 14 | 35 | 47.2 | 2.8 | 15 |
| 2.8 | 14 | 34 | 48.2 | 2.8 | 15 |
| 2.8 | 14 | 33 | 49.2 | 2.8 | 15 |
| 2.8 | 14 | 32 | 50.2 | 2.8 | 15 |
| 2.8 | 14 | 31 | 51.2 | 2.8 | 15 |
| 2.8 | 14 | 30 | 52.2 | 2.8 | 15 |
| 3 | 14 | 60 | 22 | 3 | 15 |
| 3 | 14 | 59 | 23 | 3 | 15 |
| 3 | 14 | 58 | 24 | 3 | 15 |
| 3 | 14 | 57 | 25 | 3 | 15 |
| 3 | 14 | 56 | 26 | 3 | 15 |
| 3 | 14 | 55 | 27 | 3 | 15 |
| 3 | 14 | 54 | 28 | 3 | 15 |
| 3 | 14 | 53 | 29 | 3 | 15 |
| 3 | 14 | 52 | 30 | 3 | 15 |
| 3 | 14 | 51 | 31 | 3 | 15 |
| 3 | 14 | 50 | 32 | 3 | 15 |
| 3 | 14 | 49 | 33 | 3 | 15 |
| 3 | 14 | 48 | 34 | 3 | 15 |
| 3 | 14 | 47 | 35 | 3 | 15 |
| 3 | 14 | 46 | 36 | 3 | 15 |
| 3 | 14 | 45 | 37 | 3 | 15 |
| 3 | 14 | 44 | 38 | 3 | 15 |
| 3 | 14 | 43 | 39 | 3 | 15 |
| 3 | 14 | 42 | 40 | 3 | 15 |
| 3 | 14 | 41 | 41 | 3 | 15 |
| 3 | 14 | 40 | 42 | 3 | 15 |
| 3 | 14 | 39 | 43 | 3 | 15 |
| 3 | 14 | 38 | 44 | 3 | 15 |
| 3 | 14 | 37 | 45 | 3 | 15 |
| 3 | 14 | 36 | 46 | 3 | 15 |
| 3 | 14 | 35 | 47 | 3 | 15 |
| 3 | 14 | 34 | 48 | 3 | 15 |
| 3 | 14 | 33 | 49 | 3 | 15 |
| 3 | 14 | 32 | 50 | 3 | 15 |
| 3 | 14 | 31 | 51 | 3 | 15 |
| 3 | 14 | 30 | 52 | 3 | 15 |

### Formulations

In some embodiments, the formulations comprising the LNP and the recombinant RNA molecules have a ratio of the number of nitrogen in the lipids of the LNP to the phosphorous atoms in the recombinant RNA molecules from: 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, or 12.0:1. In some embodiments, the formulations comprising the LNP and the recombinant RNA molecules have a ratio of the number of nitrogen in the lipids of the LNP to the phosphorous atoms in the recombinant RNA molecules (N:P) to: 16.0:1, 15.9:1, 15.8:1, 15.7:1, 15.6:1, 15.5:1, 15.4:1, 15.3:1, 15.2:1, 15.1:1, 15.0:1, 14.9:1, 14.8:1, 14.7:1, 14.6:1, 14.5:1, 14.4:1, 14.3:1, 14.2:1, 14.1:1, 14.0:1, 13.9:1, 13.8:1, 13.7:1, 13.6:1, 13.5:1, 13.4:1, 13.3:1, 13.2:1, 13.1:1, 12.0:1, 12.9:1, 12.8:1, 12.7:1, 12.6:1, 12.5:1, 12.4:1, 12.3:1, 12.2:1, 12.1:1, 12.0:1, 11.9:1, 11.8:1, 11.7:1, 11.6:1, 11.5:1, 11.4:1, 11.3:1, 11.2:1, 11.1:1, 11.0:1, 10.9:1, 10.8:1, 10.7:1, 10.6:1, 10.5:1, 10.4:1, 10.3:1, 10.2:1, 10.1:1, 10.0:1, 9.9:1, 9.8:1, 9.7:1, 9.6:1, 9.5:1, 9.4:1, 9.3:1, 9.2:1, 9.1:1, 9.0:1, 8.9:1, 8.8:1, 8.7:1, 8.6:1, 8.5:1, 8.4:1, 8.3:1, 8.2:1, 8.1:1, 8.0:1, 7.9:1, 7.8:1, 7.7:1, 7.6:1, 7.5:1, 7.4:1, 7.3:1, 7.2:1, 7.1:1, 7.0:1, 6.9:1, 6.8:1, 6.7:1, 6.6:1, 6.5:1, 6.4:1, 6.3:1, 6.2:1, 6.1:1, 6.0:1, 5.9:1, 5.8:1, 5.7:1, 5.6:1, 5.5:1, 5.4:1, 5.3:1, 5.2:1, 5.1:1, 5.0:1, 4.9:1, 4.8:1, 4.7:1, 4.6:1, 4.5:1, 4.4:1, 4.3:1, 4.2:1, 4.1:1, or 4.0:1. Taking the above-noted embodiments into account, it is contemplated and supported that any of the above-noted “ratio of the number of nitrogen in the lipids of the LNP to the phosphorous atoms in the recombinant RNA molecules (N:P) from” and “ratio of the number of nitrogen in the lipids of the LNP to the phosphorous atoms in the recombinant RNA molecules (N:P) to” may be combined to provide an enclosed range (i.e. ratio of the number of nitrogen in the lipids of the LNP to the phosphorous atoms in the recombinant RNA molecules (N:P) from 6:1 to 7:1). In some embodiments, the formulations comprising the LNP and the recombinant RNA molecules have a ratio of the number of nitrogen in the lipids of the LNP to the phosphorous atoms in the recombinant RNA molecules from: 2.0:1 to: 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.1:1 to: 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.2:1 to: 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.3:1 to: 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.4:1 to: 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.5:1 to: 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.6:1 to: 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.7:1 to: 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.8:1 to: 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.9:1 to: 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.0:1 to: 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.1:1 to: 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.2:1 to: 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.3:1 to: 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.4:1 to: 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.5:1 to: 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.6:1 to: 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.7:1 to: 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.8:1 to: 3.9:1, 4.0:1, 4.1:1, 4.2:1, 4.3:1, 4.4:1, 4.5:1, 4.6:1, 2.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.9:1 to: 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.0:1 to: 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.1:1 to: 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.2:1 to: 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.3:1 to: 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.4:1 to: 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.5:1 to: 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.6:1 to: 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.7:1 to: 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.8:1 to: 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.9:1 to: 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.0:1 to: 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.1:1 to: 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.2:1 to: 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.3:1 to: 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.4:1 to: 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.5:1 to: 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.6:1 to: 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.7:1 to: 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.8:1 to: 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.9:1 to: 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.0:1 to: 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.1:1 to: 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.2:1 to: 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.3:1 to: 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.4:1 to: 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.5:1 to: 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.6:1 to: 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.7:1 to: 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.8:1 to: 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.9:1 to: 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.0:1 to: 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.1:1 to: 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.2:1 to: 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.3:1 to: 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.4:1 to: 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.5:1 to: 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.6:1 to 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.7:1 to: 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.8:1 to: 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; or from 7.9:1 to: 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1. Also contemplated and supported are combinations of the above-noted “ratio of the number of nitrogen in the lipids of the LNP to the phosphorous atoms in the recombinant RNA molecules (N:P) from” and “ratio of the number of nitrogen in the lipids of the LNP to the phosphorous atoms in the recombinant RNA molecules (N:P) to” with that provided in the Examples section of this document. Also contemplated and supported are combinations of the “ratio of the number of nitrogen in the lipids of the LNP to the phosphorous atoms in the recombinant RNA molecules (N:P)” of the Example section of this document to provide a range (i.e. “ratio of the number of nitrogen in the lipids of the LNP to the phosphorous atoms in the recombinant RNA molecules (N:P) from [the N:P ratio of formulation X₁] to [N:P ratio of formulation X₂] wherein X₁ and X₂ represent any two exemplary LNP formulations of the Examples.

### Making the LNP Comprising the RNA Molecules

As noted above, In some embodiments, the recombinant RNA molecules are comprised or encapsulated within the LNPs. In some embodiments, the recombinant RNA molecules and lipids of the LNPs can be admixed and/or purified to thereby provide said comprising or encapsulating within. In some embodiments, the RNA molecules and lipids of the LNP can be admixed and/or purified to thereby provide the above-noted proportions of recombinant RNA molecules comprised or encapsulated within the LNPs.

In some aspects, a method of obtaining a composition is provided, the composition comprising the recombinant RNA molecules and LNPs, wherein the recombinant molecules are comprised or encapsulated within the LNPs in the above-noted proportions, wherein the LNPs comprise the above-noted lipids; the method comprising admixing a first solution, which comprises the recombinant RNA molecules, and a second solution, which comprises the above-noted lipids. In some embodiments, the admixing is performed by at least a T-mixer, microfluidics, or an impinging jet mixer. In some embodiments, the first solution further comprises citrate buffer (e.g. sodium citrate) or acetate buffer (e.g. sodium acetate).

In some embodiments, the second solution further comprises an organic solvent. In some embodiments, the organic solvent comprises chloroform, dichloromethane, diethylether, cyclohexane, cyclopentane, benzene, toluene, methanol, benzyl alcohol, and aliphatic alcohols (e.g. C₁ to C₈ alcohols). In some embodiments, the aliphatic alcohols comprise ethanol, propanol, isopropanol, butanol, tert-buranol, isobutanol, pentanol, benzyl alcohol, and hexanol. In some embodiments, the organic solvent comprises an alcohol solution. In some embodiments, the organic alcohol solution comprises from 70 volume % to 100 volume % ethanol.

In some embodiments, the method comprises admixing a first solution, which comprises the recombinant RNA molecules and the above-noted lipids of the LNP, and a second solution, which is an aqueous solution. In some embodiments, the RNA and lipids of the LNP are admixed in an organic solvent. In some embodiments, the organic solvent comprises chloroform, dichloromethane, diethylether, cyclohexane, cyclopentane, benzene, toluene, methanol, benzyl alcohol, and aliphatic alcohols (e.g. C₁ to C₈ alcohols). In some embodiments, the aliphatic alcohols comprise ethanol, propanol, isopropanol, butanol, tert-buranol, isobutanol, pentanol, benzyl alcohol, and hexanol. In some embodiments, the organic solvent comprises an alcohol solution. In some embodiments, the organic alcohol solution comprises from 70 volume % to 100 volume % ethanol. In some embodiments, the organic alcohol solution comprises from 70 volume % to 100 volume % ethanol and 30 volume % to 0 volume % benzyl alcohol. In some embodiments, the aqueous solution comprises a citrate buffer (e.g. sodium citrate) or an acetate buffer (e.g. sodium acetate). In some embodiments, the first and second solution are admixed at a ratio from 1:1 to 5:1, from 2:1 to 4:1, from 2.5:1 to 3.5:1, or at 3:1.

In some embodiments, the admixing of the first and second solutions (i.e. of either of the two above-noted methods) is at a pH from 4.5 to the pKa of the first lipid (e.g. the cation-ionizable lipid), thereby obtaining a first admixture. In some embodiments, the admixing of the first and second solutions is at a pH from 4.5, 4.6, 4.7, 4.8, 4.9, 5.0, 5.1, 5.2, 5.3, 5.4, 5.5, 5.6, 5.7, 5.8, 5.9, or 6.0 to the pKa of the first lipid (e.g. the cation-ionizable lipid), thereby obtaining a first admixture. In some embodiments, the method further comprises a first increasing, which is increasing the pH of the first admixture to be equal to or above the pKa of the first lipid to thereby obtain a pH-adjusted first admixture. In some embodiments, the first increasing obtains a pH-adjusted first admixture with a pH from the pKa of the first lipid (e.g. cation-ionizable lipid) to: 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, or 7.0.

In some embodiments, the first increasing or purifying comprises cross-flow filtration or tangential-flow filtration. In some embodiments, the first increasing or purifying further comprises transferring the composition comprising the LNPs and the recombinant RNA molecules into a third solution, which differs from the first solution. In some embodiments, the third solution comprises phosphate-buffered saline. In some embodiments, the transferring comprises dialysis. In some embodiments, the tangential-flow filtration comprises the use of a hollow fiber filter. In some embodiments, the hollow fiber comprises a polyethersulfone hollow fiber filter or a polysulfone hollow fiber filter. In some embodiments, the hollow fiber filter (e.g. polyethersulfone hollow fiber filter) has a pore size cutoff of at least 75 kDa, 76 kDa, 77 kDa, 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, or 120 kDa. In some embodiments, the hollow fiber filter (e.g. polyethersulfone hollow fiber filter) has a pore size cutoff of no more than 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa. Taking the above-noted embodiments into account, it is contemplated and supported that any of the above-noted pore size cut offs “of at least” and “of no more than” may be combined to provide an enclosed range (i.e. a pore size cut off from 85 kDa to 120 kDa). In some embodiments, the hollow fiber filter (e.g. polyethersulfone hollow fiber filter) has a pore size cutoff of: from 75 kDa to: 76 kDa, 77 kDa, 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 76 kDa to: 77 kDa, 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 77 kDa: to: 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 78 kDa to: 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 79 kDa to: 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 80 kDa to: 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 81 kDa to: 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 82 kDa to: 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 83 kDa to: 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 84 kDa to: 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 85 kDa to: 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 86 kDa to: 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 87 kDa to: 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 88 kDa to: 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 89 kDa to: 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 90 kDa to: 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 91 kDa to: 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 92 kDa to: 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 93 kDa to: 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 94 kDa to: 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 95 kDa to: 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 96 kDa to: 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 97 kDa to: 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 98 kDa to: 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 99 kDa to: 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 100 kDa to: 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 101 kDa to: 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 102 kDa to: 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 103 kDa to: 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 104 kDa to: 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 105 kDa to: 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 106 kDa to: 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 107 kDa to: 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 108 kDa to: 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 109 kDa to: 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 110 kDa to: 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 111 kDa to: 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 112 kDa to: 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 113 kDa to: 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 114 kDa to: 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; or from 115 kDa to: 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa.

In some embodiments, the first increasing or purifying comprises, prior to the above-noted filtrations, passing the LNP/RNA mixture through an ion exchange solid-state support. In some embodiments, the ion exchange solid-state support comprises an anion exchange column or a cation exchange column.

In some embodiments, prior to the admixing of the recombinant RNA molecules and the lipids of the LNPs, the lipids of the LNP admixed with an organic solvent to obtain a concentrated stock (e.g. a stock lipid/organic solvent mixture). In some embodiments, the admixing is (e.g. the stock lipid/organic solvent mixture is stirred, rocked, vortexed, sonicated, or agitated at from 25° C. to 37° C.) for at least 5 min, 6 min, 7 min, 8 min, 9 min, 10 min, 11 min, 12 min, 13 min, 14 min, 15 min, 16 min, 17 min, 18 min, 19 min, 20 min, 25 min, 30 min, 35 min, or 40 min to form a homogeneous stock lipid/organic solvent mixture. In some embodiments, the admixing is (e.g. the stock lipid/organic solvent mixture is stirred, rocked, vortexed, sonicated, or agitated at from 25° C. to 37° C.) for no more than 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs to form a homogeneous stock lipid/organic solvent mixture. Taking the above-noted embodiments into account, it is contemplated and supported that any of the above-noted amounts of time “of at least” and amounts of time “of no more than” may be combined to provide an enclosed range (i.e. the stock lipid/organic solvent mixture is stirred, rocked, vortexed, sonicated, or agitated at from 25° C. to 37° C. for from 5 min to 19 min). In some embodiments, the admixing is (e.g. the stock lipid/organic solvent mixture is stirred, rocked, vortexed, sonicated, or agitated at from 25° C. to 37° C.): from 5 min to: 6 min, 7 min, 8 min, 9 min, 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 6 min to: 7 min, 8 min, 9 min, 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 7 min to: 8 min, 9 min, 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 8 min to: 9 min, 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 9 min to: 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 10 min to: 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 11 min to: 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 12 min to: 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 13 min to: 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 14 min to: 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 15 min to: 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 16 min to: 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 17 min to: 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 18 min to: 19 min, 20 min, 25 min, 30 min, 35 min, 40 min, 50 min, 1 hr, 1.1 hrs, 1.2 hrs, 1.3 hrs, 1.4 hrs, or 1.5 hrs; from 19 min to: 20 min, 25 min, 30 min, 35 min, 40 min, 50 min, 1 hr, 1.1 hrs, 1.2 hrs, 1.3 hrs, 1.4 hrs, or 1.5 hrs; from 20 min to: 25 min, 30 min, 35 min, 40 min, 50 min, 1 hr, 1.1 hrs, 1.2 hrs, 1.3 hrs, 1.4 hrs, or 1.5 hrs; from 25 min to: 30 min, 35 min, 40 min, 50 min, 1 hr, 1.1 hrs, 1.2 hrs, 1.3 hrs, 1.4 hrs, or 1.5 hrs; from 30 min to: 35 min, 40 min, 50 min, 1 hr, 1.1 hrs, 1.2 hrs, 1.3 hrs, 1.4 hrs, or 1.5 hrs; from 35 min to: 35 min, 40 min, 50 min, 1 hr, 1.1 hrs, 1.2 hrs, 1.3 hrs, 1.4 hrs, or 1.5 hrs; from 40 min to: 50 min, 1 hr, 1.1 hrs, 1.2 hrs, 1.3 hrs, 1.4 hrs, or 1.5 hrs; from 50 min to: 1 hr, 1.1 hrs, 1.2 hrs, 1.3 hrs, 1.4 hrs, or 1.5 hrs; from 1 hr to: 1.1 hrs, 1.2 hrs, 1.3 hrs, 1.4 hrs, or 1.5 hrs; from 1.1 hrs to: 1.2 hrs, 1.3 hrs, 1.4 hrs, or 1.5 hrs; or from 1.2 hrs to: 1.3 hrs, 1.4 hrs, or 1.5 hrs to form a homogeneous stock lipid/organic solvent mixture. In some embodiments, the homogenous stock lipid/organic solvent mixture is further diluted in the organic solvent (i.e. ethanol) to obtain the second solution.

### Methods of Administering the RNA

In one aspect, a method of eliciting an immune response in a subject to an immunogen is provided, wherein the method comprises administering to the subject an effective amount of the recombinant RNA molecules or the formulation. In some embodiments, the immune response is a protective immune response. In some embodiments, the immune response or the protective immune response comprises a cell-mediated immune response, an antibody-response (e.g. humoral immune response), a T_{H1} immune response, or a T_{H2} immune response. In some embodiments, the immune response is a therapeutic immune response. In some embodiments, the heterologous polypeptide comprises the antibody against the immunogen. In some embodiments, the heterologous polypeptide comprises the immunogen.

In another aspect, a method of delivering to a subject a heterologous nucleic acid in the recombinant RNA or a formulation comprising the recombinant RNA is provided, wherein the method comprises administering an effective amount of the recombinant RNA.

In some embodiments, the subject is human. In some embodiments, the subject is a mammal, such as a human or a large veterinary mammal (e.g. horses, cattle, deer, goats, pigs). Where the formulation or recombinant RNA is for eliciting a protective immune response, the subject is preferably a human, such as a child (e.g. a toddler or infant), a teenager, and the recombinant RNA or the formulation comprising the recombinant RNA is formulated as a vaccine. Where the composition or recombinant RNA is used as a treatment or for therapeutic use, the human is preferably a teenager or an adult. A vaccine intended for children may also be administered to adults, with the provisio that the amount of recombinant RNA or formulation comprising the recombinant RNA may be scaled up to provide an effective amount consistent with the state of the immune system of the subject (i.e. the elderly having more difficulty eliciting certain immune responses) and the average body weight of a subject of that age or the actual body weight of the subject.

In some embodiments, the recombinant RNA or formulation comprising the recombinant RNA is administered to the subject intramuscularly, intradermally, subcutaneously, transcutaneously, topically, intraperitoneally, intrathecally, pulmonarily (i.e. inhaled), intracerebroventricularly, intravenously, intra-arterially, onto a mucosa (i.e. vaginally), buccally, sublingually, intranasally, optically, to the cornea, or into the eyeball.

In some embodiments, a method for treating cancer in a subject is provided, the subject having a tumor of the cancer, the method comprising administering to the subject the formulation comprising the recombinant RNA or the recombinant RNA or an effective amount thereof. In some embodiments, the recombinant RNA comprises a sequence that encodes a heterologous polypeptide comprising a polyepitopic peptide comprising two or more immunogenic neo-epitopes and a linker, the linker linking the two or more immunogenic neo-epitopes, the immunogenic neo-epitopes being from a first sample comprising cells from the tumor from the subject, each neo-epitope being: (a) encoded in mRNA in the first sample, (b) occurring in a protein-coding region therein, (c) being predicted to bind to a major histocompatibility complex, and (d) being capable of introducing a difference in the amino acid sequence of the neo-epitope when compared to a reference amino acid sequence or genetic sequence predicted to encode the reference amino acid sequence obtained from a second sample from a non-cancerous cell from the subject. In some embodiments, the recombinant RNA is produced from a method comprising: obtaining a first nucleic acid sequence from the first sample, obtaining a second nucleic acid sequence from the second sample, comparing the first nucleic acid sequence to the second nucleic acid sequence thereby obtaining at least two somatic mutations present in the tumor cells, identifying from the at least two somatic mutations (a)-(d), and producing the recombinant RNA.

In one aspect, a method for treating cancer in a subject is provided, the method comprising administering to the subject the recombinant RNA, a formulation comprising the recombinant RNA, or an effective amount thereof, wherein the recombinant RNA comprises a sequence encoding a heterologous polypeptide comprising IL-12 sc, IL-15sushi, IFNα, or GM-CSF. In some embodiments, the method further comprises administering an anti-PD-1/PD-L1 checkpoint inhibitor. In some embodiments, the cancer is melanoma, head and neck squamous cell cancer (HNSCC), cutaneous squamous cell carcinoma (CSCC), or advanced anti-PD-1/PD-L1 naïve cancers thereof.

In some embodiments, a method for treating cancer in a subject is provided, the method comprising administering to the subject the recombinant RNA, a formulation comprising the recombinant RNA, or an effective amount thereof. In some embodiments, the recombinant RNA comprises a sequence encoding a heterologous polypeptide comprising autogene, cevumeran, or atezolizumag. In some embodiments, the cancer is melanoma, head and neck squamous cell cancer (HNSCC), cutaneous squamous cell carcinoma (CSCC), non-small cell lung cancer (NSCLC), or advanced anti-PD-1/PD-L1 naïve cancers thereof.

In some embodiments, the effective amount comprises or is at least: 0.1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules per administration. In some embodiments, the effective amount comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg, 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82 μg, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg, 56 μg, 55 μg, 54 μg, 53 μg, 52 μg, 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, or 15 μg of recombinant RNA molecules per administration. Taking the above-noted embodiments into account, it is contemplated and supported that any of the above-noted “pg” of “at least” and any of the above-noted “pg” of “no more than” may be combined to provide an enclosed range (i.e. the effective amount comprises or is from 25 μg to 75 μg of recombinant RNA molecules per administration). In some embodiments, the effective amount comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 2 μg to: 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 4 μg to: 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 6 μg to: 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 8 μg to: 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 10 μg to: 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 11 μg to: 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 12 μg to: 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 13 μg to: 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 14 μg to: 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 16 μg to: 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 18 μg to: 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 20 μg to: 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 21 μg to: 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 22 μg to: 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 24 μg to: 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 25 μg to: 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 26 μg to: 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 28 μg to: 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 30 μg to: 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 32 μg to: 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 34 μg to: 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 35 μg to: 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 36 μg to: 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 38 μg to: 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 40 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 42 μg to: 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 44 μg to: 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 46 μg to: 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 48 μg to: 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 49 μg to: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 50 μg to: 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 52 μg to: 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 53 μg to: 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 54 μg to: 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 56 μg to: 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 58 μg to: 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to: 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 60 μg to: 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 62 μg to: 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 64 μg to: 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 66 μg to: 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 68 μg to: 69 μg 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 69 μg to: 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 70 μg to: 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 72 μg to: 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 74 μg to: 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 76 μg to: 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 77 μg to: 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 78 μg to: 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 80 μg to: 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules per administration.

Also contemplated and supported are combinations of effective amounts of “at least” and any of the above-noted effective amounts of “no more than” with the effective amounts provided in an Example of this document. Also contemplated and supported are combinations of the effective amounts of the Example section of this document to provide a range (i.e. the effective amount from [the effective amount from example X₁] to [the effective amount from example X₂] wherein X₁ and X₂ represent any two exemplary treatments of the Examples.

In some embodiments, the above-noted methods comprise, is, or consist of more than one administration (i.e. two, three, four, five, six, or seven administrations). In some embodiments, the above-noted methods comprise, is, or consist of at least 2, 3, 4, 5, 6, 7, 8, 9, or 10 administrations (i.e. two, three, four, five, six, or seven administrations). In some embodiments, the above-noted methods comprise, is, or consist of no more than 15, 14, 13, 12, 11, 10, 9, 8, 7, 6, 5, 4, 3, or 2 administrations (i.e. two, three, four, five, six, or seven administrations). Taking the above-noted embodiments into account, it is contemplated and supported that any of the above-noted number of administrations of “at least” and “no more than” may be combined to provide an enclosed range (i.e. from 8 to 15 administrations). In some embodiments, the above-noted methods comprise, is, or consists of from 1 to: 2, 3, 4, 5, 6, 7, 8, 9, 10, 11, 12, 13, 14, or 15; from 2 to: 3, 4, 5, 6, 7, 8, 9, 10, 11, 12, 13, 14, or 15; 3 to: 4, 5, 6, 7, 8, 9, 10, 11, 12, 13, 14, or 15; from 4 to: 5, 6, 7, 8, 9, 10, 11, 12, 13, 14, or 15; from 5 to: 6, 7, 8, 9, 10, 11, 12, 13, 14, or 15; or from 6 to: 7, 8, 9, 10, 11, 12, 13, 14, or 15 administrations.

Also contemplated and supported are combinations of number of administrations of “at least” and number of administrations of “no more than” with the number of administrations provided in an Example of this document. Also contemplated and supported are combinations of the number of administrations of the Example section of this document to provide a range (i.e. the number of administrations from [the number of administrations from example X₁] to [number of administrations of example X₂] wherein X₁ and X₂ represent any two exemplary treatments of the Examples.

In some embodiments, the above-noted methods comprise, is, or consist of more than one administration of an effective amount (i.e. two, three, four, five, six, or seven administrations). In some embodiments, the above-noted methods comprise, is, or consist of at least 2, 3, 4, 5, 6, 7, 8, 9, or 10 administrations of an effective amount (i.e. two, three, four, five, six, or seven administrations of an effective amount). In some embodiments, the above-noted methods comprise, is, or consist of no more than 15, 14, 13, 12, 11, 10, 9, 8, 7, 6, 5, 4, 3, or 2 administrations of an effective amount (i.e. two, three, four, five, six, or seven administrations of an effective amount). Taking the above-noted embodiments into account, it is contemplated and supported that any of the above-noted number of administrations of the effective amount of “at least” and “no more than” may be combined to provide an enclosed range (i.e. from 8 to 15 administrations of the effective amount). In some embodiments, the above-noted methods comprise, is, or consists of from 1 to: 2, 3, 4, 5, 6, 7, 8, 9, 10, 11, 12, 13, 14, or 15; from 2 to: 3, 4, 5, 6, 7, 8, 9, 10, 11, 12, 13, 14, or 15; 3 to: 4, 5, 6, 7, 8, 9, 10, 11, 12, 13, 14, or 15; from 4 to: 5, 6, 7, 8, 9, 10, 11, 12, 13, 14, or 15; from 5 to: 6, 7, 8, 9, 10, 11, 12, 13, 14, or 15; or from 6 to: 7, 8, 9, 10, 11, 12, 13, 14, or 15 administrations of the effective amount.

Also contemplated and supported are combinations of number of administrations of “at least” and number of administrations of an effective amount of “no more than” with the number of administrations of an effective amount provided in an Example of this document. Also contemplated and supported are combinations of the number of administrations of an effective amount of the Example section of this document to provide a range (i.e. the number of administrations from [the number of administrations from example X₁] to [number of administrations of example X₂] wherein X, and X₂ represent any two exemplary treatments of the Examples.

In some embodiments, the administration comprises a primary administration and a booster administration. In some embodiments, the effective amount differs between the primary administration and the booster administration.

In some embodiments, the above-noted methods comprise, is, or consist of more than one primary administration (i.e. two, three, four, five, six, or seven administrations). In some embodiments, the above-noted methods comprise, is, or consist of at least 2, 3, 4, 5, 6, 7, 8, 9, or 10 primary administrations (i.e. two, three, four, five, six, or seven administrations of an effective amount). In some embodiments, the above-noted methods comprise, is, or consist of no more than 15, 14, 13, 12, 11, 10, 9, 8, 7, 6, 5, 4, 3, or 2 primary administrations (i.e. two, three, four, five, six, or seven administrations of an effective amount). Taking the above-noted embodiments into account, it is contemplated and supported that any of the above-noted number of primary administrations of “at least” and “no more than” may be combined to provide an enclosed range (i.e. from 8 to 15 primary administrations). In some embodiments, the above-noted methods comprise, is, or consists of from 1 to: 2, 3, 4, 5, 6, 7, 8, 9, 10, 11, 12, 13, 14, or 15; from 2 to: 3, 4, 5, 6, 7, 8, 9, 10, 11, 12, 13, 14, or 15; 3 to: 4, 5, 6, 7, 8, 9, 10, 11, 12, 13, 14, or 15; from 4 to: 5, 6, 7, 8, 9, 10, 11, 12, 13, 14, or 15; from 5 to: 6, 7, 8, 9, 10, 11, 12, 13, 14, or 15; or from 6 to: 7, 8, 9, 10, 11, 12, 13, 14, or 15 primary administrations.

Also contemplated and supported are combinations of the number of primary administrations of “at least” and the number of primary administrations of “no more than” with the number of primary administrations provided in an Example of this document. Also contemplated and supported are combinations of the number of primary administrations of the Example section of this document to provide a range (i.e. the number of primary administrations from [the number of primary administrations from example X₁] to [number of primary administrations of example X₂] wherein X, and X₂ represent any two exemplary treatments of the Examples.

In some embodiments, the primary administration comprises or is at least: 0.1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules per primary administration. In some embodiments, the effective amount comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg, 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82 μg, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg, 56 μg, 55 μg, 54 μg, 53 μg, 52 μg, 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, or 15 μg of recombinant RNA molecules per primary administration. Taking the above-noted embodiments into account, it is contemplated and supported that any of the above-noted “pg” of “at least” and any of the above-noted “pg” of “no more than” may be combined to provide an enclosed range (i.e. the primary administration comprises or is from 25 μg to 75 μg of recombinant RNA molecules per primary administration). In some embodiments, the primary administration comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 2 μg to: 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 4 μg to: 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 6 μg to: 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 8 μg to: 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 10 μg to: 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 11 μg to: 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 12 μg to: 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 13 μg to: 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 14 μg to: 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 16 μg to: 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 18 μg to: 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 20 μg to: 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 21 μg to: 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 22 μg to: 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 24 μg to: 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 25 μg to: 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 26 μg to: 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 28 μg to: 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 30 μg to: 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 32 μg to: 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 34 μg to: 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 35 μg to: 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 36 μg to: 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 38 μg to: 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 40 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 42 μg to: 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 44 μg to: 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 46 μg to: 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 48 μg to: 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 49 μg to: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 50 μg to: 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 52 μg to: 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 53 μg to: 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 54 μg to: 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 56 μg to: 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 58 μg to: 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to: 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 60 μg to: 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 62 μg to: 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 64 μg to: 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 66 μg to: 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 68 μg to: 69 μg 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 69 μg to: 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 70 μg to: 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 72 μg to: 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 74 μg to: 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 76 μg to: 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 77 μg to: 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 78 μg to: 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 80 μg to: 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules per primary administration.

Also contemplated and supported are combinations of primary administrations of “at least” and any of the above-noted primary administrations of “no more than” with the primary administrations provided in an Example of this document. Also contemplated and supported are combinations of the primary administrations of the Example section of this document to provide a range (i.e. the primary administration from [the primary administration from example X₁] to [the primary administration from example X₂] wherein X₁ and X₂ represent any two exemplary treatments of the Examples.

In some embodiments, the immune response that the primary administration elicits comprises a cell-mediated immune response, an antibody-response (e.g. humoral immune response), a T_{H1} immune response, or a T_{H2} immune response. In some embodiments, the eliciting of the immune response by the primary administration is from a naïve immune response where the immune system has no detectable antibody response that is against the immunogen, cell-mediated immune response that is against the immunogen, T_{H1} immune response that is against the immunogen, or a T_{H2} immune response that is against the immunogen to a cell-mediated immune response that is against the immunogen, an antibody-response (e.g. humoral immune response) that is against the immunogen, a T_{H1} immune response that is against the immunogen, or a T_{H2} immune response that is against the immunogen. In some embodiments, the eliciting of the immune response by the primary administration is from an experienced immune response where the antibody response that is against the immunogen, the cell-mediated immune response that is against the immunogen, the T_{H1} immune response that is against the immunogen, or the T_{H2} immune response that is against the immunogen is unable to reduce the likelihood of at least one symptom of the disease or from being infectious to others by the disease to a cell-mediated immune response that is against the immunogen, an antibody-response (e.g. humoral immune response) that is against the immunogen, a T_{H1} immune response that is against the immunogen, or a T_{H2} immune response that is against the immunogen that reduces the likelihood of at least one symptom of the disease or the likelihood of being infectious to others by the disease. In some embodiments, at least one symptom of the disease comprises death, respiratory distress, reduced blood oxygen, fever, chills, a febrile response, shortness of breath, difficulty breathing, fatigue, muscle aches, body aches, headaches, new or loss of smell, mucus production, sore throat, congestion, runny nose, nausea, vomiting, diarrhea, confusion, pressure in the chest, persistent pain, inability to wake, inability to stay awake, pale skin, gray skin, blue-colored skin, pale lips, gray lips, blue-colored lips, pale nail-beds, gray nail-beds, blue-colored nail-beds, low reperfusion of extremities, low reperfusion of the body, encephalitis, stroke, ischemia, fibromyalgia, or myocarditis.

In some embodiments, the above-noted methods comprise, is, or consist of more than one booster administration (i.e. two, three, four, five, six, or seven administrations). In some embodiments, the above-noted methods comprise, is, or consist of at least 2, 3, 4, 5, 6, 7, 8, 9, or 10 booster administrations (i.e. two, three, four, five, six, or seven administrations of an effective amount). In some embodiments, the above-noted methods comprise, is, or consist of no more than 15, 14, 13, 12, 11, 10, 9, 8, 7, 6, 5, 4, 3, or 2 booster administrations (i.e. two, three, four, five, six, or seven administrations of an effective amount). Taking the above-noted embodiments into account, it is contemplated and supported that any of the above-noted number of booster administrations of “at least” and “no more than” may be combined to provide an enclosed range (i.e. from 8 to 15 booster administrations). In some embodiments, the above-noted methods comprise, is, or consists of from 1 to: 2, 3, 4, 5, 6, 7, 8, 9, 10, 11, 12, 13, 14, or 15; from 2 to: 3, 4, 5, 6, 7, 8, 9, 10, 11, 12, 13, 14, or 15; 3 to: 4, 5, 6, 7, 8, 9, 10, 11, 12, 13, 14, or 15; from 4 to: 5, 6, 7, 8, 9, 10, 11, 12, 13, 14, or 15; from 5 to: 6, 7, 8, 9, 10, 11, 12, 13, 14, or 15; or from 6 to: 7, 8, 9, 10, 11, 12, 13, 14, or 15 booster administrations.

Also contemplated and supported are combinations of the number of booster administrations of “at least” and the number of booster administrations of “no more than” with the number of booster administrations provided in an Example of this document. Also contemplated and supported are combinations of the number of booster administrations of the Example section of this document to provide a range (i.e. the number of booster administrations from [the number of booster administrations from example X₁] to [number of booster administrations of example X₂] wherein X₁ and X₂ represent any two exemplary treatments of the Examples.

In some embodiments, the booster administration comprises or is at least: 0.1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules per booster administration. In some embodiments, the effective amount comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg, 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82 μg, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg, 56 μg, 55 μg, 54 μg, 53 μg, 52 μg, 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, or 15 μg of recombinant RNA molecules per booster administration. Taking the above-noted embodiments into account, it is contemplated and supported that any of the above-noted “pg” of “at least” and any of the above-noted “μg” of “no more than” may be combined to provide an enclosed range (i.e. the booster administration comprises or is from 25 μg to 75 μg of recombinant RNA molecules per booster administration). In some embodiments, the booster administration comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 2 μg to: 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 4 μg to: 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 6 μg to: 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 8 μg to: 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 10 μg to: 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 11 μg to: 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 12 μg to: 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 13 μg to: 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 14 μg to: 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 16 μg to: 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 18 μg to: 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 20 μg to: 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 21 μg to: 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 22 μg to: 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 24 μg to: 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 25 μg to: 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 26 μg to: 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 28 μg to: 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 30 μg to: 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 32 μg to: 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 34 μg to: 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 35 μg to: 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 36 μg to: 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 38 μg to: 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 40 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 42 μg to: 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 44 μg to: 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 46 μg to: 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 48 μg to: 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 49 μg to: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 50 μg to: 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 52 μg to: 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 53 μg to: 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 54 μg to: 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 56 μg to: 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 58 μg to: 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to:60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 60 μg to: 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 62 μg to: 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 64 μg to: 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 66 μg to: 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 68 μg to: 69 μg 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 69 μg to: 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 70 μg to: 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 72 μg to: 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 74 μg to: 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 76 μg to: 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 77 μg to: 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 78 μg to: 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 80 μg to: 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules per booster administration.

Also contemplated and supported are combinations of booster administrations of “at least” and any of the above-noted booster administrations of “no more than” with the booster administrations provided in an Example of this document. Also contemplated and supported are combinations of the booster administrations of the Example section of this document to provide a range (i.e. the booster administration from [the booster administration from example X₁] to [the booster administration from example X₂] wherein X₁ and X₂ represent any two exemplary treatments of the Examples.

In some embodiments, the immune response that the booster administration elicits comprises a cell-mediated immune response, an antibody-response (e.g. humoral immune response), a T_{H1} immune response, or a T_{H2} immune response. In some embodiments, the eliciting of the immune response by the booster administration is from an experienced immune response where the antibody response that is against the immunogen, the cell-mediated immune response that is against the immunogen, the T_{H1}immune response that is against the immunogen, or the T_{H2} immune response that is against the immunogen is unable to reduce the likelihood of at least one symptom of the disease or from being infectious to others by the disease to a cell-mediated immune response that is against the immunogen, an antibody-response (e.g. humoral immune response) that is against the immunogen, a T_{H1} immune response that is against the immunogen, or a T_{H2} immune response that is against the immunogen that reduces the likelihood of at least one symptom of the disease or the likelihood of being infectious to others by the disease. In some embodiments, the eliciting of the immune response by the booster administration is from an experienced immune response where the antibody response that is against the immunogen, the cell-mediated immune response that is against the immunogen, the T_{H1} immune response that is against the immunogen, or the T_{H2} immune response that is against the immunogen is, at the time of booster administration, able to reduce the likelihood of at least one symptom of the disease or from being infectious to others by the disease, but some time thereafter and without the booster administration, would not be able to reduce the likelihood of at least one symptom of the disease or from being infectious to others by the disease (i.e. a boosting of the duration of the immune response). In such cases, the booster administration elicits an immune response when compared to the same individual who would not have received the booster administration and who would have otherwise not been able to reduce the likelihood of at least one symptom of the disease or from being infectious to others by the disease (i.e. boosted duration). In some embodiments of such cases, the eliciting of the immune response by the booster administration comprises a renewed cell-mediated immune response that is against the immunogen, a renewed antibody-response (e.g. humoral immune response) that is against the immunogen, a renewed T_{H1} immune response that is against the immunogen, or a renewed T_{H2} immune response that is against the immunogen that reduces the likelihood of at least one symptom of the disease or the likelihood of being infectious to others by the disease during the period in which, without the booster administration, the subject would have otherwise had an increased likelihood of at least one symptom of the disease or an increased likelihood of being infectious to the disease (i.e. boosted duration). In some embodiments, the booster administration elicits an immune response that is above the immune response elicited by the primary administration. In some embodiments, the immune response that is elicited by the booster administration is an antibody response that is above that elicited by the primary administration, a cell-mediated immune response that is above that elicited by the primary administration, a T_{H1} immune response that is above that elicited by the primary administration, or a T_{H2} immune response that is above that elicited by the primary administration. In some embodiments, at least one symptom of the disease comprises death, respiratory distress, reduced blood oxygen, fever, chills, a febrile response, shortness of breath, difficulty breathing, fatigue, muscle aches, body aches, headaches, new or loss of smell, mucus production, sore throat, congestion, runny nose, nausea, vomiting, diarrhea, confusion, pressure in the chest, persistent pain, inability to wake, inability to stay awake, pale skin, gray skin, blue-colored skin, pale lips, gray lips, blue-colored lips, pale nail-beds, gray nail-beds, blue-colored nail-beds, low reperfusion of extremities, low reperfusion of the body, encephalitis, stroke, ischemia, fibromyalgia, or myocarditis.

In one aspect, a method of eliciting an immune response in a subject to an immunogen is provided, wherein the method comprises administering to the subject a unit dose of the recombinant RNA molecules or the formulation. A “unit dose” is contemplated and understood to be that dose provided in an administration, and several unit doses (i.e. the unit doses from several administrations may be combined) to make a total dose administered to the subject. In some embodiments, the immune response is a protective immune response. In some embodiments, the immune response or the protective immune response comprises a cell-mediated immune response, an antibody-response (e.g. humoral immune response), a T_{H1} immune response, or a T_{H2} immune response. In some embodiments, the immune response is a therapeutic immune response. In some embodiments, the heterologous polypeptide comprises the antibody against the immunogen. In some embodiments, the heterologous polypeptide comprises the immunogen.

In another aspect, a method of delivering to a subject a heterologous nucleic acid in the recombinant RNA or a formulation comprising the recombinant RNA is provided, wherein the method comprises administering an unit dose of the recombinant RNA.

In some embodiments, the subject is human. In some embodiments, the subject is a mammal, such as a human or a large veterinary mammal (e.g. horses, cattle, deer, goats, pigs). Where the formulation or recombinant RNA is for eliciting a protective immune response, the subject is preferably a human, such as a child (e.g. a toddler or infant), a teenager, and the recombinant RNA or the formulation comprising the recombinant RNA is formulated as a vaccine. Where the composition or recombinant RNA is used as a treatment or for therapeutic use, the human is preferably a teenager or an adult. A vaccine intended for children may also be administered to adults, with the provisio that the amount of recombinant RNA or formulation comprising the recombinant RNA may be scaled up to provide an unit dose consistent with the state of the immune system of the subject (i.e. the elderly having more difficulty eliciting certain immune responses) and the average body weight of a subject of that age or the actual body weight of the subject.

In some embodiments, the recombinant RNA or formulation comprising the recombinant RNA is administered to the subject intramuscularly, intradermally, subcutaneously, transcutaneously, topically, intraperitoneally, intrathecally, pulmonarily (i.e. inhaled), intracerebroventricularly, intravenously, intra-arterially, onto a mucosa (i.e. vaginally), buccally, sublingually, intranasally, optically, to the cornea, or into the eyeball.

In some embodiments, a method for treating cancer in a subject is provided, the subject having a tumor of the cancer, the method comprising administering to the subject the formulation comprising the recombinant RNA or the recombinant RNA or an unit dose thereof. In some embodiments, the recombinant RNA comprises a sequence that encodes a heterologous polypeptide comprising a polyepitopic peptide comprising two or more immunogenic neo-epitopes and a linker, the linker linking the two or more immunogenic neo-epitopes, the immunogenic neo-epitopes being from a first sample comprising cells from the tumor from the subject, each neo-epitope being: (a) encoded in mRNA in the first sample, (b) occurring in a protein-coding region therein, (c) being predicted to bind to a major histocompatibility complex, and (d) being capable of introducing a difference in the amino acid sequence of the neo-epitope when compared to a reference amino acid sequence or genetic sequence predicted to encode the reference amino acid sequence obtained from a second sample from a non-cancerous cell from the subject. In some embodiments, the recombinant RNA is produced from a method comprising: obtaining a first nucleic acid sequence from the first sample, obtaining a second nucleic acid sequence from the second sample, comparing the first nucleic acid sequence to the second nucleic acid sequence thereby obtaining at least two somatic mutations present in the tumor cells, identifying from the at least two somatic mutations (a)-(d), and producing the recombinant RNA.

In one aspect, a method for treating cancer in a subject is provided, the method comprising administering to the subject the recombinant RNA, a formulation comprising the recombinant RNA, or an unit dose thereof, wherein the recombinant RNA comprises a sequence encoding a heterologous polypeptide comprising IL-12 sc, IL-15sushi, IFNα, or GM-CSF. In some embodiments, the method further comprises administering an anti-PD-1/PD-L1 checkpoint inhibitor. In some embodiments, the cancer is melanoma, head and neck squamous cell cancer (HNSCC), cutaneous squamous cell carcinoma (CSCC), or advanced anti-PD-1/PD-L1 naïve cancers thereof.

In some embodiments, a method for treating cancer in a subject is provided, the method comprising administering to the subject the recombinant RNA, a formulation comprising the recombinant RNA, or an unit dose thereof. In some embodiments, the recombinant RNA comprises a sequence encoding a heterologous polypeptide comprising autogene, cevumeran, or atezolizumag. In some embodiments, the cancer is melanoma, head and neck squamous cell cancer (HNSCC), cutaneous squamous cell carcinoma (CSCC), non-small cell lung cancer (NSCLC), or advanced anti-PD-1/PD-L1 naïve cancers thereof.

In some embodiments, the unit dose comprises or is at least: 0.1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules. In some embodiments, the unit dose comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg, 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82 μg, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg, 56 μg, 55 μg, 54 μg, 53 μg, 52 μg, 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, or 15 μg of recombinant RNA molecules. Taking the above-noted embodiments into account, it is contemplated and supported that any of the above-noted “μg” of “at least” and any of the above-noted “μg” of “no more than” may be combined to provide an enclosed range (i.e. the unit dose comprises or is from 25 μg to 75 μg). In some embodiments, the unit dose comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from2 μg to: 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 4 μg to: 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 6 μg to: 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 8 μg to: 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 10 μg to: 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 11 μg to: 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 12 μg to: 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 13 μg to: 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 14 μg to: 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 16 μg to: 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 18 μg to: 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 20 μg to: 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 21 μg to: 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 22 μg to: 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 24 μg to: 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 25 μg to: 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 26 μg to: 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 28 μg to: 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 30 μg to: 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 32 μg to: 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 34 μg to: 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 35 μg to: 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 36 μg to: 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 38 μg to: 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 40 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 42 μg to: 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 44 μg to: 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 46 μg to: 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 48 μg to: 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 49 μg to: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 50 μg to: 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 52 μg to: 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 53 μg to: 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 54 μg to: 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 56 μg to: 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 58 μg to: 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to:60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 60 μg to: 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 62 μg to: 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 64 μg to: 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 66 μg to: 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 68 μg to: 69 μg 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 69 μg to: 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 70 μg to: 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 72 μg to: 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 74 μg to: 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 76 μg to: 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 77 μg to: 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 78 μg to: 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 80 μg to: 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules.

Also contemplated and supported are combinations of unit doses of “at least” and any of the above-noted unit doses of “no more than” with the unit doses provided in an Example of this document. Also contemplated and supported are combinations of the unit doses of the Example section of this document to provide a range (i.e. the unit dose from [the unit dose from example X₁] to [the unit dose from example X₂] wherein X₁ and X₂ represent any two exemplary treatments of the Examples.

### Uses to Form Medicaments Comprising the Recombinant RNA

In one aspect, a use of the recombinant RNA molecules for the manufacture of a medicament for delivering the segment that encodes the heterologous polypeptide is provided; the use comprising admixing the recombinant RNA molecules with the lipids of the lipid nanoparticle (LNP), thereby obtaining the formulation, including the formulation described above.

In another aspect, a use of the formulation for the manufacture of a medicament for delivering the segment that encodes the heterologous polypeptide is provided; the use comprising admixing the formulation comprising the recombinant RNA molecules and optionally the LNPs, including formulation, the recombinant RNA molecule, and the LNPs described above, with a pharmaceutically acceptable vehicle. In some embodiments, the pharmaceutically acceptable vehicle is a buffer or saline. In some embodiments, the buffer is phosphate buffered saline.

In another aspect, a use of the formulation for the manufacture of a medicament for delivering the segment that encodes the heterologous polypeptide is provided; the use comprising admixing the formulation, including the formulation described above, with LNPs that do not comprise RNA, LNPs that comprise RNA that does not comprise the segment that encodes the heterologous polypeptide, or LNPs that comprise recombinant RNA that comprises a segment that encodes a second heterologous polypeptide.

In one aspect, a use of the recombinant RNA molecules for the manufacture of a medicament for preventing a disease caused by a pathogen is provided; the pathogen comprising the immunogen; the use comprising admixing the recombinant RNA molecules with the lipids of the lipid nanoparticle (LNP), thereby obtaining the formulations described above. In some embodiments, the segment that encodes a heterologous polypeptide comprises a segment that encodes the immunogen or an antibody that is against the immunogen.

In one aspect, a use of the recombinant RNA molecules for the manufacture of a medicament for treating a disease caused by a pathogen is provided; the pathogen comprising the immunogen; the use comprising admixing the recombinant RNA molecules with the lipids of the lipid nanoparticle (LNP), thereby obtaining the formulations described above. In some embodiments, the segment that encodes a heterologous polypeptide comprises a segment that encodes the immunogen or an antibody that is against the immunogen.

In another aspect, a use of the formulation for the manufacture of a medicament for preventing a disease caused by a pathogen is provided; the pathogen comprising the immunogen; the use comprising admixing the formulation, including the formulation described above, with a pharmaceutically acceptable vehicle. In some embodiments, the pharmaceutically acceptable vehicle is a buffer or saline. In some embodiments, the buffer is phosphate buffered saline.

In another aspect, a use of the formulation for the manufacture of a medicament for treating a disease caused by a pathogen is provided; the pathogen comprising the immunogen; the use comprising admixing the formulation, including the formulation described above, with a pharmaceutically acceptable vehicle. In some embodiments, the pharmaceutically acceptable vehicle is a buffer or saline. In some embodiments, the buffer is phosphate buffered saline.

In another aspect, a use of the formulation for the manufacture of a medicament for preventing a disease caused by a pathogen is provided; the pathogen comprising the immunogen; the use comprising admixing the formulation, including the formulation described above, with LNPs that do not comprise RNA, LNPs that comprise RNA that does not comprise the segment that encodes the heterologous polypeptide, or LNPs that comprise recombinant RNA that comprises a segment that encodes a second heterologous polypeptide. In some embodiments, the segment that encodes a heterologous polypeptide comprises a segment that encodes the immunogen or an antibody that is against the immunogen.

In another aspect, a use of the formulation for the manufacture of a medicament for treating a disease caused by a pathogen is provided; the pathogen comprising the immunogen; the use comprising admixing the formulation, including the formulation described above, with LNPs that do not comprise RNA, LNPs that comprise RNA that does not comprise the segment that encodes the heterologous polypeptide, or LNPs that comprise recombinant RNA that comprises a segment that encodes a second heterologous polypeptide. In some embodiments, the segment that encodes a heterologous polypeptide comprises a segment that encodes the immunogen or an antibody that is against the immunogen.

### Formulation for Use in Delivering, Eliciting an Immune Response, Preventing an Infection by a Pathogen

In another aspect, a formulation for use in delivering nucleic acid that encodes a heterologous polypeptide is provided; the formulation comprising the recombinant RNA molecules and the LNP, including the recombinant RNA molecules and the LNPs described above; the recombinant RNA molecules comprising the segment that encodes the heterologous polypeptide. In some embodiments, the subject is a mammal, such as a human or a large veterinary mammal (e.g. horses, cattle, deer, goats, pigs). Where the formulation or recombinant RNA is for eliciting a protective immune response, the subject is preferably a human, such as a child (e.g. a toddler or infant), a teenager, and the recombinant RNA or the formulation comprising the recombinant RNA is formulated as a vaccine. Where the composition or recombinant RNA is used as a treatment or for therapeutic use, the human is preferably a teenager or an adult. A vaccine intended for children may also be administered to adults, with the provisio that the amount of recombinant RNA or formulation comprising the recombinant RNA may be scaled up to provide an effective amount consistent with the state of the immune system of the subject (i.e. the elderly having more difficulty eliciting certain immune responses) and the average body weight of a subject of that age or the actual body weight of the subject.

In another aspect, a formulation for expressing a heterologous polypeptide is provided; the formulation comprising the recombinant RNA molecules and the LNP, including the recombinant RNA molecules and the LNPs described above; the recombinant RNA molecules comprising the segment that encodes the heterologous polypeptide. In some embodiments, the subject is a mammal, such as a human or a large veterinary mammal (e.g. horses, cattle, deer, goats, pigs). Where the formulation or recombinant RNA is for eliciting a protective immune response, the subject is preferably a human, such as a child (e.g. a toddler or infant), a teenager, and the recombinant RNA or the formulation comprising the recombinant RNA is formulated as a vaccine. Where the composition or recombinant RNA is used as a treatment or for therapeutic use, the human is preferably a teenager or an adult. A vaccine intended for children may also be administered to adults, with the provisio that the amount of recombinant RNA or formulation comprising the recombinant RNA may be scaled up to provide an effective amount consistent with the state of the immune system of the subject (i.e. the elderly having more difficulty eliciting certain immune responses) and the average body weight of a subject of that age or the actual body weight of the subject.

In another aspect, a formulation for use in preventing infection by a pathogen in a subject is provided; the formulation comprising the recombinant RNA molecules and the LNPs, including the recombinant RNA molecules and the LNPs described above; the segment that encodes a heterologous polypeptide comprising a segment that encodes an immunogen or an antibody against the immunogen; the pathogen comprising the immunogen. In some embodiments, the subject is a mammal, such as a human or a large veterinary mammal (e.g. horses, cattle, deer, goats, pigs). Where the formulation or recombinant RNA is for eliciting a protective immune response, the subject is preferably a human, such as a child (e.g. a toddler or infant), a teenager, and the recombinant RNA or the formulation comprising the recombinant RNA is formulated as a vaccine. Where the composition or recombinant RNA is used as a treatment or for therapeutic use, the human is preferably a teenager or an adult. A vaccine intended for children may also be administered to adults, with the provisio that the amount of recombinant RNA or formulation comprising the recombinant RNA may be scaled up to provide an effective amount consistent with the state of the immune system of the subject (i.e. the elderly having more difficulty eliciting certain immune responses) and the average body weight of a subject of that age or the actual body weight of the subject.

In another aspect, a formulation for use in eliciting an immune response in a subject is provided; the formulation comprising the recombinant RNA molecules and the LNPs, including the recombinant RNA molecules and the LNPs described above; the segment that encodes a heterologous polypeptide comprising a segment that encodes an immunogen or an antibody against the immunogen. In some embodiments, the subject is a mammal, such as a human or a large veterinary mammal (e.g. horses, cattle, deer, goats, pigs). Where the formulation or recombinant RNA is for eliciting a protective immune response, the subject is preferably a human, such as a child (e.g. a toddler or infant), a teenager, and the recombinant RNA or the formulation comprising the recombinant RNA is formulated as a vaccine. Where the composition or recombinant RNA is used as a treatment or for therapeutic use, the human is preferably a teenager or an adult. A vaccine intended for children may also be administered to adults, and it is contemplated and supported that the amount of recombinant RNA or formulation comprising the recombinant RNA may be scaled up to provide an effective amount consistent with the state of the immune system of the subject (i.e. the elderly having more difficulty eliciting certain immune responses) and the average body weight of a subject of that age or the actual body weight of the subject.

### CLAUSES

The aspects and embodiments of the invention are further illustrated by reference to the following clauses:
Clause 1 A recombinant ribonucleic acid (RNA) molecule comprising an optional 5′ cap nucleoside (i.e. a 7-methylguanosine or a modified 7-methylguanosine), an optional linker, an optional first 5′ ribonucleoside, a 5′ untranslated region (5′ UTR), a segment that encodes a heterologous polypeptide, a 3′ untranslated region (3′ UTR), and an optional 3′ poly(adenosine monophosphate) (poly(A)) tail; wherein:
   (a) the 5′ UTR is 5′ of the segment that encodes the heterologous polypeptide;
   (b) the 3′ UTR is 3′ of the segment that encodes the heterologous polypeptide;
   (c) the optional 3′ poly(A) is 3′ of the 3′ UTR; and
   (d) (i) the 5′ UTR comprises a sequence that has at least 70% sequence identity to SEQ ID NO: 1, SEQ ID NO: 3, SEQ ID NO: 5, SEQ ID NO: 7, SEQ ID NO: 9, SEQ ID NO: 11, or SEQ ID NO: 13; or
   (ii) the 3′ UTR comprises a sequence that has at least 70% sequence identity to SEQ ID NO: 2, SEQ ID NO: 4, SEQ ID NO: 6, SEQ ID NO: 8, SEQ ID NO: 10, SEQ ID NO: 12, or SEQ ID NO: 14.
Clause 2 The RNA molecule according to clause 1, wherein the 5′ UTR comprises a sequence that has at least 70% sequence identity to SEQ ID NO: 1, SEQ ID NO: 3, SEQ ID NO: 5, SEQ ID NO: 7, SEQ ID NO: 9, SEQ ID NO: 11, or SEQ ID NO: 13.
Clause 3 The RNA molecule according to clause 1, wherein the 3′ UTR comprises a sequence that has at least 70% sequence identity to SEQ ID NO: 2, SEQ ID NO: 4, SEQ ID NO: 6, SEQ ID NO: 8, SEQ ID NO: 10, SEQ ID NO: 12, or SEQ ID NO: 14.
Clause 4 The RNA molecule according to clause 1, wherein the 5′ UTR comprises a sequence that has at least 70% sequence identity to SEQ ID NO: 1, SEQ ID NO: 3, SEQ ID NO: 5, SEQ ID NO: 7, SEQ ID NO: 9, SEQ ID NO: 11, or SEQ ID NO: 13; and the 3′ UTR comprises a sequence that has at least 70% sequence identity to SEQ ID NO: 2, SEQ ID NO: 4, SEQ ID NO: 6, SEQ ID NO: 8, SEQ ID NO: 10, SEQ ID NO: 12, or SEQ ID NO: 14.
Clause 5 The RNA molecule according to any one of clauses 1 to 4, wherein the 5′ UTR comprises a sequence that has at least 70% sequence identity to SEQ ID NO: 1.
Clause 6 The RNA molecule according to any one of clauses 1 to 4, wherein the 5′ UTR consists of a sequence that has at least 70% sequence identity to SEQ ID NO: 1.
Clause 7 The RNA molecule according to any one of clauses 1 to 4, wherein the 5′ UTR comprises a sequence that has at least 70% sequence identity to SEQ ID NO: 3.
Clause 8 The RNA molecule according to any one of clauses 1 to 4, wherein the 5′ UTR consists of a sequence that has at least 70% sequence identity to SEQ ID NO: 3.
Clause 9 The RNA molecule according to any one of clauses 1 to 4, wherein the 5′ UTR comprises a sequence that has at least 70% sequence identity to SEQ ID NO: 5.
Clause 10 The RNA molecule according to any one of clauses 1 to 4, wherein the 5′ UTR consists of a sequence that has at least 70% sequence identity to SEQ ID NO: 5.
Clause 11 The RNA molecule according to any one of clauses 1 to 4, wherein the 5′ UTR comprises a sequence that has at least 70% sequence identity to SEQ ID NO: 7.
Clause 12 The RNA molecule according to any one of clauses 1 to 4, wherein the 5′ UTR consists of a sequence that has at least 70% sequence identity to SEQ ID NO: 7.
Clause 13 The RNA molecule according to any one of clauses 1 to 4, wherein the 5′ UTR comprises a sequence that has at least 70% sequence identity to SEQ ID NO: 9.
Clause 14 The RNA molecule according to any one of clauses 1 to 4, wherein the 5′ UTR consists of a sequence that has at least 70% sequence identity to SEQ ID NO: 9.
Clause 15 The RNA molecule according to any one of clauses 1 to 4, wherein the 5′ UTR comprises a sequence that has at least 70% sequence identity to SEQ ID NO: 11.
Clause 16 The RNA molecule according to any one of clauses 1 to 4, wherein the 5′ UTR consists of a sequence that has at least 70% sequence identity to SEQ ID NO: 11.
Clause 17 The RNA molecule according to any one of clauses 1 to 4, wherein the 5′ UTR comprises a sequence that has at least 70% sequence identity to SEQ ID NO: 13.
Clause 18 The RNA molecule according to any one of clauses 1 to 4, wherein the 5′ UTR consists of a sequence that has at least 70% sequence identity to SEQ ID NO: 13.
Clause 19 The RNA molecule according to any one of clauses 1 to 18, wherein the 3′ UTR comprises a sequence that has at least 70% sequence identity to SEQ ID NO: 2.
Clause 20 The RNA molecule according to any one of clauses 1 to 18, wherein the 3′ UTR consists of a sequence that has at least 70% sequence identity to SEQ ID NO: 2.
Clause 21 The RNA molecule according to any one of clauses 1 to 18, wherein the 3′ UTR comprises a sequence that has at least 70% sequence identity to SEQ ID NO: 4.
Clause 22 The RNA molecule according to any one of clauses 1 to 18, wherein the 3′ UTR consists of a sequence that has at least 70% sequence identity to SEQ ID NO: 4.
Clause 23 The RNA molecule according to any one of clauses 1 to 18, wherein the 3′ UTR comprises a sequence that has at least 70% sequence identity to SEQ ID NO: 6.
Clause 24 The RNA molecule according to any one of clauses 1 to 18, wherein the 3′ UTR consists of a sequence that has at least 70% sequence identity to SEQ ID NO: 6.
Clause 25 The RNA molecule according to any one of clauses 1 to 18, wherein the 3′ UTR comprises a sequence that has at least 70% sequence identity to SEQ ID NO: 8.
Clause 26 The RNA molecule according to any one of clauses 1 to 18, wherein the 3′ UTR consists of a sequence that has at least 70% sequence identity to SEQ ID NO: 8.
Clause 27 The RNA molecule according to any one of clauses 1 to 18, wherein the 3′ UTR comprises a sequence that has at least 70% sequence identity to SEQ ID NO: 10.
Clause 28 The RNA molecule according to any one of clauses 1 to 18, wherein the 3′ UTR consists of a sequence that has at least 70% sequence identity to SEQ ID NO: 10.
Clause 29 The RNA molecule according to any one of clauses 1 to 18, wherein the 3′ UTR comprises a sequence that has at least 70% sequence identity to SEQ ID NO: 12.
Clause 30 The RNA molecule according to any one of clauses 1 to 18, wherein the 3′ UTR consists of a sequence that has at least 70% sequence identity to SEQ ID NO: 12.
Clause 31 The RNA molecule according to any one of clauses 1 to 18, wherein the 3′ UTR comprises a sequence that has at least 70% sequence identity to SEQ ID NO: 14.
Clause 32 The RNA molecule according to any one of clauses 1 to 18, wherein the 3′ UTR consists of a sequence that has at least 70% sequence identity to SEQ ID NO: 14.
Clause 33 The RNA molecule according to any one of clauses 1 to 32, wherein the 5′ UTR comprises a sequence that has at least 80% sequence identity to the reference sequence.
Clause 34 The RNA molecule according to clause 33, wherein the 5′ UTR comprises a sequence that has at least 90% sequence identity to the reference sequence.
Clause 35 The RNA molecule according to clause 34, wherein the 5′ UTR comprises a sequence that has at least 95% sequence identity to the reference sequence.
Clause 36 The RNA molecule according to clause 35, wherein the 5′ UTR comprises a sequence that has at least 98% sequence identity to the reference sequence.
Clause 37 The RNA molecule according to clause 36, wherein the 5′ UTR comprises a sequence that has at least 99% sequence identity to the reference sequence.
Clause 38 The RNA molecule according to clause 37, wherein the 5′ UTR comprises a sequence that has 100% sequence identity to the reference sequence.
Clause 39 The RNA molecule according to any one of clauses 1 to 38, wherein the 3′ UTR has at least 80% sequence identity.
Clause 40 The RNA molecule according to clause 39, wherein the 3′ UTR comprises a sequence that has at least 90% sequence identity to the reference sequence.
Clause 41 The RNA molecule according to clause 40, wherein the 3′ UTR comprises a sequence that has at least 95% sequence identity to the reference sequence.
Clause 42 The RNA molecule according to clause 41, wherein the 3′ UTR comprises a sequence that has at least 98% sequence identity to the reference sequence.
Clause 43 The RNA molecule according to clause 42, wherein the 3′ UTR comprises a sequence that has at least 99% sequence identity to the reference sequence.
Clause 44 The RNA molecule according to clause 43, wherein the 3′ UTR comprises a sequence that has 100% sequence identity to the reference sequence.
Clause 45 The RNA molecule according to clause 1, wherein the 5′ UTR comprises a sequence that has at least 95% identity to SEQ ID NO: 5 and the 3′ UTR comprises a sequence that has at least 95% identity to SEQ ID NO: 6.
Clause 45 The RNA molecule according to clause 1, wherein the 5′ UTR comprises a sequence that has at least 95% identity to SEQ ID NO: 7 and the 3′ UTR comprises a sequence that has at least 95% identity to SEQ ID NO: 8.
Clause 45 The RNA molecule according to clause 1, wherein the 5′ UTR comprises a sequence that has at least 95% identity to SEQ ID NO: 13 and the 3′ UTR a sequence that has at least 95% identity to SEQ ID NO: 14.
Clause 46 The RNA molecule according to any one of clauses 1 to 45, wherein the heterologous polypeptide comprises an immunogen.
Clause 47 The RNA molecule according to clause 46, wherein the heterologous polypeptide consists of an immunogen.
Clause 48 The RNA molecule according to any one of clauses 1 to 47, wherein the heterologous polypeptide comprises an antibody against an immunogen.
Clause 49 The RNA molecule according to clause 48, wherein the heterologous polypeptide consists of an antibody against an immunogen.
Clause 50 The RNA molecule according to any one of clauses 1 to 49, wherein the immunogen elicits an immune response against a bacterium.
Clause 51 The RNA molecule according to any one of clauses 1 to 50, wherein the immunogen is derived from a bacterium.
Clause 52 The RNA molecule according to either clause 51 or 52, wherein the bacterium is selected from: Neisseria meningitidis including, but are not limited to, membrane proteins such as adhesins, autotransporters, toxins, iron acquisition proteins, and factor H binding protein; Streptococcus pneumoniae including, but are not limited to, the RrgB pilus subunit, the beta-N-acetyl-hexosaminidase precursor (spr0057), spr0096, general stress protein GSP-781 (spr2021, SP2216), serine/threonine kinase StkP (SP1732), and pneumococcal surface adhesin PsaA; Streptococcus pyogenes; Moraxella catarrhalis; Bordetella pertussis including but are not limited to, pertussis toxin or toxoid (PT), filamentous haemagglutinin (FHA), pertactin, and agglutinogens 2 and 3; Staphylococcus aureus including a hemolysin, esxA, esxB, ferrichrome-binding protein (sta006), and the sta011 lipoprotein; Clostridium tetani including tetanus toxoid immunogen; Cornynebacterium diphtheriae including diphtheria toxoid immunogen; Haemophilus influenzae; Pseudomonas aeruginosa; Streptococcus agalactiae; Chlamydia trachomatis including, but are not limited to, PepA, LcrE, ArtJ, DnaK, CT398, OmpH-like, L7/L12, OmcA, AtoS, CT547, Eno, HtrA,and MurG; Chlamydia pneumoniae; Helicobacter pylori; Escherichia coli, including the following pathological subforms enterotoxigenic E. coli (ETEC), enteroaggregative E. coli (EAggEC), diffusely adhering E. coli (DAEC), enteropathogenic E. coli (EPEC), enterohemorrhagic E. coli (EHEC), and extraintestinal pathogenic E. coli (ExPEC), including therein uropathogenic E. coli (UPEC) and meningitis/sepsis-associated E. coli (MNEC); Bacillus anthracis; Yersinia pestis; Staphylococcus epidermis; Clostridium difficile; Clostridium perfringens; Clostridium botulinums; Legionella pneumophila; Coxiella burnetiid; Brucella, including B. abortus, B. canis, B. melitensis, B. neotomae, B. ovis, B. suis, B. pinnipediae; Francisella, including F. novicida, F. philomiragia, F. tularensis. Neisseria gonorrhoeae, including polypeptides of the outer membrane vesicles; Treponema pallidum; Haemophilus ducreyi; Enterococcus faecalis; Enterococcus faecium; Staphylococcus saprophyticus; Yersinia enterocolitica; Mycobacterium tuberculosis; Rickettsia; Listeria monocytogenes; Vibrio cholerae; Salmonella including Salmonella typhii; Borrelia burgdorferi; Porphyromonas gingivalis; and Klebsiella.
Clause 53 The RNA molecule according to any one of clauses 1 to 49, wherein the immunogen elicits an immune response against a virus.
Clause 54 The RNA molecule according to any one of clauses 1 to 49 or 53, wherein the immunogen is derived from a virus.
Clause 55 The RNA molecule according to either clause 53 or 54, wherein the virus is selected from Orthomyxovirus including influenza A, B, or C virus, including from influenza A virus subtypes H1, H2, H3, H4, H5, H6, H7, H8, H9, H10, H11, H12, H13, H14, H15, and H16 and including the immunogens of neuraminidase matrix M2 proteins, and hemagglutinin; Paramyxoviridae viruses including, but are not limited to, those derived from Pneumoviruses including respiratory syncytial virus (RSV), Rubulaviruses including mumps virus, Paramyxoviruses including parainfluenza virus, Metapneumoviruses, Morbilliviruses including measles, including the spike-like proteins immunogens thereof, membrane fusion proteins, (i.e. encoded as proteins by the RNA but becoming glycoproteins once translated by the mammalian cell expressing said protein; e.g. RSV F and RSV pre-F) and attachment proteins (i.e. encoded as proteins by the RNA but becoming glycoproteins once translated by the mammalian cell expressing said protein; e.g. RSV G); Poxviridae: Viral immunogens include, but are not limited to, those derived from Orthopoxvirus such as Variola vera, including but not limited to, Variola major and Variola minor; Picornavirus including Rhinoviruses, Heparnavirus, Cardioviruses, Aphthoviruses, and Enteroviruses including EV71 enterovirus, coxsackie A virus, coxsackie B virus, type 1 poliovirus, type 2 poliovirusand type 3 poliovirus; Bunyavirus including Orthobunyavirus such as California encephalitis virus, a Phlebovirus such as Rift Valley Fever virus, and a Nairovirus such as Crimean-Congo hemorrhagic fever virus; Heparnavirus including hepatitis A virus (HAV); Filovirus including Marburg virus and Ebolavirus including Zaire ebolavirus, Tai Forest ebolavirus (nee Ivory Coast ebolavirus), Sudan ebolavirus, Reston ebolavirus, Bundibugyo ebolavirus, and Bombali ebolavirus; Togavirus including, Alphavirus, Arterivirus, and Rubivirus including Rubivirus rubella, Rubivirus ruteetense, and Rubivirus strelense; Flavivirus including Tick-borne encephalitis (TBE) virus, Dengue virus (e.g. types 1, 2, 3 or 4), Yellow Fever virus, Japanese encephalitis virus, Kyasanur Forest Virus, West Nile encephalitis virus, St. Louis encephalitis virus, Russian spring-summer encephalitis virus, Powassan encephalitis virus, and Zikavirus; Pestivirus including Bovine viral diarrhea (BVDV), Classical swine fever (CSFV), and Border disease (BDV); Hepadnavirus including Hepatitis B virus, such as hepatitis B virus surface antigen (HBsAg); other hepatitis viruses, including hepatitis C virus, delta hepatitis virus, hepatitis E virus, and hepatitis G virus; Rhabdovirus including alpharahabdovirinae, Almendravirus, Alphanemrhavirus, Alphapaprhavirus, Alpharicinrhavirus, Arurhavirus, Barhavirus, Caligrhavirus, Curiovirus, Ephemerovirus, Hapavirus, Ledantevirus, Lostrhavirus, Lyssavirus such as rabies virus, Merhavirus, Mousrhavirus, Ohlsrhavirus, Perhabdovirus, Sawgrhavirus, Sigmavirus, Sprivivirus, Sripuvirus, Sunrhavirus, Tibrovirus, Tupavirus, Vesiculovirus, Zarhavirus, Betarhabdovirinae, Alphanucleorhabdovirus, Betanucleorhabdovirus, Cytorhabdovirus, Dichorhavirus, Gammanucleorhabdovirus, Varicosavirus, Gammarhabdovirinae, Novirhabdovirus, Alphacrustrhavirus, Alphadrosrhavirus, Alphahymrhavirus, Betahymrhavirus, Betanemrhavirus, Betapaprhavirus, Betaricinrhavirus; Caliciviridae including Norwalk virus (Norovirus) and Norwalk-like Viruses, such as Hawaii Virus and Snow Mountain Virus; Coronavirus including alpha-, beta-, gamma-, and delta-coronaviruses, including severe acute respiratory syndrome (SARS) coronavirus, SARS-CoV-2, Middle East respiratory syndrome coronavirus, human coronavirus HKU1, coronavirus avian infectious bronchitis (IBV), Mouse hepatitis virus (MHV), and Porcine transmissible gastroenteritis virus (TGEV), including a spike polypeptide immunogen, nucleoprotein immunogen, membrane protein immunogen, and ORF3a protein immunogen; Retrovirus including Oncovirus, Lentivirus, such as human immunodeficiency virus 1 and 2, and Spumavirus; Reovirus including Orthoreovirus, Rotavirus, Orbivirus, and Coltivirus; Parvovirus including Parvovirus B19; Herpesvirus including a human herpesvirus including Herpes Simplex Viruses (HSV), such as HSV types 1 and 2, Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8), including such immunogens as gB, gEv(e.g. VZV gE), gH (e.g. CMV gH), gl, gL (e.g. CMV gL), gO; gM, gN; UL128, UL130, and UL131A (e.g. CMV UL128, UL130, and UL131A); Papovaviruses including Polyomaviruses and Papillomaviruses, including 1, 2, 4, 5, 6, 8, 11, 13, 16, 18, 31, 33, 35, 39, 41, 42, 47, 51, 57, 58, 63, and 65 thereof; and Adenovirus including Adenovirus A such as adenoviruses 12, 18, 31, Adenovirus B such as adenoviruses 3, 7, 11, 14, 16, 21, 34, 35, 50, and 55, Adenovirus C such as adenoviruses 1, 2, 5, 6, and 57, Adenovirus D such as adenoviruses 8,9,10, 13,15,17,19,20,22,23,24, 25, 26, 27, 28, 29, 30, 32, 33, 36, 37, 38, 39, 42, 43, 44, 45, 46, 47, 48, 49, 51, 53, 54, 56, 58, 59, 60, 62, 63, 64, 65, 67, 69, 70, 71, 72, 73, 74, and 75, Adenovirus E such as adenovirus 4, Adenovirus F such as adenoviruses 40 and 41, and Adenovirus G such as adenovirus 52.
Clause 56 The RNA molecule according to any one of clauses 1 to 49, wherein the immunogen elicits an immune response against a fungi.
Clause 57 The RNA molecule according to any one of clauses 1 to 49 or 56, wherein the immunogen is derived from a fungi.
Clause 58 The RNA molecule according to either clause 56 or 57, wherein the fungi is selected from Dermatophytres, Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis; Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.
Clause 59 The RNA molecule according to any one of clauses 1 to 49, wherein the immunogen elicits an immune response against a parasite.
Clause 60 The RNA molecule according to any one of clauses 1 to 49 or 59, wherein the immunogen is derived from a parasite.
Clause 61 The RNA molecule according to either clause 59 or 60, wherein the parasite is selected from Plasmodium, such as P. falciparum, P. vivax, P. malariae, and P. ovale and Caligidae family, particularly those from the Lepeophtheirus and Caligus genera, such as sea lice such as Lepeophtheirus salmonis or Caligus rogercresseyi.
Clause 62 The RNA molecule according to any one of clauses 1 to 49, wherein the immunogen elicits an immune response against an allergen.
Clause 63 The RNA molecule according to any one of clauses 1 to 49 or 62, wherein the immunogen is derived from an allergen.
Clause 64 The RNA molecule according to either clause 62 or 63, wherein the allergen is selected from pollen allergens (tree, herb, weed, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).
Clause 65 The RNA molecule according to any one of clauses 1 to 49, wherein the immunogen elicits an immune response against a cancer.
Clause 66 The RNA molecule according to any one of clauses 1 to 49 or 65, wherein the immunogen is a cancer antigen.
Clause 67 The RNA molecule according to either clause 65 or 66, wherein the cancer antigen is selected from (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH—P1, PSM-P1, PSM-P2, associated with e.g., prostate cancer; (f) immunoglobulin idiotypes (associated with myeloma and B cell lymphomas, for example).
Clause 68 The RNA molecule according to any one of clauses 1 to 49, wherein the segment that encodes an immunogen comprises a sequence that has at least 70% identity to at least one of SEQ ID NOs: 29-31 and 123-133.
Clause 69 A recombinant RNA molecule comprising a sequence that has at least 70% identity to at least one of SEQ ID NOs: 29-31 and 123-133.
Clause 70 The recombinant RNA molecule according to clause 69, comprising a 5′ UTR.
Clause 71 The recombinant RNA molecule according to either clause 69 or 70, comprising a 3′ UTR.
Clause 72 The RNA molecule according any one of clauses 68 to 71, comprising a sequence that has at least 70% identity to SEQ ID NO: 29.
Clause 73 The RNA molecule according to clause 72, wherein the segment that encodes an immunogen consists of a sequence that has at least 70% identity to SEQ ID NO: 29.
Clause 74 The RNA molecule according any one of clauses 68 to 71, comprising a sequence that has at least 70% identity to SEQ ID NO: 30.
Clause 75 The RNA molecule according to clause 74, wherein the segment that encodes an immunogen consists of a sequence that has at least 70% identity to SEQ ID NO: 30.
Clause 76 The RNA molecule according any one of clauses 68 to 71, comprising a sequence that has at least 70% identity to SEQ ID NO: 31.
Clause 77 The RNA molecule according to clause 76, wherein the segment that encodes an immunogen consists of a sequence that has at least 70% identity to SEQ ID NO: 31.
Clause 78 The RNA molecule according any one of clauses 68 to 71, comprising a sequence that has at least 70% identity to SEQ ID NO: 123.
Clause 79 The RNA molecule according to clause 78, wherein the segment that encodes an immunogen consists of a sequence that has at least 70% identity to SEQ ID NO: 123.
Clause 80 The RNA molecule according to any one of clauses 68 to 71, comprising a sequence that has at least 70% identity to SEQ ID NO: 124.
Clause 81 The RNA molecule according to clause 80, wherein the segment that encodes an immunogen consists of a sequence that has at least 70% identity to SEQ ID NO: 124.
Clause 82 The RNA molecule according to any one of clauses 68 to 71, comprising a sequence that has at least 70% identity to SEQ ID NO: 125.
Clause 83 The RNA molecule according to clause 82, wherein the segment that encodes an immunogen consists of a sequence that has at least 70% identity to SEQ ID NO: 125.
Clause 84 The RNA molecule according to any one of clauses 68 to 71, comprising a sequence that has at least 70% identity to SEQ ID NO: 126.
Clause 85 The RNA molecule according to clause 84, wherein the segment that encodes an immunogen consists of a sequence that has at least 70% identity to SEQ ID NO: 126.
Clause 86 The RNA molecule according to any one of clauses 68 to 71, comprising a sequence that has at least 70% identity to SEQ ID NO: 127.
Clause 87 The RNA molecule according to clause 86, wherein the segment that encodes an immunogen consists of a sequence that has at least 70% identity to SEQ ID NO: 127.
Clause 88 The RNA molecule according to any one of clauses 68 to 71, comprising a sequence that has at least 70% identity to SEQ ID NO: 128.
Clause 89 The RNA molecule according to clause 88, wherein the segment that encodes an immunogen consists of a sequence that has at least 70% identity to SEQ ID NO: 128.
Clause 90 The RNA molecule according to any one of clauses 68 to 71, comprising a sequence that has at least 70% identity to SEQ ID NO: 129.
Clause 91 The RNA molecule according to clause 90, wherein the segment that encodes an immunogen consists of a sequence that has at least 70% identity to SEQ ID NO: 129.
Clause 92 The RNA molecule according to any one of clauses 68 to 71, comprising a sequence that has at least 70% identity to SEQ ID NO: 130.
Clause 93 The RNA molecule according to clause 92, wherein the segment that encodes an immunogen consists of a sequence that has at least 70% identity to SEQ ID NO: 130.
Clause 94 The RNA molecule according to any one of clauses 68 to 71, comprising a sequence that has at least 70% identity to SEQ ID NO: 131.
Clause 95 The RNA molecule according to clause 94, wherein the segment that encodes an immunogen consists of a sequence that has at least 70% identity to SEQ ID NO: 131.
Clause 96 The RNA molecule according to any one of clauses 68 to 71, comprising a sequence that has at least 70% identity to SEQ ID NO: 132.
Clause 97 The RNA molecule according to clause 96, wherein the segment that encodes an immunogen consists of a sequence that has at least 70% identity to SEQ ID NO: 132.
Clause 98 The RNA molecule according to any one of clauses 68 to 71, comprising a sequence that has at least 70% identity to SEQ ID NO: 133.
Clause 99 The RNA molecule according to clause 98, wherein the segment that encodes an immunogen consists of a sequence that has at least 70% identity to SEQ ID NO: 133.
Clause 100 The RNA molecule according to any one of clauses 68 to 99, comprising a sequence that has at least 80% sequence identity to the reference sequence.
Clause 101 The RNA molecule according to clause 100, comprising a sequence that has at least 90% sequence identity to the reference sequence.
Clause 102 The RNA molecule according to clause 101, comprising a sequence that has at least 95% sequence identity to the reference sequence.
Clause 103 The RNA molecule according to clause 102, comprising a sequence that has at least 97% sequence identity to the reference sequence.
Clause 104 The RNA molecule according to clause 103, comprising a sequence that has at least 98% sequence identity to the reference sequence.
Clause 105 The RNA molecule according to clause 104, comprising a sequence that has at least 99% sequence identity to the reference sequence.
Clause 106 The RNA molecule according to clause 105, comprising a sequence that has 100% sequence identity to the reference sequence.
Clause 107 The RNA molecule according to any preceding claim, comprising further segments that encode other heterologous polypeptides, such as one further segment.
Clause 108 The RNA molecule according to any preceding claim comprising a sequence that has at least 70% identity to at least one of SEQ ID NOs: 32-40, 45-62, 69-77, 82-90, 134-173, and 178-197.
Clause 109 The RNA molecule according to clause 108, comprising a sequence that has at least 80% sequence identity to the reference sequence.
Clause 110 The RNA molecule according to clause 108, comprising a sequence that has at least 90% sequence identity to the reference sequence.
Clause 111 The RNA molecule according to clause 108, comprising a sequence that has at least 95% sequence identity to the reference sequence.
Clause 112 The RNA molecule according to clause 108, comprising a sequence that has at least 97% sequence identity to the reference sequence.
Clause 113 The RNA molecule according to clause 108, comprising a sequence that has at least 98% sequence identity to the reference sequence.
Clause 114 The RNA molecule according to clause 108, comprising a sequence that has at least 99% sequence identity to the reference sequence.
Clause 115 The RNA molecule according to clause 108, comprising a sequence that has 100% sequence identity to the reference sequence.
Clause 116 The RNA molecule according to any one of clauses 1 to 115, comprising a 3′ poly(A).
Clause 117 The RNA molecule according to clause 116, wherein the 3′ poly(A) is 3′ of a 3′ UTR.
Clause 118 The RNA molecule according to either clause 116 or 117, wherein the 3′ poly(A)) tail is at the 3′ end of the recombinant RNA.
Clause 119 The RNA molecule according to any one of clauses 116 to 118, wherein the 3′ poly(A) comprises a first segment of consecutive adenosine monophosphate residues, a spacer, and a second segment of consecutive adenosine monophosphate residues and wherein the spacer comprises at least one nucleotide other than adenosine monophosphate.
Clause 120 The RNA molecule according to any one of clauses 116 to 119, wherein the 3′ poly(A) comprises, or where the 3′ poly(A) comprises a first and a second segment of consecutive adenosine monophosphates the first segment comprises, 20 to 150 consecutive adenosine monophosphates.
Clause 121 The RNA molecule according to clause 120, wherein the 3′ poly(A) comprises, or where the 3′ poly(A) comprises a first and a second segment of consecutive adenosine monophosphates the first segment comprises, 50 to 120 consecutive adenosine monophosphates.
Clause 122 The RNA molecule according to clause 121, wherein the 3′ poly(A) comprises, or where the 3′ poly(A) comprises a first and a second segment of consecutive adenosine monophosphates the first segment comprises, 70 to 100 consecutive adenosine monophosphates.
Clause 123 The RNA molecule according to any one of clauses 116 to 122, wherein the second segment comprises 20 to 150 consecutive adenosine monophosphates.
Clause 124 The RNA molecule according to clause 123, wherein the second segment comprises 50 to 120 consecutive adenosine monophosphates.
Clause 125 The RNA molecule according to clause 123, wherein the second segment comprises 70 to 100 consecutive adenosine monophosphates.
Clause 126 The RNA molecule according to any one of clauses 1 to 125 comprising a 5′ cap.
Clause 127 The RNA molecule according to clause 126, wherein the 5′ cap comprises a 5′ cap nucleoside (e.g. a 7-methylguanosine or a modified 7-methylguanosine) and a first 5′ ribonucleoside; wherein the 5′ cap nucleoside (e.g. the 7-methylguanosine or the modified 7-methylguanosine) is linked 5′-to-5′ to the first 5′ ribonucleoside.
Clause 128 The RNA molecule according to clause 127, wherein the first 5′ ribonucleoside comprises a 2′ methylated ribose.
Clause 129 The RNA molecule according to either clause 127 or 128, comprising a linker; wherein the 5′ cap nucleoside (e.g. the 7-methylguanosine or the modified 7-methylguanosine) is linked 5′-to-5′ to the first 5′ ribonucleoside by the linker.
Clause 130 The RNA molecule according to clause 129, wherein the linker is a triphosphate.
Clause 131 The RNA molecule according to any one of clauses 1 to 130, comprising at least one modified nucleotide.
Clause 132 The RNA molecule according to clause 131, comprising pseudouridine, N1-methylpseudouridine, or N1-ethylpseudouridine.
Clause 133 The RNA molecule according to clause 132, wherein at least 80% of the uridine residues are replaced with pseudouridine, N1-methylpseudouridine, or N1-ethylpseudouridine.
Clause 134 The RNA molecule according to clause 132, wherein at least 90% of the uridine residues are replaced with pseudouridine, N1-methylpseudouridine, or N1-ethylpseudouridine.
Clause 135 The RNA molecule according to clause 132, wherein at least 95% of the uridine residues are replaced with pseudouridine, N1-methylpseudouridine, or N1-ethylpseudouridine.
Clause 136 The RNA molecule according to clause 132, wherein at least 99% of the uridine residues are replaced with pseudouridine, N1-methylpseudouridine, or N1-ethylpseudouridine.
Clause 137 The RNA molecule according to any one of clauses 1 to 136, comprising a sequence that has at least 95% identity to at least one of SEQ ID NOs: 32, 33, 35, 36, 38, 39, 45, 46, 48, 49, 51, 52, 54, 55, 57, 58, 60, 61, 69, 70, 72, 73, 75, 76, 82, 83, 85, 86, 89, 134, 135, 138, 139, 142, 143, 146, 147, 150, 151, 154, 155, 158, 159, 162, 163, 166, 167, 170, 171, 174, 175, 178, 179, 182, 183, 186, 187, 190, 191, 194, and 195.
Clause 138 The RNA molecule according to clause 137, consisting of a sequence that has at least 95% identity to at least one of SEQ ID NOs: 32, 33, 35, 36, 38, 39, 45, 46, 48, 49, 51, 52, 54, 55, 57, 58, 60, 61, 69, 70, 72, 73, 75, 76, 82, 83, 85, 86, 89, 134, 135, 138, 139, 142, 143, 146, 147, 150, 151, 154, 155, 158, 159, 162, 163, 166, 167, 170, 171, 174, 175, 178, 179, 182, 183, 186, 187, 190, 191, 194, and 195.
Clause 139 The RNA molecule according to either clause 137 or 138, comprising a sequence that has at least 98% identity to the reference sequence.
Clause 140 The RNA molecule according to clause 139, comprising a sequence that has at least 99% identity to the reference sequence.
Clause 141 The RNA molecule according to clause 139, comprising a sequence that has at least 100% identity to the reference sequence.
Clause 142 The RNA molecule according to any one of clauses 1 to 141, comprising a sequence that has at least 95% identity to at least one of SEQ ID NOs: 32-40, 45-62, 69-77, 82-90, 134-173, and 178-197.
Clause 143 The RNA molecule according to clause 142, consisting of a sequence that has at least 95% identity to at least one of SEQ ID NOs: 32-40, 45-62, 69-77, 82-90, 134-173, and 178-197.
Clause 144 The RNA molecule according to either clause 141 or 143, comprising a sequence that has at least 98% identity to the reference sequence.
Clause 145 The RNA molecule according to clause 144, comprising a sequence that has at least 99% identity to the reference sequence.
Clause 146 The RNA molecule according to clause 145, comprising a sequence that has at least 100% identity to the reference sequence.
Clause 147 The RNA molecule according to any one of clauses 1 to 146, wherein the RNA molecule is self-replicating.
Clause 148 The RNA molecule according to any one of clauses 1 to 147, wherein the RNA molecule is not self-replicating.
Clause 149 The RNA molecule according to any one of clauses 1 to 148, wherein the encoded heterologous polypeptide contains 100 to 1500 amino acid residues.
Clause 150 The RNA molecule according to clause 149, wherein the encoded heterologous polypeptide contains 100 to 500 amino acid residues.
Clause 151 The RNA molecule according to clause 149, wherein the encoded heterologous polypeptide contains 500 to 1000 amino acid residues.
Clause 152 The RNA molecule according to clause 149, wherein the encoded heterologous polypeptide contains 1000 to 1500 amino acid residues.
Clause 153 The RNA molecule according to any one of clauses 1 to 152, wherein the RNA molecule is 1500-15000 nucleotides long.
Clause 154 The RNA molecule according to clause 149, wherein the RNA molecule is 2000-5000 nucleotides long.
Clause 155 The RNA molecule according to clause 149, wherein the RNA molecule is 3000-6000 nucleotides long.
Clause 156 The RNA molecule according to clause 149, wherein the RNA molecule is 8000-15000 nucleotides long.
Clause 157 A formulation comprising lipid nanoparticles (LNPs) and recombinant RNA molecules of any one of clauses 1 to 156; wherein:
   i) the LNPs comprise lipids;
   ii) the lipids comprise first lipids and cholesterol;
   iii) the first lipid comprises a tertiary amine and has a pKa from 5.0 to 7.6;
   iv) at least half of the first lipids are neutrally charged when the first lipids are at a pH that is above the pKa; and
   v) at least half of the first lipids are positively charged when the first lipids are at a pH that is below the pKa.
Clause 158 The formulation of clause 157, wherein at least 80% of the recombinant RNA molecules are encapsulated by the LNPs, such as at least 90%, especially at least 95%.
Clause 159 The formulation of either clause 157 or 158, wherein the Z-average diameter is from 50 to 200 nm.
Clause 160 The formulation of clause 159, wherein the Z-average diameter is from 60 to 180 nm.
Clause 161 The formulation of clause 160, wherein the Z-average diameter is from 80 to 160 nm.
Clause 162 The formulation of any one of clauses 157 to 161, wherein the polydispersity index is 0.3 or lower.
Clause 163 The formulation of clause 162, wherein the polydispersity index is 0.2 or lower.
Clause 164 The formulation of any one of clauses 157 to 163, wherein the first lipid has a pKa from 5.2 to 7.0 Clause 165 The formulation of clause 164, wherein the first lipid has a pKa from 5.5 to 6.8.
Clause 166 The formulation of any one of clauses 157 to 165, wherein the first lipids comprise the following:

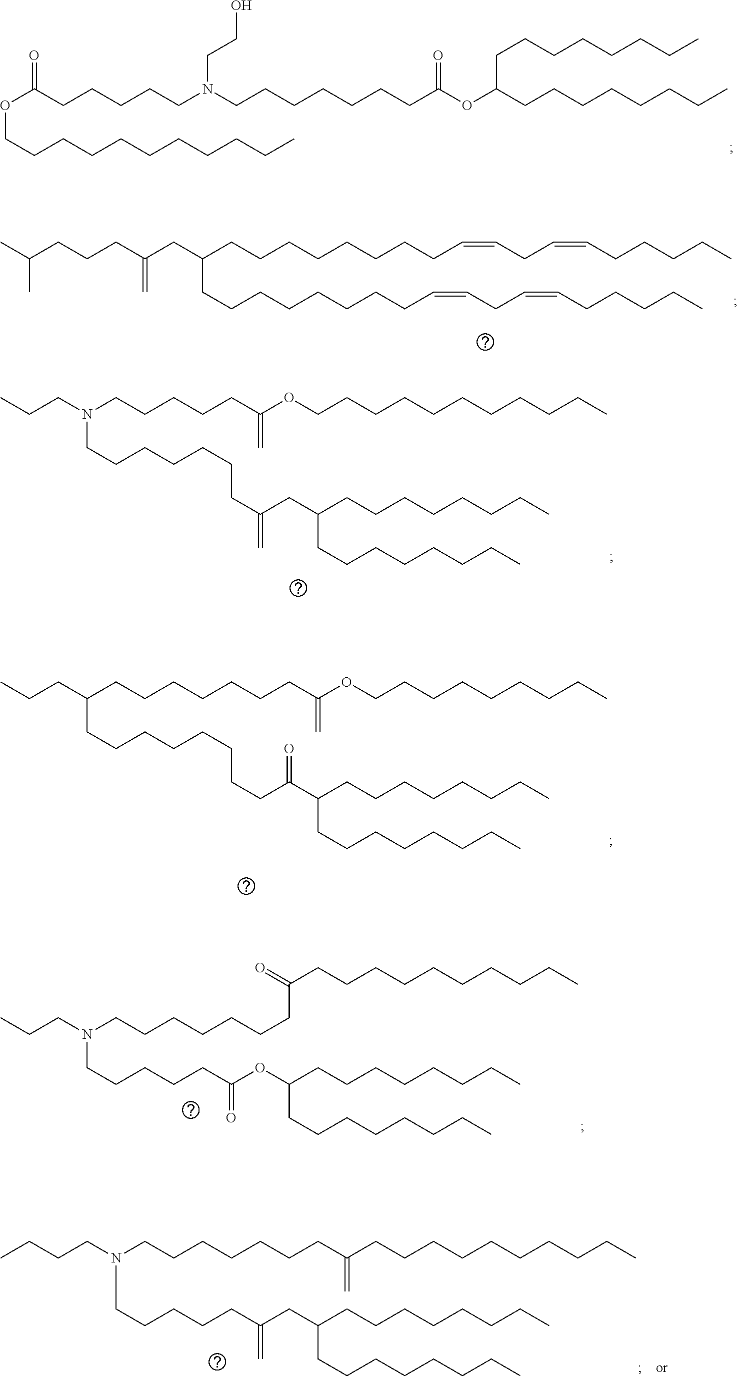

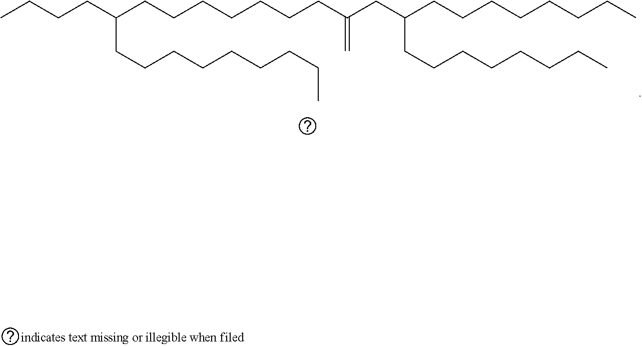 [SMILES: C=C(CCCCCCCC(CCCC)CCCCCCCCC)CC(CCCCCCCC)CCCCCCCC]

Clause 167 The formulation of any one of clauses 157 to 166, wherein the first lipids consist of the following:

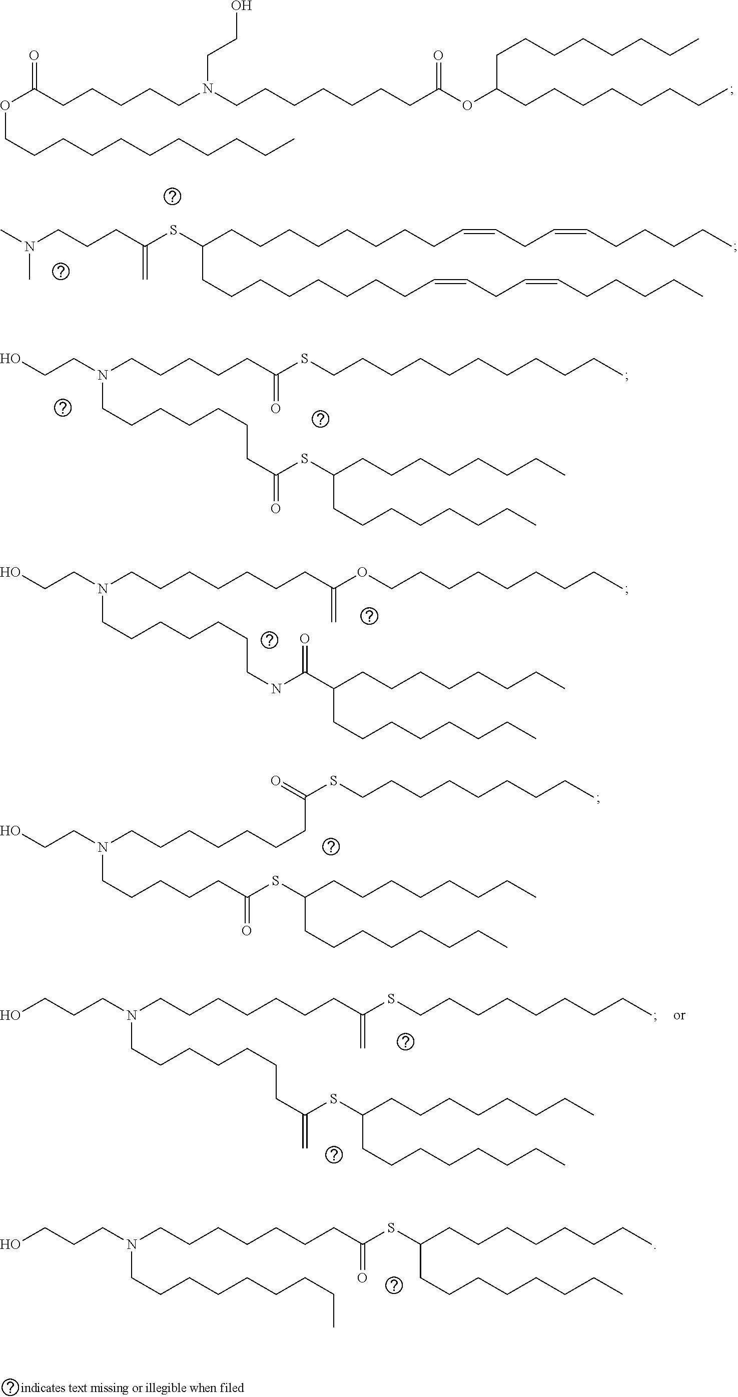

Clause 168 The formulation of any one of clauses 157 to 166, wherein the first lipids comprise

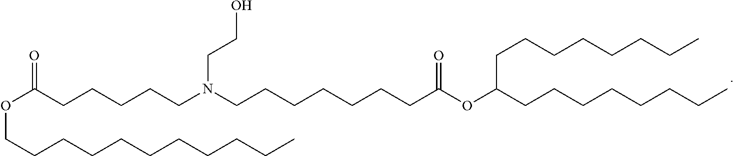 [SMILES: CCCCCCCCCCCOC(=O)CCCCCN(CCO)CCCCCCCC(=O)OC(CCCCCCCC)CCCCCCCC]

Clause 169 The formulation of any one of clauses 157 to 166, wherein the first lipids comprise:

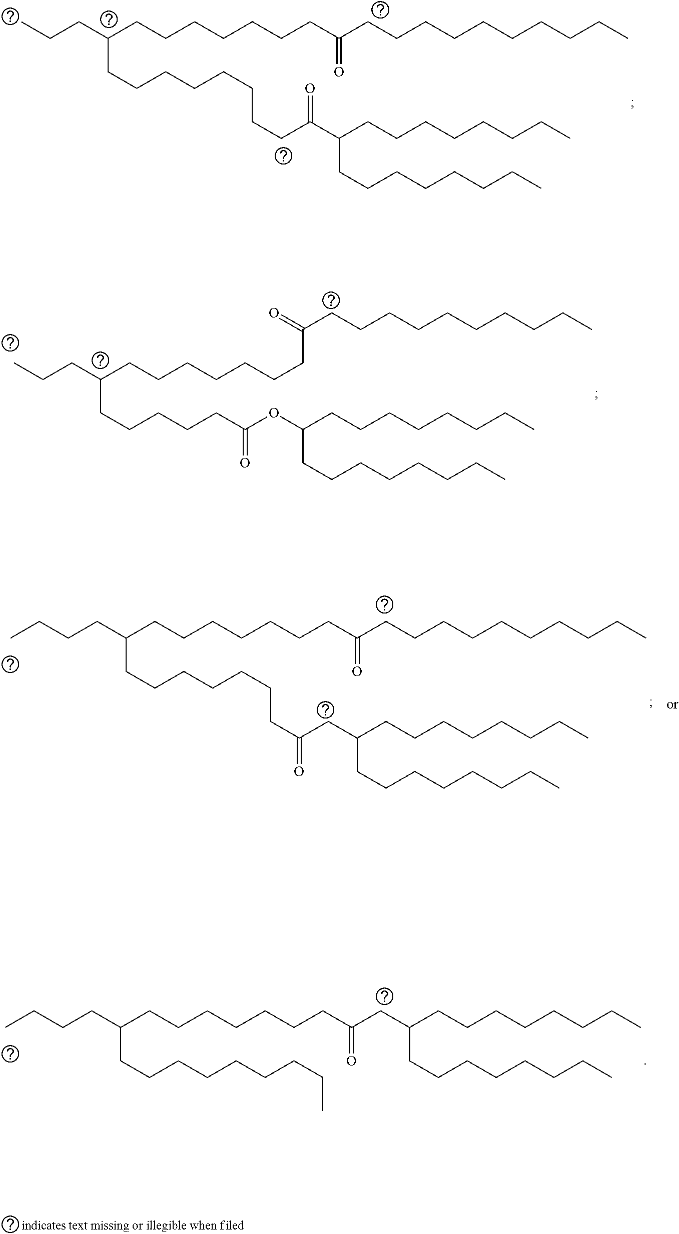

Clause 170 The formulation of any one of clauses 157 to 166, wherein the first lipids comprise:

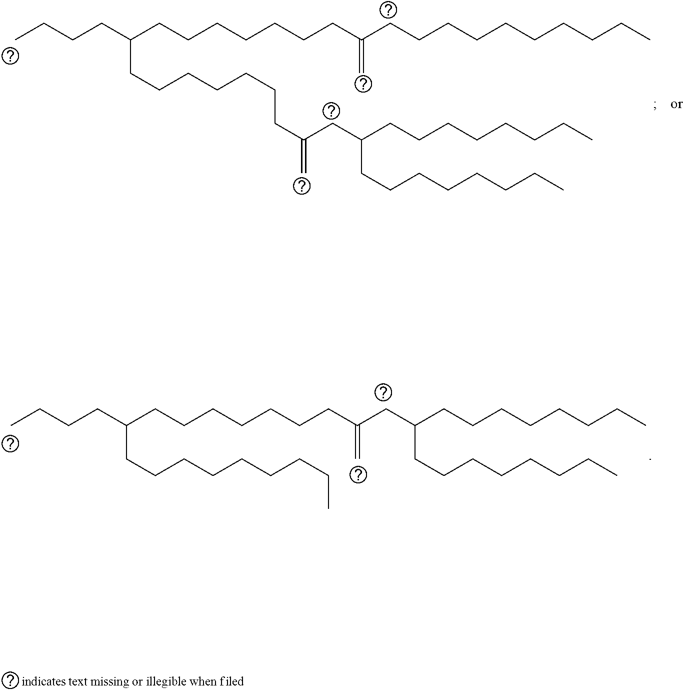

Clause 171 The formulation of any one of clauses 157 to 170, wherein the lipids comprise polyethylene glycol-conjugated (PEG-conjugated) lipids.
Clause 172 The formulation of clause 171, wherein the PEG-conjugated lipid comprises 1,2-dimyristoyl-rac-glycero-3-methoxypolyethylene glycol.
Clause 173 The formulation of clause 171, wherein the PEG-conjugated lipid comprises 2-[(polyethylene glycol)-2000]—N,N-ditetradecylacetamide.
Clause 174 The formulation of any one of clauses 157 to 173, wherein the lipids comprise a neutral lipid or a zwitterionic lipid.
Clause 175 The formulation of clause 174, wherein the zwitterionic lipid comprises 1,2-distearoyl-sn-glycero-3-phosphocholine (DSPC).
Clause 176 A method of eliciting an immune response against the immunogen in a subject, the method comprising administering to the subject an effective amount to elicit the immune response of the recombinant RNA molecule of any one of clauses 1 to 156 comprising a segment encoding an immunogen, or the formulation of any one of clauses 157 to 175 comprising a recombinant RNA molecule comprising a segment encoding an immunogen.
Clause 177 Use of the RNA molecule of any one of clauses 1 to 156 for the manufacture of a medicament.
Clause 178 Use of the RNA molecule of any one of clauses 1 to 156 comprising a segment encoding an immunogen for the manufacture of a medicament for eliciting an immune response to the immunogen in a subject.
Clause 179 Use of the RNA molecule of any one of clauses 1 to 156 comprising a segment encoding an immunogen derived from a pathogen for the manufacture of a medicament for prophylaxis of infection by the pathogen in a subject.
Clause 180 Use of the RNA molecule of any one of clauses 1 to 156 comprising a segment encoding an immunogen derived from a pathogen for the manufacture of a medicament for treatment of infection by the pathogen in a subject.
Clause 181 Use of the RNA molecule of any one of clauses 1 to 156 comprising a segment encoding an heterologous polypeptide for the manufacture of a medicament for the in vivo expression of the heterologous polypeptide in a subject.
Clause 182 The RNA of any one of clauses 1 to 156 for use as a medicament.
Clause 183 The RNA molecule of any one of clauses 1 to 156 comprising a segment encoding an immunogen for use in eliciting an immune response to the immunogen in a subject.
Clause 184 The RNA molecule of any one of clauses 1 to 156 comprising a segment encoding an immunogen derived from a pathogen for use in prophylaxis of infection by the pathogen in a subject.
Clause 185 The RNA molecule of any one of clauses 1 to 156 comprising a segment encoding an immunogen derived from a pathogen for use in treatment of infection by the pathogen in a subject.
Clause 186 The RNA molecule of any one of clauses 1 to 156 comprising a segment encoding an heterologous polypeptide for use in the in vivo expression of the heterologous polypeptide in a subject.
Clause 187 The RNA molecule, method or use according to any one of clauses 176 to 186 for a human subject.
Clause 188 The RNA molecule, method or use according to clause 187 for a human subject less than 18 years old.
Clause 189 The RNA molecule, method or use according to clause 187 for a human subject at least 18 years old.
Clause 190 The RNA molecule, method or use according to clause 187 for a human subject at least 60 years old.
Clause 191 The RNA molecule, method or use according to any one of clauses 176 to 190 for eliciting an antibody response.
Clause 192 The RNA molecule, method or use according to any one of clauses 176 to 191 for eliciting a cellular immune response.
Clause 193 The RNA molecule, method or use according to any one of clauses 176 to 192 for eliciting a protective immune response.
Clause 194 The RNA molecule, method or use according to any one of clauses 176 to 193 for intramuscular administration.
Clause 195 The RNA molecule, method or use according to any one of clauses 176 to 194 for administration at a dose of 1 to 150 μg of RNA.
Clause 196 The RNA molecule, method or use according to clause 195 for administration art a dose of 10 to 100 μg of RNA.
Clause 197 The RNA molecule, method or use according to clause 196 for administration art a dose of 10 to 80 μg of RNA, such as 10 to 60 ug.
Clause 198 The RNA molecule according to any one of clauses 1 to 156, wherein the heterologous polypeptide is a fusion protein of a plurality of immunogens.
Clause 199 The RNA molecule according to any one of clauses 1 to 156 or 198, comprising a plurality of segments encoding heterologous polypeptides.
Clause 200 The formulation according to any one of clauses 157 to 175, comprising a plurality of RNA molecules comprising segments encoding different heterologous polypeptides.
Clause 201 A composition comprising a plurality of formulations according to any one of clauses 156 to 175 or 200, the formulations comprising RNA molecules comprising segments encoding different heterologous polypeptides.
Clause 202 A method of manufacturing the recombinant RNA molecule of any one of clauses 1 to 156, 199 or 200, the method comprising admixing an RNA polymerase, triphosphate nucleotides, and a template nucleic acid comprising a sequence of the recombinant RNA molecule, thereby obtaining an admixture; the admixing being under conditions wherein the RNA polymerase produces the recombinant RNA molecule from the template nucleic acid.
Clause 203 A method of manufacturing the formulation of any one of clauses 156 to 175 or 200, the method comprising:
   a. admixing the recombinant RNA molecules with an aqueous buffer, thereby obtaining an aqueous recombinant RNA solution;
   b. admixing the lipids in an organic solvent, thereby obtaining a lipid solution;
   c. admixing the lipid solution and the recombinant RNA solution with at least a T-mixer, a microfluidics mixer, or an impinging jet mixer, thereby obtaining a first solution, wherein the first solution comprises the LNPs, wherein at least some of the recombinant RNA molecules are encapsulated in the LNPs; and d. purifying the LNPs to obtain the at least 80% of the recombinant RNA molecules
being encapsulated by the LNPs.

### EXAMPLES

### General In Vitro Methods

### Design and In Vitro Transcription of RNA

For the experiments with firefly luciferase, UTRs from beta-actin gene (ACTB, Accession NO: NM_001101.5; SEQ ID NOs: 15 & 16), albumin gene (ALB or UTR1, Accession NOs: NG_056261.1 & NM_000477.7; SEQ ID NOs: 1 & 2), adenosine triphosphate (ATP) synthase beta subunit gene (ATPSB or UTR2, Accession NOs: AH002618.2 & NM_001686.4; SEQ ID NOs: 3 & 4), fibroblast activation protein messenger ribonucleic acid (mRNA) (FAP or UTR 3, SEQ ID NOs: 5 & 6, Accession NOs: U09278.2 & NM_001291807.3), H4 clustered histone 15 mRNA (HIST2H4A or UTR4, SEQ ID NOs: 7 & 8; Accession NO: NR_168448.1), glyceraldehyde-3-phosphate dehydrogenase gene (GAPDH or UTR5, Accession NOs: NG_007073.2 & NR_152150.2; SEQ ID NOs: 9 & 10), heat shock protein family A (Hsp70) member 8 gene (HSPA8 or UTR6, Accession NOs: NG_029473.1 & NM_153201.4; SEQ ID NOs: 11 & 12), interleukin-2 gene (IL-2 or UTR7; Accession NOs: AH002842.2 & NM_000586.4; SEQ ID NOs: 13 & 14), transferrin gene (TF, Accession NO: NG_013080.2; SEQ ID NOs: 17 & 18), and eukaryotic translation elongation factor 2 (EEF2) mRNA (Accession NO: NM_001961.4; SEQ ID NOs: 19 & 20) surrounded the gene from firefly luciferase and were imported into plasmid DNA. For UTR1-7, the resulting plasmid DNAs were SEQ ID NOs: 104-110 respectively. For the ACTB UTR, TF UTR, and EEF2 UTRS, the resulting plasmid DNAs were SEQ ID NOs: 111-113 respectively. In vitro transcription of the plasmid DNAs containing SEQ ID NOs: 104-113 were carried out to obtain RNA, which was then transfected into the cells as described below.

For the experiments with a SARS-CoV-2 spike protein from either the omicron strain or Wuhan strain encoded within the RNA, sequences were designed that incorporated one of UTR1-7, the UTRs in Moderna's 1273-mRNA, the UTRs in Pfizer/BioNTech's BNT-161, or the UTRs in CureVac's as described in positions 17-3838 of the sequence in The World Health Organization: International Nonproprietary Names Programme. Messenger RNA Encoding the Full-Length SARS-CoV-2 Spike Glycoprotein; p. 11868, which is further described in Edo Kon, Uri Elia, and Dann Peer, “Principles for designing an optimal mRNA lipid nanoparticle vaccine,” Curr. Opin. Biotechnol. 2022, 73:329-336.

All of the SARS-CoV-2 spike proteins from either the omicron strain or Wuhan strain were designed with two substitutions of the wild-type amino acids for prolines to result in a proline-proline prefusion stabilization. For the omicron strain, these are at amino acid positions 983 and 984 of Accession No. UF069279.1, or in the case of the BA.5 strain these are amino acid positions 981 and 982 of the spike glycoprotein, and for the Wuhan strain, they are located at amino acid positions 986 and 987 of the spike glycoprotein. For the SARS-CoV-2 omicron and Wuhan strain spike proteins, the proteins with the proline-proline substitution were simultaneously reverse translated and optimized by 1) a commercial software that only optimized for a codon adaptation index of 0.7 or greater (see e.g. SEQ ID NO: 31); 2) IDT technologies commercial software (see e.g. SEQ ID NO: 118); 3) a proprietary algorithm referred to as CR1 (see e.g. SEQ ID NO: 29); or 4) a proprietary algorithm referred to as CR1, CR2 (see e.g. SEQ ID NO: 30; see e.g. constructs LL34-38), CR3, CR4, CR5, CR6, or CR7 respectively. Constructs LL40-44 and LL60-67 were also optimized by CR3-7, respectively.

To allow for adjustment of the 3′ poly(adenosine monophosphate) (poly(A)) tail, a BsrDI restriction site, 5′-UUCAUUGC-3′ was added downstream (3′) of the 3′ UTR but before the 3′ poly(A) tail if those UTRs did not already terminate with a BsrDI restriction site. To allow for a Trilink CLEAN CAP® recognition site, an adenosine monophosphate (A) and/or one or more guanosine monophosphates (G) were added 5′ of the 5′ UTR in one of the following patterns “A,” “AG,” or “AGG.” In some embodiments, a representative split 3′ poly(A) tail was provided, and in other embodiments, the 3′ poly(A) tail consisted of As. Accordingly, following sequences were designed, and produced as identified in the examples below:

| | | | SEQ ID NO. of the complete molecule | | |
|---|---|---|---|---|---|
| | | | | With BsrD1 | |
| | | | | restriction site, | |
| | | | Without: BsrD1 | without | With: BsrD1 |
| | | | restriction site, | representative | restriction site, |
| | | | representative | 3′ poly(A) tail, | representative |
| | | | 3′ poly(A) tail, | without CLEAN | 3′ poly(A) tail, |
| Construct | 3′ and 5′ | Coding | CLEAN CAP ® | CAP ® | CLEAN CAP ® |
| Name | UTRs | Sequence | recognition site | recognition site | recognition site |
| KM61 | UTR3 | SARS-CoV-2 | SEQ ID NO: 32 | SEQ ID NO: 33 | SEQ ID NO: 34 |
| | (SEQ ID | omicron spike | | | |
| | NOs: | protein optimized | | | |
| | 5 & 6) | by commercial | | | |
| | | software with codon | | | |
| | | adaptation index | | | |
| | | (CAI) ≥ 0.7 | | | |
| | | (SEQ ID NO: 31) | | | |
| KM62 | UTR4 | SARS-CoV-2 | SEQ ID NO: 35 | SEQ ID NO: 36 | SEQ ID NO: 37 |
| | (SEQ ID | omicron spike | | | |
| | NOs: | protein optimized | | | |
| | 7 & 8) | by commercial | | | |
| | | software with | | | |
| | | CAI ≥ 0.7 | | | |
| | | (SEQ ID NO: 31) | | | |
| KM63 | UTR7 | SARS-CoV-2 | SEQ ID NO: 38 | SEQ ID NO: 39 | SEQ ID NO: 40 |
| | (SEQ ID | omicron spike | | | |
| | NOs: | protein optimized | | | |
| | 13 & 14) | by commercial | | | |
| | | software with | | | |
| | | CAI ≥ 0.7 | | | |
| | | (SEQ ID NO: 31) | | | |
| KM64 | BioNTech- | SARS-CoV-2 | | SEQ ID NO: 41 | SEQ ID NO: 42 |
| | 161 (SEQ | omicron spike | | | |
| | ID NOS: | protein optimized | | | |
| | 21 & 22) | by commercial | | | |
| | | software with | | | |
| | | CAI ≥ 0.7 | | | |
| | | (SEQ ID NO: 31) | | | |
| KM65 | Moderna | SARS-CoV-2 | | SEQ ID NO: 43 | SEQ ID NO: 44 |
| | 1273- | omicron spike | | | |
| | mRNA | protein optimized | | | |
| | UTRs | by commercial | | | |
| | (SEQ ID | software with | | | |
| | NOs: | CAI ≥ 0.7 | | | |
| | 23 & 24) | (SEQ ID NO: 31) | | | |
| KM66 | UTR3 | SARS-CoV-2 | SEQ ID NO: 45 | SEQ ID NO: 46 | SEQ ID NO: 47 |
| | (SEQ ID | omicron spike | | | |
| | NOs: | protein optimized | | | |
| | 5 & 6) | by CR1 (SEQ | | | |
| | | ID NO: 29) | | | |
| KM67 | UTR4 | SARS-CoV-2 | SEQ ID NO: 48 | SEQ ID NO: 49 | SEQ ID NO: 50 |
| | (SEQ ID | omicron spike | | | |
| | NOs: | protein optimized | | | |
| | 7 & 8) | by CR1 (SEQ | | | |
| | | ID NO: 29) | | | |
| KM68 | UTR7 | SARS-CoV-2 | SEQ ID NO: 51 | SEQ ID NO: 52 | SEQ ID NO: 53 |
| | (SEQ ID | omicron spike | | | |
| | NOs: | protein optimized | | | |
| | 13 & 14) | by CR1 (SEQ | | | |
| | | ID NO: 29) | | | |
| KM69 | UTR3 | SARS-CoV-2 | SEQ ID NO: 54 | SEQ ID NO: 55 | SEQ ID NO: 56 |
| | (SEQ ID | omicron spike | | | |
| | NOs: | protein optimized | | | |
| | 5 & 6) | by CR2 (SEQ | | | |
| | | ID NO: 30) | | | |
| KM70 | UTR4 | SARS-CoV-2 | SEQ ID NO: 57 | SEQ ID NO: 58 | SEQ ID NO: 59 |
| | (SEQ ID | omicron spike | | | |
| | NOs: | protein optimized | | | |
| | 7 & 8) | by CR2 (SEQ | | | |
| | | ID NO: 30) | | | |
| KM71 | UTR7 | SARS-CoV-2 | SEQ ID NO: 60 | SEQ ID NO: 61 | SEQ ID NO: 62 |
| | (SEQ ID | omicron spike | | | |
| | NOs: | protein optimized | | | |
| | 13 & 14) | by CR2 (SEQ | | | |
| | | ID NO: 30) | | | |
| KM72 | CureVac | SARS-CoV-2 | | SEQ ID NO: 63 | SEQ ID NO: 64 |
| | (SEQ ID | omicron spike | | | |
| | NOs: | protein optimized | | | |
| | 27 & 28) | by commercial | | | |
| | | software with | | | |
| | | CAI ≥ 0.7 | | | |
| | | (SEQ ID NO: 31) | | | |
| KM80 | Moderna | SARS-CoV-2 | | SEQ ID NO: 65 | SEQ ID NO: 66 |
| | 1273- | Wuhan spike | | | |
| | mRNA | protein optimized | | | |
| | UTRs | by commercial | | | |
| | (SEQ ID | software with | | | |
| | NOs: | CAI ≥ 0.7 | | | |
| | 23 & 24) | (SEQ ID NO: 117) | | | |
| KM81 | BioNTech | SARS-CoV-2 | | SEQ ID NO: 67 | SEQ ID NO: 68 |
| | (SEQ ID | Wuhan spike | | | |
| | NOs: | protein optimized | | | |
| | 21 & 22) | by commercial | | | |
| | | software with | | | |
| | | CAI ≥ 0.7 | | | |
| | | (SEQ ID NO: 117) | | | |
| KM82 | UTR3 | SARS-CoV-2 | SEQ ID NO: 69 | SEQ ID NO: 70 | SEQ ID NO: 71 |
| | (SEQ ID | Wuhan spike | | | |
| | NOs: | protein optimized | | | |
| | 5 & 6) | by commercial | | | |
| | | software with | | | |
| | | CAI ≥ 0.7 | | | |
| | | (SEQ ID NO: 117) | | | |
| KM83 | UTR4 | SARS-CoV-2 | SEQ ID NO: 72 | SEQ ID NO: 73 | SEQ ID NO: 74 |
| | (SEQ ID | Wuhan spike | | | |
| | NOs: | protein optimized | | | |
| | 7 & 8) | by commercial | | | |
| | | software with | | | |
| | | CAI ≥ 0.7 | | | |
| | | (SEQ ID NO: 117) | | | |
| KM84 | UTR7 | SARS-CoV-2 | SEQ ID NO: 75 | SEQ ID NO: 76 | SEQ ID NO: 77 |
| | (SEQ ID | Wuhan spike | | | |
| | NOs: | protein optimized | | | |
| | 13 & 14) | by commercial | | | |
| | | software with | | | |
| | | CAI ≥ 0.7 | | | |
| | | (SEQ ID NO: 117) | | | |
| KM85 | Moderna | SARS-CoV-2 | | SEQ ID NO: 78 | SEQ ID NO: 79 |
| | 1273- | Wuhan spike | | | |
| | mRNA | protein optimized | | | |
| | (SEQ ID | by commercial | | | |
| | NOs: | software with | | | |
| | 23 & 24) | CAI ≥ 0.7 | | | |
| | | (SEQ ID NO: 97) | | | |
| KM86 | BioNTech | SARS-CoV-2 | | SEQ ID NO: 80 | SEQ ID NO: 81 |
| | (SEQ ID | Wuhan spike | | | |
| | NOs: | protein optimized | | | |
| | 21 & 22) | by commercial | | | |
| | | software with | | | |
| | | CAI ≥ 0.7 | | | |
| | | (SEQ ID NO: 97) | | | |
| KM87 | UTR3 | SARS-CoV-2 | SEQ ID NO: 82 | SEQ ID NO: 83 | SEQ ID NO: 84 |
| | (SEQ ID | Wuhan spike | | | |
| | NOs: | protein optimized | | | |
| | 5 & 6) | by commercial | | | |
| | | software with | | | |
| | | CAI ≥ 0.7 | | | |
| | | (SEQ ID NO: 97) | | | |
| KM88 | UTR4 | SARS-CoV-2 | SEQ ID NO: 85 | SEQ ID NO: 86 | SEQ ID NO: 87 |
| | (SEQ ID | Wuhan spike | | | |
| | NOs: | protein optimized | | | |
| | 7 & 8) | by commercial | | | |
| | | software with | | | |
| | | CAI ≥ 0.7 | | | |
| | | (SEQ ID NO: 97) | | | |
| KM89 | UTR7 | SARS-CoV-2 | SEQ ID NO: 88 | SEQ ID NO: 89 | SEQ ID NO: 90 |
| | (SEQ ID | Wuhan spike | | | |
| | NOs: | protein optimized | | | |
| | 13 & 14) | by commercial | | | |
| | | software with | | | |
| | | CAI ≥ 0.7 | | | |
| | | (SEQ ID NO: 97) | | | |
| JW48 | Moderna- | SARS-CoV-2 | | SEQ ID NO: 91 | SEQ ID NO: 92 |
| | like UTR | Wuhan spike | | | |
| | | protein designed | | | |
| | | by commercial | | | |
| | | software | | | |
| | | (SEQ ID NO: 118) | | | |
| KM96 | BioNTech | SARS-CoV-2 | | SEQ ID NO: 93 | SEQ ID NO: 94 |
| | (SEQ ID | omicron spike | | | |
| | NOs: | protein optimized | | | |
| | 21 & 22) | by CR2 (SEQ | | | |
| | | ID NO: 30) | | | |
| VAC3 | CureVac | SARS-CoV-2 | | SEQ ID NO: 95 | SEQ ID NO: 96 |
| | (SEQ ID | Wuhan spike | | | |
| | NOs: | protein from The | | | |
| | 27 & 28) | World Health | | | |
| | | Organization: | | | |
| | | International | | | |
| | | Nonproprietary | | | |
| | | Names | | | |
| | | Programme. | | | |
| | | Messenger RNA | | | |
| | | Encoding the | | | |
| | | Full-Length | | | |
| | | SARS-CoV-2 Spike | | | |
| | | Glycoprotein; p. | | | |
| | | 11868, which is | | | |
| | | further described in | | | |
| | | Edo Kon, Uri Elia, | | | |
| | | and Dann Peer, | | | |
| | | “Principles for | | | |
| | | designing an | | | |
| | | optimal mRNA lipid | | | |
| | | nanoparticle | | | |
| | | vaccine,” Curr. | | | |
| | | Opin. Biotechnol. | | | |
| | | 2022, 73: 329-336 | | | |
| | | (SEQ ID NO: 116) | | | |

KM63T was a replicated batch of KM63.

The following sequences were also designed, and produced as identified in the examples below:

| | | | SEQ ID NO. of the complete molecule | | | |
|---|---|---|---|---|---|---|
| | | | | | With Xba | With Xba |
| | | | | With Xba | Restriction | Restriction |
| | | | | Restriction | Site and | Site and |
| | | | | Site, Without: | Illustrative | Illustrative |
| | | | | Illustrative | Cap | Cap |
| | | | | Cap | Consensus | Consensus |
| | | | | Consensus | Sequence, | Sequence, |
| | 3′ and | | | Sequence, | Without: | Without: |
| Construct | 5′ | Coding | Without Xba | representative | representative | representative |
| Name | UTRs | Sequence | Restriction Site | 3′ poly(A) tail, | 3′ poly(A) tail, | 3′ poly(A) tail, |
| LL34 | UTR4 | SARS-CoV- | SEQ ID NO: | SEQ ID NO: | SEQ ID NO: | SEQ ID NO: |
| | (SEQ | 2 omicron | 44 | 45 | 46 | 47 |
| | ID | BA1 spike | | | | |
| | NOs: | protein | | | | |
| | 7 & 8) | optimized | | | | |
| | | by | | | | |
| | | CR3 (SEQ | | | | |
| | | ID NO: 33) | | | | |
| LL35 | UTR4 | SARS-CoV- | SEQ ID NO: | SEQ ID NO: | SEQ ID NO: | SEQ ID NO: |
| | (SEQ | 2 omicron | 48 | 49 | 50 | 51 |
| | ID | BA1 spike | | | | |
| | NOs: | protein | | | | |
| | 7 & 8) | optimized | | | | |
| | | by | | | | |
| | | CR4 (SEQ | | | | |
| | | ID NO: 34) | | | | |
| LL36 | UTR4 | SARS-CoV- | SEQ ID NO: | SEQ ID NO: | SEQ ID NO: | SEQ ID NO: |
| | (SEQ | 2 omicron | 52 | 53 | 54 | 55 |
| | ID | BA1 spike | | | | |
| | NOs: | protein | | | | |
| | 7 & 8) | optimized | | | | |
| | | by | | | | |
| | | CR5 (SEQ | | | | |
| | | ID NO: 35) | | | | |
| LL37 | UTR4 | SARS-CoV- | SEQ ID NO: | SEQ ID NO: | SEQ ID NO: | SEQ ID NO: |
| | (SEQ | 2 omicron | 56 | 57 | 58 | 59 |
| | ID | BA1 spike | | | | |
| | NOs: | protein | | | | |
| | 7 & 8) | optimized | | | | |
| | | by | | | | |
| | | CR6 (SEQ | | | | |
| | | ID NO: 36) | | | | |
| LL38 | UTR4 | SARS-CoV- | SEQ ID NO: | SEQ ID NO: | SEQ ID NO: | SEQ ID NO: |
| | (SEQ | 2 omicron | 60 | 61 | 62 | 63 |
| | ID | BA1 spike | | | | |
| | NOs: | protein | | | | |
| | 7 & 8) | optimized | | | | |
| | | by | | | | |
| | | CR7 (SEQ | | | | |
| | | ID NO: 37) | | | | |
| LL40 | UTR7 | SARS-CoV- | SEQ ID NO: | SEQ ID NO: | SEQ ID NO: | SEQ ID NO: |
| | (SEQ | 2 omicron | 64 | 65 | 66 | 67 |
| | ID | BA1 spike | | | | |
| | NOs: | protein | | | | |
| | 13 & | optimized | | | | |
| | 14) | by | | | | |
| | | CR3 (SEQ | | | | |
| | | ID NO: 33) | | | | |
| LL41 | UTR7 | SARS-CoV- | SEQ ID NO: | SEQ ID NO: | SEQ ID NO: | SEQ ID NO: |
| | (SEQ | 2 omicron | 68 | 69 | 70 | 71 |
| | ID | BA1 spike | | | | |
| | NOs: | protein | | | | |
| | 13 & | optimized | | | | |
| | 14) | by | | | | |
| | | CR4 (SEQ | | | | |
| | | ID NO: 34) | | | | |
| LL42 | UTR7 | SARS-CoV- | SEQ ID NO: | SEQ ID NO: | SEQ ID NO: | SEQ ID NO: |
| | (SEQ | 2 omicron | 72 | 73 | 74 | 75 |
| | ID | BA1 spike | | | | |
| | NOs: | protein | | | | |
| | 13 & | optimized | | | | |
| | 14) | by | | | | |
| | | CR5 (SEQ | | | | |
| | | ID NO: 35) | | | | |
| LL43 | UTR7 | SARS-CoV- | SEQ ID NO: | SEQ ID NO: | SEQ ID NO: | SEQ ID NO: |
| | (SEQ | 2 omicron | 76 | 77 | 78 | 79 |
| | ID | BA1 spike | | | | |
| | NOs: | protein | | | | |
| | 13 & | optimized | | | | |
| | 14) | by | | | | |
| | | CR6 (SEQ | | | | |
| | | ID NO: 36) | | | | |
| LL44 | UTR7 | SARS-CoV- | SEQ ID NO: | SEQ ID NO: | SEQ ID NO: | SEQ ID NO: |
| | (SEQ | 2 omicron | 80 | 81 | 82 | 83 |
| | ID | BA1 spike | | | | |
| | NOs: | protein | | | | |
| | 13 & | optimized | | | | |
| | 14) | by | | | | |
| | | CR7 (SEQ | | | | |
| | | ID NO: 37) | | | | |
| LL59 | UTR4 | SARS-CoV- | SEQ ID NO: | SEQ ID NO: | SEQ ID NO: | SEQ ID NO: |
| (a.k.a. | (SEQ | 2 omicron | 84 | 85 | 86 | 87 |
| KM70 | ID | BA5 spike | | | | |
| Prime) | NOs: | protein | | | | |
| | 7 & 8) | optimized | | | | |
| | | by | | | | |
| | | CR2 (SEQ | | | | |
| | | ID NO: 38) | | | | |
| LL60 | UTR4 | SARS-CoV- | SEQ ID NO: | SEQ ID NO: | SEQ ID NO: | SEQ ID NO: |
| | (SEQ | 2 omicron | 88 | 89 | 90 | 91 |
| | ID | BA5 spike | | | | |
| | NOs: | protein | | | | |
| | 7 & 8) | optimized | | | | |
| | | by | | | | |
| | | CR3 (SEQ | | | | |
| | | ID NO: 39) | | | | |
| LL61 | UTR4 | SARS-CoV- | SEQ ID NO: | SEQ ID NO: | SEQ ID NO: | SEQ ID NO: |
| | (SEQ | 2 omicron | 92 | 93 | 94 | 95 |
| | ID | BA5 spike | | | | |
| | NOs: | protein | | | | |
| | 7 & 8) | optimized | | | | |
| | | by | | | | |
| | | CR4 (SEQ | | | | |
| | | ID NO: 40) | | | | |
| LL62 | UTR4 | SARS-CoV- | SEQ ID NO: | SEQ ID NO: | SEQ ID NO: | SEQ ID NO: |
| | (SEQ | 2 omicron | 96 | 97 | 98 | 99 |
| | ID | BA5 spike | | | | |
| | | protein | | | | |
| | NOs: | optimized | | | | |
| | 7 & 8) | by | | | | |
| | | CR5 (SEQ | | | | |
| | | ID NO: 41) | | | | |
| LL66 | UTR4 | SARS-CoV- | SEQ ID NO: | SEQ ID NO: | SEQ ID NO: | SEQ ID NO: |
| | (SEQ | 2 omicron | 100 | 101 | 102 | 103 |
| | ID | BA5 spike | | | | |
| | NOs: | protein | | | | |
| | 7 & 8) | optimized | | | | |
| | | by | | | | |
| | | CR6 (SEQ | | | | |
| | | ID NO: 42) | | | | |
| LL67 | UTR4 | SARS-CoV- | SEQ ID NO: | SEQ ID NO: | SEQ ID NO: | SEQ ID NO: |
| | (SEQ | 2 omicron | 104 | 105 | 106 | 107 |
| | ID | BA5 spike | | | | |
| | NOs: | protein | | | | |
| | 7 & 8) | optimized | | | | |
| | | by | | | | |
| | | CR7 (SEQ | | | | |
| | | ID NO: 43) | | | | |

### Cell Culture and mRNA Transfection Conditions

Baby hamster kidney (BHK) cells and human embryonic kidney 293 (HEK293) cells were maintained by routine passaging in growth media (Dulbecco's Modified Eagle Medium (DMEM, Lonza 12-614F) supplemented with 10 v/v % fetal bovine serum (FBS) (Corning 35-016-CV), antibiotic (Gibco 15140-122) and 0.1% v/v 0.1 M glutamine (Gibco 25030-081)) and grown at 37° C. and 5% CO₂.

BHK and HEK293 cells were seeded in growth media at 5×10⁵ cells/mL onto either 24-well tissue culture plates (Costar 3524) or collagen coated (2.5 mg/well, Gibco A10483-01) 96-well, clear-bottom, black-walled imaging microwell plates (Corning 3904). The following day, target mRNAs are complexed with TransiT mRNA transfection reagent (Mirus mir2250) in OptiMEM (Gibco 31985-070). Each target mRNA is forward transfected into HEK293 monolayers using 0.327% transfection reagent (final concentration) with mRNAs diluted in a 2x serial dilution ranging from 0.909 ng/mL to 0.114 ng/mL (final concentration), or water-only negative control. The transfected HEK293 cells are incubated overnight at standard tissue culture conditions.

### Fluorescence-Activated Cell Sorting of Luciferase Positive Cells

Cells treated with RNA encoding nanoluciferase and under the control of UTRs were removed from the plate using 0.01% trypsin for less than 1 minute and then fluorescence-activated cell sorted (FACS). The following UTRs were used: beta-actin gene (ACTB, SEQ ID NOs: 15 & 16), albumin gene (ALB or UTR1, SEQ ID NOs: 1 & 2), adenosine triphosphate (ATP) synthase beta subunit gene (ATPSB or UTR2, SEQ ID NOs: 3 & 4), fibroblast activation protein messenger ribonucleic acid (mRNA) (FAP or UTR 3, SEQ ID NOs: 5 & 6), H4 clustered histone 15 mRNA (HIST2H4A or UTR4, SEQ ID NOs: 7 & 8), glyceraldehyde-3-phosphate dehydrogenase gene (GAPDH or UTR5, SEQ ID NOs: 9 & 10), heat shock protein family A (Hsp70) member 8 gene (HSPA8 or UTR6, SEQ ID NOs: 11 & 12), interleukin-2 gene (IL-2 or UTR7, SEQ ID NOs: 13 & 14), transferrin gene (TF, SEQ ID NOs: 17 & 18), and eukaryotic translation elongation factor 2 (EEF2) mRNA (SEQ ID NOs: 19 & 20). Whether the RNA comprised modified nucleotides or cap-1 capping are provided in the examples below.

### For Whole-Cell and Surface SARS-CoV-2 Spike Protein Expression Assay

All recombinant RNA molecules were produced by in vitro transcription using N1-methyl pseudouridine to replace all uridines. All recombinant RNA molecules comprised a cap-1 5′ cap and a 3′ poly(A) tail. The mRNAs were purified and evaluated for mRNA integrity (by capillary gel electrophoresis), double stranded RNA (dsRNA) content (by Homogeneous Time Resolved Fluorescence [HTRF]), poly-A tail length, and percent capping. The specific RNA designs (i.e. KM71 RNA) are provided in the examples below.

Approximately, 18 hours post-transfection the cell media was removed from cells in 96-well format and cell monolayers were rinsed once with phosphate buffered saline (PBS) with calcium and magnesium (ThermoFisher Scientific #14080055). The cell monolayers were fixed in 4% paraformaldehyde (ThermoFisher Scientific J19943-K2), followed by rinsing twice with PBS (VWR #02-0119-1000).

For the whole-cell SARS-CoV-2 spike protein expression assay, fixed HEK293 cell monolayers in one set of 96 well plates were permeabilized with 0.5% Triton-X-100 (Ricca Chemical Co. 8698.5-16) in PBS for 15 min. Cell monolayers were rinsed 3 times with PBS-0.1% Tween20 (Sigma p7949-500 mL). Nonspecific antibody-binding was blocked using 1% horse serum (Gibco 16050-130) in PBS-0.1% Tween20 (blocking solution). SARS-CoV-2 spike protein was labeled by incubating cell monolayers with human anti-SARS-CoV-2 spike protein primary antibody (S309: Lot #N77817-1, Carrie Saucedo 27aug.2020) diluted 1:1500 in blocking solution. Then, cell monolayers were rinsed 3 times with PBS-0.1% Tween20. Indirect immunofluorescent detection of SARS-CoV-2 spike protein expression assay was completed by incubating cell monolayers with goat anti-human antibody with Alexa647 (ThermoFisher Scientific A-21445) diluted 1:2000 in blocking solution. Additionally, cell nuclei were co-labeled with DyeCycle Violet (ThermoFisher Scientific V35003), following manufacturer's recommendations. Cell monolayers were rinsed 3 times with PBS-0.1% tween then cells were stored in PBS for imaging.

SARS-CoV-2 spike protein expression was evaluated at the cell membrane surface for a separate set of 96 well plates. Nonspecific antibody-binding for fixed cell monolayers was blocked using 0.1% bovine serum albumin (BSA) (AlfaAesar J64655) in PBS (0.1% BSA-PBS). SARS-CoV-2 spike protein was labeled by incubating cell monolayers with human anti-SARS-CoV-2 spike protein primary antibody (S309) diluted 1:1500 in 0.1% BSA-PBS. Then, cell monolayers were rinsed 3 times with 0.1% BSA-PBS. Indirect immunofluorescent detection of SARS-CoV-2 spike protein was completed by incubating cell monolayers with goat anti-human antibody with Alexa647 diluted 1:2000 in 0.1% BSA-PBS. Additionally, cell nuclei were co-labeled with DyeCycle Violet, following manufacturer's recommendations. Cell monolayers were rinsed 3 times with 0.1% BSA-PBS then cells were stored in PBS for imaging.

Six fields per well were imaged using the 10× objective on the ThermoFishe rScientific Cell Insight CX7 automated imaging system in the DyeCycle Violet and Alexa647 fluorescent channels. Image analysis was completed using the Target Activation protocol associated with the Cellomics (HCS Navigator Ver 6.6.2 Build 8533) image analysis system. Data analysis was completed using Microsoft Excel and Prism Graphpad.

### For SARS2 Spike-ACE2 Binding and Detection Assay

Approximately, 18 hours post transfection the HEK293 cell suspension was created from transfected cell monolayers in 24-well format by rinsing once with PBS (VWR #02-0119-1000) followed by incubating cells in cell dissociation buffer (Gibco 13151-014) while moderately tritrating cells until many cells were observed as singlets.

The live, HEK293 cell-suspension was equilibrated in ice-cold PBS-2.5% FBS (rinse buffer). Cell-surface SARS-CoV-2 spike protein was bound to angiotensin-converting enzyme 2 (ACE2) soluble protein by bathing cells in ice-cold rinse buffer with 1:150 dilution of ACE2 (Acro Biosystems AC2-H82E6) followed by one rinse in ice-cold rinse buffer. The bound ACE2 soluble protein was labeled using the primary anti-ACE2 (R&D Systems AF933) antibody diluted 1:200 in ice cold rinse buffer. The cell suspension was rinsed once in ice-cold rinse buffer. Following primary antibody, cell suspensions were incubated with secondary anti-goat Alexa488 antibody (Invitrogen A11055) at a 1:1000 dilution in ice cold rinse buffer. The cell suspension was rinsed once in rinse buffer. Next, cell suspension was equilibrated in QSOL (Sartorius 91304) and the cells were analyzed using the Sartorius iQ3 flow cytometer and associated ForeCyte software. Data analysis was completed using Microsoft Excel and Prism Graphpad.

### Total In Vivo SARS-CoV-2 Spike Protein-Specific Antibody Titers

A multiplex assay was performed to evaluate SARS-Cov2 spike-specific antibodies in the serum of the immunized mice. LUMINEX™ microspheres (MAGPLEX™ microspheres, LUMINEX™ Corp from Austin, TX) were coupled with SARS-Cov2 spike antigen (lot: N79663) at 10 μg/mL reaction by chemical coupling according to manufacturer instructions. In 96 well plates, about 1000 microspheres/well were added in a volume of 50 μL phosphate-buffered saline (PBS) with 1% bovine serum albumin (BSA)+0.05% sodium azide (assay buffer) into five-fold serial dilutions of mouse serum. The microspheres and serum were incubated on an orbital shaker (covered) for 60 minutes at room temperature. The microspheres were washed one time three times with 200 μL per well of PBS with 0.05% Tween-20 (wash buffer) on a plate washer using a magnet to allow settling of beads between washes. Following the wash, 50 μL per well of r-Phycoerythrin (r-PE) conjugated anti-mouse immunoglobulin G (IgG) (Jackson Immunoresearch) was added at a 1:50 dilution, and plates were incubated (covered) on an orbital shaker at room temperature for 60 minutes. The plates were washed with the methods described above and incubated with PBS (covered) at room temperature on an orbital shaker for 20 minutes. Fluorescent intensity was measured using a Luminex INTELLIFLEX™ FLEXMAP 3D™ (Life Technologies model INTELLIFLEX FM3D000). The raw data was analyzed using a Softmax Pro template, where the serum sample binding potency was interpolated based on a five-parameter logistic fit of the standard curve. Serum anti-SARS-CoV2 spike potency was calculated in terms of Assay Units (AU) using a reference standard. In brief, a cocktail of monoclonal antibodies (mAb) specific for SARS-CoV-2 spike were prepared (1A9 Genetex #75870-168, mCR 3022 Absolute Antibody #AB1680-3.0, and MP 0142 MP Biologicals #8720412) at an optimized dilution (1:17.36 for 1A9, 1:11.6 for mCR 3022, and 1:9.63 for MP 0142). The readout from this mAb cocktail was assigned a concentration of 100 AU for standard normalization purposes.

### Anti-SARS-CoV-2 Neutralization Antibody Titers

Serum samples were analyzed using a validated pseudovirion neutralization assay (PNA) against the Wuhan (WT), Delta, or Omicron variants. On day 1 of the neutralization assay (which is discrete from the days of in vivo treatments), Vero E6 cells expressing the ACE-2 receptor were seeded in 96-well white plates at 20,000 cells/well to reach a cell confluence of 80% next day. On day 2, serum samples and controls were serially diluted. In parallel, SARS-CoV-2 pseudovirus was diluted to reach the desired concentration (according to pre-determined TU/mL). Pseudovirus was added to diluted serum samples and pre-incubated for 1 hour at 37° C. with 5% CO2. The mixture was added to the pre-seeded Vero E6 cell layers and plates were incubated for 18-22 hours at 37° C. with 5% CO2. On day 3, following incubation and removal of media, One-Glo EX luciferase assay substrate was added to cells and incubated for 3 minutes at RT with shaking. Luminescence of the luciferase was measured using a SPECTRAMAX™ iD3 microplate reader and SoftMax Pro v7.0.1. Luminescence results for each dilution were used to generate a titration curve using a 4-parameter logistic regression (4PL). The titers were defined as the reciprocal dilution of the sample for which the luminescence is equal to a pre-determined cut-point of 50, corresponding to 50% neutralization. This cut-point was established using linear regression using 50% flanking points. Samples were analyzed using an array of acceptance criteria and were repeated if an acceptance criterion is not met.

### Live Virus Antibody Neutralization Titers

A Microneutralization (MN) assay was used for serological detection of SARS-CoV-2 specific neutralizing antibodies in mouse serum samples, against SARS-CoV-2 Omicron variant (SARS-CoV-2 ß.1.1.529 Omicron) (Refer to the link to “ANALYTICAL STUDY REPORT FOR MICRONEUTRALIZATION CPE-BASED ASSAY FOR SARS-COV-2 FOR GSK PROTOCOL NUMBER AP33647/ELN44471/B-000-G5Q” in Annexes, or to the raw data, Table 27, in the Annexes.) Vismederi, Siena, Italy.

### Intracellular Cytokine Staining by Flow Cytometry

Multi-parameter flow-cytometry with intracellular cytokine staining (ICS) was used to assess SARS-CoV-2 Spike-specific CD4 and CD8 T cell responses. Spleens were harvested on day 35 from 5 mice per group. Splenocytes from individual mice were plated in round-bottom 96 well tissue culture plates at 1.5×10⁶ viable cells/well and stimulated with one of the following pools of spike peptides (from JPT Peptide Technologies Gmbh, Berlin, Germany or Genscript):

| | No. | | Vendor | |
|---|---|---|---|---|
| | Peptides | Stimulation | (Catalog | Lot |
| Stimulation | in Pool | Concentration | Number) | No. |
| Media | N/A | N/A | N/A | N/A |
| Spike Peptide | 158 | 1.0 μg/mL | JPT Peptide | 47886VSi-01 |
| Pool 1 70% | | | Technologies | |
| Purity | | | Gmbh (PM-WCPV- | |
| | | | S-2, vial 1) | |
| Spike Peptide | 157 | 1.0 μg/mL | JPT Peptide | 47886VSi-02 |
| Pool 2 70% | | | Technologies | |
| Purity | | | Gmbh (PM-WCPV- | |
| | | | S-2, vial 2) | |
| Spike full | 6 | 1.0 μg/mL | Genscript | U260NFE270 |
| length gap | | | | |
| peptides | | | | |
| Spike Ecto gap | 8 | 1.0 μg/mL | Genscript | U260NFE270 |
| peptides | | | | |

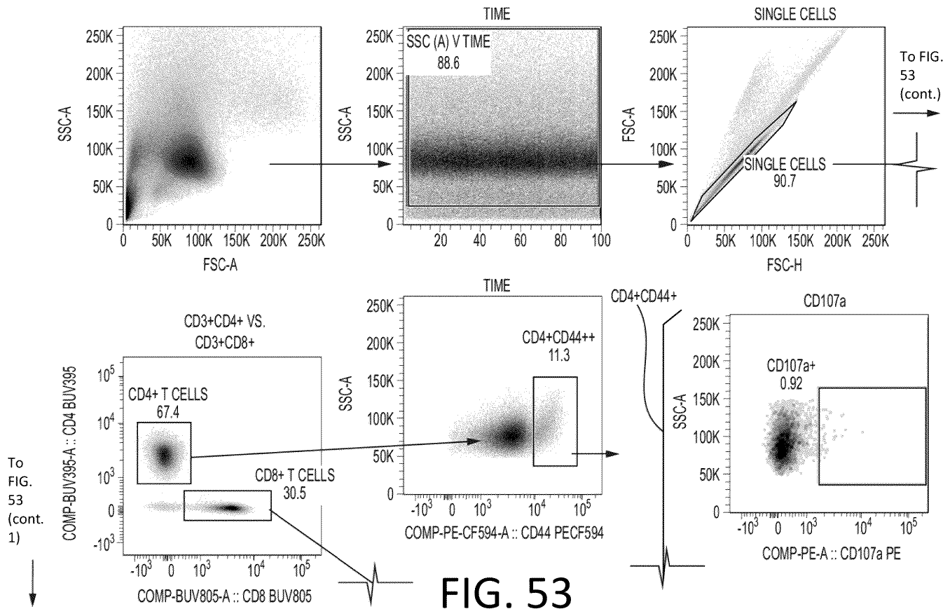

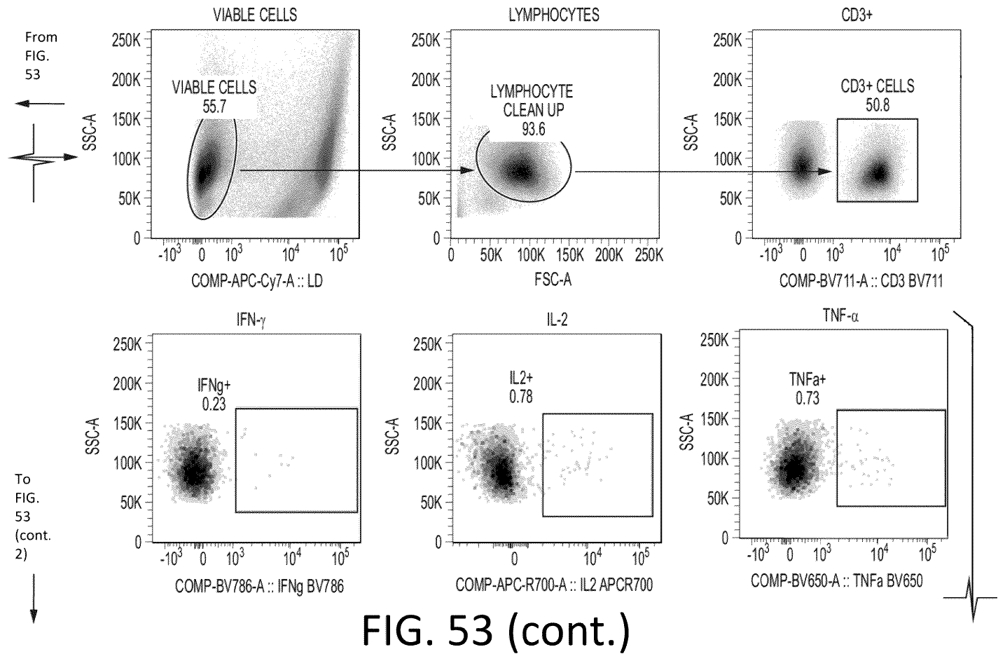

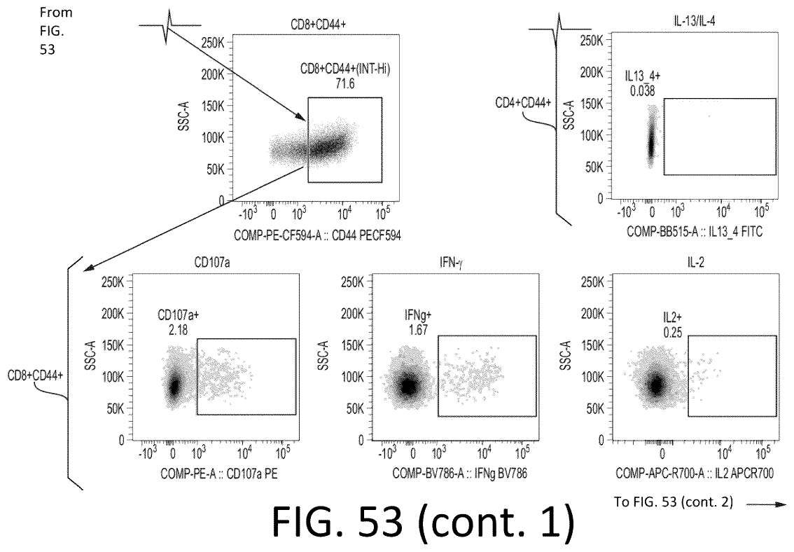

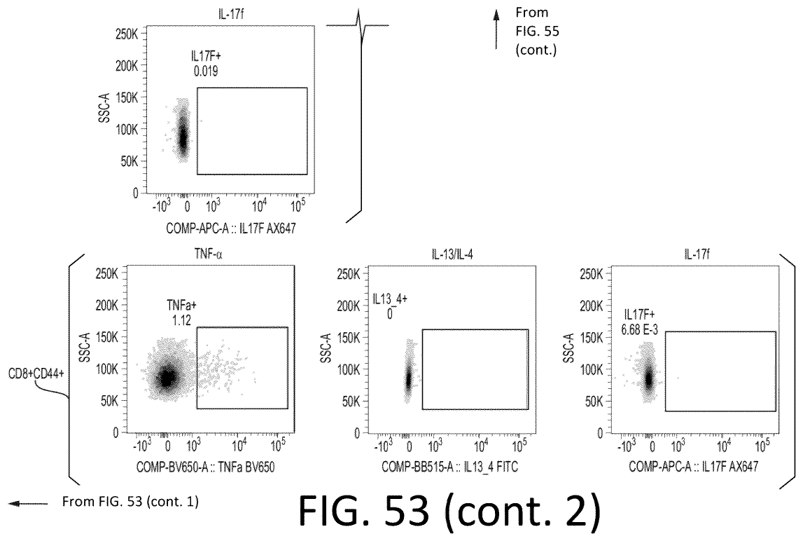

All samples were co-stimulated with the anti-CD28 clone 37.51 monoclonal antibody (mAb, ThermoFisher, Catalog No. 14-0281-82) and stained for the degranulation marker CD107α, 12-14 hours, in culture (37° C., 5% CO2) for 12-14 hours, thereby performing a stimulatory incubation. Two hours into the stimulatory incubation, BD GOLGIPLUG™, a protein transport inhibitor containing Brefeldin A, was added to all samples to block cytokine secretion from the cell. Upon completion of the stimulatory incubation, all samples were stained for: 1) viability, 2) phenotypic markers a) CD3, b) CD4, c) CD8, and d) CD44, and 3) cytokines a) IFN-γ, b) IL-2, c) TNF-α, d) IL-17F, and e) IL-4/IL-13. The data was acquired on the BD FAC SYMPHONY™ A5 special order research product cell analyzer using the High Throughput Sampler (HTS) option for acquisition in a 96 well plate format. Prior to analysis, the raw .fcs data files were analyzed by the computational algorithm Flow AI, which is a quality control process that identifies and removes aberrant events caused by anomalies in fluorescence signal across all acquired parameters or flow rate over time of data acquisition. The flow cytometry data analysis was performed using FLOWJO™ Vi0.8.0. FIG. 53 illustrates the gating strategy used to identify activated CD4 and CD8 T cells. First, gates were defined that identified cytokine and CD107a expressing positive populations within the viable, activated, CD4 and CD8 T cell populations. Next, the Boolean combination gate tool was used to automatically generate all possible combinations (positive and negative) of cytokine and CD107a expressing cells. For activated CD4 and CD8 T cells, all six analytes were included in the Boolean analysis generating a total of 64 multi-functional subsets. The raw data was exported into Microsoft Excel for further analysis. The response to peptide pool stimulation was determined by subtracting the response in the unstimulated media control for each sample. Negative values resulting from this subtraction were identified and valued as null (“zero”). The multi-functional subsets were used to categorize the data into CD4 T helper (Th) and CD8 T cytotoxic (Tc) phenotypic subsets based on production of IFN-γ, IL-13/IL-4, and IL-17F cytokines, and these subsets are defined in Table 4.

TABLE 4

| Phenotype | IFN-γ | IL-13/IL-4 | IL-17F | IL-2 | TNF-α | CD107a |
|---|---|---|---|---|---|---|
| Tₕ/T_{c}0 | − | − | − | +/− | +/− | +/− |
| Tₕ/T_{c}1 | + | − | − | +/− | +/− | +/− |
| Tₕ/T_{c}2 | − | + | − | +/− | +/− | +/− |
| Tₕ/T_{c}17 | − | − | + | +/− | +/− | +/− |

All graphs of the intracellular cytokine staining by flow cytometry were generated with GraphPad Prism v8.0.0.

### Tfh Staining by Flow Cytometry

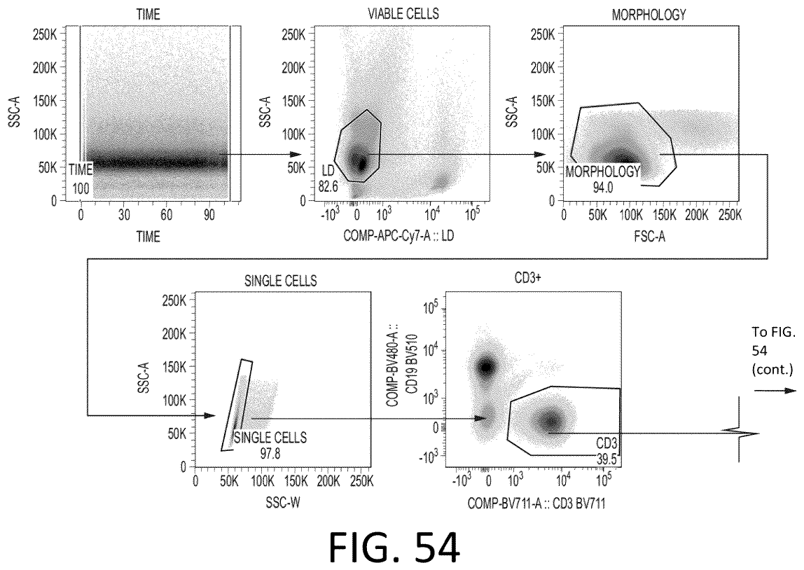

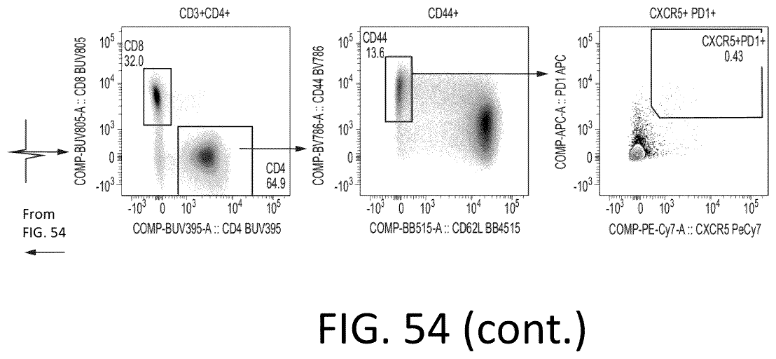

Spleens were collected from 5 mice per group at day 35. Splenocytes from individual mice were plated in round-bottom 96 well tissue culture plates at 1.5×10⁶ viable cells per well. Cells were stained with 1:1000 near IR Live/Dead stain (Invitrogen) for 20 min at room temperature and then treated with 1:15 diluted Fc block (BD Biosciences) in fluorescence-activated single cell sorting (FACS) wash buffer (PBS plus 1 volume % Fetal Bovine Serum, Thermo Scientific) for 10 min at 4° C. to avoid non-specific binding. Cells were then stained with the following extracellular markers: CD19-BV510, CD62L-BB515, CD127-BUV737, CXCR5-PECy7, PD-1-APC, CXCR3-BB700, CCR6-BV605, and ICOS—BV421, in Brilliant Stain Buffer (BSB) and FACS Wash Buffer. Cells were incubated for 20 minutes in the dark and fixed and permeabilized using BD cytofix/cytoperm solution for 20 minutes at 4° C. in the dark. After incubation, the cells were treated with 1:15 dilution of Fc Block for 10 min, and then stained with an intracellular antibody mix: CD3-BV711, CD4-BUV395, CD8-BUV805, CD44-BV786, and Bcl6-PE-CF594 in BSB in FACS Perm/Wash Buffer (10× BD Perm/Wash diluted to 1× in Ultra Pure Distilled Water) for 30 minutes at room temperature (in the dark). The cells were subsequently washed then resuspended in FACS Wash Buffer and acquired on the BD FACSYMPHONY™ A5 special order research product cell analyzer. Data was analyzed using FlowJo v10.8.0. Tfh cells were identified according to the gating strategy in FIG. 54.

### Example 1

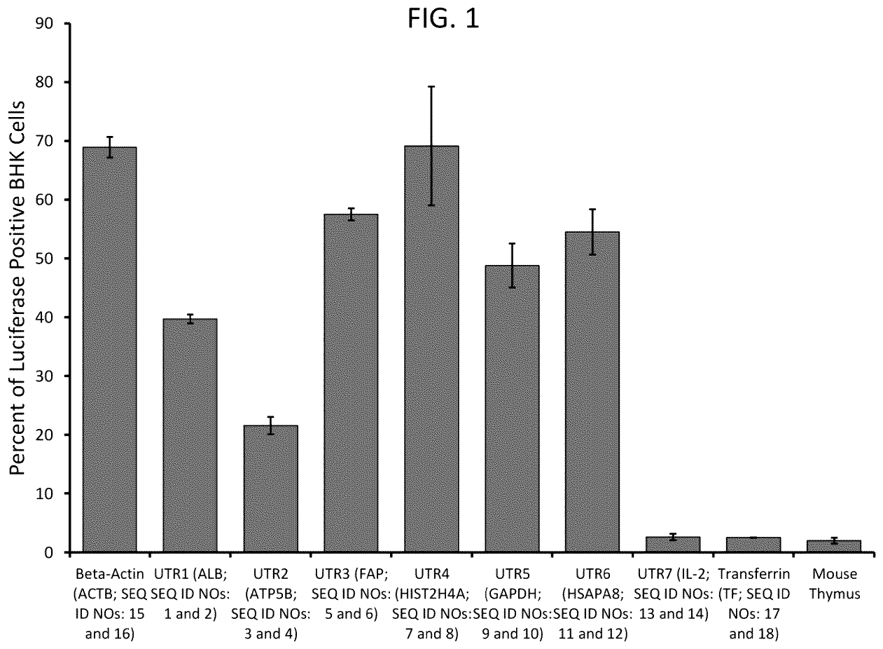

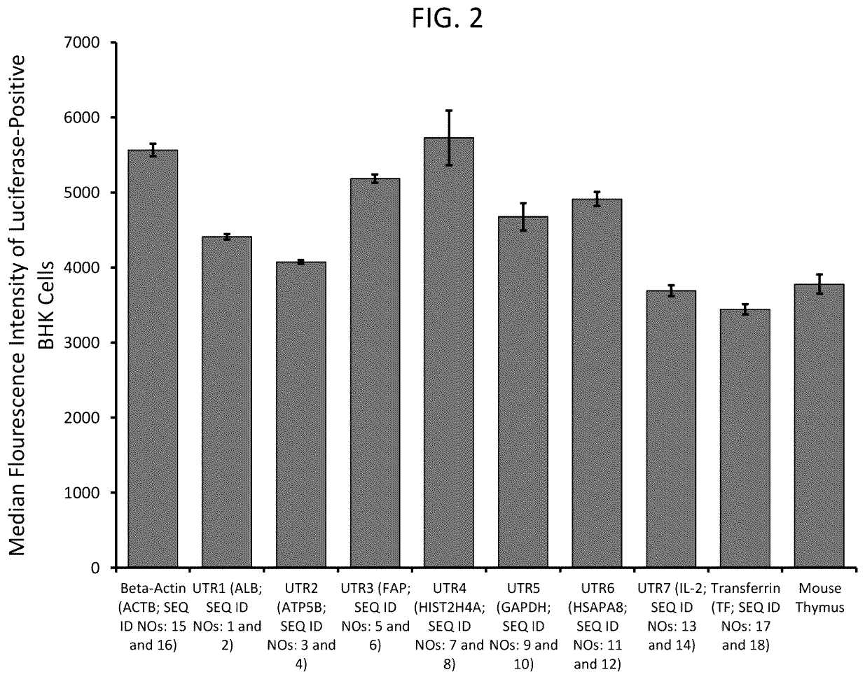

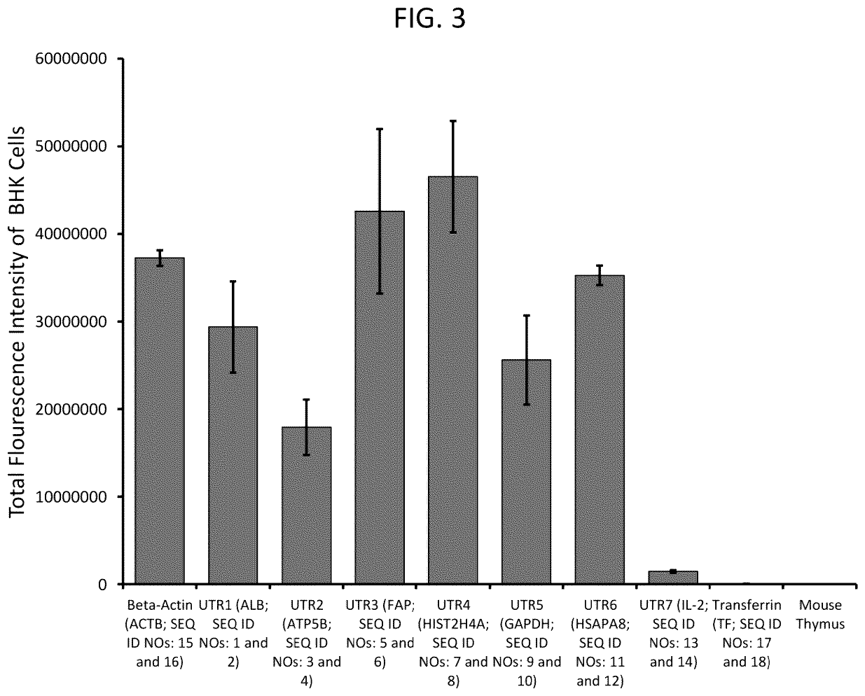

Baby hamster kidney cells were transfected with RNA encoding firefly luciferase under the control of the following human UTRs: beta-actin gene (ACTB, Accession NO: NM_001101.5; SEQ ID NOs: 15 & 16, complete mRNA in vitro transcribed from SEQ ID NO: 111), albumin gene (ALB or UTR1, Accession NOs: NG_056261.1 & NM_000477.7; SEQ ID NOs: 1 & 2, complete mRNA in vitro transcribed from SEQ ID NO: 104), adenosine triphosphate (ATP) synthase beta subunit gene (ATPSB or UTR2, Accession NOs: AH002618.2 & NM_001686.4; SEQ ID NOs: 3 & 4, complete mRNA in vitro transcribed from SEQ ID NO: 105), fibroblast activation protein messenger ribonucleic acid (mRNA) (FAP or UTR 3, SEQ ID NOs: 5 & 6, Accession NOs: U09278.2 & NM_001291807.3, complete mRNA in vitro transcribed from SEQ ID NO: 106), H4 clustered histone 15 mRNA (HIST2H4A or UTR4, SEQ ID NOs: 7 & 8; Accession NO: NR_168448.1, complete mRNA in vitro transcribed from SEQ ID NO: 107), glyceraldehyde-3-phosphate dehydrogenase gene (GAPDH or UTR5, Accession NOs: NG_007073.2 & NR_152150.2; SEQ ID NOs: 9 & 10, complete mRNA in vitro transcribed from SEQ ID NO: 108), heat shock protein family A (Hsp70) member 8 gene (HSPA8 or UTR6, Accession NOs: NG_029473.1 & NM_153201.4; SEQ ID NOs: 11 & 12, complete mRNA in vitro transcribed from SEQ ID NO: 109), interleukin-2 gene (IL-2 or UTR7; Accession NOs: AH002842.2 & NM_000586.4;; SEQ ID NOs: 13 & 14, complete mRNA in vitro transcribed from SEQ ID NO: 110), and transferrin gene (TF, Accession NO: NG_013080.2; SEQ ID NOs: 17 & 18, complete mRNA in vitro transcribed from SEQ ID NO: 112), with untransfected mouse thymus cells and BHK cells as controls. Baby hamster kidney cells lack an innate immune response and thereby demonstrate gene expression from the UTR without inhibitory influence from the innate immune response. As shown in FIGS. 1-3, UTRs 3 and 4 stimulate firefly luciferase expression as well as ACTB UTR does when an innate immune response is not present. Beta actin is expressed ubiquitously in cells. Firefly luciferase fluorescence was detected 18 hours after transfection. The mRNA had cap-0 capping and uridines, but no N1-methylpseudouridines.

### Example 2

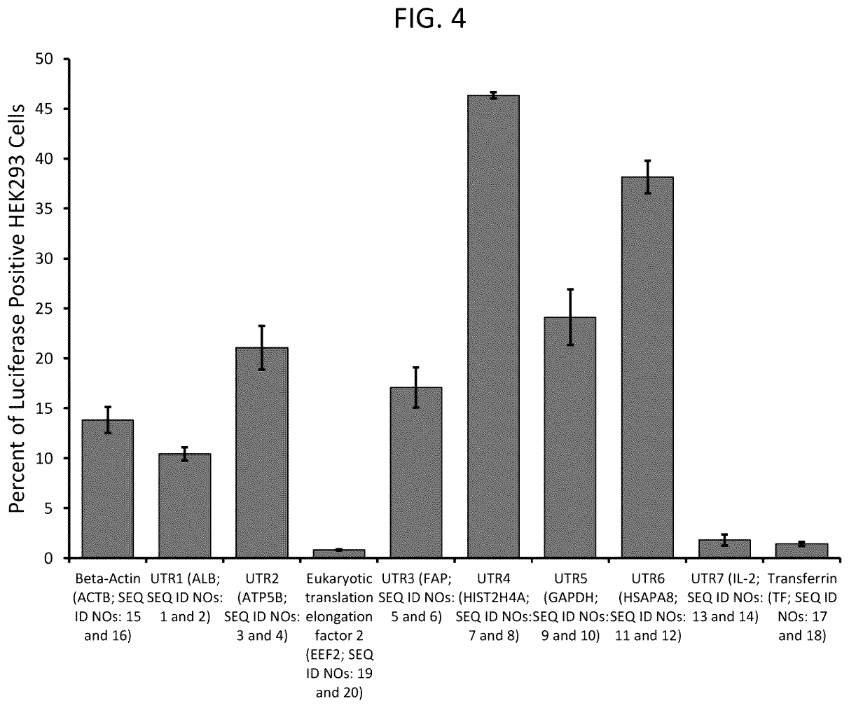

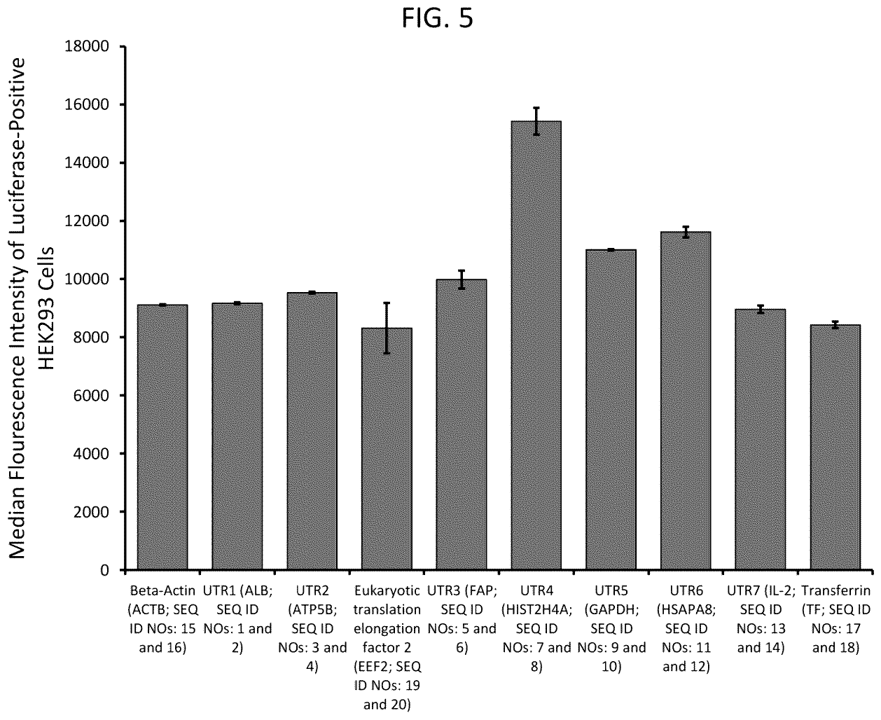

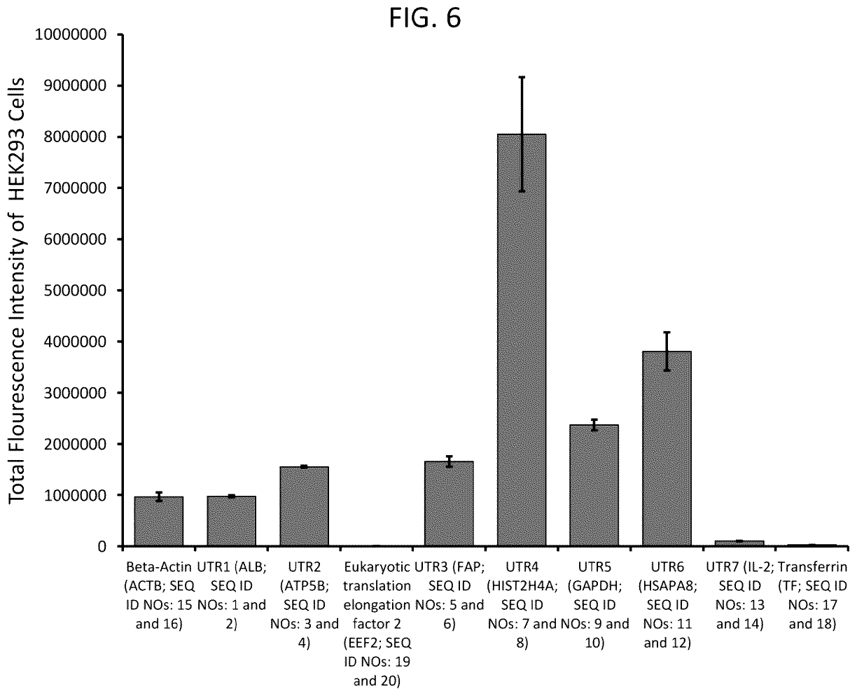

Human embryonic kidney 293 cells were transfected with RNA encoding firefly luciferase under the control of the following human UTRs: ACTB, UTRs1-7, TF, and eukaryotic translation elongation factor 2 (EEF2) mRNA (Accession NO: NM_001961.4; SEQ ID NOs: 19 & 20, complete mRNA in vitro transcribed from SEQ ID NO: 113). Firefly luciferase fluorescence was detected 18 hours after transfected. The mRNA had cap-0 capping and uridines, but no N1-methylpseudouridines. As shown in FIGS. 4-6, UTRs 2-6 outperformed ACTB UTRs in triggering more cells to express firefly luciferase.

### Example 3

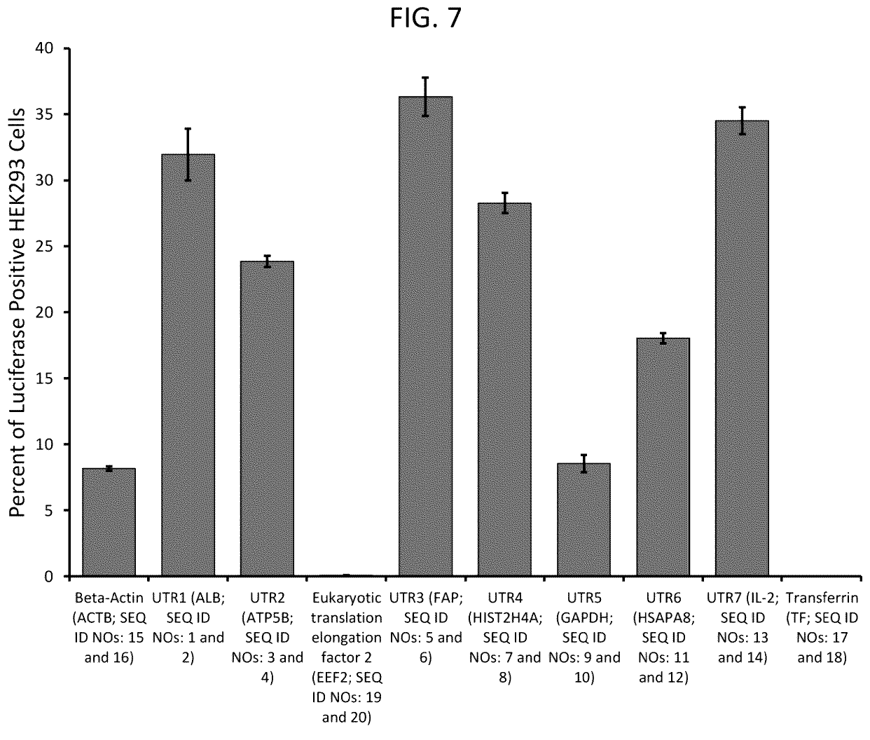

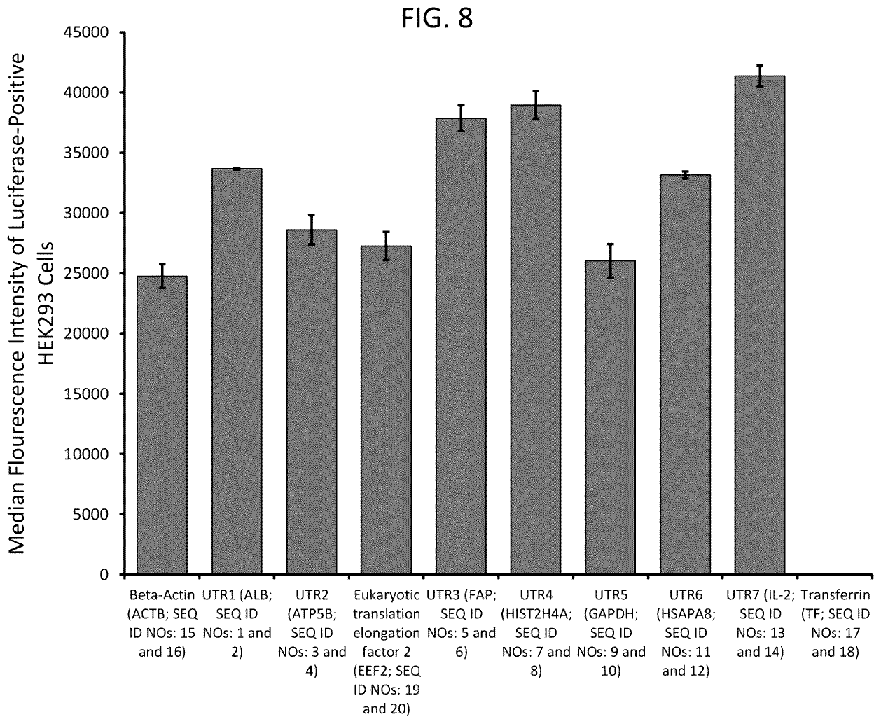

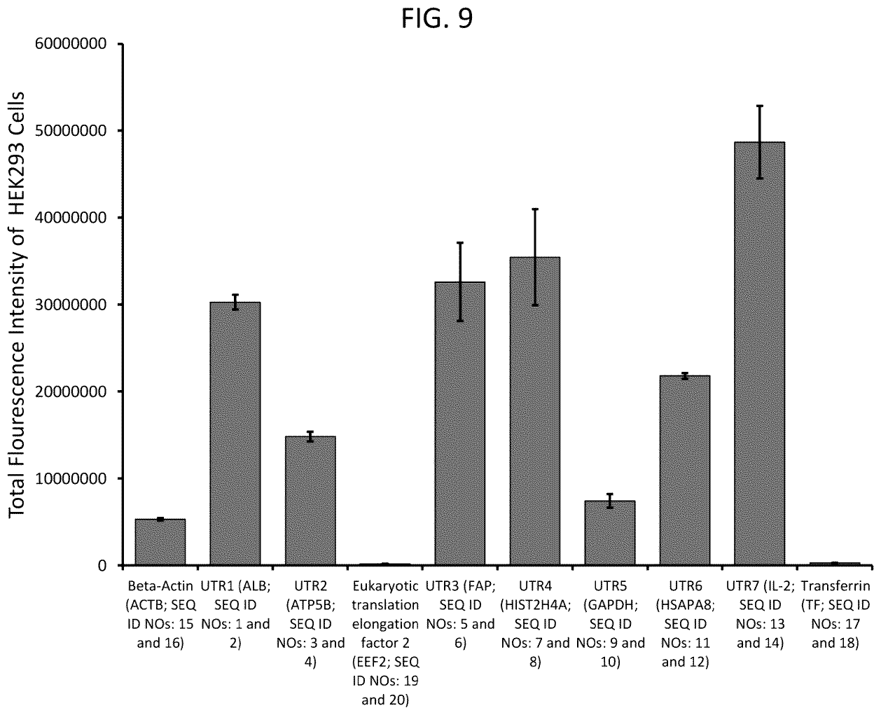

Human embryonic kidney 293 cells were transfected with RNA encoding firefly luciferase under the control of the following human UTRs: ACTB, UTRs1-7, TF, and eukaryotic translation elongation factor 2 (EEF2) mRNA. Firefly luciferase fluorescence was detected 18 hours after transfection. The mRNA had cap-1 capping and N1-methylpseudouridines, but no uridines. As shown in FIG. 7, UTRs 1, 2, 3, 4, 6, and 7 outperformed ACTB UTRs in triggering more cells to express firefly luciferase. As shown in FIGS. 8 and 9, at least cells treated with mRNA comprising UTRs 1, 2, 3, 4, 6, and 7 expressed more firefly luciferase than those cells treated with mRNA comprising ACTB UTRs. Cells treated with mRNA comprising UTR 5 and EEF UTRs had expression that was equal to or greater than that of cells treated with mRNA comprising ACTB UTRs.

### Example 4A

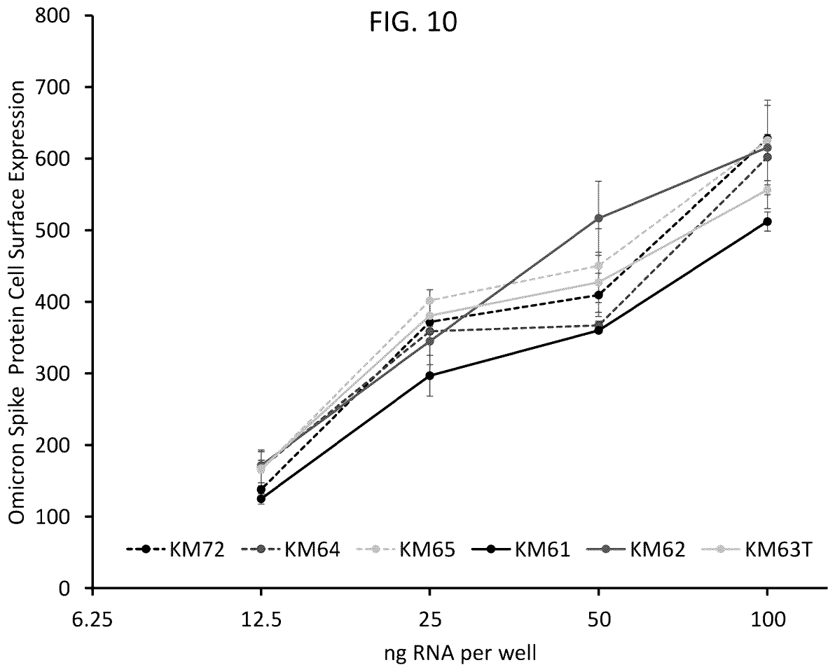

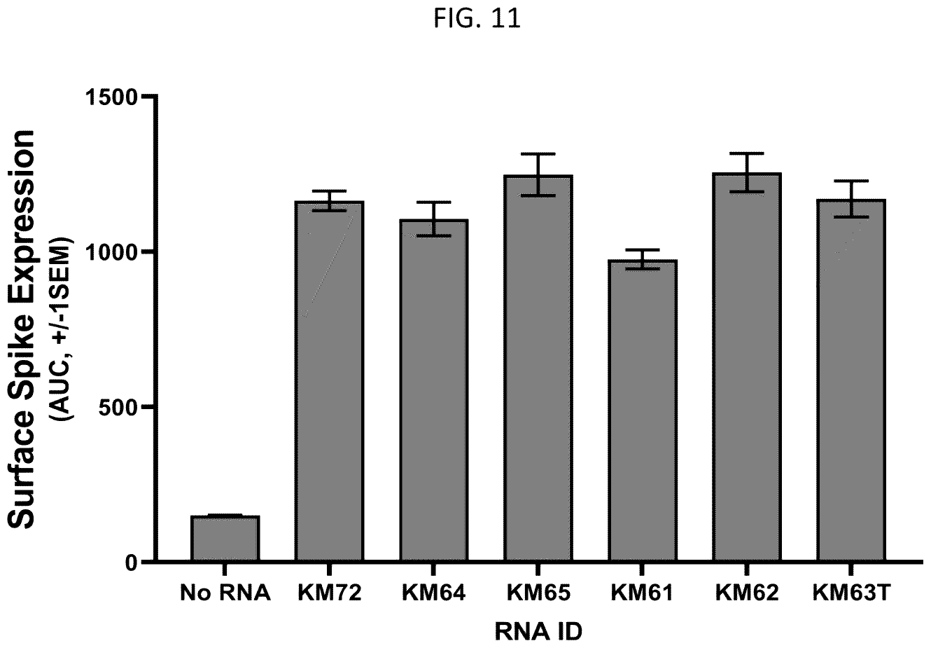

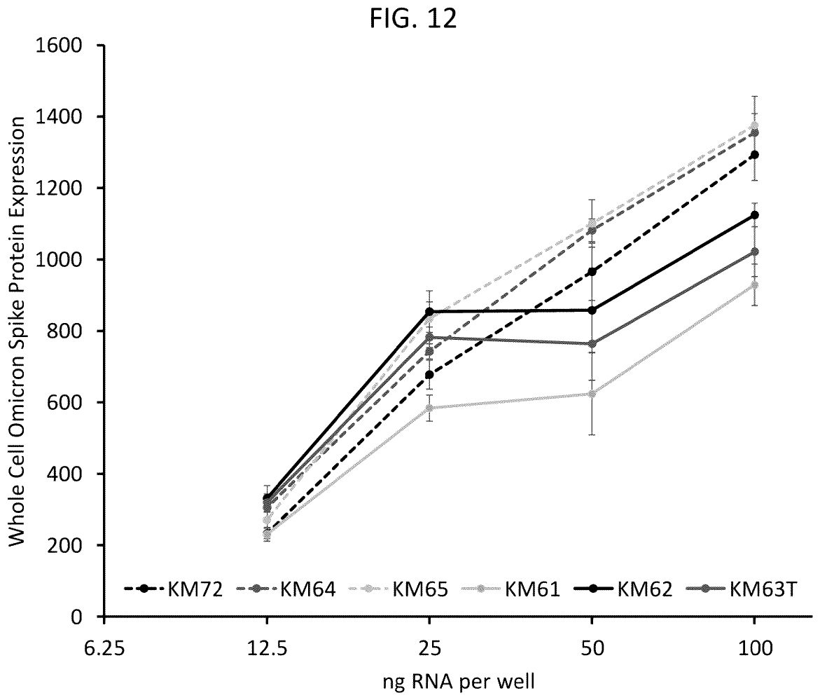

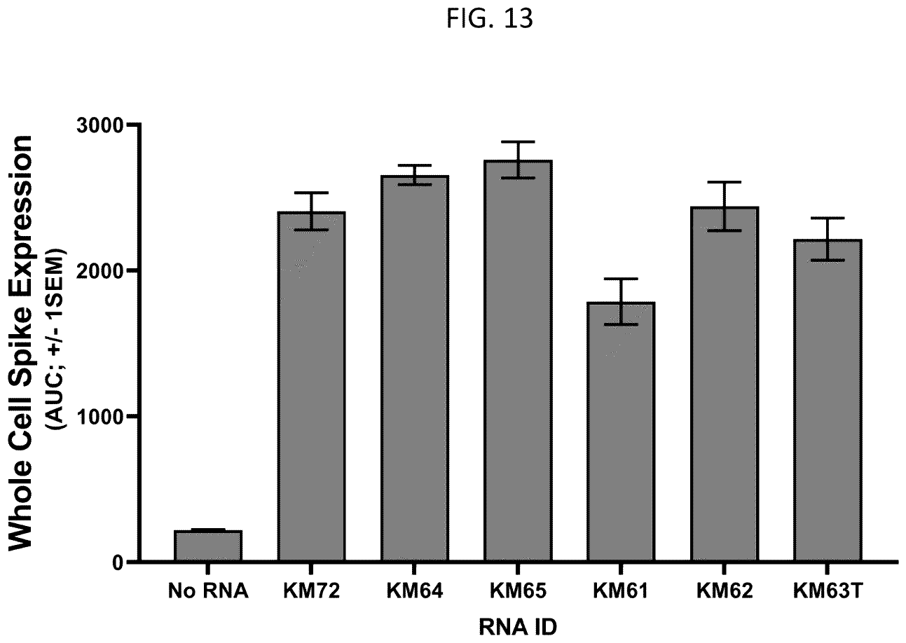

HEK293 cells were transfected with 12.5 ng, 25 ng, 50 ng, and 100 ng of KM72 (SEQ ID NO: 64), KM64 (SEQ ID NO: 42), KM65 (SEQ ID NO: 44), KM61 (SEQ ID NO: 34), KM62 (SEQ ID NO: 37), or KM63 (SEQ ID NO: 40) RNA and were assessed for surface and whole cell SARS-CoV-2 omicron spike protein as described above. FIGS. 10 and 11 depict the dose response curve and area under the curve, respectively, of expression of spike protein from SARS-CoV-2 omicron strain on the cells' surface. FIGS. 12 and 13 show the dose response curve and area under the curve, respectively, of expression of whole cell spike protein. Three prime and 5′ UTRs 4 and 7 (in KM62 and KM63 respectively) drove cell membrane surface expression of SARS CoV-2 omicron spike protein as well as UTRs from CureVac's second SARS-CoV-2 candidate (KM72, SEQ ID NOs: 27 and 28), BioNTech's BNT162b2 SARS-CoV-2 FDA-approved product (Accession NOs: NM_000558, OL521838, NM_001130, SEQ ID NOs: 19 and 20, in KM64), and Moderna's mRNA-1273 SARS-CoV-2 FDA-approved product (SEQ ID NOs: 23 and 24, in KM65). The SARS-COV-2 omicron spike protein mRNA sequences were the same across constructs and were designed using commercially available software that optimized only for a codon adaptation index of greater than or equal to 0.7.

### Example 4B

HEK293 cells were transfected with 12.5 ng, 25 ng, 50 ng, and 100 ng of KM62 (SEQ ID NO: 37), KM63 (SEQ ID NO: 40), KM67 (SEQ ID NO: 50), KM68 (SEQ ID NO: 53), KM69 (SEQ ID NO: 56), KM70 (SEQ ID NO: 59), KM71 (SEQ ID NO: 62), or KM96 (SEQ ID NO: 94) RNA per well and were assessed for surface and whole cell SARS-CoV-2 omicron spike protein as described above. The SARS-COV-2 omicron spike protein mRNA sequences were the same across constructs and were designed using the CR2 algorithm.

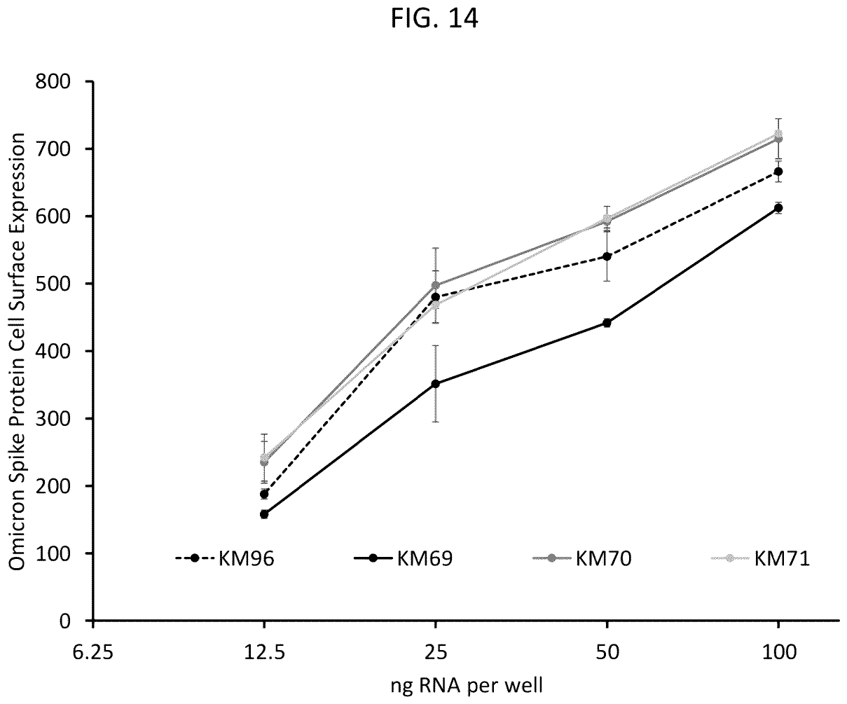

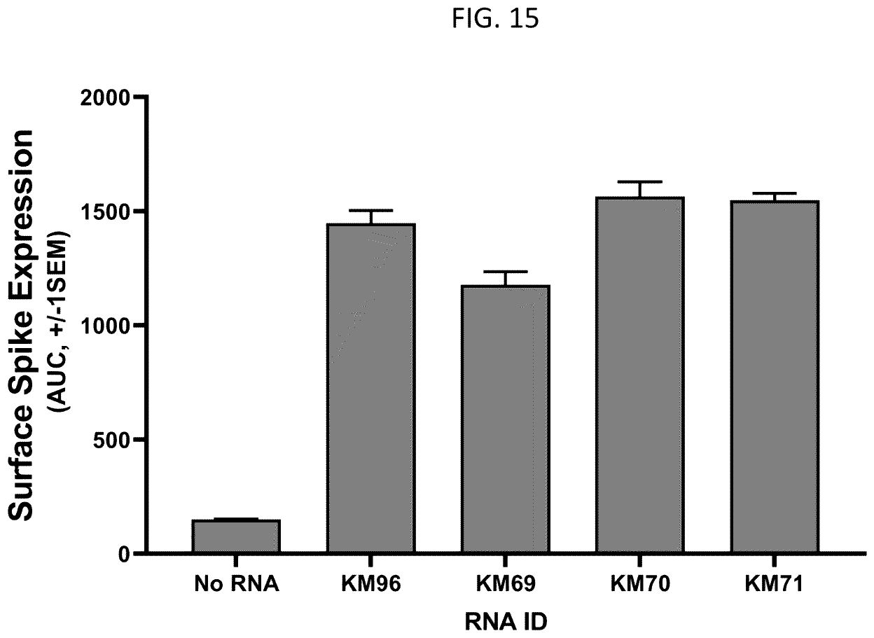

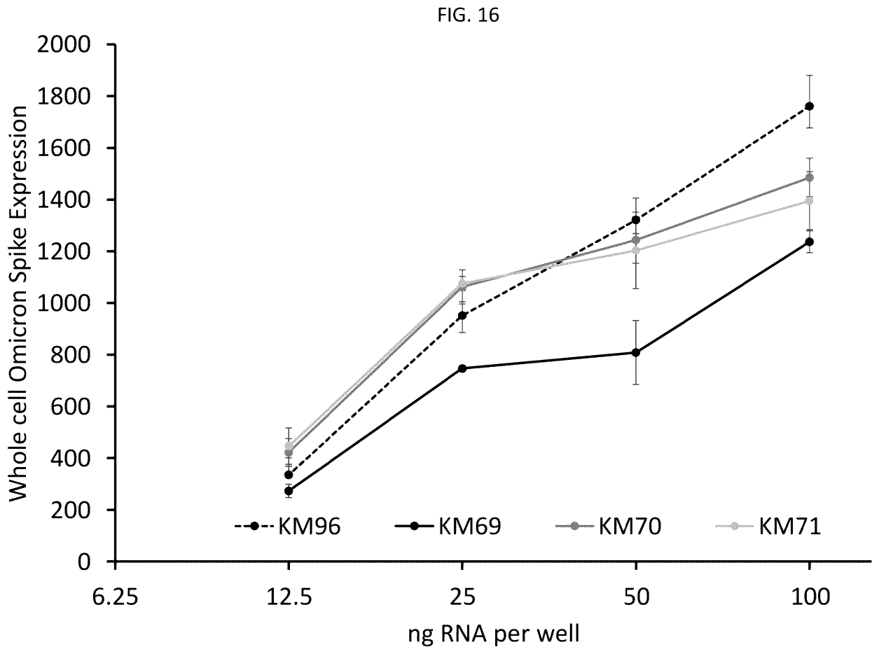

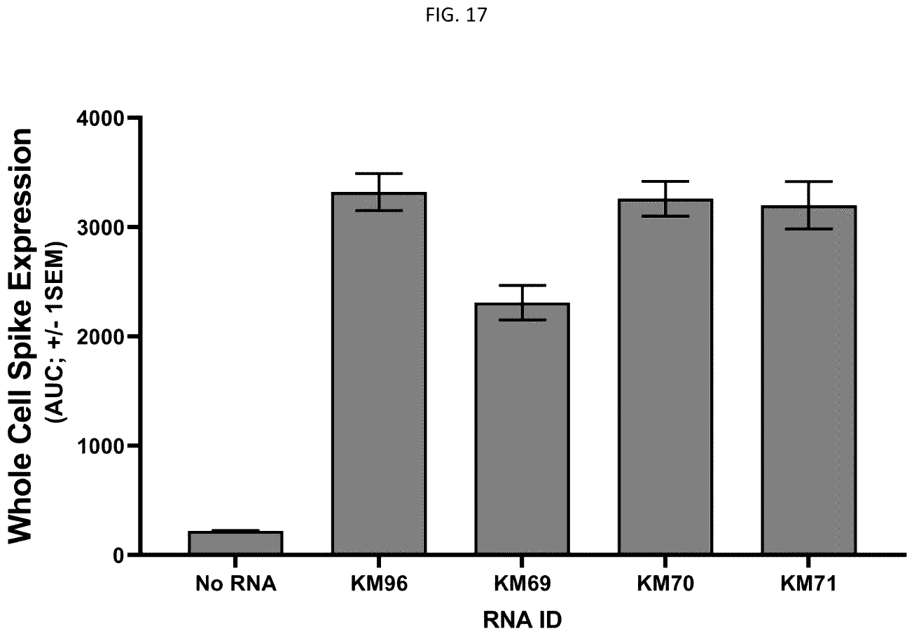

FIGS. 14 and 15 depict the dose response curve and area under the curve, respectively, of expression of spike protein from SARS-CoV-2 omicron strain on the cells' surface with KM69, KM70, KM71, or KM96 RNA transfection. FIGS. 16 and 17 show the dose response curve and area under the curve, respectively, of expression of whole cell spike protein with KM69, KM70, KM71, or KM96 transfection. Three prime and 5′ UTR 4 (KM70) and 3′ and 5′ UTR 7 (KM71) drove cell membrane surface expression of SARS CoV-2 omicron spike protein as well as a UTR from BioNTech's BNT162b2 SARS-CoV-2 FDA-approved product (Accession NOs: NM_000558, OL521838, NM_001130, SEQ ID NOs: 19 and 20, in KM96).

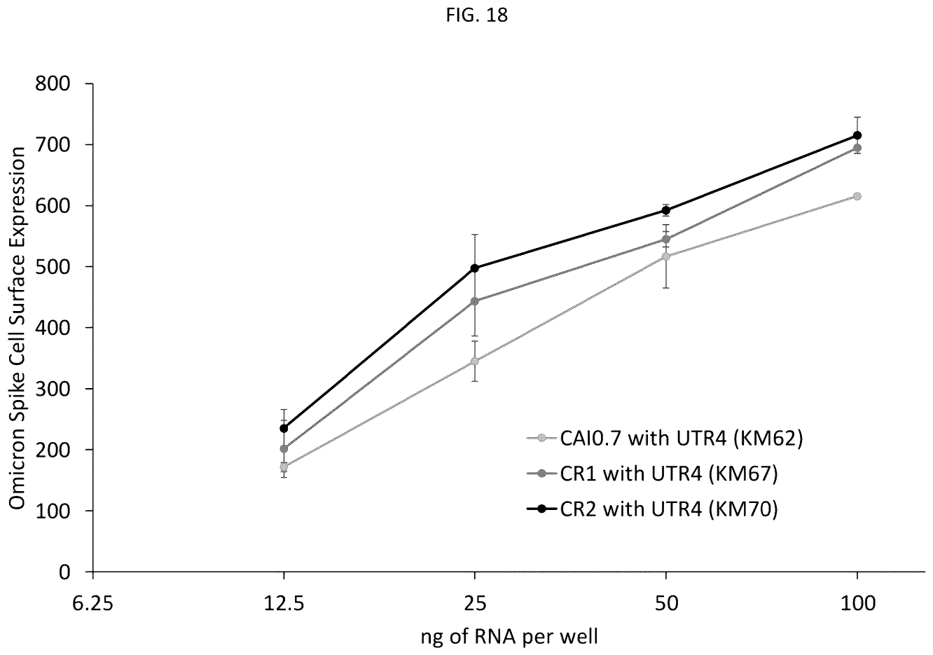

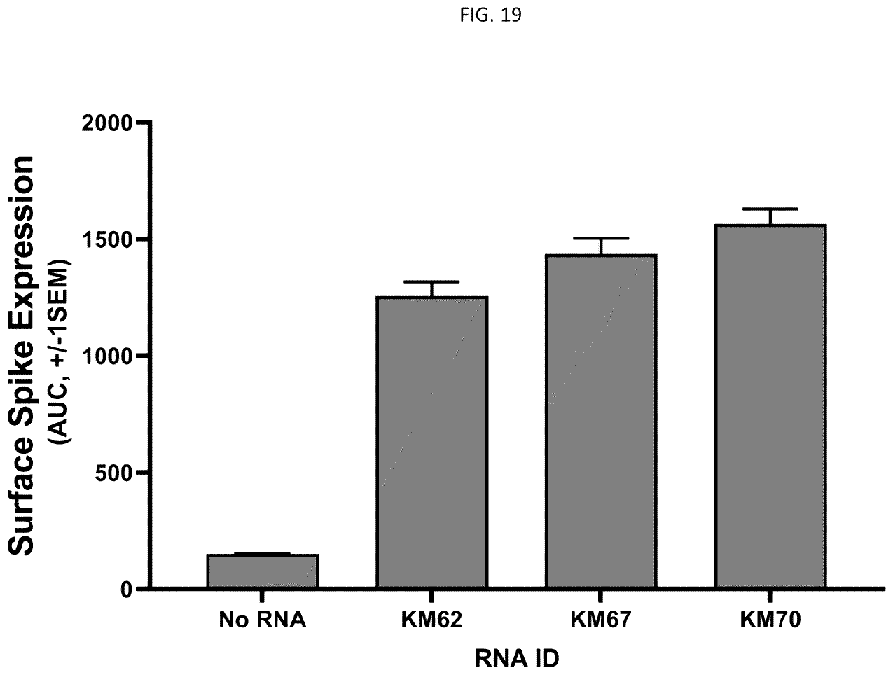

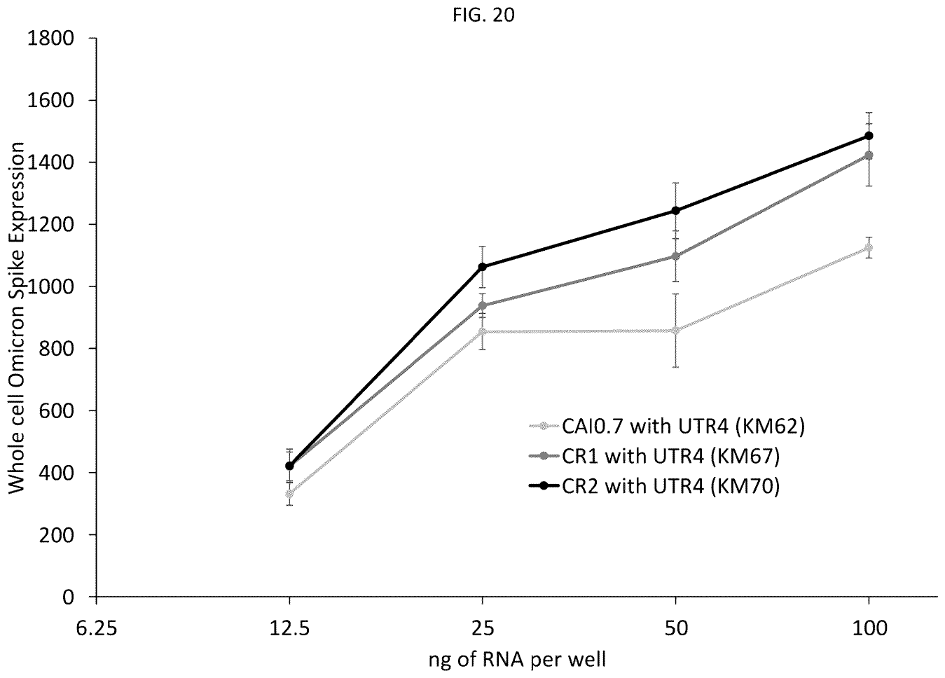

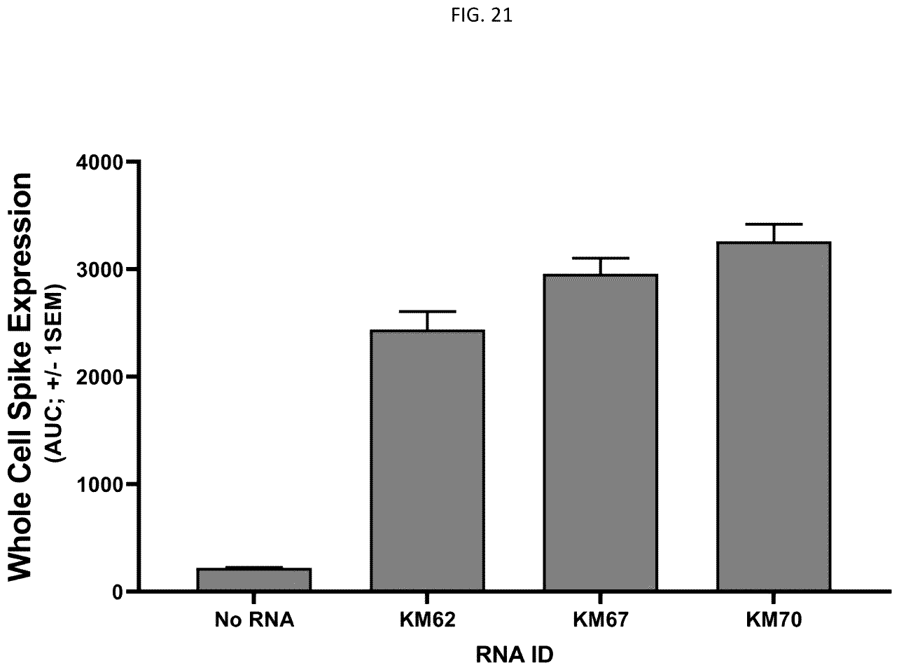

FIGS. 18 and 19 depict the dose response curve and area under the curve, respectively, of expression of spike protein from SARS-CoV-2 omicron strain on the cells' membranes with KM62, KM67, or KM70 RNA transfection the cell membrane surface of HEK293 cells. KM62, KM67, and KM70 comprise 5′ and 3′ UTR4. Design with the CR1 (KM67) and CR2 (KM70) algorithms resulted in significantly more surface expression of SARS-CoV-2 omicron strain than achieved with CAI 0.7 (KM62), a commercially available software that optimized only for a codon adaptation index of greater than or equal to 0.7. FIGS. 20 and 21 show the dose response curve and area under the curve, respectively, of expression of whole cell spike protein from the same treatments as in FIGS. 18 and 19.

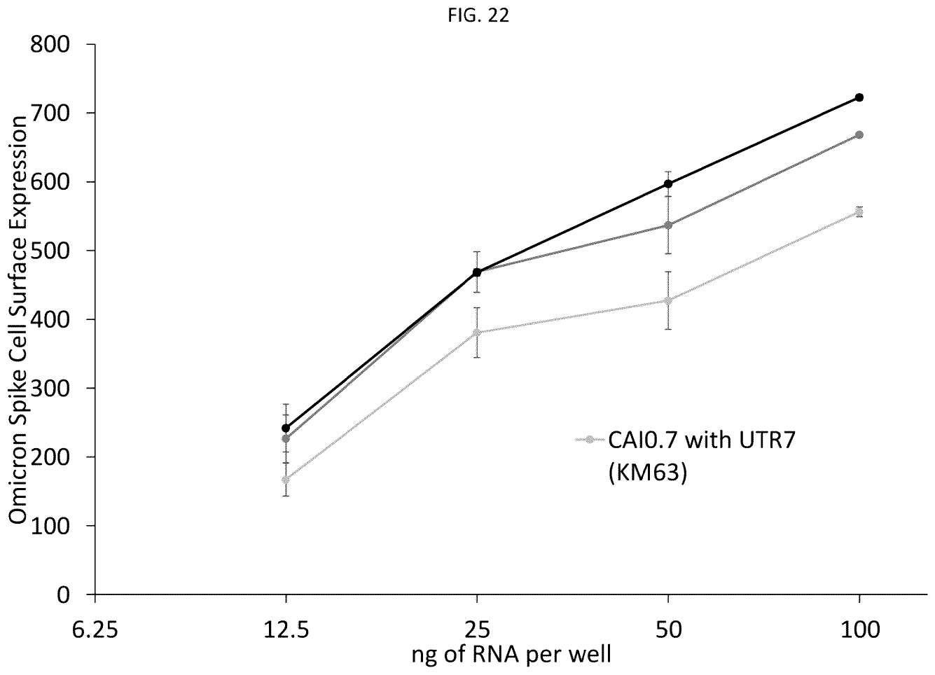

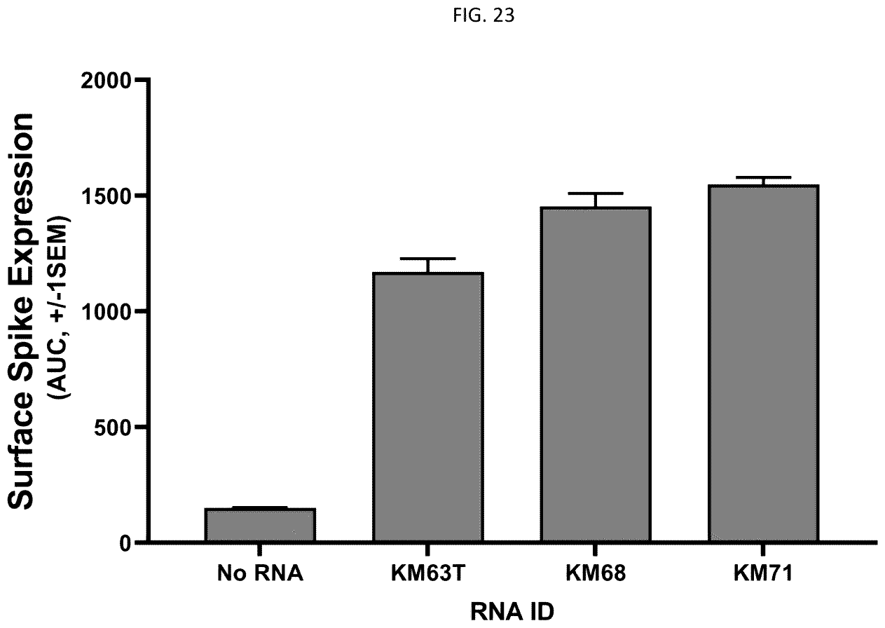

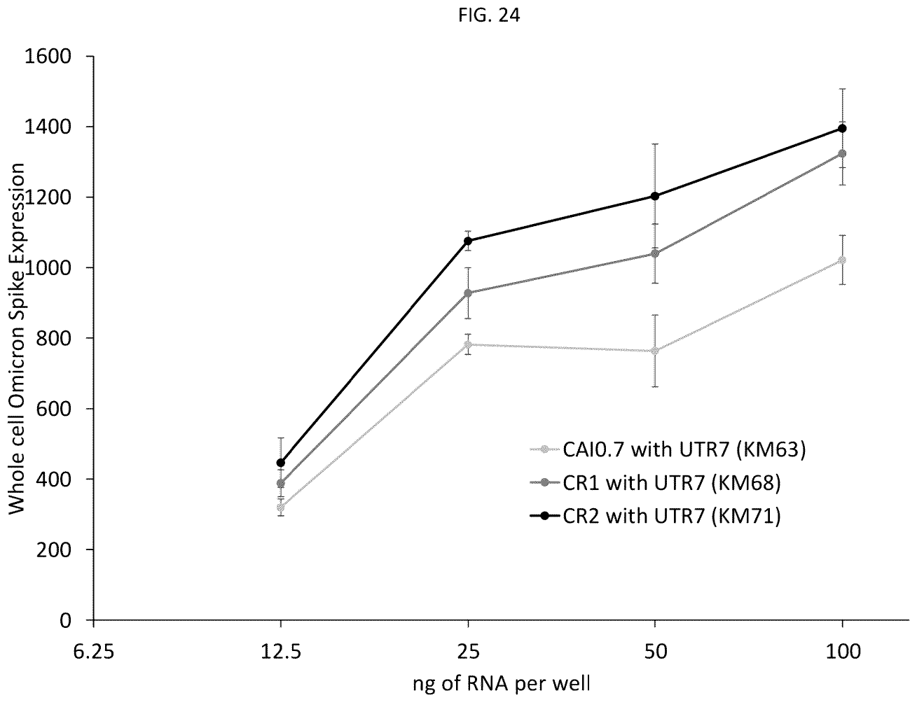

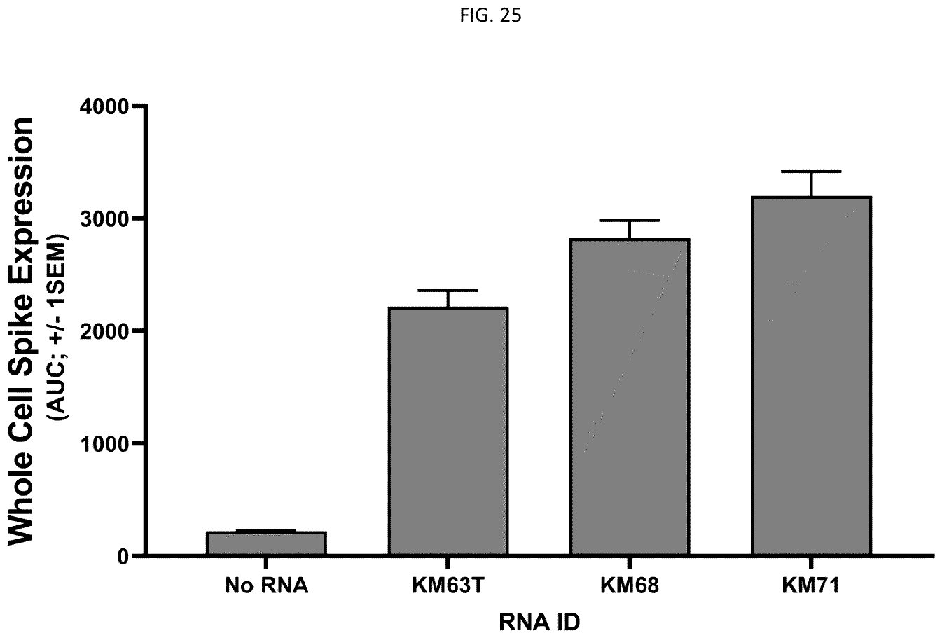

FIGS. 22 and 23 depict the dose response curve and area under the curve, respectively, of expression of spike protein from SARS-CoV-2 omicron strain on the cell membrane surface of HEK293 cells with KM63, KM68, and KM71 RNA transfection. KM63, KM68, and KM71 comprise 5′ and 3′ UTR7. Design with the CR1 (KM68) and CR2 (KM71) algorithms resulted in significantly more surface expression of SARS-CoV-2 omicron strain than achieved with CAI 0.7 (KM63), a commercially available software that optimized only for a codon adaptation index of greater than or equal to 0.7. FIGS. 24 and 25 show the dose response curve and area under the curve, respectively, of expression of whole cell spike protein from the same treatments as in FIGS. 22 and 23.

### Example 5

HEK293 cells were transfected with 12.5 ng, 25 ng, 50 ng, and 100 ng of VAC3, KM87, KM88, KM89, JW48, KM71, KM68, KM70, KM62, KM65, KM63, KM66, or KM61 RNA per well. The mRNA sequences for KM87, KM88, and KM89 that encoded SARS-CoV-2 Wuhan spike protein were the same and were designed using commercially available software that optimized only for a codon adaptation index of greater than or equal to 0.7. The mRNA sequence for JW48 that encoded SARS-CoV-2 Wuhan spike protein (SEQ ID NO: 118) was designed using IDT's online codon optimization tool. The mRNA sequences for VAC3 that encoded SARS-CoV-2 Wuhan spike protein were obtained from positions 17-3838 of the sequence in The World Health Organization: International Nonproprietary Names Programme. Messenger RNA Encoding the Full-Length SARS-CoV-2 Spike Glycoprotein; p. 11868, which is further described in Edo Kon, Uri Elia, and Dann Peer, “Principles for designing an optimal mRNA lipid nanoparticle vaccine,” Curr. Opin. Biotechnol. 2022, 73:329-336.

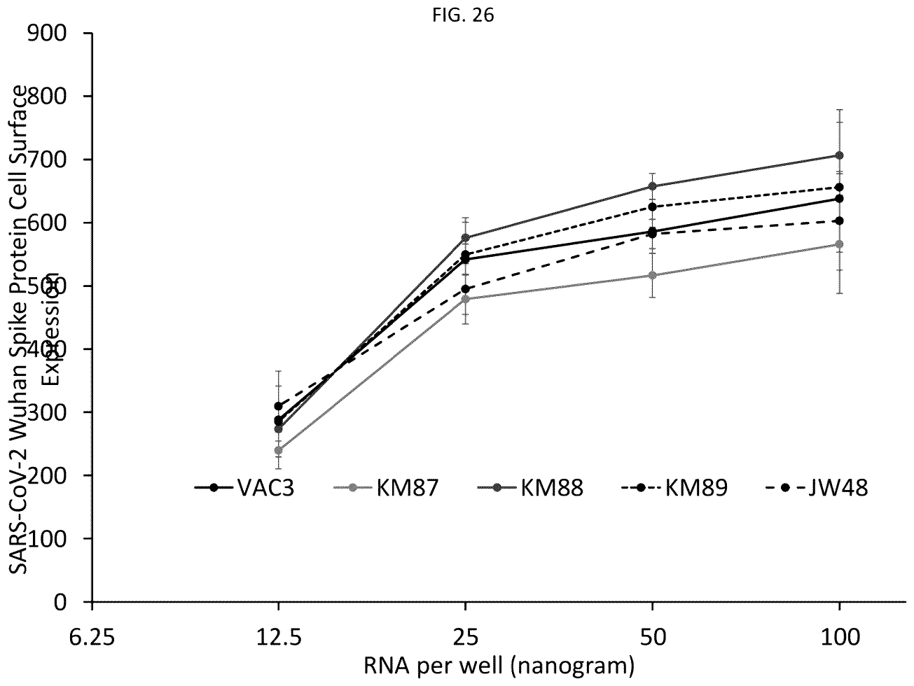

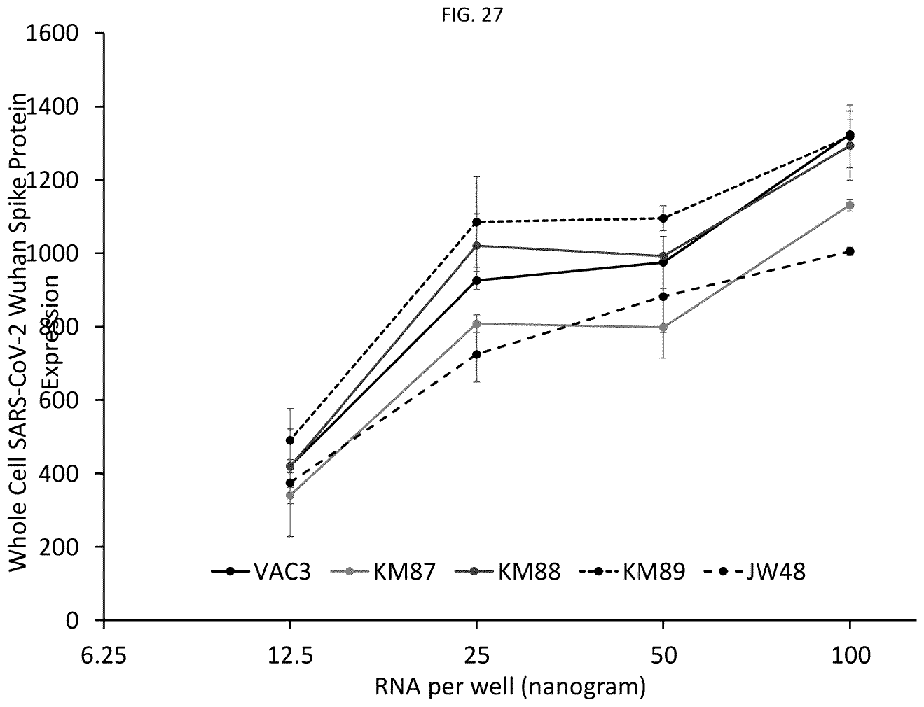

Eighteen hours later they were assessed for SARS-CoV-2 Wuhan strain expression on the cells' surface and the entirety of the cells as described above. FIGS. 26 and 27 show the dose response curve of spike protein from SARS-CoV-2 Wuhan strain expressed from VAC3, KM87, KM88, KM89, JW48 on the cell membrane surface of HEK293 cells and on the whole cells, respectively. Three prime and 5′ UTRs 4 and 7 (in KM88 and KM89 respectively) drove cell membrane surface expression and whole cell expression of SARS CoV-2 Wuhan spike protein as well as UTRs from CureVac's second SARS-CoV-2 candidate (in VAC3) and Moderna's mRNA-1273 SARS-CoV-2 FDA-approved product (in JW48).

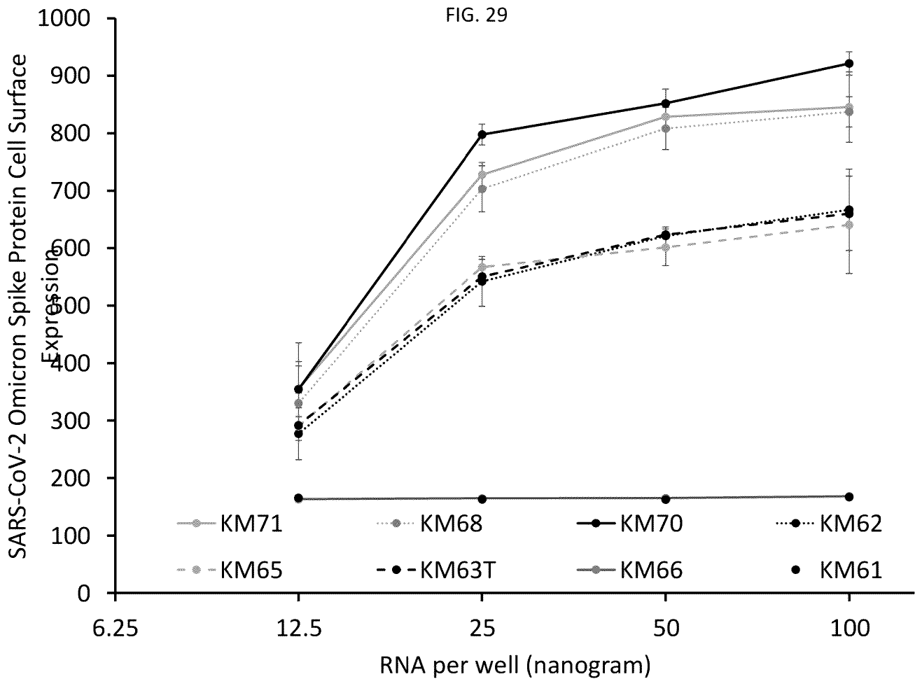

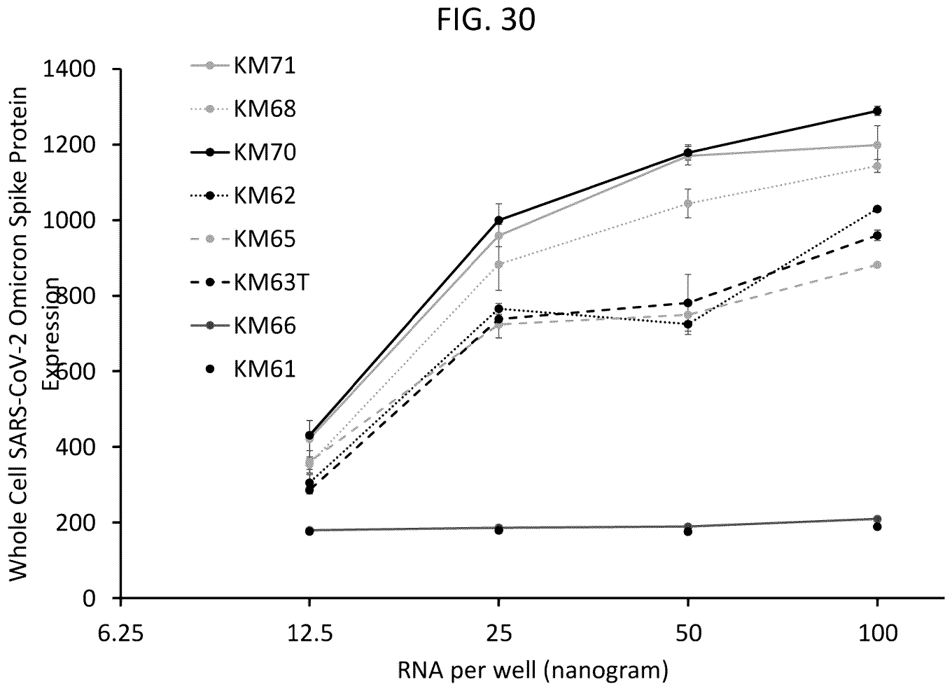

FIGS. 29 and 30 show the dose response curve of spike protein from SARS-CoV-2 omicron strain encoded by 12.5 ng, 25 ng, 50 ng, and 100 ng of KM71, KM68, KM70, KM62, KM65, KM63, KM66, or KM61 RNA per well on the cell membrane surface and the whole cell, respectively, of HEK293 cells. Encoding with CR1 (in KM68) and CR2 (in KM70 and 71) with 3′ and 5′ UTR4 (in KM70) and 3′ and 5′ UTR7 (in KM71 and KM68) drove significantly more cell membrane surface expression and whole cell expression of SARS CoV-2 omicron spike protein cell when compared to when the expression was driven by omicron spike protein sequences that were designed using commercially available software that optimized only for a codon adaptation index of greater than or equal to 0.7 and where the expression was driven by UTR3, UTR4, UTR7, and Moderna UTRs in mRNA-1273 SARS-CoV-2 FDA-approved product (in KM61, KM62, KM63, and KM65 respectively).

### Example 6

HEK293 cells were administered 62.5 ng, 125 ng, 250 ng, and 500 ng of VAC3, KM87, KM88, KM89, JW48, KM71, KM68, KM70, KM62, KM65, KM63, KM66, or KM61 RNA per well and were assessed for angiotensin-converting enzyme 2 (ACE2) protein binding with the SARS-CoV-2 Wuhan spike protein released from the transfected cells, as described above.

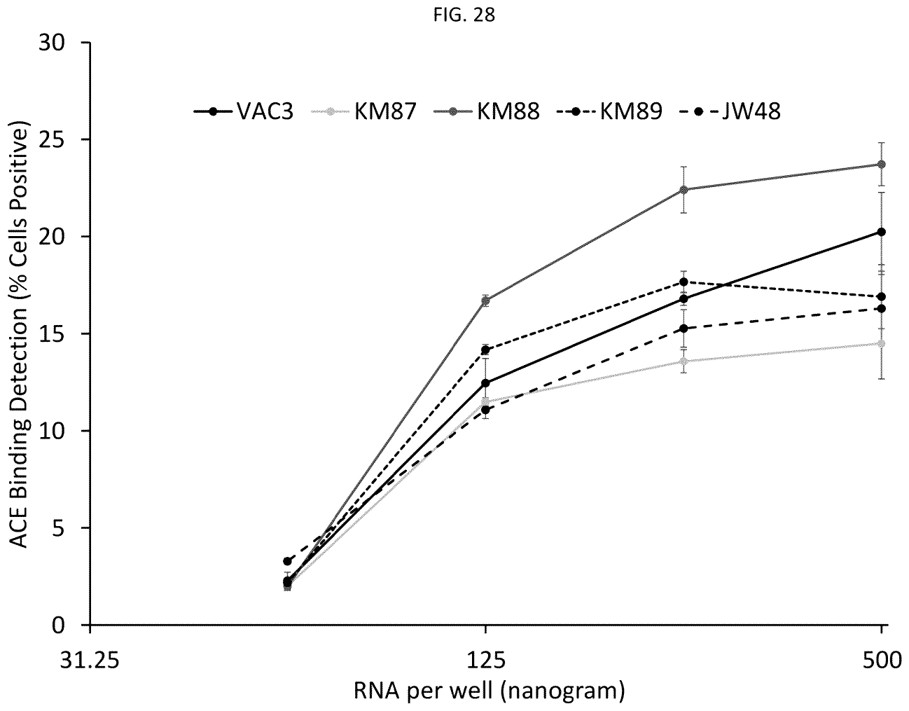

FIG. 28 depicts the dose response curve of ACE2 binding after VAC3, KM87, KM88, KM89, or JW48 RNA transfection. Three prime and 5′ UTRs 4 and 7 (in KM88 and KM89 respectively) drove Wuhan spike protein expression as well as UTRs from CureVac's second SARS-CoV-2 candidate (in VAC3) and Moderna's mRNA-1273 SARS-CoV-2 FDA-approved product (in JW48).

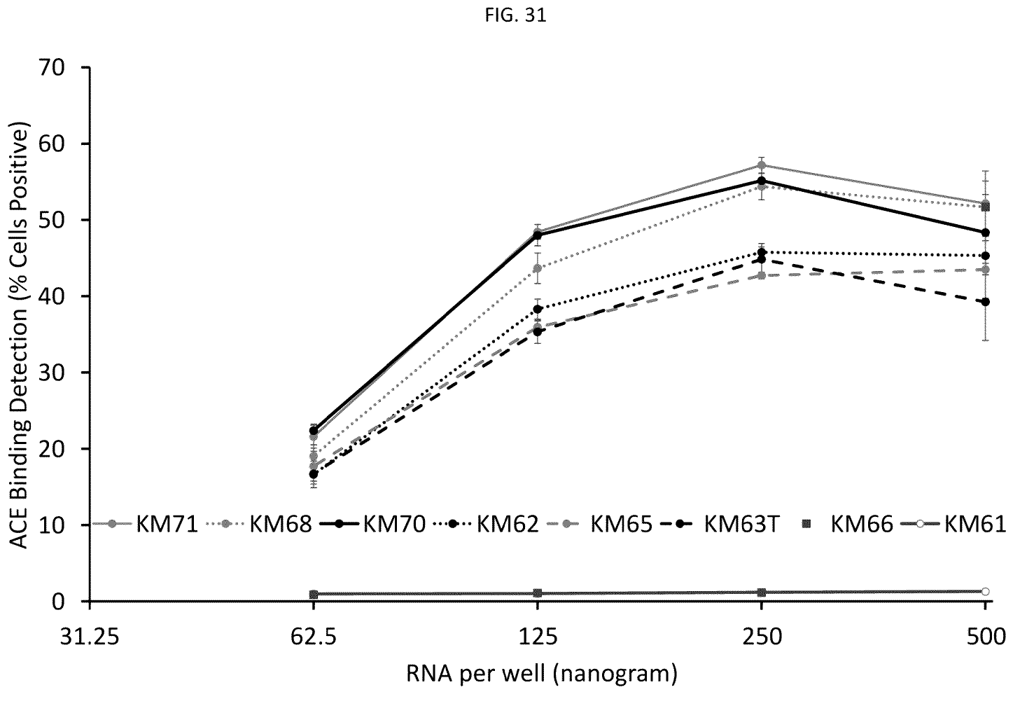

FIG. 31 depicts the dose response curve of angiotensin-converting enzyme 2 (ACE2) protein binding with SARS-CoV-2 Wuhan spike protein after transfection with KM71, KM68, KM70, KM62, KM65, KM63, KM66, or KM61 RNA. Encoding with CR1 (in KM68) and CR2 (in KM70 and 71) with 3′ and 5′ UTR4 (in KM70) and UTR7 (in KM71 and KM68) drove significantly more omicron spike protein expression as measured by ACE2 binding when compared to when the expression was driven by omicron spike protein sequences that were designed using commercially available software that optimized only for a codon adaptation index of greater than or equal to 0.7 and where the expression was driven by UTR3, UTR4, UTR7, and Moderna UTRs in mRNA-1273 SARS-CoV-2 FDA-approved product (in KM61, KM62, KM63, and KM65 respectively).

### Example 7: In Vivo Mouse Studies

All recombinant RNA molecules were produced by in vitro transcription using N1-methyl pseudouridine to replace all uridines. All recombinant RNA molecules comprised a cap-1 5′ cap and a 3′ poly(A) tail. The mRNAs were purified and evaluated for mRNA integrity (by capillary gel electrophoresis), double stranded RNA (dsRNA) content (by Homogeneous Time Resolved Fluorescence [HTRF]), poly-A tail length, and percent capping.

The mRNA constructs were then formulated in LNPs comprising 46.3 mol % cation-ionizable lipid, RV39; 1.6 mol % PEG-conjugated lipid; 42.7 mol % cholesterol; and 9.4 mol % 1,2-diastearoyl-sn-glycero-3-phosphocholine (DSPC).

Female BALB/c mice were 7 or 8 weeks old at day 0 of the study. On day 0 and day 21 of the study were injected with a 28 gauge needle into both hindleg thigh muscles with a vehicle or a unit dose per animal of 5.0 μg, 1.0 μg, or 0.2 μg of KM65, KM70, KM71, or KM68 in 50 μL (25 μL per thigh muscle). The groups of animals, the lots of formulations, stock concentrations of formulations, vials of formulations, and storage temperatures of formulations were:

| | | Test | | | | | Storage |
|---|---|---|---|---|---|---|---|
| | | Article | Lot | Stock | Number of | Number of | Temperature |
| Group | Immunogen | Label | Number | Concentration | vials supplied | animals | (° C.) |
| 1 | Saline | NA | NA | As | NA | 4 | RT |
| | | | | Supplied | | | |
| | | | | from | | | |
| | | | | Manufacturer | | | |
| 2 | Moderna | Moderna | 24805A | 100 μg/mL | 2 vials 5 ug | 8 | −80° C. |
| | UTRs- | control | | | dose (1 mL | | |
| | Omicron | KM65 | | | material per | | |
| | Control | LNPs | | | vial) | | |
| 3 | (KM65) | (RV39) | | | 2 vials 1 ug | 8 | |
| | | | | | dose (0.4 mL | | |
| | | | | | material per | | |
| | | | | | vial) | | |
| 4 | | | | | 2 vials 0.2 ug | 8 | |
| | | | | | dose (0.1 mL | | |
| | | | | | per vial) | | |
| 5 | KM70 | KM70 | 24805C | 100 μg/mL | 2 vials 5 ug | 8 | −80° C. |
| | | LNPs | | | dose (1 mL | | |
| | | (RV39) | | | material per | | |
| | | | | | vial) | | |
| 6 | | | | | 2 vials 1 ug | 8 | |
| | | | | | dose (0.4 mL | | |
| | | | | | material per | | |
| | | | | | vial) | | |
| 7 | | | | | 2 vials 0.2 ug | 8 | |
| | | | | | dose (0.1 mL | | |
| | | | | | per vial) | | |
| 8 | KM71 | KM71 | 24805D | 100 μg/mL | 2 vials 5 ug | 8 | −80° C. |
| | | LNPs | | | dose (1 mL | | |
| | | (RV39) | | | material per | | |
| | | | | | vial) | | |
| 9 | | | | | 2 vials 1 ug | 8* | |
| | | | | | dose (0.4 mL | | |
| | | | | | material per | | |
| | | | | | vial) | | |
| 10 | | | | | 2 vials 0.2 ug | 8 | |
| | | | | | dose (0.1 mL | | |
| | | | | | per vial) | | |
| 11 | KM68 | KM68 | 24805B | 100 μg/mL | 2 vials 5 ug | 8 | −80° C. |
| | | LNPs | | | dose (1 mL | | |
| | | (RV39) | | | material per | | |
| | | | | | vial) | | |
| 12 | | | | | 2 vials 1 ug | 8 | |
| | | | | | dose (0.4 mL | | |
| | | | | | material per | | |
| | | | | | vial) | | |
| 13 | | | | | 2 vials 0.2 ug | 8 | |
| | | | | | dose (0.1 mL | | |
| | | | | | per vial) | | |
| 14 | Moderna | LNP | FM-1991A | 500 μg/mL | 2 vials 5 ug | 8 | −80° C. |
| | UTRs- | KM65 | | | dose (0.3 mL | | |
| | Omicron | mRNA | | | material per | | |
| | Control | FM-1991A | | | vial) | | |
| 15 | (KM65) | | | | 2 vials 1 ug | 8 | |
| | | | | | dose (0.08 mL | | |
| | | | | | material per | | |
| | | | | | vial) | | |
| 16 | | | | | 2 vials 0.2 ug | 8 | |
| | | | | | dose (0.02 mL | | |
| | | | | | per vial) | | |

On day 14, 25 μL of whole blood, which is around 10 μL of serum, was obtained from the tail tip of each animal. On day 21, 100 μL of whole blood, which is around 40 μL of serum, was obtained retro-orbitally. On day 35, mice were anesthetized under isoflurane and terminally exsanguinated by cardiac stick to obtain from around 200 μL to around 500 μL of whole blood, which never resulted in less than 100 μL serum. *Due to an error in retro-orbital serum collection one animal was humanely killed in group 9 on day 21, and no serum was obtained for day 35 from that animal. On day 14, one animal from group 14 had its serum lost due to a centrifuge error during serum separation.

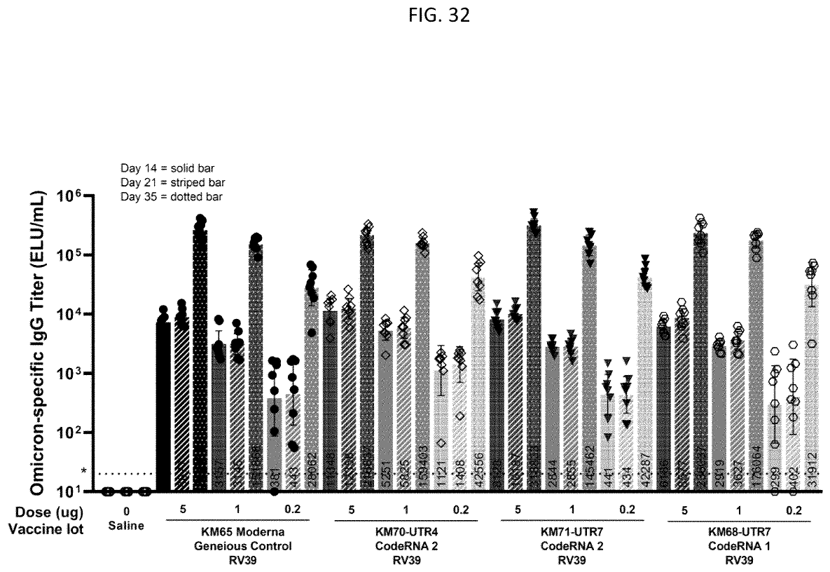

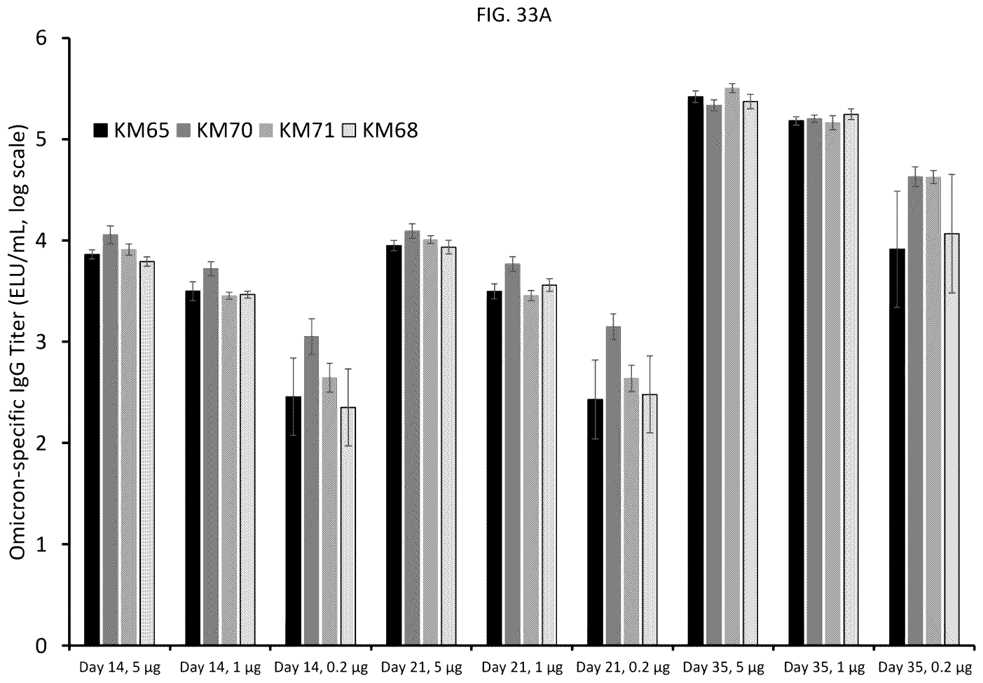

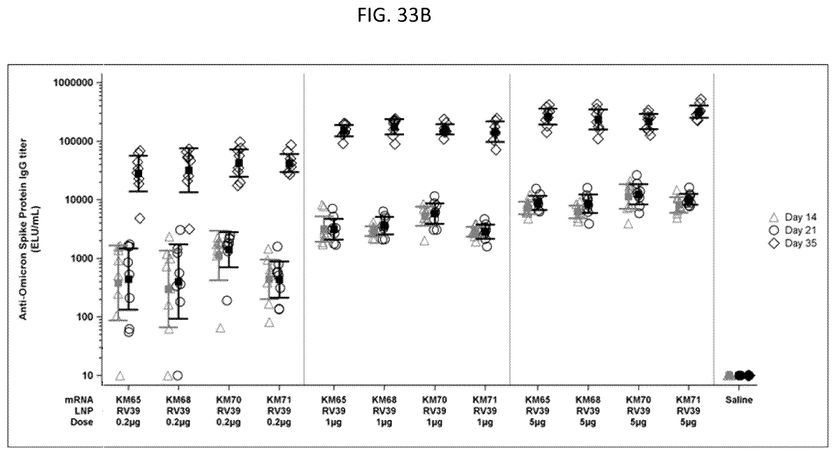

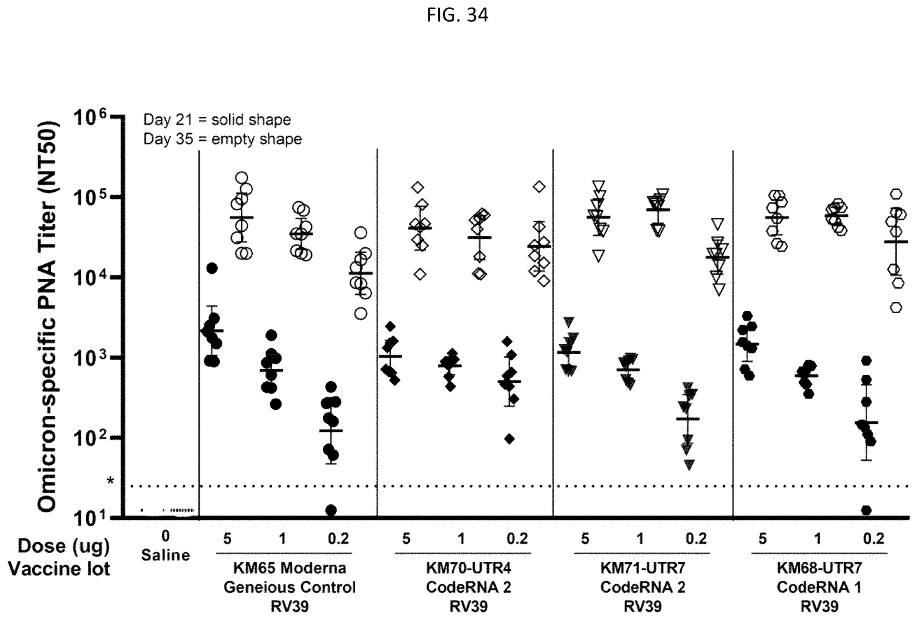

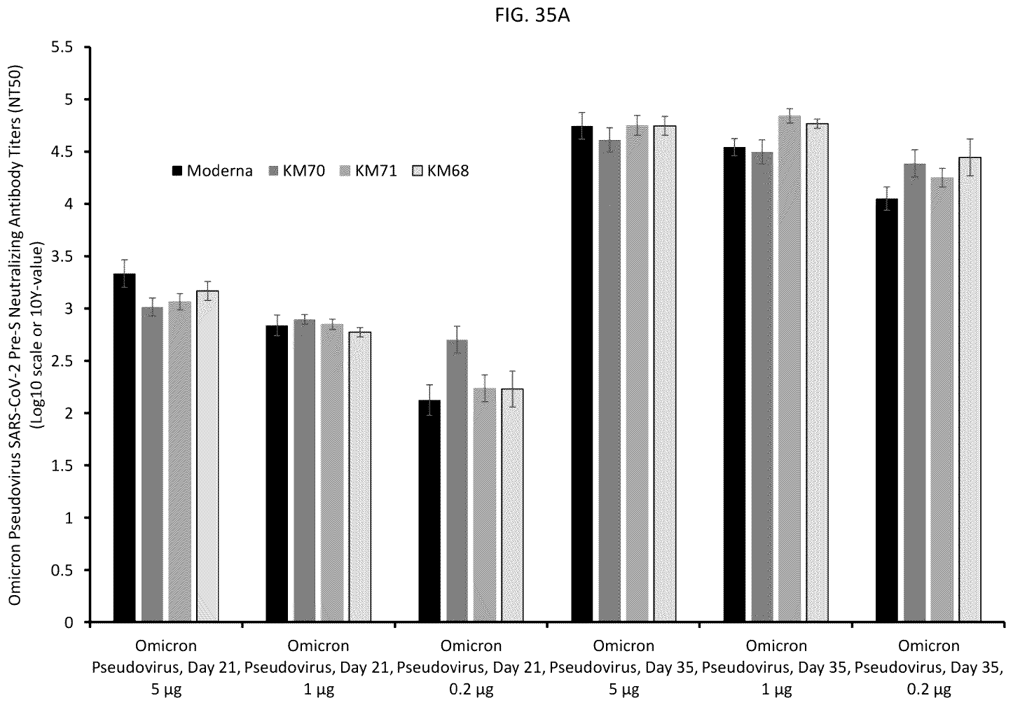

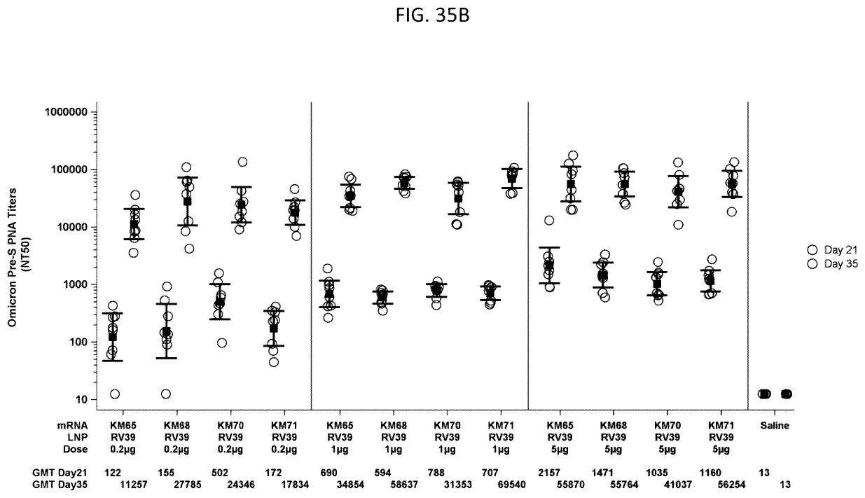

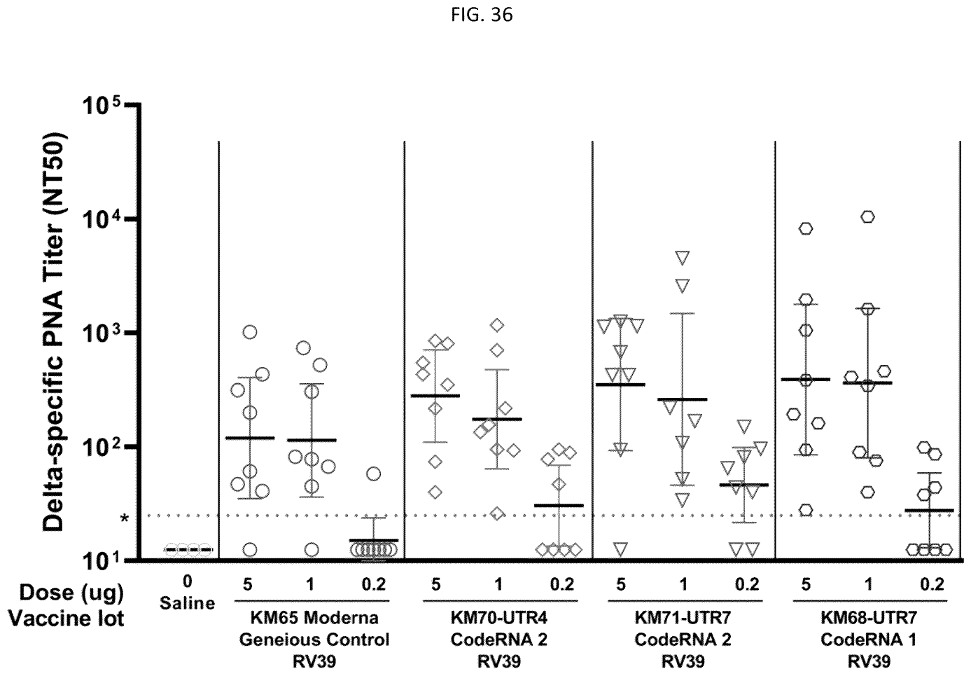

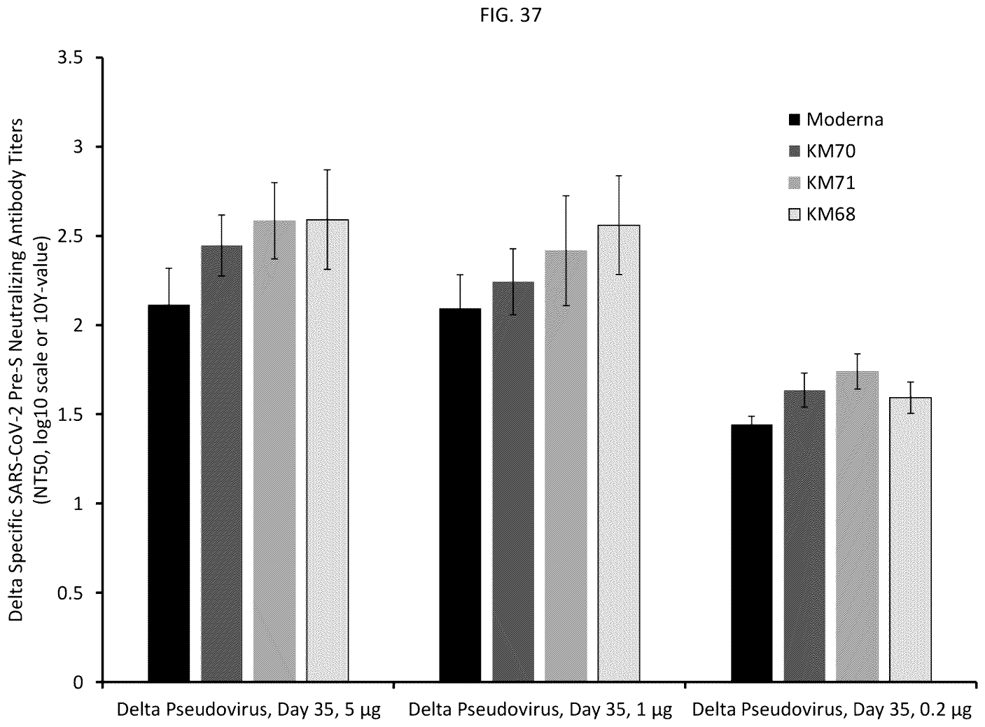

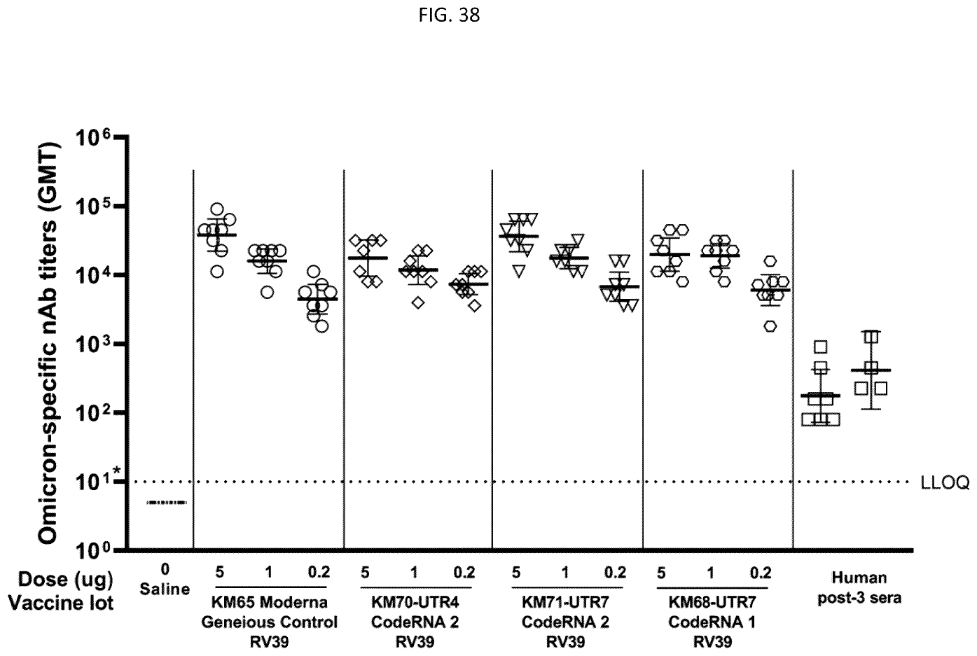

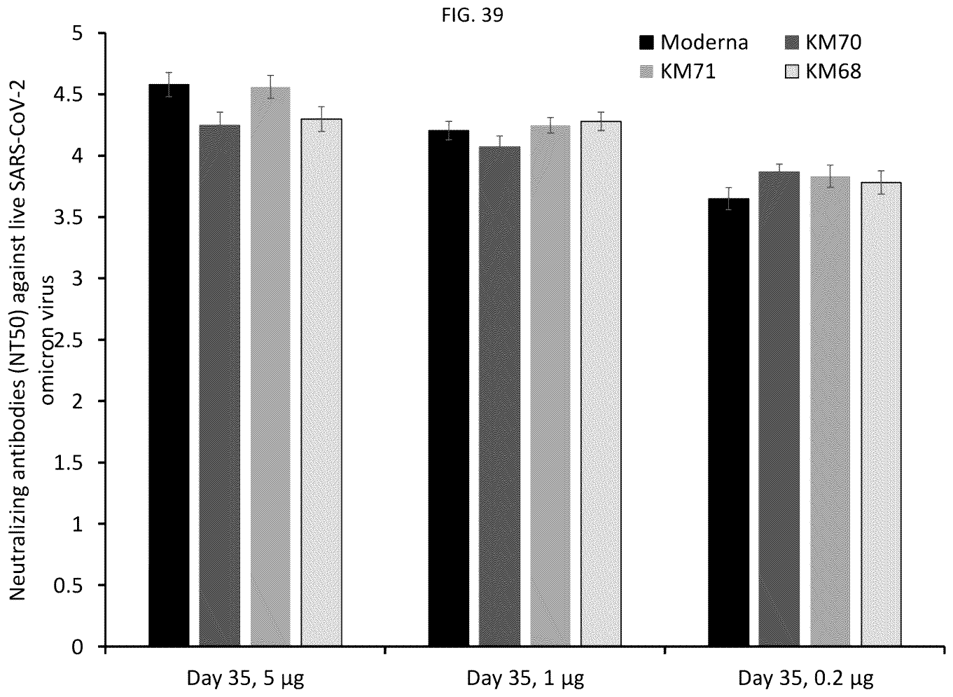

Anti-spike protein IgG binding antibody titers were determined by ELISA from day 14, 21, and 35 serum samples (see FIGS. 32 and 33). SARS-CoV-2 omicron pseudovirus neutralization antibody titers (NT 50) were obtained from day 21 and 35 serum samples (see FIGS. 34 and 35). SARS-CoV-2 delta pseudovirus neutralization antibody titers (NT 50) were obtained from day 35 serum samples (see FIGS. 36 and 37). Live SARS-CoV-2 omicron neutralization antibody titers (NT 50) were obtained from day 35 serum samples (see FIGS. 38 and 39). The KM70, KM71, and KM68 mRNA constructs were immunogenic compared to the saline control. Administration of the KM70, KM71, and KM68 mRNA constructs induced a response that was not below the response after KM65 administration. All constructs were formulated in the RV39 at the majority of the dosages and timepoints (excluding the 5.0 μg dosage at Day 35 for KM68 and KM70 when compared to the KM65 RV39 formulation. KM71 produced significantly higher titers (˜2-fold) at the 5.0 μg dosage (Post-2, Day 35) when compared to KM68 and KM70. At the 0.2 and 1.0 μg dosage Post-2, the mRNA constructs performed comparably. A dose response was observed across most constructs.

FIGS. 32, 33A, and 33B show the geometric mean IgG titers on day 14, day 21, and day 35 from female mice treated on day 0 and day 21 of the study with 5 μg, 1 μg, or 0.2 μg of KM65, KM70, or KM71 RNA encapsulated in RV39 LNPs. The individual points are the IgG titers from each individual animal. FIG. 32 organizes the geometric means by RNA treatment, then by dose, then by day of serum extraction for IgG titers. FIGS. 33A and 33B organizes the geometric means by day of serum extraction, then by dose, then by RNA treatment. Treatment with RNA encoded by CR2 optimization of SARS-CoV-2 omicron spike protein, which is driven by UTR4, elicited higher IgG titers at days 14 and 21 than treatment with KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. Treatment with RNA encoded by CR2 optimization of SARS-CoV-2 omicron spike protein, which is driven by UTR7, elicited higher IgG titers at days 14 and 21 at 5 μg doses and at day 35 at 5 μg and 0.2 μg than treatment with KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. Treatment with RNA encoded by CR1 optimization of SARS-CoV-2 omicron spike protein, which is driven by UTR7, elicited higher IgG titers at days 21 and 35 at 1 μg doses than treatment with KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product.

FIGS. 34, 35A, and 35B depict neutralizing antibody titers (NT50) against a pseudovirus comprising SARS-CoV-2 omicron spike protein on days 21 and 35 of the same animals treated in FIGS. 32 and 33. At day 21 and a dose of 0.2 μg, administration of KM70 elicited higher neutralizing antibody titers than administration of KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. At day 23 and a dose of 1 μg, administration of KM71 or KM68 elicited higher neutralizing antibody titers than administration of KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. At day 23 and a dose of 0.2 μg, administration of KM70, KM71, or KM68 elicited higher neutralizing antibody titers than administration of KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product.

FIGS. 36 and 37 show neutralizing antibody titers (NT50) against a pseudovirus comprising SARS-CoV-2 delta spike protein on day 35 of the same animals treated in FIGS. 32, 33A, and 33B. At every dose, administration with KM68, KM70, or KM71 elicited higher neutralizing antibody titers than administration of KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product.

FIGS. 38 and 39 depict neutralizing antibody titers (NT50) against live SARS-CoV-2 virus on day 35 of the same animals treated in FIGS. 32, 33A, and 33B. Administration of KM68, KM70, and KM71 elicited neutralizing antibodies (NT50) against live SARS-CoV-2 omicron virus at the same levels as those elicited by administration of KM65, RNA encoding SARS-CoV-2 omicron spike protein optimized by commercially available software that optimized only for a codon adaptation index of greater than or equal to 0.7 and which was driven by Moderna UTRs in mRNA-1273 SARS-CoV-2 FDA-approved product. At the lowest dose, 0.2 μg, UTRs 4 and 7 outperformed the Moderna UTRs. At the lowest dose, 0.2 μg, the code RNA 1 and 2 optimized SARS-CoV-2 omicron spike protein RNA sequences outperformed SARS-CoV-2 omicron spike protein RNA sequence that was optimized with the commercially available software.

### Example 8

KM70 RNA was synthesized with 3′ poly(A) tails consisting of 40 adenosine monophosphates (As), 60 As, 80 As, 100 As, 120 As, or 150 As, and 12.5 ng, 25 ng, 50 ng, or 100 ng of the RNA were transfected into HEK293 cells as described above. Eighteen hours after transfection, the HEK293 cells were assessed for SARS-CoV-2 omicron spike protein surface expression.

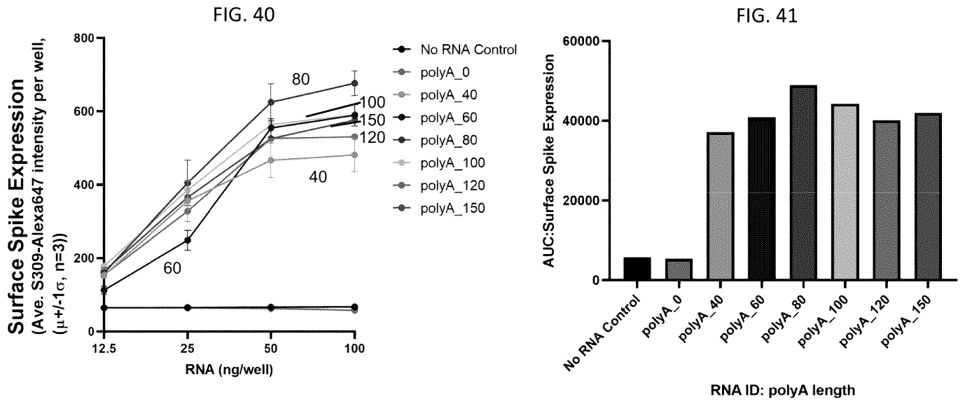

FIGS. 40 and 41 show the dose response curve and area under the curve, respectively, of the HEK293 cell membrane surface expression of SARS-CoV-2 omicron spike protein encoded by 12.5 ng, 25 ng, 50 ng, and 100 ng of KM70, where the length of the 3′ poly(A) tail consisted of 40 As, 60 As, 80 As, 100 As, 120 As, or 150 As.

KM70 RNA with a 3′ poly(A) tail consisting of 80 As had the highest overall expression of SARS-CoV-2 spike protein on the HEK293 cell membrane surface, followed by KM70 RNA with a 3′ poly(A) tail consisting of 100 As, then by KM70 RNA with a 3′ poly(A) tail consisting of 150 As, 120 As, or 60 As, then by KM70 RNA with a 3′ poly(A) tail consisting of 40 As. The lowest expression was observed with KM70 having no 3′ poly(A) tail (“polyA_0”) and with transfection with no RNA at all.

### Example 9

KM65, KM68, KM70, and KM71 RNA was synthesized with 3′ poly(A) tails consisting of around 70 adenosine monophosphates (As) or exactly 100 As, and 12.5 ng, 25 ng, 50 ng, or 100 ng of the RNA were transfected into HEK293 cells as described above.

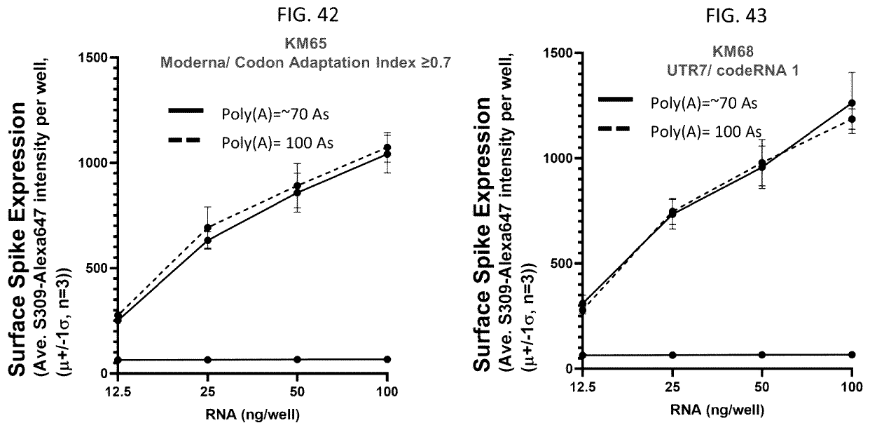

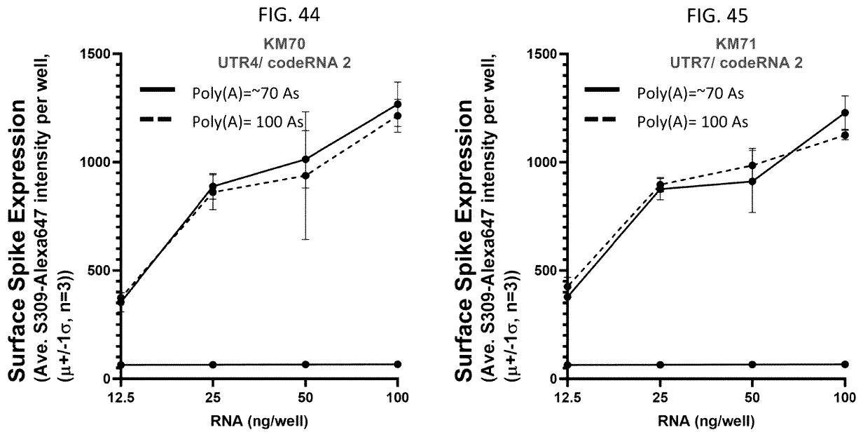

FIGS. 42-45 depict the dose response curve of HEK293 cell membrane surface expression of SARS-CoV-2 omicron spike protein encoded by 12.5 ng, 25 ng, 50 ng, and 100 ng of KM65, KM68, KM70, and KM71 RNA, respectively, where the length of the 3′ poly(A) tail consisted of around 70 As (solid lines) or exactly 100 As (dotted lines).

Regardless of the construct, 3′ poly(A) tails consisting of around 70 As slightly outperformed 3′ poly(A) tails consisting of exactly 100 As. Together with Example 8, this suggests highest expression with a 3′ poly(A) tail consisting of from around 70 As to 80As when compared to the expression of a 3′ poly(A) tail consisting of exactly 100 As.

### Example 10a

KM70 (as described in U.S. Ser. No. 63/413,844) and LL34-38 (see above) RNA was synthesized with 3′ poly(A) tails consisting of, from 5′ to 3′, 30 adenosine monophosphates (As), a linker, and 38 As. All recombinant RNA molecules were produced by in vitro transcription using N1-methyl pseudouridine to replace all uridines. All recombinant RNA molecules comprised a cap-1 5′ cap and a 3′ poly(A) tail. The mRNAs were purified and evaluated for mRNA integrity (by capillary gel electrophoresis), double stranded RNA (dsRNA) content (by Homogeneous Time Resolved Fluorescence [HTRF]), poly-A tail length, and percent capping.

Each RNA encapsulated in then formulated in LNPs comprising 46.3 mol % cation-ionizable lipid, RV39; 1.6 mol % PEG-conjugated lipid; 42.7 mol % cholesterol; and 9.4 mol % 1,2-diastearoyl-sn-glycero-3-phosphocholine (DSPC), i.e., the RV39 formulation.

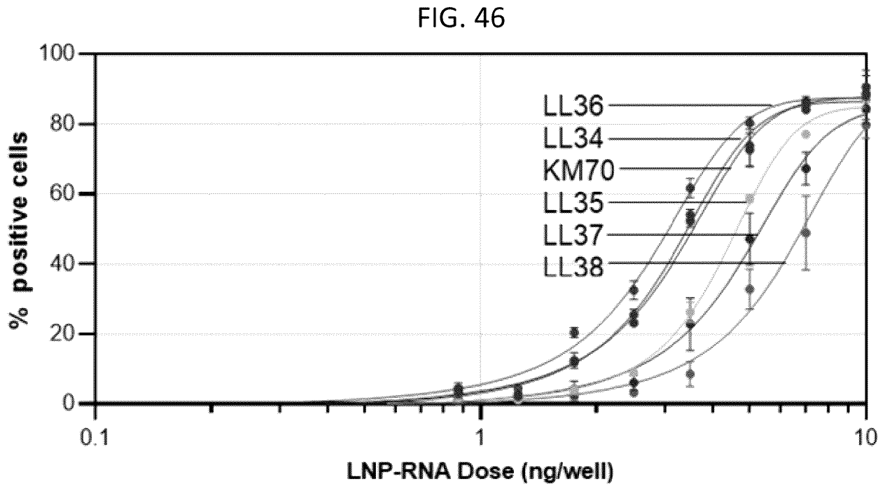

A potency assay of the LNP encapsulated RNAs was carried out. HEK 293 cells were seeded at 3×10⁴ cells/well in a 96-well plate and incubated overnight. The cells were transduced with Omicron RNA LNP samples at 10, 7, 3.5, 2.5, 1.75, 1.25, and 0.8765 ng/well. Cells were stained with anti-SARS-CoV2 Spike antibody and a secondary antibody. Expression was detected by flow cytometry. FIG. 46 depicts the dose response curve of expression of spike protein from SARS-CoV2 spike protein, with percent of positive cells on the y-axis and Dose on the x-axis. EC50 values (ng/well) were calculated, as shown in the following table:

| | | | Coding | | EC50 |
|---|---|---|---|---|---|
| ID | Constuct | UTR | Sequence | Formulation | (ng/well) |
| 1 | KM70 | UTR4 | OmicronBA1/codeRNA 2 | RV39 | 3.34 |
| 2 | LL34 | UTR4 | OmicronBA1/codeRNA 3 | RV39 | 3.19 |
| 3 | LL35 | UTR4 | OmicronBA1/codeRNA 4 | RV39 | 4.34 |
| 4 | LL36 | UTR4 | OmicronBA1/codeRNA 5 | RV39 | 2.86 |
| 5 | LL37 | UTR4 | OmicronBA1/codeRNA 6 | RV39 | 5.03 |
| 6 | LL38 | UTR4 | OmicronBA1/codeRNA 7 | RV39 | 7.39 |

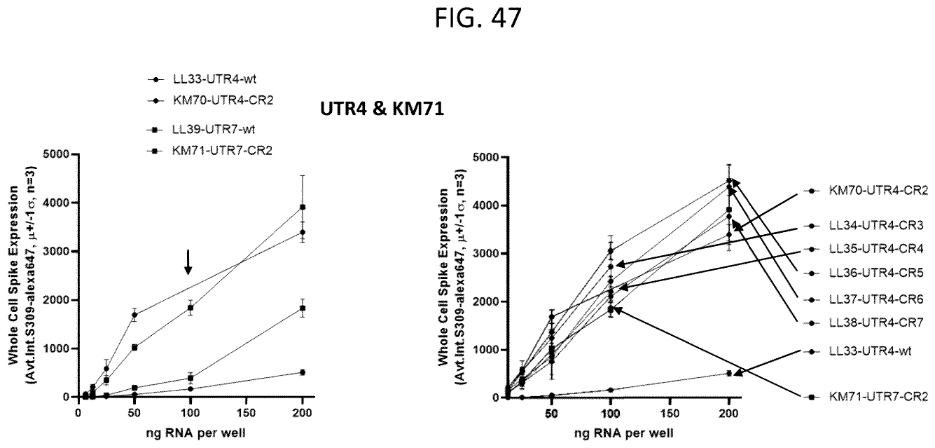

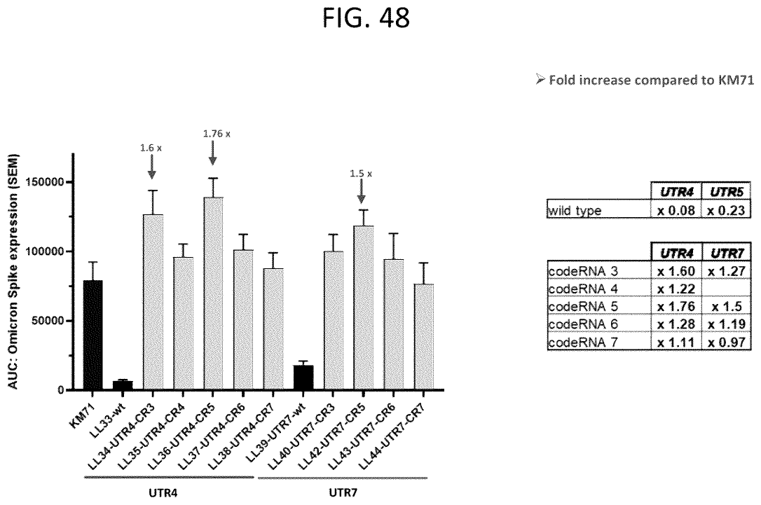

Assays were carried out twice with the same results. Drug product generated from LL34 and LL36 had EC50s lower than that observed for the KM70 drug product. UTR7 (SEQ ID NOs: 13 & 14). FIG. 47 depicts whole cell SARS-CoV2 Spike protein expression at each dose, and FIG. 48 depicts the area under the curve of all doses from FIG. 47. LL34 had 1 0.6 times larger area under the curve as that for KM71. LL36 had 1.76 times larger area under the curve as that for KM71. LL52 had 1.5 times larger area under the curve as that for KM71.

### Example 10b

KM70 and LL34-38 encapsulated in LNP as described in Example 10a were evaluated for immunogenicity in mice. Female BALB/c mice were 7 or 8 weeks old at day 0 of the study. On day 0 and day 21 of the study were injected using a 28 gauge needle into both hindleg thigh muscles with a vehicle or a unit dose per animal of 5.0 μg, 1 0.0 μg, or 0.5 μg of KM70 or LL34-38 in 50 μL (25 μL per thigh muscle). The groups of animals, numbers, construct, dose, and formulations were as follows:

| | | Construct | | | Dose | |
|---|---|---|---|---|---|---|
| Group | N | Name | UTR | Coding Sequence | (ug) | Formulation |
| 1 | 4 | N/A | N/A | Saline | N/A | N/A |
| 2 | 8 | KM70 | UTR4 | SARS-CoV2 Omicron BA1 spike protein, CR2 | 5.0 | RV39 |
| | | | | optimized | | |
| 3 | 8 | KM70 | UTR4 | Same as above | 1.0 | RV39 |
| 4 | 8 | KM70 | UTR4 | Same as above | 0.5 | RV39 |
| 5 | 8 | LL34 | UTR4 | SARS-CoV2 Omicron BA1 spike protein, CR3 | 5.0 | RV39 |
| | | | | optimized | | |
| 6 | 8 | LL34 | UTR4 | Same as above | 1.0 | RV39 |
| 7 | 8 | LL34 | UTR4 | Same as above | 0.5 | RV39 |
| 8 | 8 | LL35 | UTR4 | SARS-CoV2 Omicron BA.1 spike protein, CR4 | 5.0 | RV39 |
| | | | | optimized | | |
| 9 | 8 | LL35 | UTR4 | Same as above | 1.0 | RV39 |
| 10 | 8 | LL35 | UTR4 | Same as above | 0.5 | RV39 |
| 11 | 8 | LL36 | UTR4 | SARS-CoV2 Omicron BA.1 spike protein, CR5 | 5.0 | RV39 |
| | | | | optimized | | |
| 12 | 8 | LL36 | UTR4 | Same as above | 1.0 | RV39 |
| 13 | 8 | LL36 | UTR4 | Same as above | 0.5 | RV39 |
| 14 | 8 | LL37 | UTR4 | SARS-CoV2 Omicron BA.1 spike protein, CR6 | 5.0 | RV39 |
| | | | | optimized | | |
| 15 | 8 | LL37 | UTR4 | Same as above | 1.0 | RV39 |
| 16 | 8 | LL37 | UTR4 | Same as above | 0.5 | RV39 |
| 17 | 8 | LL38 | UTR4 | SARS-CoV2 Omicron BA.1 spike protein, CR7 | 5.0 | RV39 |
| | | | | optimized | | |
| 18 | 8 | LL38 | UTR4 | Same as above | 1.0 | RV39 |
| 19 | 8 | LL38 | UTR4 | Same as above | 0.5 | RV39 |

Samples were taken as per (A)-(C)(below):

### (A) Early Timepoint Readouts:

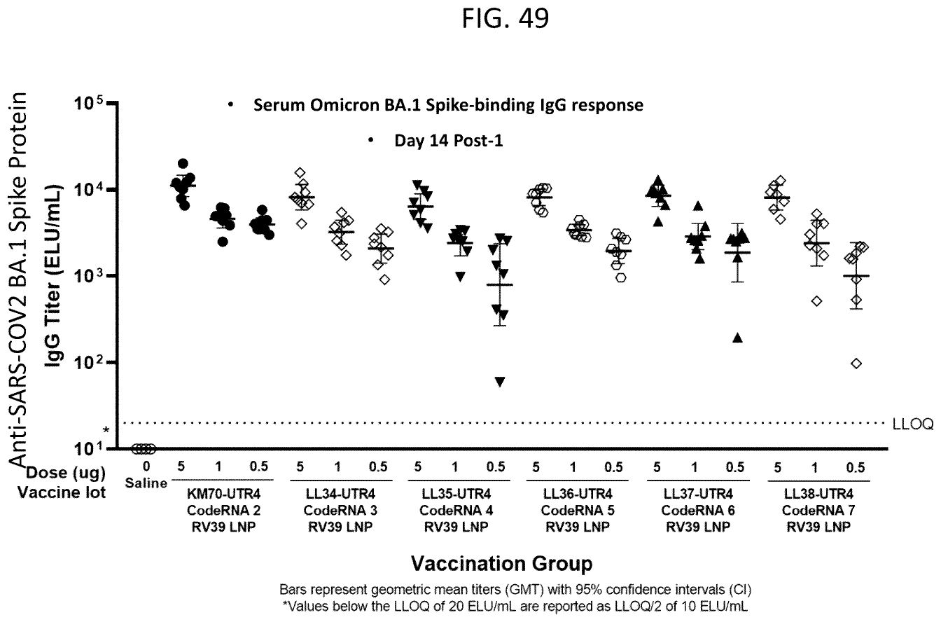

Day 14 Blood Draw
   Anti-S protein IgG binding antibody titers (Omicron S protein ELISA) (See FIG. 49)
Day 21 Blood Draw
   Anti-S protein IgG binding antibody titers (Omicron S protein ELISA)
   Pseudovirus neutralization Ab titers (Omicron pVNT)

### (B) Late Timepoint Readouts:

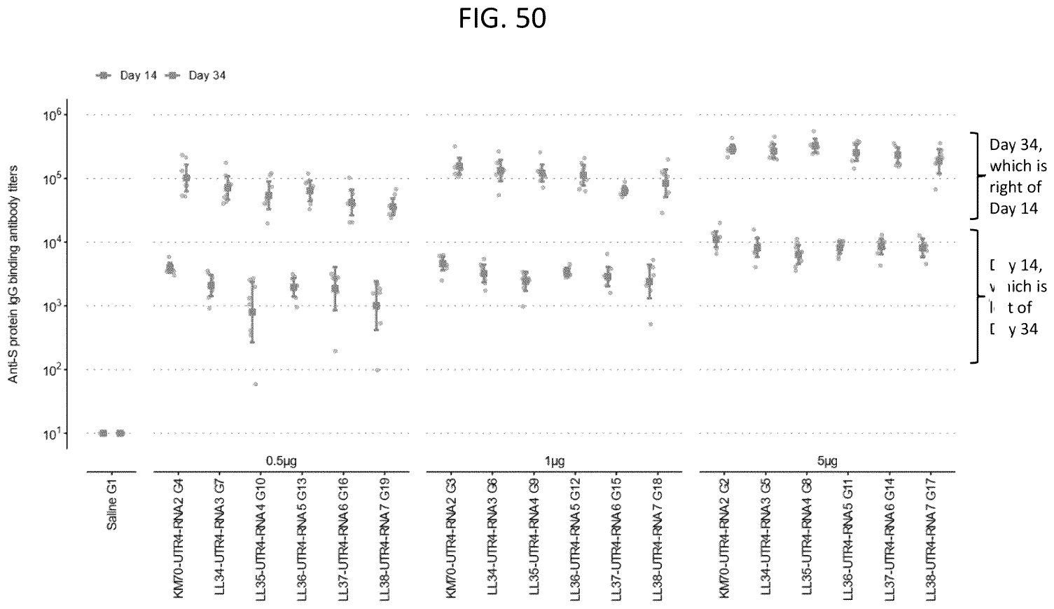

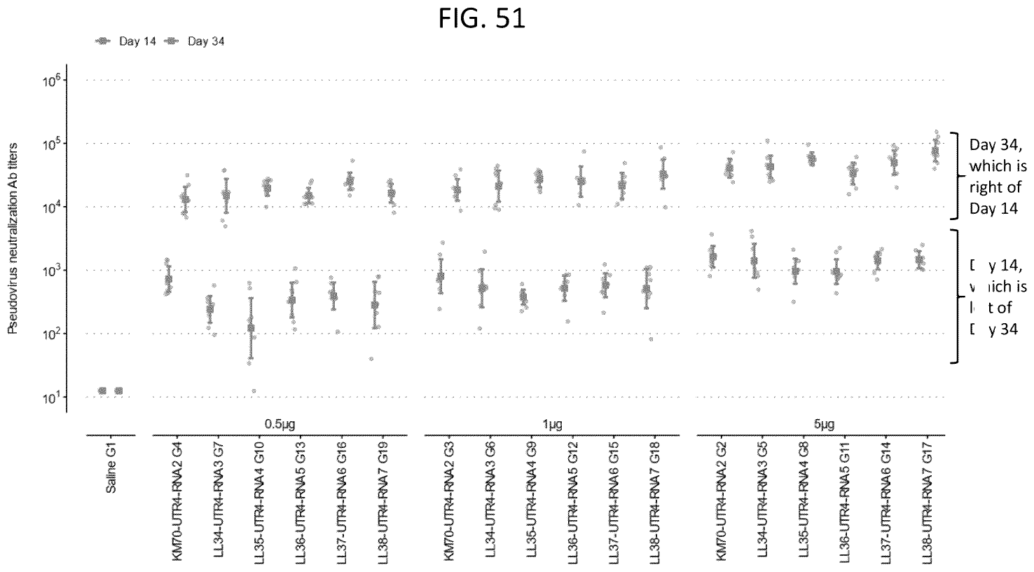

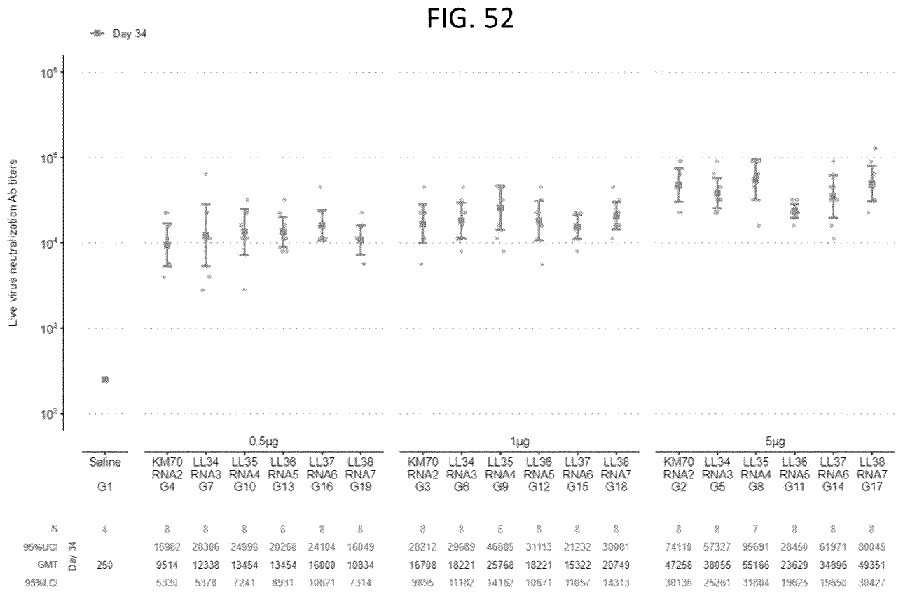

Day 35 Blood Draw and Spleen Harvest
   Anti-S protein IgG binding antibody titers (Omicron S protein ELISA) (See FIGS. 49 and 50)
   Pseudovirus neutralization Ab titers (Omicron pVNT) (See FIG. 51)
   Live virus neutralization Ab titers (See FIG. 52)

### (C) CMI Analysis:

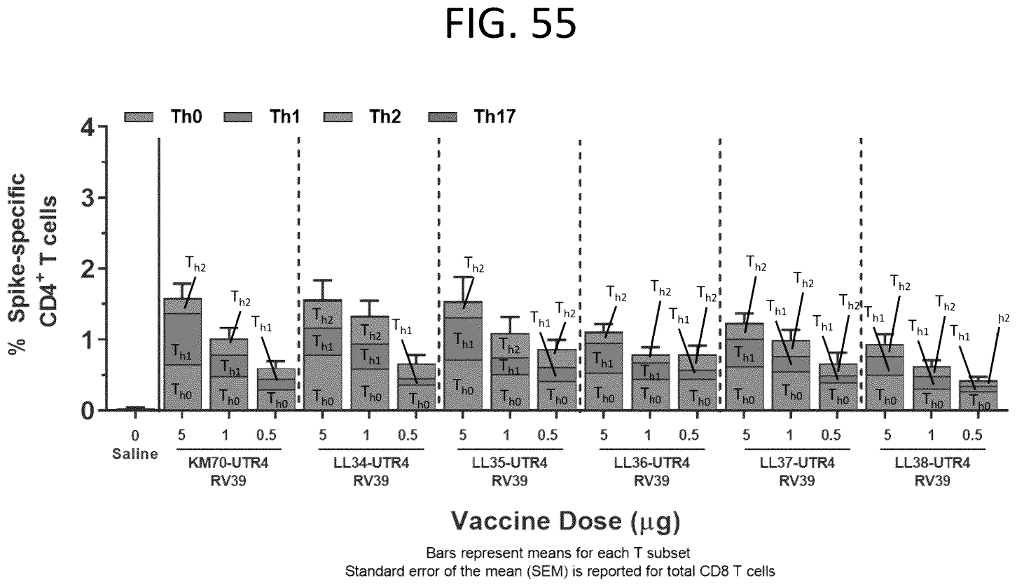

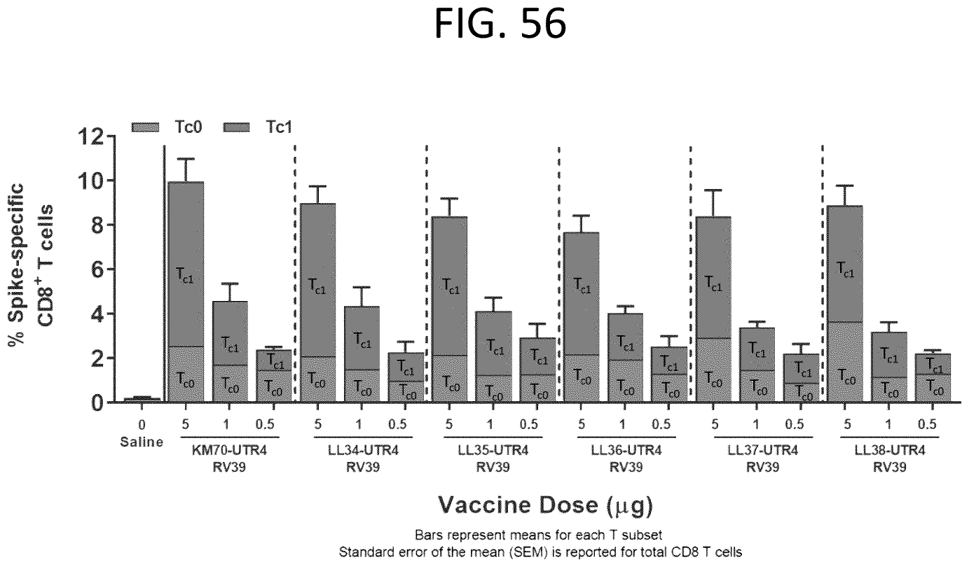

Day 35 Spleen Harvest (See FIG. 55 for CD4 positive cells, see FIG. 56 for CD8 positive cells)

KM70, LL34, LL35, LL36, LL37, and LL38 administration when compared to the same doses of the other constructs at the same day of assay all resulted in statistically insignificant differences in levels of total IgG against anti-SARS-CoV2 BA.1 spike protein (FIGS. 49 and 50), in pseudovirus neutralization antibody titers (FIG. 51), in live virus neutralization antibody titers (FIG. 52), in SARS-COV-2 spike protein specific CD4-positive T cell responses (FIG. 55), and in SARS-COV-2 spike protein specific CD8-positive T cell responses (FIG. 56).

### Example 11a

KM70 prime (also known as LL59, as described in U.S. Ser. No. 63/413,844 as SEQ ID NOs: 119-121 (SEQ ID NO: 121 used)), LL60-62, LL66, and LL67 (see above) RNA were synthesized with 3′ poly(A) tails consisting of, from 5′ to 3′, 30 adenosine monophosphates (As), a linker, and 38 As. All recombinant RNA molecules were produced by in vitro transcription using N1-methyl pseudouridine to replace all uridines. All recombinant RNA molecules comprised a cap-1 5′ cap and a 3′ poly(A) tail. The mRNAs were purified and evaluated for mRNA integrity (by capillary gel electrophoresis), double stranded RNA (dsRNA) content (by Homogeneous Time Resolved Fluorescence [HTRF]), poly-A tail length, and percent capping.

Each RNA encapsulated in then formulated in LNPs comprising 46.3 mol % cation-ionizable lipid, RV39; 1.6 mol % PEG-conjugated lipid; 42.7 mol % cholesterol; and 9.4 mol % 1,2-diastearoyl-sn-glycero-3-phosphocholine (DSPC), i.e., the RV39 formulation.

A potency assay of the LNP encapsulated RNAs was carried out. HEK 293 cells were seeded at 3×10⁴ cells/well in a 96-well plate and incubated overnight. The cells were transduced with Omicron RNA LNP samples at 25, 50, 75, and 100 ng/well. Cells were stained with anti-SARS-CoV2 Spike antibody and a secondary antibody. Expression was detected by flow cytometry.

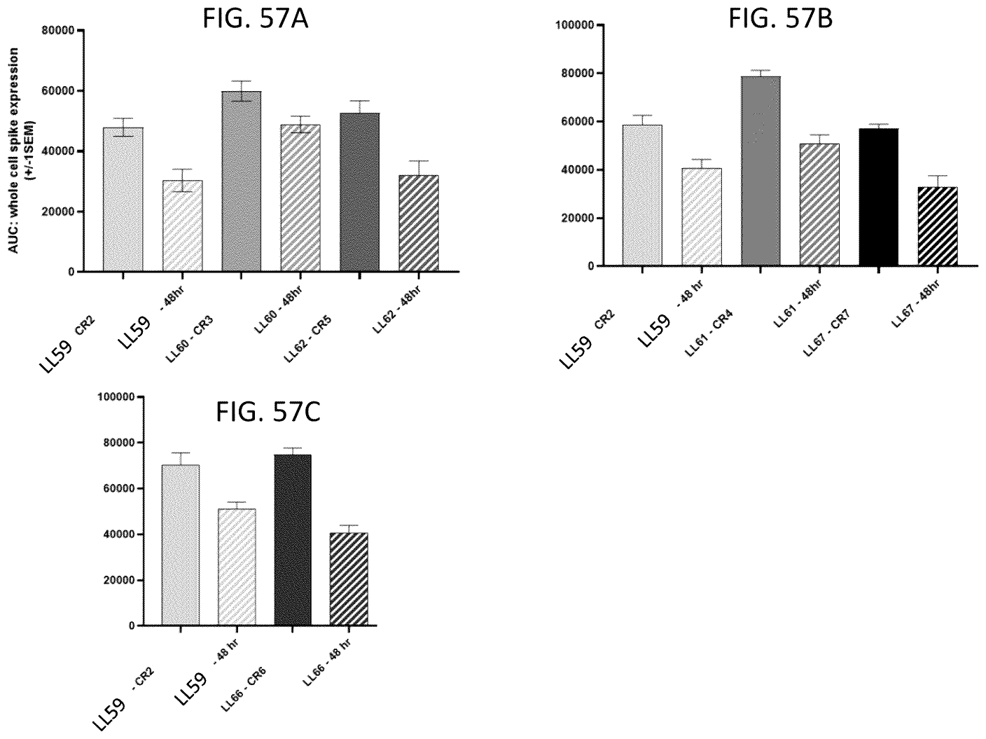

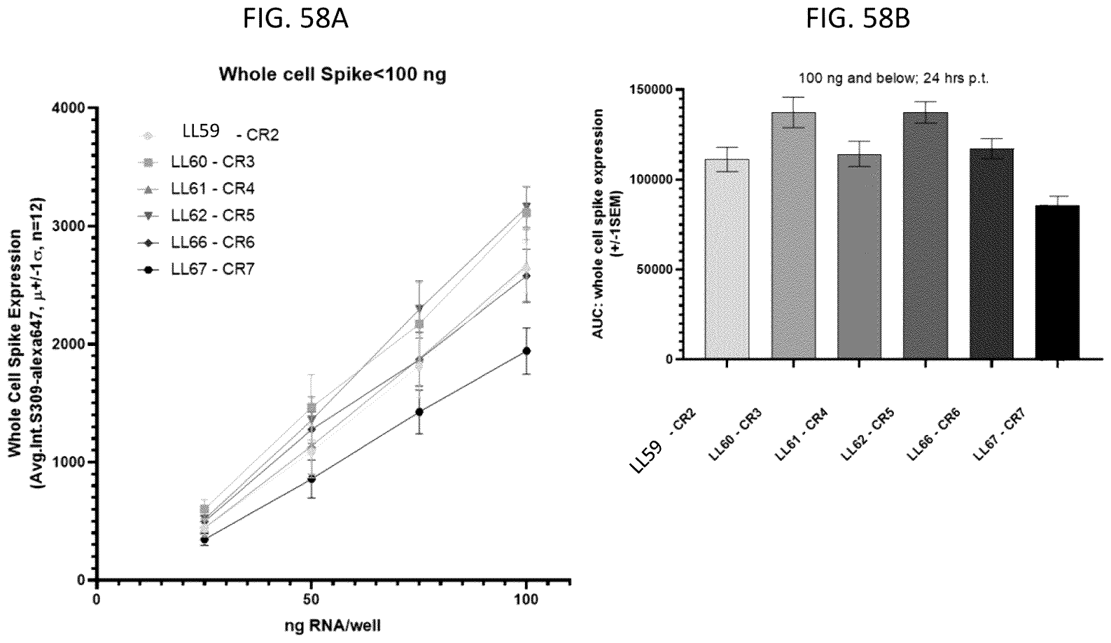

Assays were carried out twice with the same results. FIGS. 57A, 57B, 57C, 58A, and 58B show that HEK293 cells administered with LL59, LL60, LL61, LL62, LL66, or LL67 RNA construct had comparable whole cell spike protein expression 24 hrs (FIGS. 57A, 57B, 57C, 58A, and 58B) and 48 hrs (FIGS. 58A and 58B) after administration as measured by area under the curve of the dose response of 25, 50, 75, and 100 ng of RNA construct per well.

The fold increases and area under the curve for FIG. 58B are as follows:

| | | Fold increase |
|---|---|---|
| | Total | compared to |
| <100 ng/well | AUC | SPK001 |
| SPK001 CR2 | 111186 | 1.00 |
| LL60 CR3 | 137365 | 1.24 |
| LL61 CR4 | 114310 | 1.03 |
| LL62 CR5 | 137334 | 1.24 |
| LL66 CR6 | 117187 | 1.05 |
| LL67 CR7 | 85753 | 0.77 |

### Example 11b

KM70 prime (also known as LL159, as described in U.S. Ser. No. 63/413,844 as SEQ ID NOs: 119-121 (SEQ ID NO: 121 used)), LL60-62, LL66, and LL67 (see above) encapsulated in LNP as described in Example 11a were evaluated for immunogenicity in mice. Female BALB/c mice were 7 or 8 weeks old at day 0 of the study. On day 0 and day 21 of the study were injected using a 28 gauge needle into both hindleg thigh muscles with a vehicle or a unit dose per animal of 5.0 μg, 1.0 μg, or 0.5 μg of KM70 prime, LL60-62, LL66, or LL67 in 50 μL (25 μL per thigh muscle). The groups of animals, numbers, construct, dose, and formulations were as follows:

| | | Construct | | | Dose | |
|---|---|---|---|---|---|---|
| Group | N | Name | UTR | Coding Sequence | (ug) | Formulation |
| 1 | 4 | N/A | N/A | Saline | N/A | N/A |
| 2 | 8 | KM70 | UTR4 | SARS-CoV2 Omicron BA1 spike protein, CR2 | 5.0 | RV39 |
| | | | | optimized | | |
| 3 | 8 | KM70 | UTR4 | Same as above | 1.0 | RV39 |
| 4 | 8 | KM70 | UTR4 | Same as above | 0.5 | RV39 |
| 5 | 8 | LL60 | UTR4 | SARS-CoV2 Omicron BA1 spike protein, CR3 | 5.0 | RV39 |
| | | | | optimized | | |
| 6 | 8 | LL60 | UTR4 | Same as above | 1.0 | RV39 |
| 7 | 8 | LL60 | UTR4 | Same as above | 0.5 | RV39 |
| 8 | 8 | LL61 | UTR4 | SARS-CoV2 Omicron BA.1 spike protein, CR4 | 5.0 | RV39 |
| | | | | optimized | | |
| 9 | 8 | LL61 | UTR4 | Same as above | 1.0 | RV39 |
| 10 | 8 | LL61 | UTR4 | Same as above | 0.5 | RV39 |
| 11 | 8 | LL62 | UTR4 | SARS-CoV2 Omicron BA.1 spike protein, CR5 | 5.0 | RV39 |
| | | | | optimized | | |
| 12 | 7 | LL62 | UTR4 | Same as above | 1.0 | RV39 |
| 13 | 8 | LL62 | UTR4 | Same as above | 0.5 | RV39 |
| 14 | 8 | LL66 | UTR4 | SARS-CoV2 Omicron BA.1 spike protein, CR6 | 5.0 | RV39 |
| | | | | optimized | | |
| 15 | 8 | LL66 | UTR4 | Same as above | 1.0 | RV39 |
| 16 | 8 | LL66 | UTR4 | Same as above | 0.5 | RV39 |
| 17 | 8 | LL67 | UTR4 | SARS-CoV2 Omicron BA.1 spike protein, CR7 | 5.0 | RV39 |
| | | | | optimized | | |
| 18 | 8 | LL67 | UTR4 | Same as above | 1.0 | RV39 |
| 19 | 8 | LL67 | UTR4 | Same as above | 0.5 | RV39 |

### Day 35 Blood Draw and Spleen Harvest

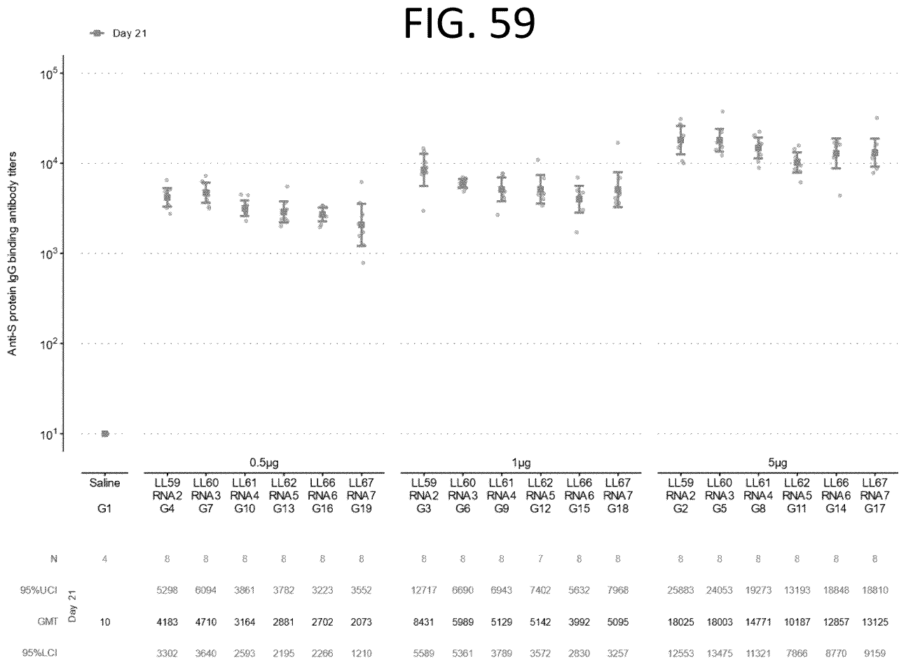

Anti-S protein IgG binding antibody titers (Omicron S protein ELISA) (See FIG. 59) FIG. 59 show statistically insignificant differences in serum levels of total IgG against anti-SARS-CoV2 BA.5 spike protein at day 21 after the first administration of one of RNA constructs LL59 (a.k.a. KM70 prime), 1LL60, 1LL61, LL162, LL166, and LL67 formulated in RV39 at doses of 0.5 μg, 1 μg, or 5 μg to mice. A second administration of the same doses occurred at day 21 (21 days after the first dose (day 0)). Statistical significance was found in measures between saline treated and construct treated animals.

### Example 12a

KM70 prime (also known as 1LLS9, as described in U.S. Ser. No. 63/413,844 as SEQ ID NOs: 119-121 (SEQ ID NO: 121 used, also known as SPK001 or SPK007 depending upon the poly(A) tail)) and a new construct SP035 or SP045, which consisted of, from 5′ to 3′, SEQ ID NO: 13, then SEQ NO: 128, then SEQ ID NO: 7, RNA were synthesized with 3′ poly(A) tails consisting of, from 5′ to 3′, 30 adenosine monophosphates (As), a linker, and 87 As or with 3′ poly(A) tails consisting of, from 5′ to 3′, 30 adenosine monophosphates (As), a linker, and 37 As. SPK001 is KM70 prime ending with 37 As. SPK007 is KM70 prime ending with 87 As. SP035 ends with 87 As. SP045 ends with 37 As. All recombinant RNA molecules were produced by in vitro transcription using N1-methyl pseudouridine to replace all uridines. All recombinant RNA molecules comprised a cap-1 5′ cap and a 3′ poly(A) tail. The mRNAs were purified and evaluated for mRNA integrity (by capillary gel electrophoresis), double stranded RNA (dsRNA) content (by Homogeneous Time Resolved Fluorescence [HTRF]), poly-A tail length, and percent capping.

Each RNA encapsulated in then formulated in LNPs comprising 46.3 mol % cation-ionizable lipid, RV39; 1.6 mol % PEG-conjugated lipid; 42.7 mol % cholesterol; and 9.4 mol % 1,2-diastearoyl-sn-glycero-3-phosphocholine (DSPC), i.e., the RV39 formulation.

A potency assay of the LNP encapsulated RNAs was carried out. HEK 293 cells were seeded at 3×10⁴ cells/well in a 96-well plate and incubated overnight. The cells were transduced with Omicron RNA LNP samples at 25, 50, and 100 ng/well. Cells were stained with anti-SARS-CoV2 Spike antibody and a secondary antibody. Expression was detected by flow cytometry.

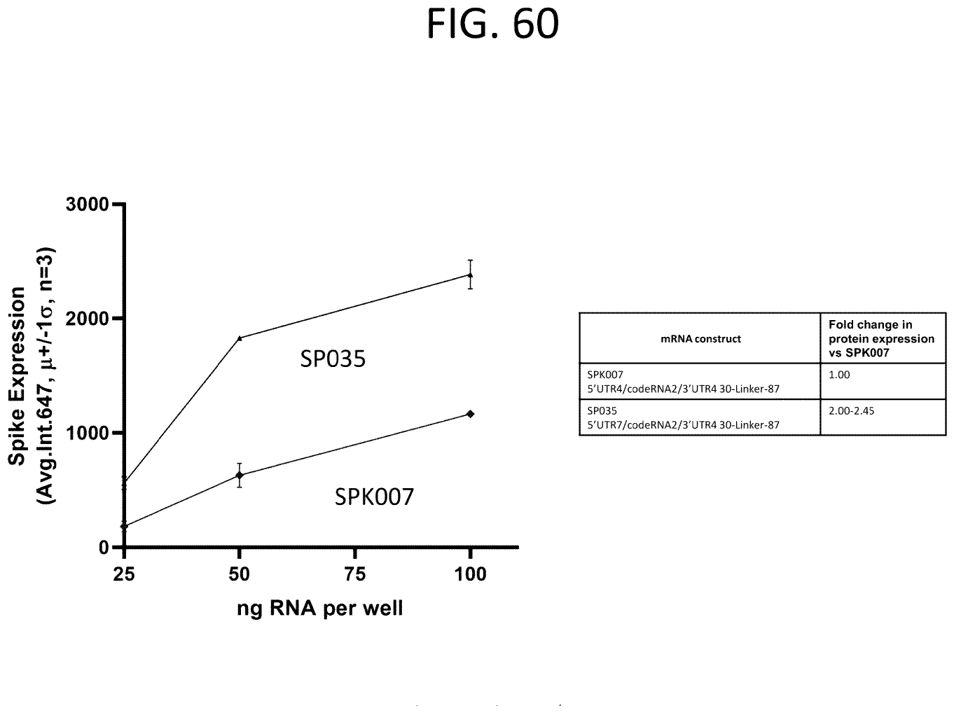

FIG. 60 shows that adding 5′ UTR7 and 3′ UTR4 into a construct, SP035, results in 2-2.45 higher area under the curve as obtained with LL59, which has 5′ UTR4 and 3′ UTR4. In both constructs, the 3′ poly(A) tail had 30 As, a linker, then 87 As.

The results for the other two constructs are as follows:

| | Fold change in protein |
|---|---|
| mRNA construct | expression vs SPK001 |
| SPK001 | 1.00 |
| 5′UTR4/codeRNA2/3′UTR4/30- | |
| Linker-37 | |
| SPK007 | 0.91 |
| 5′UTR4/codeRNA2/3′UTR4 | |
| 30-Linker-87 | |
| SP045 | 1.36 |
| 5′UTR7/codeRNA2/3′UTR4 | |
| 30-Linker-37 | |
| SP035 | 1.82 |
| 5′UTR7/codeRNA2/3′UTR4 | |
| 30-Linker-87 | |

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, publication of a patent application, and publication cited herein are hereby incorporated herein by reference in its entirety. While this invention has been disclosed with reference to specific embodiments, it is apparent that other embodiments and variations of this invention may be devised without departing from the true spirit and scope of the invention. The appended claims are intended to be construed to include all such embodiments and equivalent variations.

### REFERENCES

All references cited in this document are incorporated by reference in their entirety for all purposes and for convenience have been listed below. The omission of any reference in the list below after being cited in this document does not negate the above-noted incorporation by reference. List of references:
NCBI (www.ncbi.nlm.nih.gov) Accession NOs: NM_001101.5, NG_056261.1, NM_000477.7, AH002618.2, NM_001686.4, U09278.2, NM_001291807.3, NR_168448.1, NG_007073.2, NR_152150.2, NG_029473.1, NM_153201.4, AH002842.2, NM_000586.4, NG_013080.2, NM_001961.4, NM_000558, OL521838, and NM_001130;
The World Health Organization: International Nonproprietary Names Programme. Messenger RNA Encoding the Full-Length SARS-CoV-2 Spike Glycoprotein; p. 11868;
Edo Kon, Uri Elia, and Dann Peer, “Principles for designing an optimal mRNA lipid nanoparticle vaccine,” Curr. Opin. Biotechnol. 2022, 73:329-336;
Harlow et al., 1999, In: Using Antibodies: A Laboratory Manual, Cold Spring Harbor Laboratory Press, NY;
Harlow et al., 1989, In: Antibodies: A Laboratory Manual, Cold Spring Harbor, N.Y.;
Houston et al., 1988, Proc. Natl. Acad. Sci. USA 85:5879-5883; Bird et al., 1988, Science 242:423-426;
Zhang et al., 2001, “Ionization behavior of amino lipids for siRNA delivery: determination of ionization constants, SAR, and the impact of lipid pKa on cationic lipid-biomembrane interactions, LANGMUIR 27: 1907-1914;
WO2012/006376;
WO2012/030901;
WO2012/031046;
WO2012/031043;
WO2012/006378;
WO2011/076807;
WO2013/033563;
WO2013/006825;
WO2014/136086;
WO2015/095340;
WO2015/095346;
WO2016/037053;
WO2017/075531;
WO2018/081480;
WO2015/074085;
WO2018/170332;
WO2010/053572;
WO2012/170930;
WO2014/197651;
WO2015/123767;
WO2019/064115;
U.S. Patent Application Publication NOs.: 20220081392, 20220072155, 20220040285, 20210395188, 20210251898, 20210128488, 20210122703, 20210122702, 20210107861, 20200283372,20200172472,20200163878,20200121809,20200046838,20190359556, 20190314524, 20190274968, 20190270697, 20190022247, 20180185516, 20170283367, 20170157268,20170119904,20160376224,20160317676,20150376115, 20150140070A1, and 20100324120;
U.S. Application Nos. 61/905,724 and Ser. No. 15/614,499;
U.S. Pat. Nos. 8,802,863, 9,458,090, 9,593,077, 9,567,296, 9,604,908, 9,643,916, 9,669,097, 9,670,487, 9,737,619, 9,738,593, 9,725,720, 9,796,977,10,106,490, 10,166,298, 10,221,127, 10,723,692, 11,040,112, 11,168,051, and 11,285,222; and
Geall et al. (2012) PNAS USA. September 4; 109(36): 14604-9.

### Interpretation of Sequence Listing

In the paper copy and ASCII-format electronic copy of the Sequence Listing, it is to be understood that any “U” or “u” (uridine) depicted therein may be replaced with any of the uridine-substitutable modified nucleotides noted above, including a N1-methylpseudouridine, pseudouridine, N1-ethylpseudouridine, and that the percentage “u” replaced with uridine-substitutable modified nucleotides corresponds with those described in the embodiments above. For example, a mole percentage of the N1-methylpseudouridines to the total of the N1-methylpseudouridines and the uridines of 50% contemplates and supports substitution of 50% of the “u” with “N1ψ.” For example, a mole percentage of the N1-methylpseudouridines to the total of the N1-methylpseudouridines and the uridines of 25% contemplates and supports substitution of 25% of the “u” with “N1ψ.” For example, a mole percentage of the N1-methylpseudouridines to the total of the N1-methylpseudouridines and the uridines of 75% contemplates and supports substitution of 75% of the “u” with “N1ψ.” In the paper copy and ASCII-format electronic copy of the Sequence Listing, it is to be understood that any “A” or “a” (adenosine) depicted therein may be replaced with any of the adenosine-substitutable modified nucleotides noted above, and that the percentage “A” or “a” replaced with adenosine-substitutable modified nucleotides corresponds with those described in the embodiments above. In the paper copy and ASCII-format electronic copy of the Sequence Listing, it is to be understood that any “T” or “t” (thymidine) depicted therein may be replaced with any of the thymidine-substitutable modified nucleotides noted above, and that the percentage “T” or “t” replaced with adenosine-substitutable modified nucleotides corresponds with those described in the embodiments above. In the paper copy and ASCII-format electronic copy of the Sequence Listing, it is to be understood that any “C” or “c” (cytosine) depicted therein may be replaced with any of the cytosine-substitutable modified nucleotides noted above, and that the percentage “C” or “c” replaced cytosine-substitutable modified nucleotides corresponds with those described in the embodiments above. In the paper copy and ASCII-format electronic copy of the Sequence Listing, it is to be understood that any “G” or “g” (guanosine) depicted therein may be replaced with any of the guanosine-substitutable modified nucleotides noted above, and that the percentage “G” or “g” replaced guanosine-substitutable modified nucleotides corresponds with those described in the embodiments above.

### Sequence Identity

Identity or homology (i.e. percent identity) with respect to an amino acid sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Identity or homology (i.e. percent identity) with respect to a nucleic acid sequence is defined herein as the percentage of nucleotides in the candidate sequence that are identical with the reference nucleic acid sequence after aligning the sequences and introducing gaps, if necessary, to achieve the maximum percent sequence identity. The exchange of uridine and thymidine shall not be taken into account for calculating percent identity. The substitution of uridine or thymidine with a uridine- or thymidine-substitutable modified nucleotide (i.e. U substituted with N1ψ) shall not be taken into account for calculating percent identity. The substitution of adenosine with an adenosine-substitutable modified nucleotide shall not be taken into account for calculating percent identity. The substitution of guanosine with a guanosine-substitutable modified nucleotide shall not be taken into account for calculating percent identity. The substitution of cytosine with a cytosine-substitutable modified nucleotide shall not be taken into account for calculating percent identity.

Where the present disclosure refers to a sequence by reference to a UniProt, Genbank, National Center for Biotechnology Information (NCBI, www.ncbi.nlm.nih.gov) reference sequence, accession number (No.), or accession code, the sequence referred to is the current version at the filing date of the earliest effective filing date. Herein a “reference sequence” and “accession code” is referred to as “accession code” for the sole purpose of simplicity and without regard to whether the original Uniprot, Genebank, or NCBI listing recites “reference sequence,” “accession no.,” or “accession code.”

1. A recombinant ribonucleic acid (RNA) molecule comprising a 5′ untranslated region (5′ UTR), a segment that encodes a heterologous polypeptide, a 3′ untranslated region (3′ UTR), and a 3′ poly(adenosine monophosphate) (poly(A)) tail; wherein:
a) the 5′ UTR is 5′ of the segment that encodes the heterologous polypeptide;
b) the 3′ UTR is 3′ of the segment that encodes the heterologous polypeptide;
c) the 3′ poly(A) is 3′ of the 3′ UTR; and
d) i) the 5′ UTR comprises a sequence that has at least 95% identity to SEQ ID NO: 5 and the 3′ UTR comprises a sequence that has at least 95% identity to SEQ ID NO: 6;
ii) the 5′ UTR comprises a sequence that has at least 95% identity to SEQ ID NO: 7 and the 3′ UTR comprises a sequence that has at least 95% identity to SEQ ID NO: 8; or
iii) the 5′ UTR comprises a sequence that has at least 95% identity to SEQ ID NO: 13 and the 3′ UTR a sequence that has at least 95% identity to SEQ ID NO: 14.

2. The recombinant RNA molecule of claim 1, the heterologous polypeptide comprising an immunogen or an antibody against the immunogen.

3. The recombinant RNA molecule of claim 1 further comprising a 5′ cap nucleoside and a first 5′ ribonucleoside; wherein the 5′ cap nucleoside is linked 5′-to-5′ to the first 5′ ribonucleoside.

4. The recombinant RNA molecule of claim 3, wherein the first 5′ ribonucleoside comprises a 2′ methylated ribose.

5. The recombinant RNA molecule of claim 3 further comprising a linker; wherein the 5′ cap nucleoside is linked 5′-to-5′ to the first 5′ ribonucleoside by the linker.

6. The recombinant RNA molecule of claim 5, wherein the linker is a triphosphate.

7. The recombinant RNA molecule of claim 1 comprising a modified nucleotide.

8. The recombinant RNA molecule of claim 7, wherein the modified nucleotide comprises: pseudouridine, N1-methylpseudouridine, or N1-ethylpseudouridine.

9. The recombinant RNA molecule of claim 8, wherein substantially all uridines are replaced with at least one of: the pseudouridine, the N1-methylpseudouridine, and the N1-ethylpseudouridine.

10. The recombinant RNA molecule of claim 1, wherein the 3′ poly(A) comprises from 70 to 100 consecutive adenosine monophosphates.

11. The recombinant RNA molecule of claim 1, wherein the 3′ poly(A) tail comprises a 3′ segmented poly(A) tail comprising a first segment of consecutive adenosine monophosphate residues, a spacer, and a second segment of consecutive adenosine monophosphate residues and wherein the spacer comprises at least one nucleotide other than adenosine monophosphate.

12. The recombinant RNA molecule of claim 11, wherein the first segment of consecutive adenosine monophosphate residues comprises from 10 to 30 adenosine monophosphates and the second segment of consecutive adenosine monophosphate residues comprises from 70 to 100 adenosine monophosphates.

13. The recombinant RNA molecule of claim 1 comprising a sequence that has at least 95% identity to at least one of SEQ ID NOs: 32, 35, 38, 45, 48, 51, 54, 57, 60, 69, 72, 75, 82, 85, 88, 134, 138, 142, 146,150,154,158,162,166,170,174,178,182,186,190, and 194.

14. A formulation comprising lipid nanoparticles (LNPs) and recombinant RNA molecules of claim 1, wherein:
i) the LNPs comprise lipids;
ii) the lipids comprise first lipids and cholesterol;
iii) the first lipid comprises a tertiary amine and has a pKa from 5.0 to 7.6;
iv) at least half of the first lipids are neutrally charged when the first lipids are at a pH that is above the pKa; and
v) at least half of the first lipids are positively charged when the first lipids are at a pH that is below the pKa.

15. The formulation of claim 14, wherein at least 80% of the recombinant RNA molecules are encapsulated by the LNPs.

16. The formulation of claim 15, wherein the lipids further comprise polyethylene glycol-conjugated (PEG-conjugated) lipids.

17. The formulation of claim 16, wherein the PEG-conjugated lipids comprise 1,2-dimyristoyl-rac-glycerol-3-methoxypolyethylene glycol.

18. The formulation of claim 16, wherein the lipids further comprise second lipids and the second lipids comprise a neutral lipid or zwitterionic lipid.

19. The formulation of claim 18, wherein the second lipids comprise the zwitterionic lipid and the zwitterionic lipid comprises 1,2-distearoyl-sn-glycero-3-phosphocholine (DSPC).

20. (canceled)

21. A method of eliciting an immune response against an immunogen in a subject, the method comprising administering to the subject an effective amount to elicit the immune response of the formulation of claim 16, the heterologous polypeptide comprising the immunogen or an antibody against the immunogen.

22. (canceled)

23. The method of claim 21, the immune response being a therapeutic immune response.

24. (canceled)

25. The method of claim 21, the heterologous polypeptide comprising the antibody against the immunogen.

26. The method of claim 21, the heterologous polypeptide comprising the immunogen.

27. The method of claim 21, the subject being human.

28. A method of manufacturing the recombinant RNA molecule of claim 1, the method comprising admixing an RNA polymerase, triphosphate nucleotides, and a template nucleic acid comprising a sequence of the recombinant RNA molecule, thereby obtaining an admixture; the admixing being under conditions wherein the RNA polymerase produces the recombinant RNA molecule from the template nucleic acid.

29. A method of manufacturing the formulation of claim 16, the method comprising:
a. admixing the recombinant RNA molecules with an aqueous buffer, thereby obtaining an aqueous recombinant RNA solution;
b. admixing the lipids in an organic solvent, thereby obtaining a lipid solution;
c. admixing the lipid solution and the aqueous recombinant RNA solution with at least one of: a T-mixer, a microfluidics mixer, and an impinging jet mixer, thereby obtaining a first solution, wherein the first solution comprises the LNPs; and
d. purifying the LNPs to obtain the at least 80% of the recombinant RNA molecules being encapsulated by the LNPs.

30.-38. (canceled)

39. A recombinant RNA molecule comprising a first sequence or a second sequence, wherein the first sequence has at least 90% identity to SEQ ID NO: 29 and the second sequence has at least 97% identity to SEQ ID NO: 30.

40.-68. (canceled)
